SEQUENCE LISTING (14 sequences)

SEQ ID NO: 1 - protein, 11 aa, Artificial Sequence
  source: /note=“Description of Artificial Sequence Synthetic peptide”
VSGWRLFKKIS

SEQ ID NO: 2 - DNA, 19 bp, Artificial Sequence
  source: /note=“Description of Artificial Sequence Synthetic primer”
ctacattttacatgatagg

SEQ ID NO: 3 - DNA, 21 bp, Artificial Sequence
  source: /note=“Description of Artificial Sequence Synthetic oligonucleotide”
agcccacaatgccacggaaat

SEQ ID NO: 4 - DNA, 21 bp, Artificial Sequence
  source: /note=“Description of Artificial Sequence Synthetic oligonucleotide”
gcaacatctacaccctcaaat

SEQ ID NO: 5 - DNA, 21 bp, Artificial Sequence
  source: /note=“Description of Artificial Sequence Synthetic oligonucleotide”
tgaccgagtggtacgtcaatg

SEQ ID NO: 6 - DNA, 21 bp, Artificial Sequence
  source: /note=“Description of Artificial Sequence Synthetic oligonucleotide”
agcggcagaacatcaacaaat

SEQ ID NO: 7 - RNA, 67 nt, Artificial Sequence
  source: /note=“Description of Artificial Sequence Synthetic oligonucleotide”
agcauagcaaguuaaaauaaggcuaguccguuaucaacuugaaaaaguggcaccgagucggugcuuu

SEQ ID NO: 8 - DNA, 20 bp, Artificial Sequence
  source: /note=“Description of Artificial Sequence Synthetic oligonucleotide”
acggaggctaagcgtcgcaa

SEQ ID NO: 9 - DNA, 20 bp, Artificial Sequence
  source: /note=“Description of Artificial Sequence Synthetic oligonucleotide”
aacatctttcgggccgtagg

SEQ ID NO: 10 - DNA, 20 bp, Artificial Sequence
  source: /note=“Description of Artificial Sequence Synthetic oligonucleotide”
gacatccgctgcgagttctg

SEQ ID NO: 11 - DNA, 20 bp, Artificial Sequence
  source: /note=“Description of Artificial Sequence Synthetic oligonucleotide”
tgcagcgtcccgggcagagc

SEQ ID NO: 12 - DNA, 20 bp, Artificial Sequence
  source: /note=“Description of Artificial Sequence Synthetic oligonucleotide”
caagccgtgcctcatcaaga

SEQ ID NO: 13 - DNA, 20 bp, Artificial Sequence
  source: /note=“Description of Artificial Sequence Synthetic oligonucleotide”
cgggcacacctgcggcagtt

SEQ ID NO: 14 - DNA, 20 bp, Artificial Sequence
  source: /note=“Description of Artificial Sequence Synthetic oligonucleotide”
agtgggtgcggcacttacag

(19) United States
(12) Patent Application Publication
(10) Pub. No.: US 2025/0276964 A1
(43) Pub. Date: Sep. 4, 2025

(54) Benzisoxazole Derivatives and Uses thereof

(71) Applicant: Novartis AG, Basel (CH)

(72) Inventors: Rohan Eric John BECKWITH, San Diego, CA (US); Simone BONAZZI, Cambridge, MA (US); Artiom CERNIJENKO, Cambridge, MA (US); Jennifer Stroka COBB, Stow, MA (US); Natalie Alysia DALES, Arlington, MA (US); Janetta DEWHURST, Framingham, MA (US); Aleem FAZAL, Wilmington, MA (US); Matthew James HESSE, Pittsburgh, PA (US); Rama JAIN, Danville, CA (US); John Ryan KERRIGAN, Wakefield, MA (US); Hasnain Ahmed MALIK, Boston, MA (US); James R. MANNING, Emeryville, CA (US); Gary O'BRIEN, Maynard, MA (US); Andrew W. PATTERSON, Somerville, MA (US); Noel Marie-France THOMSEN, Chelmsford, MA (US); Pamela YF TING, Somerville, MA (US)

(21) Appl. No.: 18/550,806

(22) PCT Filed: Mar. 14, 2022
(86) PCT No.: PCT/IB2022/000134

§ 371 Date: Sep. 15, 2023

Related U.S. Application Data
(60) Provisional application No. 63/164,125, filed on Mar. 22, 2021.
(60) Provisional application No. 63/161,141, filed on Mar. 15, 2021.

(51) Int. Cl. C07D 413/14; A61K 31/513; A61K 31/5383; A61K 31/551; A61P 7/06; C07D 451/02; C07D 471/08; C07D 487/08; C07D 498/04; C07D 513/04

(52) U.S. Cl. CPC C07D 413/14; A61K 31/513; A61K 31/5383; A61K 31/551; A61P 7/06; C07D 451/02; C07D 471/08; C07D 487/08; C07D 498/04; C07D 513/04

(57) ABSTRACT

The present disclosure relates to compounds of formula (I) and pharmaceutical compositions and their use in reducing Widely Interspaced Zinc Finger Motifs (WIZ) expression levels, or inducing fetal hemoglobin (HbF) expression, and in the treatment of inherited blood disorders (e.g., hemoglobinopathies, e.g., beta-hemoglobinopathies), such as sickle cell disease and beta-thalassemia.

### CLAIM OF PRIORITY

This application claims the benefit of priority to U.S. Provisional Application No. 63/161,141 filed Mar. 15, 2021, and U.S. Provisional Application No. 63/164,125 filed Mar. 22, 2021, the disclosures of which are incorporated by reference herein in their entirety.

### SEQUENCE LISTING

The instant application contains a Sequence Listing which has been submitted electronically in ASCII format and is hereby incorporated by reference in its entirety. Said ASCII copy, created on Jul. 16, 2021 is named PAT069043-US-PSP02_SL.txt and is 4,096 bytes in size.

### FIELD OF THE DISCLOSURE

The present disclosure relates to benzisoxazole derivatives compounds and pharmaceutical compositions and their use in reducing Widely Interspaced Zinc Finger Motifs (WIZ) protein expression levels and/or inducing fetal hemoglobin (HbF) protein expression levels, and in the treatment of inherited blood disorders (hemoglobinopathies, e.g., beta-hemoglobinopathies), such as sickle cell disease and beta-thalassemia.

### BACKGROUND OF THE DISCLOSURE

Sickle cell disease (SCD) is a group of severe inherited blood disorders that cause red blood cells to contort into a sickle shape. These cells can cause blockages in blood flow, leading to intense pain, organ damage and premature death. Beta thalassemias are a group of inherited blood disorders that are caused by reduced or absent synthesis of beta globin, causing anemia.

Fetal hemoglobin (HbF) induction is known to ameliorate symptoms in SCD and beta-thalassemia patients, with both genetic (single nucleotide polymorphisms in the globin control locus & BCL11A) and pharmacologic (hydroxyurea) validation in the clinic (Vinjamur, D. S., et al. (2018), The British Journal of Haematology, 180(5), 630-643). Hydroxyurea is the current standard of care for SCD and is thought to provide benefit via induction of HbF, but is genotoxic, causes dose-limiting neutropenia and has a response rate of less than 40%. Other mechanisms being targeted clinically and preclinically include inhibition of HDAC1/2 (Shearstone et al., 2016, PLoS One, 11(4), e0153767), LSD1 (Rivers et al., 2018, Experimental Hematology, 67, 60-64), DNMT1, PDE9a (McArthur et al., 2019, Haematologica. doi:10.3324/haematol.2018.213462), HRI kinase (Grevet et al., 2018, Science, 361(6399), 285-290) and G9a/GLP (Krivega et al., 2015, Blood, 126(5), 665-672; Renneville et al., 2015, Blood, 126(16), 1930-1939). Additionally, the immunomodulators pomalidomide and lenalidomide induce HbF ex vivo in human primary erythroid cells (Moutouh-de Parseval, L. A. et al. (2008), The Journal of Clinical Investigation, 118(1), 248-258) and in vivo (Meiler, S. E. et al. (2011), Blood, 118(4), 1109-1112). WIZ is ubiquitously expressed and plays a role in targeting the G9a/GLP histone methyltransferases to genomic loci to regulate chromatin structure and transcription (Bian, Chen, et al. (2015), eLife 2015; 4:e05606.

### SUMMARY OF THE DISCLOSURE

The disclosure relates to a therapeutic agent, which is effective in reducing WIZ protein expression levels and/or inducing fetal hemoglobin (HbF) expression. The disclosure further relates to benzisoxazole compounds, which are effective in reducing WIZ protein expression levels and/or inducing fetal hemoglobin (HbF) expression, pharmaceutically acceptable salts thereof, compositions thereof, and their use in therapies for the conditions and purposes detailed above.

The disclosure provides, in a first aspect, a compound of formula (I″) or a pharmaceutically acceptable salt thereof, wherein:

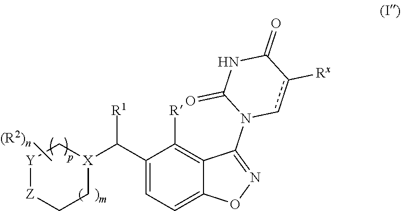

[symbol] is a single bond or a double bond;
X is selected from CH, CF, and N;
Y is selected from CH₂, CR^{Y}R² and N—R³;
Z is selected from CH₂, CR^{Y}R² and N—R³,
wherein at least one of Y and Z is N—R³ such that when Y is N—R³, Z is selected from CH₂, and CR^{Y}R², and when Z is N—R³, Y is selected from CH₂, and CR^{Y}R²,
and wherein when R² of CR^{Y}R² of Y or Z is oxo, R^{Y} is absent;
R^{x} is selected from hydrogen, C₁-C₆alkyl, halo (e.g., F, Cl), C₁-C₆alkoxyl, and C₃-C₈cycloalkyl;
R^{Y} is selected from hydrogen and C₁-C₆alkyl,
R′ is selected from hydrogen and C₁-C₆alkyl;
R¹ is selected from hydrogen and C₁-C₆alkyl;
each R² is independently selected from C₁-C₆alkyl, C₁-C₆haloalkyl, halo, and oxo, wherein the C₁-C₆alkyl is substituted with 0-1 occurrence of R^{2a}; or 2 R² on non-adjacent carbon atoms together with the non-adjacent carbon atoms to which they are attached form a bridging ring;
R^{2a} is selected from C₁-C₆alkoxyl and hydroxyl;
R³ is selected from hydrogen, C₁-C₈alkyl, C₂-C₆alkenyl, —SO₂R⁴, C₁-C₆haloalkyl, a 4- to 6-membered heterocyclyl comprising 1-2 heteroatoms independently selected from N, O and S, C₃-C₁₀cycloalkyl, —C(═O)—O—(R⁵) and —C(═O)—(R⁶), wherein the C₁-C₈alkyl and C₁-C₆haloalkyl are independently substituted with 0-3 occurrences of R^{3a};
or
R³ together with the nitrogen atom to which it is attached and R² together with the carbon atom to which it is attached form a 5- or 6-membered heterocyclyl comprising 0-1 additional heteroatom selected from N, O and S;
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S, C₆-C₁₀aryl, C₁-C₆alkoxyl, hydroxyl, and —C(═O)—NR⁷R⁸, wherein the C₃-C₁₀cycloalkyl, 4- to 6-membered heterocyclyl, 5- to 10-membered heteroaryl and C₆-C₁₀aryl are substituted with 0-4 occurrences of R^{3b};
each R^{3b} is independently selected from C₁-C₆alkoxyl, halo, C₁-C₆haloalkyl, C₁-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁵ is selected from C₁-C₆alkyl, C₃-C₈cycloalkyl, and C₆-C₁₀aryl;
R⁶ is selected from C₁-C₆alkyl, C₃-C₈cycloalkyl, NR⁷R⁸, and C₆-C₁₀aryl, wherein the C₁-C₆alkyl is substituted with 0-1 occurrence of R^{6a} and the C₃-C₈cycloalkyl is substituted with 0-1 occurrence of R^{6b};
R^{6a} is selected from C₆-C₁₀aryl and C₃-C₈cycloalkyl;
R^{6b} is selected from halo, C₁-C₆haloalkyl, C₁-C₆haloalkoxyl, and C₁-C₆alkyl;
R⁷ is selected from hydrogen and C₁-C₆alkyl;
R⁸ is selected from hydrogen, C₃-C₈cycloalkyl and C₁-C₆alkyl;
or
R⁷ and R⁸ together with the nitrogen atom to which they are attached form a 5- or 6-membered heterocyclyl comprising 0-1 additional heteroatom selected from N, O, and S;
n is 0, 1, 2, 3 or 4;
m is 0, 1 or 2; and
p is 0 or 1.

The disclosure provides, in a second aspect, a compound of formula (I′) or a pharmaceutically acceptable salt thereof, wherein:

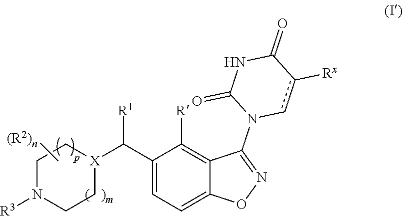

[symbol] is a single bond or a double bond;
X is selected from CH, CF, and N;
R′ is selected from hydrogen and C₁-C₆alkyl;
R¹ is selected from hydrogen and C₁-C₆alkyl;
each R² is independently selected from C₁-C₆alkyl, C₁-C₆haloalkyl, halo, and oxo, wherein the C₁-C₆alkyl is substituted with 0-1 occurrence of R^{2a}; or 2 R² on non-adjacent carbon atoms together with the non-adjacent carbon atoms to which they are attached form a bridging ring;
R^{2a} is selected from C₁-C₆alkoxyl and hydroxyl;
R³ is selected from hydrogen, C₁-C₈alkyl, C₂-C₆alkenyl, —SO₂R⁴, C₁-C₆haloalkyl, —C(═O)—O—(R⁵) and —C(═O)—(R⁶), wherein the C₁-C₈alkyl and C₁-C₆haloalkyl are independently substituted with 0-3 occurrences of R^{3a};
or
R³ together with the nitrogen atom to which it is attached and R² together with the carbon atom to which it is attached form a 5- or 6-membered heterocyclyl comprising 0-1 additional heteroatom selected from N, O and S;
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S, C₆-C₁₀aryl, C₁-C₆alkoxyl, hydroxyl, and —C(═O)—NR⁷R⁸, wherein the C₃-C₁₀cycloalkyl, 4- to 6-membered heterocyclyl, 5- to 10-membered heteroaryl and C₆-C₁₀aryl are substituted with 0-4 occurrences of R^{3b};
each R^{3b} is independently selected from C₁-C₆alkoxyl, halo, C₁-C₆haloalkyl, C-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁵ is selected from C₁-C₆alkyl, C₃-C₈cycloalkyl, and C₆-C₁₀aryl;
R⁶ is selected from C₁-C₆alkyl, C₃-C₈cycloalkyl, and C₆-C₁₀aryl, wherein the C₁-C₆alkyl is substituted with 0-1 occurrence of R^{6a} and the C₃-C₈cycloalkyl is substituted with 0-1 occurrence of R^{6b};
R^{6a} is selected from C₆-C₁₀aryl and C₃-C₈cycloalkyl;
R^{6b} is selected from halo, C₁-C₆haloalkyl, C₁-C₆haloalkoxyl, and C₁-C₆alkyl;
R⁷ is selected from hydrogen and C₁-C₆alkyl;
R⁸ is selected from hydrogen and C₁-C₆alkyl;
or
R⁷ and R⁸ together with the nitrogen atom to which they are attached form a 5- or 6-membered heterocyclyl comprising 0-1 additional heteroatom selected from N, O, and S;
n is 0, 1, 2, 3 or 4;
m is 0, 1 or 2; and
p is 0 or 1.

The disclosure provides, in a third aspect, a compound of formula (I) or a pharmaceutically acceptable salt thereof, wherein:

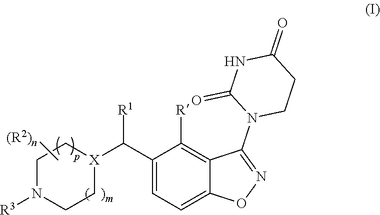

X is selected from CH, CF, and N;
R′ is selected from hydrogen and C₁-C₆alkyl;
R¹ is selected from hydrogen and C₁-C₆alkyl;
each R² is independently selected from C₁-C₆alkyl, C₁-C₆haloalkyl, halo, and oxo, wherein the C₁-C₆alkyl is substituted with 0-1 occurrence of R^{2a}; or 2 R² on non-adjacent carbon atoms together with the non-adjacent carbon atoms to which they are attached form a bridging ring;
R^{2a} is selected from C₁-C₆alkoxyl and hydroxyl;
R³ is selected from hydrogen, C₁-C₈alkyl, C₂-C₆alkenyl, —SO₂R⁴, C₁-C₆haloalkyl, —C(═O)—O—(R⁵) and —C(═O)—(R⁶), wherein the C₁-C₅alkyl and C₁-C₆haloalkyl are independently substituted with 0-3 occurrences of R^{3a};
or
R³ together with the nitrogen atom to which it is attached and R² together with the carbon atom to which it is attached form a 5- or 6-membered heterocyclyl comprising 0-1 additional heteroatom selected from N, O and S;
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S, C₆-C₁₀aryl, C₁-C₆alkoxyl, hydroxyl, and —C(═O)—NR⁷R⁸, wherein the C₃-C₁₀cycloalkyl, 4- to 6-membered heterocyclyl, 5- to 10-membered heteroaryl and C₆-C₁₀aryl are substituted with 0-4 occurrences of R^{3b};
each R^{3b} is independently selected from C₁-C₆alkoxyl, halo, C₁-C₆haloalkyl, C₁-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁵ is selected from C₁-C₆alkyl, C₃-C₈cycloalkyl, and C₆-C₁₀aryl;
R⁶ is selected from C₁-C₆alkyl, C₃-C₈cycloalkyl, and C₆-C₁₀aryl, wherein the C₁-C₆alkyl is substituted with 0-1 occurrence of R^{6a} and the C₃-C₈cycloalkyl is substituted with 0-1 occurrence of R^{6b};
R^{6a} is selected from C₆-C₁₀aryl and C₃-C₈cycloalkyl;
R^{6b} is selected from halo, C₁-C₆haloalkyl, C₁-C₆haloalkoxyl, and C₁-C₆alkyl;
R⁷ is selected from hydrogen and C₁-C₆alkyl;
R⁸ is selected from hydrogen and C₁-C₆alkyl;
or
R⁷ and R⁸ together with the nitrogen atom to which they are attached form a 5- or 6-membered heterocyclyl comprising 0-1 additional heteroatom selected from N, O, and S;
n is 0, 1, 2, 3 or 4;
m is 0, 1 or 2; and
p is 0 or 1.

In a further aspect, the disclosure provides a pharmaceutical composition comprising a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof, and a pharmaceutically acceptable carrier or excipient.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use as a medicament.

In a further aspect, the disclosure provides a method of treating or preventing a disease or disorder in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof.

In a further aspect, the disclosure provides a method of treating or preventing a disorder that is affected by the reduction or modulation of WIZ protein levels, in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In a further aspect, the disclosure provides a method of inhibiting WIZ protein expression in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof.

In a further aspect, the disclosure provides a method of degrading WIZ protein in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof.

In an further aspect, the disclosure provides a method of inhibiting, reducing, or eliminating the activity of WIZ protein or WIZ protein expression, the method comprising administering to the subject a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof.

In a further aspect, the disclosure provides a method of inducing or promoting fetal hemoglobin in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof.

In a further aspect, the disclosure provides a method of reactivating fetal hemoglobin production or expression in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof.

In an further aspect, the disclosure provides a method of increasing fetal hemoglobin expression in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof.

In a further aspect, the disclosure provides a method of treating a hemoglobinopathy, e.g., a beta-hemoglobinopathy, in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In a further aspect, the disclosure provides a method of treating a sickle cell disease in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof.

In a further aspect, the disclosure provides a method of treating beta-thalassemia in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use in the treatment of a disease or disorder.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use in the treatment of a disease or disorder selected from sickle cell disease and beta-thalassemia.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use in the treatment or prevention of a disease or disorder that is affected by the reduction of WIZ protein levels.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use in the treatment or prevention of a disease or disorder that is affected by the inhibition or reduction of WIZ protein expression.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use in the treatment or prevention of a disease or disorder that is affected by the degradation of WIZ protein.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use in inhibiting, reducing, or eliminating the activity of WIZ protein or WIZ protein expression.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use in inducing or promoting fetal hemoglobin.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use in reactivating fetal hemoglobin production or expression.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use in increasing fetal hemoglobin expression.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use in the treatment of a hemoglobinopathy.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use in the treatment of a sickle cell disease.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use in the treatment of beta-thalassemia.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use in the treatment of a disease or disorder affected by an increase in fetal hemoglobin expression.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use in the treatment of a disease or disorder affected by the inhibition, reduction, or elimination of the activity of WIZ protein or WIZ protein expression.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use in the treatment of a disease or disorder affected by the induction or promotion of fetal hemoglobin.

In a further aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof, for use in the treatment of a disease or disorder affected by the reactivation of fetal hemoglobin production or expression.

Various other aspects of the disclosure are described herein and in the claims.

Unless otherwise defined, all technical and scientific terms used herein have the same meaning as commonly understood by one of ordinary skill in the art to which this disclosure belongs. In the specification and claims, the singular forms also include the plural unless the context clearly dictates otherwise. Although methods and materials similar or equivalent to those described herein can be used in the practice or testing of the disclosure, suitable methods and materials are described below. All publications, patent applications, patents, and other references mentioned herein are incorporated by reference in their entireties for all purposes. The references cited herein are not admitted to be prior art to the claimed disclosure. In the case of conflict, the present specification, including definitions, will control. In addition, the materials, methods, and examples are illustrative only and are not intended to be limiting.

Other features and advantages of compounds, compositions, and methods disclosed herein will be apparent from the following detailed description and claims.

### BRIEF DESCRIPTION OF THE DRAWINGS

FIG. 1A depicts a volcano plot of differentially expressed genes from WIZ KO cells as compared to a scrambled gRNA control. Each dot represents a gene. HBG1/2 genes are differentially upregulated with WIZ_6 and WIZ_18 gRNA targeting WIZ KO.

FIG. 1B depicts a bar graph showing the frequency of HbF+ cells due to shRNA− mediated loss of WIZ in human mobilized peripheral blood CD34+ derived erythroid cells.

FIG. 1C depicts a bar graph showing the frequency of HbF+ cells due to CRISPR/Cas9-mediated loss of WIZ in human mobilized peripheral blood CD34+ derived erythroid cells.

### DETAILED DESCRIPTION OF THE DISCLOSURE

The compounds disclosed herein are effective in reducing WIZ protein expression levels, or inducing fetal hemoglobin (HbF) expression. Without wishing to be bound by any theory, it is believed that the disclosed compounds may treat blood disorders, such as inherited blood disorders, e.g., sickle cell disease, and beta-thalassemia by inducing fetal hemoglobin HbF expression.

### Definitions

Unless specified otherwise, the terms “compounds of the present disclosure,” “compounds of the disclosure,” or “compound of the disclosure” refer to compounds of formulae (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), exemplified compounds, salts thereof, particularly pharmaceutically acceptable salts thereof, hydrates, solvates, prodrugs, as well as all stereoisomers (including diastereoisomers and enantiomers), rotamers, tautomers, and isotopically labeled compounds (including deuterium substitutions), as well as inherently formed moieties.

In the groups, radicals, or moieties defined below, the number of carbon atoms is often specified preceding the group, for example, C₁-C₈alkyl means an alkyl group or radical having 1 to 8 carbon atoms. In general, for groups comprising two or more subgroups, the last named group is the radical attachment point, for example, “alkylaryl” means a monovalent radical of the formula alkyl-aryl-, while “arylalkyl” means a monovalent radical of the formula aryl-alkyl-.

Furthermore, the use of a term designating a monovalent radical where a divalent radical is appropriate shall be construed to designate the respective divalent radical and vice versa. Unless otherwise specified, conventional definitions of terms control and conventional stable atom valences are presumed and achieved in all formulas and groups. The articles “a” and “an” refer to one or more than one (e.g., to at least one) of the grammatical object of the article. By way of example, “an element” means one element or more than one element.

The term “and/or” means either “and” or “or” unless indicated otherwise.

The term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein the term “C₁-C₅alkyl” refers to a straight or branched hydrocarbon chain radical consisting solely of carbon and hydrogen atoms, containing no unsaturation, having from one to eight carbon atoms, and which is attached to the rest of the molecule by a single bond.

The terms “C₁-C₃alkyl”, “C₁-C₄alkyl”, “C₁-C₆alkyl”, are to be construed accordingly. Examples of C₁-C₅alkyl include, but are not limited to, methyl, ethyl, n-propyl, 1-methylethyl (iso-propyl), n-butyl, 1-methylpropyl (sec-butyl), 2-methylpropyl (iso-butyl or i-butyl), 1,1-dimethylethyl (t-butyl), n-pentyl, 3-pentyl, n-hexyl, n-heptyl, 4-heptyl, n-octyl, 2-isopropyl-3-methylbutyl.

As used herein, the term “C₁-C₆alkoxyl” refers to a radical of the formula —OR, where Rₐ is a C₁-C₆alkyl radical as generally defined above. Examples of C₁-C₆alkoxyl include, but are not limited to, methoxy, ethoxy, propoxy, iso-propoxy, butoxy, iso-butoxy, tert-butoxy, sec-butoxy, pentoxy, and hexoxy.

As used herein, the term “C₁-C₆haloalkyl” refers to C₁-C₆alkyl radical, as defined above, substituted by one or more halo radicals, as defined herein. Examples of C₁-C₆haloalkyl include, but are not limited to, trifluoromethyl, difluoromethyl, fluoromethyl, trichloromethyl, 1,1-difluoroethyl, 2,2-difluoroethyl, 2,2,2-trifluoroethyl, 2-fluoropropyl, 1,1,1-trifluoropropyl, 2,2-difluoropropyl, 3,3-difluoropropyl and 1-fluoromethyl-2-fluoroethyl, 1,3-dibromopropan-2-yl, 3-bromo-2-fluoropropyl, 1,1,2,2-tetrafluoropropyl, and 1,4,4-trifluorobutan-2-yl.

As used herein, the term “C₁-C₆haloalkoxyl” means a C₁-C₆alkoxyl group as defined herein substituted with one or more halo radicals. Examples of C₁-C₆haloalkoxyl groups include, but are not limited to, trifluoromethoxy, difluoromethoxy, fluoromethoxy, trichloromethoxy, 1,1-difluoroethoxy, 2,2-difluoroethoxy, 2,2,2-trifluoroethoxy, 1-fluoromethyl-2-fluoroethoxy, pentafluoroethoxy, 2-fluoropropoxy, 3,3-difluoropropoxy and 3-dibromopropoxy. Preferably, the one or more halo radicals of C₁-C₆haloalkoxyl is fluoro. Preferably, C₁-C₆haloalkoxyl is selected from trifluoromethoxy, difluoromethoxy, fluoromethoxy, 1,1-difluoroethoxy, 2,2-difluoroethoxy, 2,2,2-trifluoroethoxy, 1-fluoromethyl-2-fluoroethoxy, and pentafluoroethoxy.

The term “halogen” or “halo” means fluoro, chloro, bromo or iodo.

As used herein, the term “cycloalkyl” means a monocyclic or polycyclic saturated or partially unsaturated carbon ring containing 3-18 carbon atoms wherein there are no delocalized pi electrons (aromaticity) shared among the ring carbon. The terms “C₃-C₁₀cycloalkyl”, “C₃-C₈cycloalkyl”, “C₄-C₁₀cycloalkyl” and “C₄-C₇cycloalkyl” are to be construed accordingly. The term polycyclic encompasses bridged (e.g., norbonane), fused (e.g., decalin) and spirocyclic cycloalkyl. Preferably, cycloalkyl, e.g., C₃-C₁₀cycloalkyl, is a monocyclic, bridged or spirocyclic hydrocarbon group of 3 to 10 carbon atoms.

Examples of cycloalkyl groups include, without limitations, cyclopropenyl, cyclopropyl cyclobutyl, cyclobutenyl, cyclopentyl, cyclohexyl, cycloheptanyl, cyclooctanyl, norboranyl, norborenyl, spiro[3.3]heptanyl (e.g., spiro[3.3]heptan-6-yl), bicyclo[2.2.2]octanyl, bicyclo[2.2.2]octenyl, adamantyl and derivatives thereof. Preferably, the cycloalkyl group is saturated.

Preferred examples of C₃-C₁₀cycloalkyl include, but are not limited to, cyclopropyl, cyclobutyl, cyclopentyl, cyclohexyl, spiro[3.3]heptanyl (e.g., spiro[3.3]heptan-6-yl), bicyclo[1.1.1]pentyl, bicyclo[2.1.1]hexyl, bicyclo[2.1.1]heptyl, bicyclo[2.2.2]octyl and adamantyl.

“Heterocyclyl” means a saturated or partially saturated monocyclic or polycyclic ring containing carbon and at least one heteroatom selected from oxygen, nitrogen, and sulfur (O, N, and S) and wherein there are no delocalized pi electrons (aromaticity) shared among the ring carbon or heteroatoms. The terms “4- to 10-membered heterocyclyl”, “4- to 6-membered heterocyclyl” and “5- or 6-membered heterocyclyl” are to be construed accordingly. The heterocyclyl ring structure may be substituted by one or more substituents. The substituents can themselves be optionally substituted. The heterocyclyl may be bonded via a carbon atom or heteroatom. The term polycyclic encompasses bridged, fused and spirocyclic heterocyclyl.

Examples of heterocyclyl rings include, but are not limited to, oxetanyl, azetidinyl, tetrahydrofuranyl, tetrahydropyranyl, pyrrolidinyl, oxazolinyl, isoxazolinyl, oxazolidinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, 1,4-dioxanyl, dihydrofuranyl, 1,3-dioxolanyl, imidazolidinyl, dihydroisoxazolinyl, pyrrolinyl, pyrazolinyl, oxazepinyl, dithiolanyl, homotropanyl, dihydropyranyl (e.g., 3,6-dihydro-2H-pyranyl), oxaspiroheptanyl (e.g., 2-oxaspiro[3.3]heptan-6-yl), diazabicyclo[3.2.1]octan-3-yl), 2-azaspiro[3.3]heptanyl (e.g., 2-azaspiro[3.3]heptan-6-yl), and the like.

Preferred examples of heterocyclyl include, without limitations, oxetanyl, azetidinyl, pyrrolidinyl, tetrahydrofuranyl, tetrahydrothienyl, piperidinyl, piperazinyl, dihydroisoxazolinyl, tetrahydropyranyl, morpholinyl, dihydropyranyl (e.g., 3,6-dihydro-2H-pyranyl), 2-azaspiro[3.3]heptanyl (e.g., 2-azaspiro[3.3]heptan-6-yl), and oxaspiroheptanyl (e.g., 2-oxaspiro[3.3]heptan-6-yl).

As used herein, the term “aryl” as used herein means monocyclic, bicyclic or polycyclic carbocyclic aromatic rings. Examples of aryl include, but are not limited to, phenyl, naphthyl (e.g., naphth-1-yl, naphth-2-yl), anthryl (e.g., anthr-1-yl, anthr-9-yl), phenanthryl (e.g., phenanthr-1-yl, phenanthr-9-yl), and the like. Aryl is also intended to include monocyclic, bicyclic or polycyclic carbocyclic aromatic rings substituted with carbocyclic aromatic rings. Representative examples are biphenyl (e.g., biphenyl-2-yl, biphenyl-3-yl, biphenyl-4-yl), phenylnaphthyl (e.g., 1-phenylnaphth-2-yl, 2-phenylnaphth-1-yl), and the like. Aryl is also intended to include partially saturated bicyclic or polycyclic carbocyclic rings with at least one unsaturated moiety (e.g., a benzo moiety). Representative examples are, indanyl (e.g., indan-1-yl, indan-5-yl), indenyl (e.g., inden-1-yl, inden-5-yl), 1,2,3,4-tetrahydronaphthyl (e.g., 1,2,3,4-tetrahydronaphth-1-yl, 1,2,3,4-tetrahydronaphth-2-yl, 1,2,3,4-tetrahydronaphth-6-yl), 1,2-dihydronaphthyl (e.g., 1,2-dihydronaphth-1-yl, 1,2-dihydronaphth-4-yl, 1,2-dihydronaphth-6-yl), fluorenyl (e.g., fluoren-1-yl, fluoren-4-yl, fluoren-9-yl), and the like. Aryl is also intended to include partially saturated bicyclic or polycyclic carbocyclic aromatic rings containing one or two bridges. Representative examples are, benzonorbornyl (e.g., benzonorborn-3-yl, benzonorborn-6-yl), 1,4-ethano-1,2,3,4-tetrahydronapthyl (e.g., 1,4-ethano-1,2,3,4-tetrahydronapth-2-yl, 1,4-ethano-1,2,3,4-tetrahydronapth-10-yl), and the like. The term “C₆-C₁₀aryl” is to be construed accordingly.

Preferred examples of aryl include, but are not limited to, indenyl, (e.g., inden-1-yl, inden-5-yl) phenyl (C₆H₅), naphthyl (C₁₀H₇) (e.g., naphth-1-yl, naphth-2-yl), indanyl (e.g., indan-1-yl, indan-5-yl), and tetrahydronaphthalenyl (e.g., 1,2,3,4-tetrahydronaphthalenyl).

Preferably, C₆-C₁₀aryl refers to a monocyclic or bicyclic carbocyclic aromatic ring.

Preferred examples of C₆-C₁₀aryl include, but are not limited to, phenyl and naphthyl. In an embodiment, C₆-C₁₀aryl is phenyl.

As used herein, the term “heteroaryl” as used herein is intended to include monocyclic heterocyclic aromatic rings containing one or more heteroatoms selected from oxygen, nitrogen, and sulfur (O, N, and S). Representative examples are pyrrolyl, furanyl, thienyl, oxazolyl, thiazolyl, imidazolyl, pyrazolyl, isothiazolyl, isooxazolyl, triazolyl, (e.g., 1,2,4-triazolyl), oxadiazolyl, (e.g., 1,2,3-oxadiazolyl, 1,2,4-oxadiazolyl, 1,2,5-oxadiazolyl, 1,3,4-oxadiazolyl), thiadiazolyl (e.g., 1,2,3-thiadiazolyl, 1,2,4-thiadiazolyl, 1,2,5-thiadiazolyl, 1,3,4-thiadiazolyl), tetrazolyl, pyranyl, pyridinyl, pyridazinyl, pyrimidinyl, pyrazinyl, 1,2,3-triazinyl, 1,2,4-triazinyl, 1,3,5-triazinyl, thiadiazinyl, azepinyl, azecinyl, and the like.

Heteroaryl is also intended to include bicyclic heterocyclic aromatic rings containing one or more heteroatoms selected from oxygen, nitrogen, and sulfur (0, N, and S). Representative examples are indolyl, isoindolyl, benzofuranyl, benzothiophenyl, indazolyl, benzopyranyl, benzimidazolyl, benzothiazolyl, benzisothiazolyl, benzoxazolyl, benzisoxazolyl, benzoxazinyl, benzotriazolyl, naphthyridinyl, phthalazinyl, pteridinyl, purinyl, quinazolinyl, cinnolinyl, quinolinyl, isoquinolinyl, quinoxalinyl, oxazolopyridinyl, isooxazolopyridinyl, pyrrolopyridinyl, furopyridinyl, thienopyridinyl, imidazopyridinyl, imidazopyrimidinyl, pyrazolopyridinyl, pyrazolopyrimidinyl, pyrazolotriazinyl, thiazolopyridinyl, thiazolopyrimidinyl, imdazothiazolyl, triazolopyridinyl, triazolopyrimidinyl, and the like.

Heteroaryl is also intended to include polycyclic heterocyclic aromatic rings containing one or more heteroatoms selected from oxygen, nitrogen, and sulfur (0, N, and S). Representative examples are carbazolyl, phenoxazinyl, phenazinyl, acridinyl, phenothiazinyl, carbolinyl, phenanthrolinyl, and the like.

Heteroaryl is also intended to include partially saturated monocyclic, bicyclic or polycyclic heterocyclyls containing one or more heteroatoms selected oxygen, nitrogen, and sulfur (0, N, and S). Representative examples are imidazolinyl, indolinyl, dihydrobenzofuranyl, dihydrobenzothienyl, dihydrobenzopyranyl, dihydropyridooxazinyl, dihydrobenzodioxinyl (e.g., 2,3-dihydrobenzo[b][1,4]dioxinyl), benzodioxolyl (e.g., benzo[d][1,3]dioxole), dihydrobenzooxazinyl (e.g., 3,4-dihydro-2H-benzo[b][1,4]oxazine), tetrahydroindazolyl, tetrahydrobenzimidazolyl, tetrahydroimidazo[4,5-c]pyridyl, tetrahydroquinolinyl, tetrahydroisoquinolinyl, tetrahydroquinoxalinyl, and the like.

The heteroaryl ring structure may be substituted by one or more substituents. The substituents can themselves be optionally substituted. The heteroaryl ring may be bonded via a carbon atom or heteroatom.

The term “5-10 membered heteroaryl” is to be construed accordingly.

Examples of 5-10 membered heteroaryl include, but are not limited to, indolyl, imidazopyridyl, isoquinolinyl, benzooxazolonyl, pyridinyl, pyrimidinyl, pyridinonyl, benzotriazolyl, pyridazinyl, pyrazolotriazinyl, indazolyl, benzimidazolyl, quinolinyl, triazolyl, (e.g., 1,2,4-triazolyl), pyrazolyl, thiazolyl, oxazolyl, isooxazolyl, pyrrolyl, oxadiazolyl, (e.g., 1,2,3-oxadiazolyl, 1,2,4-oxadiazolyl, 1,2,5-oxadiazolyl, 1,3,4-oxadiazolyl), imidazolyl, pyrrolopyridinyl, tetrahydroindazolyl, quinoxalinyl, thiadiazolyl (e.g., 1,2,3-thiadiazolyl, 1,2,4-thiadiazolyl, 1,2,5-thiadiazolyl, 1,3,4-thiadiazolyl), pyrazinyl, oxazolopyridinyl, pyrazolopyrimidinyl, benzoxazolyl, indolinyl, isooxazolopyridinyl, dihydropyridooxazinyl, tetrazolyl, dihydrobenzodioxinyl (e.g., 2,3-dihydrobenzo[b][1,4]dioxinyl), benzodioxolyl (e.g., benzo[d][1,3]dioxole) and dihydrobenzooxazinyl (e.g., 3,4-dihydro-2H-benzo[b][1,4]oxazine).

As used herein, the term “oxo” refers to the radical ═O.

“Cyano” or “—CN” means a substituent having a carbon atom joined to a nitrogen atom by a triple bond, e.g., C≡N.

The term “C₂-C₆alkenyl” as used herein represents a branched or straight hydrocarbon group having from 2 to 6 carbon atoms and at least one double bond. Representative examples are ethenyl (or vinyl), propenyl (e.g., prop-1-enyl, prop-2-enyl), 2-methylprop-1-enyl, 2-methylprop-2-enyl, 1,1-(dimethyl)prop-2-enyl, butadienyl (e.g., buta-1,3-dienyl), butenyl (e.g., but-1-en-1-yl, but-2-en-1-yl), 2-methylbut-1-enyl, pentenyl (e.g., pent-1-enyl, pent-2-enyl), hexenyl (e.g., hex-1-enyl, hex-2-enyl, hex-3-enyl), 2-methylpent-3-enyl, and the like.

As used herein, the term “bridging ring” refers to a ring formed at two non-adjacent carbon atoms of the heterocycloalkyl moiety of formula (I), linked to form a C₁-C₃ alkylene linker, wherein one of the carbon atoms of said linker is optionally replaced by a heteroatom selected from nitrogen, oxygen and sulfur. In a preferred embodiment, the alkylene linker comprises carbon atoms only.

As used herein, the term “C₁-C₃alkylene” refers to a straight hydrocarbon chain bivalent radical consisting solely of carbon and hydrogen atoms, containing no unsaturation, having from one to two carbon atoms.

As used herein, the term “optionally substituted” includes unsubstituted or substituted.

As used herein, “/*” denotes the point of attachment to the other part of the molecule.

As used herein, the term nitrogen protecting group (PG) in a compound of Formula (X) or any intermediates in any of the general schemes 1 to 4 and subformulae thereof refers to a group that should protect the functional groups concerned against unwanted secondary reactions, such as acylations, etherifications, esterifications, oxidations, solvolysis and similar reactions. It may be removed under deprotection conditions. Depending on the protecting group employed, the skilled person would know how to remove the protecting group to obtain the free amine NH₂ group by reference to known procedures. These include reference to organic chemistry textbooks and literature procedures such as J. F. W. McOmie, “Protective Groups in Organic Chemistry”, Plenum Press, London and New York 1973; T. W. Greene and P. G. M. Wuts, “Greene's Protective Groups in Organic Synthesis”, Fourth Edition, Wiley, New York 2007; in “The Peptides”; Volume 3 (editors: E. Gross and J. Meienhofer), Academic Press, London and New York 1981; P. J. Kocienski, “Protecting Groups”, Third Edition, Georg Thieme Verlag, Stuttgart and New York 2005; and in “Methoden der organischen Chemie” (Methods of Organic Chemistry), Houben Weyl, 4th edition, Volume 15/I, Georg Thieme Verlag, Stuttgart 1974.

Preferred nitrogen protecting groups generally comprise: C₁-C₆alkyl (e.g., tert-butyl), preferably C₁-C₄alkyl, more preferably C₁-C₂alkyl, most preferably C₁alkyl which is mono-, di- or tri-substituted by trialkylsilyl-C₁-C₇alkoxy (e.g., trimethylsilylethoxy), aryl, preferably phenyl, or a heterocyclic group (e.g., benzyl, cumyl, benzhydryl, pyrrolidinyl, trityl, pyrrolidinylmethyl, 1-methyl-1,1-dimethylbenzyl, (phenyl)methylbenzene) wherein the aryl ring or the heterocyclic group is unsubstituted or substituted by one or more, e.g., two or three, residues, e.g., selected from the group consisting of C₁-C₇alkyl, hydroxy, C₁-C₇alkoxy (e.g., para-methoxy benzyl (PMB)), C₂-C₈-alkanoyl-oxy, halogen, nitro, cyano, and CF₃, aryl-C₁-C₂-alkoxycarbonyl (preferably phenyl-C₁-C₂-alkoxycarbonyl (e.g., benzyloxycarbonyl (Cbz), benzyloxymethyl (BOM), pivaloyloxymethyl (POM)), C₁-C₁₀-alkenyloxycarbonyl, C₁-C₆alkylcarbonyl (e.g., acetyl or pivaloyl), C₆-C₁₀-arylcarbonyl; C₁-C₆-alkoxycarbonyl (e.g., tertbutoxycarbonyl (Boc), methylcarbonyl, trichloroethoxycarbonyl (Troc), pivaloyl (Piv), allyloxycarbonyl), C₆-C₁₀-arylC₁-C₆-alkoxycarbonyl (e.g., 9-fluorenylmethyloxycarbonyl (Fmoc)), allyl or cinnamyl, sulfonyl or sulfenyl, succinimidyl group, silyl groups (e.g., triarylsilyl, trialkylsilyl, triethylsilyl (TES), trimethylsilylethoxymethyl (SEM), trimethylsilyl (TMS), triisopropylsilyl or tertbutyldimethylsilyl).

According to the disclosure, the preferred nitrogen protecting group (PG) can be selected from the group comprising tert-butyloxycarbonyl (Boc), benzyloxycarbonyl (Cbz), para-methoxy benzyl (PMB), 2,4-dimethoxybenzyl (DMB), methyloxycarbonyl, trimethylsilylethoxymethyl (SEM) and benzyl. The nitrogen protecting group (PG) is preferably an acid labile protecting group, e.g., tert-butyloxycarbonyl (Boc), 2,4-dimethoxybenzyl (DMB).

In some embodiments, the compounds of the disclosure are selective over other proteins.

As used herein, the term “therapeutic agent” in connection with methods of reducing WIZ protein expression levels and/or inducing fetal hemoglobin (HbF) expression, refers to a substance that results in a detectably lower expression of WIZ gene or WIZ protein or lower activity level of WIZ proteins as compared to those levels without such substance.

As used herein “modulator” or “degrader”, means, for example, a compound of the disclosure, that effectively modulates, decreases, or reduces the levels of a specific protein (e.g., WIZ) or degrades a specific protein (e.g., WIZ). The amount of a specific protein (e.g., WIZ) degraded can be measured by comparing the amount of the specific protein (e.g., WIZ) remaining after treatment with a compound of the disclosure as compared to the initial amount or level of the specific protein (e.g., WIZ) present as measured prior to treatment with a compound of the disclosure.

As used herein “selective modulator”, “selective degrader”, or “selective compound” means, for example, a compound of the disclosure, that effectively modulates, decreases, or reduces the levels of a specific protein (e.g., WIZ) or degrades a specific protein (e.g., WIZ) to a greater extent than any other protein. A “selective modulator”, “selective degrader”, or “selective compound” can be identified, for example, by comparing the ability of a compound to modulate, decrease, or reduce the levels of or to degrade a specific protein (e.g., WIZ) to its ability to modulate, decrease, or reduce the levels of or to degrade other proteins. In some embodiments, the selectivity can be identified by measuring the EC₅₀ or IC₅₀ of the compounds. Degradation may be achieved through mediation of an E3 ligase, e.g., E3-ligase complexes comprising the protein Cereblon.

In one embodiment, the specific protein degraded is WIZ protein. In an embodiment, at least about 30% of WIZ is degraded compared to initial levels. In an embodiment, at least about 40% of WIZ is degraded compared to initial levels. In an embodiment, at least about 50% of WIZ is degraded compared to initial levels. In an embodiment, at least about 60% of WIZ is degraded compared to initial levels. In an embodiment, at least about 70% of WIZ is degraded compared to initial levels. In an embodiment, at least about 75% of WIZ is degraded compared to initial levels. In an embodiment, at least about 80% of WIZ is degraded compared to initial levels. In an embodiment, at least about 85% of WIZ is degraded compared to initial levels. In an embodiment, at least about 90% of WIZ is degraded compared to initial levels. In an embodiment, at least about 95% of WIZ is degraded compared to initial levels. In an embodiment, over 95% of WIZ is degraded compared to initial levels. In an embodiment, at least about 99% of WIZ is degraded compared to initial levels.

In an embodiment, the WIZ is degraded in an amount of from about 30% to about 99% compared to initial levels. In an embodiment, the WIZ is degraded in an amount of from about 40% to about 99% compared to initial levels. In an embodiment, the WIZ is degraded in an amount of from about 50% to about 99% compared to initial levels. In an embodiment, the WIZ is degraded in an amount of from about 60% to about 99% compared to initial levels. In an embodiment, the WIZ is degraded in an amount of from about 70% to about 99% compared to initial levels. In an embodiment, the WIZ is degraded in an amount of from about 80% to about 99% compared to initial levels. In an embodiment, the WIZ is degraded in an amount of from about 90% to about 99% compared to initial levels. In an embodiment, the WIZ is degraded in an amount of from about 95% to about 99% compared to initial levels. In an embodiment, the WIZ is degraded in an amount of from about 90% to about 95% compared to initial levels.

As used herein, the terms “inducing fetal hemoglobin”, “fetal hemoglobin induction”, or “increasing fetal hemoglobin expression” refer to increasing the percentage of HbF in the blood of a subject. In an embodiment, the amount of total HbF in the blood of the subject increases. In an embodiment, the amount of total hemoglobin in the blood of the subject increases. In an embodiment, the amount of HbF is increased by at least about 10%, or at least about 20%, or at least about 30%, or at least about 40%, or at least about 50%, or at least about 60%, or at least about 70%, or at least about 80%, or at least about 90%, or at least about 100%, or more than 100%, for example, at least about 2-fold, or at least about 3-fold, or at least about 4-fold, or at least about 5-fold, or at least about 6-fold, or at least about 7-fold, or at least about 8-fold, or at least about 9-fold, or at least about 10-fold, or more than 10-fold as compared to either in the absence of a compound disclosed herein.

In an embodiment, the total hemoglobin in the blood, e.g., the blood in a subject, is increased by at least about 10%, or at least about 20%, or at least about 30%, or at least about 40%, or at least about 50%, or at least about 60%, or at least about 70%, or at least about 80%, or at least about 90%, or at least about 100%, or more than 100%, for example, at least about 2-fold, or at least about 3-fold, or at least about 4-fold, or at least about 5-fold, or at least about 6-fold, or at least about 7-fold, or at least about 8-fold, or at least about 9-fold, or at least about 10-fold, or more than 10-fold as compared to either in the absence of a compound disclosed herein.

The term “a therapeutically effective amount” of a compound of the disclosure refers to an amount of the compound of the disclosure that will elicit the biological or medical response of a subject, for example, reduction or inhibition of an enzyme or a protein activity, or ameliorate symptoms, alleviate conditions, slow or delay disease progression, or prevent a disease, etc. In one embodiment, the term “a therapeutically effective amount” refers to the amount of the compound of the disclosure that, when administered to a subject, is effective to (1) at least partially alleviate, prevent and/or ameliorate a condition, or a disorder or a disease (i) mediated by WIZ, or (ii) associated with WIZ activity, or (iii) characterized by activity (normal or abnormal) of WIZ: (2) reduce or inhibit the activity of WIZ; or (3) reduce or inhibit the expression of WIZ. In another embodiment, the term “a therapeutically effective amount” refers to the amount of the compound of the disclosure that, when administered to a cell, or a tissue, or a non-cellular biological material, or a medium, is effective to at least partially reducing or inhibiting the activity of WIZ; or at least partially reducing or inhibiting the expression of WIZ.

“HbF-dependent disease or disorder” means any disease or disorder which is directly or indirectly affected by the modulation of HbF protein levels.

As used herein, the term “subject” refers to primates (e.g., humans, male or female), dogs, rabbits, guinea pigs, pigs, rats and mice. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder As used herein, a subject is “in need of” a treatment if such subject would benefit biologically, medically or in quality of life from such treatment.

As used herein, the term “a,” “an,” “the” and similar terms used in the context of the disclosure (especially in the context of the claims) are to be construed to cover both the singular and plural unless otherwise indicated herein or clearly contradicted by the context.

Various enumerated embodiments of the disclosure are described herein. It will be recognized that features specified in each embodiment may be combined with other specified features to provide further embodiments of the disclosure.

### Enumerated Embodiments

Embodiment 1. A compound of formula (I″) or a pharmaceutically acceptable salt thereof, wherein:

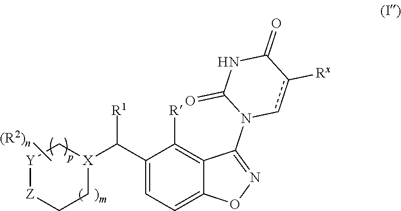

[symbol] is a single bond or a double bond;
X is selected from CH, CF, and N;
Y is selected from CH₂, CR^{Y}R² and N—R³;
Z is selected from CH₂, CR^{Y}R² and N—R³,
wherein at least one of Y and Z is N—R³ such that when Y is N—R³, Z is selected from CH₂, and CR^{Y}R², and when Z is N—R³, Y is selected from CH₂, and CR^{Y}R²,
and wherein when R² of CR^{Y}R² of Y or Z is oxo, R^{Y} is absent;
R^{x} is selected from hydrogen, C₁-C₆alkyl, halo (e.g., F, Cl), C₁-C₆alkoxyl, and C₃-C₈cycloalkyl;
R^{Y} is selected from hydrogen and C₁-C₆alkyl,
R′ is selected from hydrogen and C₁-C₆alkyl;
R¹ is selected from hydrogen and C₁-C₆alkyl;
each R² is independently selected from C₁-C₆alkyl, C₁-C₆haloalkyl, halo, and oxo, wherein the C₁-C₆alkyl is substituted with 0-1 occurrence of R^{2a}; or 2 R² on non-adjacent carbon atoms together with the non-adjacent carbon atoms to which they are attached form a bridging ring;
R^{2a} is selected from C₁-C₆alkoxyl and hydroxyl;
R³ is selected from hydrogen, C₁-C₈alkyl, C₂-C₆alkenyl, —SO₂R⁴, C₁-C₆haloalkyl, a 4- to 6-membered heterocyclyl comprising 1-2 heteroatoms independently selected from N, O and S, C₃-C₁₀cycloalkyl, —C(═O)—O—(R⁵) and —C(═O)—(R⁶), wherein the C₁-C₈alkyl and C₁-C₆haloalkyl are independently substituted with 0-3 occurrences of R^{3a};
or
R³ together with the nitrogen atom to which it is attached and R² together with the carbon atom to which it is attached form a 5- or 6-membered heterocyclyl comprising 0-1 additional heteroatom selected from N, O and S;
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S, C₆-C₁₀aryl, C₁-C₆alkoxyl, hydroxyl, and —C(═O)—NR⁷R⁸, wherein the C₃-C₁₀cycloalkyl, 4- to 6-membered heterocyclyl, 5- to 10-membered heteroaryl and C₆-C₁₀aryl are substituted with 0-4 occurrences of R^{3b};
each R^{3b} is independently selected from C₁-C₆alkoxyl, halo, C₁-C₆haloalkyl, C₁-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁵ is selected from C₁-C₆alkyl, C₃-C₈cycloalkyl, and C₆-C₁₀aryl;
R⁶ is selected from C₁-C₆alkyl, C₃-C₈cycloalkyl, NR⁷R⁸, and C₆-C₁₀aryl, wherein the C-C₆alkyl is substituted with 0-1 occurrence of R^{6a} and the C₃-C₈cycloalkyl is substituted with 0-1 occurrence of R^{6b};
R^{6a} is selected from C₆-C₁₀aryl and C₃-C₈cycloalkyl;
R^{6b} is selected from halo, C₁-C₆haloalkyl, C₁-C₆haloalkoxyl, and C₁-C₆alkyl;
R⁷ is selected from hydrogen and C₁-C₆alkyl;
R⁸ is selected from hydrogen, C₃-C₈cycloalkyl and C₁-C₆alkyl;
or
R⁷ and R⁸ together with the nitrogen atom to which they are attached form a 5- or 6-membered heterocyclyl comprising 0-1 additional heteroatom selected from N, O, and S;
n is 0, 1, 2, 3 or 4;
m is 0, 1 or 2; and
p is 0 or 1.
   Embodiment 2. A compound according to Embodiment 1, or a pharmaceutically acceptable salt thereof, having Formula (I′), wherein:

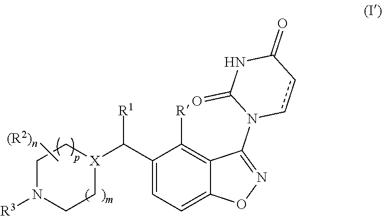

[symbol] is a single bond or a double bond;
X is selected from CH, CF, and N;
R′ is selected from hydrogen and C₁-C₆alkyl;
R¹ is selected from hydrogen and C₁-C₆alkyl;
each R² is independently selected from C₁-C₆alkyl, C₁-C₆haloalkyl, halo, and oxo, wherein the C₁-C₆alkyl is substituted with 0-1 occurrence of R^{2a}; or 2 R² on non-adjacent carbon atoms together with the non-adjacent carbon atoms to which they are attached form a bridging ring;
R^{2a} is selected from C₁-C₆alkoxyl and hydroxyl;
R³ is selected from hydrogen, C₁-C₈alkyl, C₂-C₆alkenyl, —SO₂R⁴, C₁-C₆haloalkyl, —C(═O)—O—(R⁵) and —C(═O)—(R⁶), wherein the C₁-C₈alkyl and C₁-C₆haloalkyl are independently substituted with 0-3 occurrences of R^{3a};
or
R³ together with the nitrogen atom to which it is attached and R² together with the carbon atom to which it is attached form a 5- or 6-membered heterocyclyl comprising 0-1 additional heteroatom selected from N, O and S;
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S, C₆-C₁₀aryl, C₁-C₆alkoxyl, hydroxyl, and —C(═O)—NR⁷R⁸, wherein the C₃-C₁₀cycloalkyl, 4- to 6-membered heterocyclyl, 5- to 10-membered heteroaryl and C₆-C₁₀aryl are substituted with 0-4 occurrences of R^{3b};
each R^{3b} is independently selected from C₁-C₆alkoxyl, halo, C₁-C₆haloalkyl, C₁-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁵ is selected from C₁-C₆alkyl, C₃-C₈cycloalkyl, and C₆-C₁₀aryl;
R⁶ is selected from C₁-C₆alkyl, C₃-C₈cycloalkyl, and C₆-C₁₀aryl, wherein the C₁-C₆alkyl is substituted with 0-1 occurrence of R^{6a} and the C₃-C₈cycloalkyl is substituted with 0-1 occurrence of R^{6b};
R^{6a} is selected from C₆-C₁₀aryl and C₃-C₈cycloalkyl;
R^{6b} is selected from halo, C₁-C₆haloalkyl, C₁-C₆haloalkoxyl, and C₁-C₆alkyl;
R⁷ is selected from hydrogen and C₁-C₆alkyl;
R⁸ is selected from hydrogen and C₁-C₆alkyl;
or
R⁷ and R⁸ together with the nitrogen atom to which they are attached form a 5- or 6-membered heterocyclyl comprising 0-1 additional heteroatom selected from N, O, and S;
n is 0, 1, 2, 3 or 4;
m is 0, 1 or 2; and
p is 0 or 1.
   Embodiment 3. The compound according to any one of Embodiments 1 and 2, or a pharmaceutically acceptable salt thereof, having Formula (I), wherein:

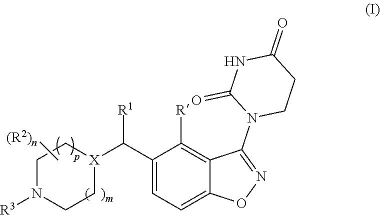

X is selected from CH, CF, and N;
R′ is selected from hydrogen and C₁-C₆alkyl;
R¹ is selected from hydrogen and C₁-C₆alkyl;
each R² is independently selected from C₁-C₆alkyl, C₁-C₆haloalkyl, halo, and oxo, wherein the C₁-C₆alkyl is substituted with 0-1 occurrence of R^{2a}; or 2 R² on non-adjacent carbon atoms together with the non-adjacent carbon atoms to which they are attached form a bridging ring;
R^{2a} is selected from C₁-C₆alkoxyl and hydroxyl;
R³ is selected from hydrogen, C₁-C₈alkyl, C₂-C₆alkenyl, —SO₂R⁴, C₁-C₆haloalkyl, —C(═O)—O—(R⁵) and —C(═O)—(R⁶), wherein the C₁-C₈alkyl and C₁-C₆haloalkyl are independently substituted with 0-3 occurrences of R^{3a};
or
R³ together with the nitrogen atom to which it is attached and R² together with the carbon atom to which it is attached form a 5- or 6-membered heterocyclyl comprising 0-1 additional heteroatom selected from N, O and S;
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S, C₆-C₁₀aryl, C₁-C₆alkoxyl, hydroxyl, and —C(═O)—NR⁷R⁸, wherein the C₃-C₁₀cycloalkyl, 4- to 6-membered heterocyclyl, 5- to 10-membered heteroaryl and C₆-C₁₀aryl are substituted with 0-4 occurrences of R^{3b};
each R^{3b} is independently selected from C₁-C₆alkoxyl, halo, C₁-C₆haloalkyl, C-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁵ is selected from C₁-C₆alkyl, C₃-C₈cycloalkyl, and C₆-C₁₀aryl;
R⁶ is selected from C₁-C₆alkyl, C₃-C₈cycloalkyl, and C₆-C₁₀aryl, wherein the C₁-C₆alkyl is substituted with 0-1 occurrence of R^{6a} and the C₃-C₈cycloalkyl is substituted with 0-1 occurrence of R^{6b};
R^{6a} is selected from C₆-C₁₀aryl and C₃-C₈cycloalkyl;
R^{6b} is selected from halo, C₁-C₆haloalkyl, C₁-C₆haloalkoxyl, and C₁-C₆alkyl;
R⁷ is selected from hydrogen and C₁-C₆alkyl;
R⁸ is selected from hydrogen and C₁-C₆alkyl;
or
R⁷ and R⁸ together with the nitrogen atom to which they are attached form a 5- or 6-membered heterocyclyl comprising 0-1 additional heteroatom selected from N, O, and S;
n is 0, 1, 2, 3 or 4;
m is 0, 1 or 2; and
p is 0 or 1.
   Embodiment 4. The compound according to any one of the preceding Embodiments, or a pharmaceutically acceptable salt thereof, wherein
X is selected from CH and N;
R′ is hydrogen;
R¹ is selected from hydrogen and C₁-C₃alkyl;
each R² is independently selected from unsubstituted C₁-C₆alkyl, C₁-C₆haloalkyl and halo; or 2 R² on non-adjacent carbon atoms together with the non-adjacent carbon atoms to which they are attached form a bridging ring;
R³ is selected from hydrogen, C₁-C₈alkyl, C₂-C₆alkenyl, —SO₂R⁴, C₁-C₆haloalkyl and —C(═O)—(R⁶), wherein the C₁-C₈alkyl and C₁-C₆haloalkyl are independently substituted with 0-3 occurrences of R^{3a};
or
R³ together with the nitrogen atom to which it is attached and R² together with the carbon atom to which it is attached form a 5- or 6-membered heterocyclyl comprising 0-1 additional heteroatom selected from N and 0;
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S, C₆-C₁₀aryl, C₁-C₆alkoxyl, hydroxyl, and —C(═O)—NR⁷R⁸, wherein the C₃-C₁₀cycloalkyl, 4- to 6-membered heterocyclyl, 5- to 10-membered heteroaryl and C₆-C₁₀aryl are substituted with 0-4 occurrences of R^{3b};
each R^{3b} is independently selected from C₁-C₆alkoxyl, halo, C₁-C₆haloalkyl, C₁-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁶ is selected from C₁-C₆alkyl, C₃-C₈cycloalkyl, and C₆-C₁₀aryl, wherein the C₁-C₆alkyl is substituted with 0-1 occurrence of R^{6a} and the C₃-C₈cycloalkyl is substituted with 0-1 occurrence of R^{6b};
R^{6a} is selected from C₆-C₁₀aryl and C₃-C₈cycloalkyl;
R^{6b} is selected from chloro, fluoro, C₁-C₆haloalkyl, C₁-C₆haloalkoxyl, and C₁-C₆alkyl;
R⁷ is selected from hydrogen and C₁-C₆alkyl;
R⁸ is selected from hydrogen and C₁-C₆alkyl;
or
R⁷ and R⁸ together with the nitrogen atom to which they are attached form a 5- or 6-membered heterocyclyl comprising 0-1 additional heteroatom selected from N, O, and S;
n is 0, 1, 2 or 3;
m is 0, 1 or 2; and
p is 0 or 1.
   Embodiment 5. The compound according to any one of the preceding Embodiments, or a pharmaceutically acceptable salt thereof, wherein
X is selected from CH and N;
R′ is hydrogen;
R¹ is hydrogen;
each R² is independently selected from unsubstituted C₁-C₆alkyl and halo; or 2 R² on non-adjacent carbon atoms together with the non-adjacent carbon atoms to which they are attached form a C₁-C₂alkylene bridging ring;
R³ is selected from C₁-C₈alkyl, —SO₂R⁴, C₁-C₆haloalkyl and —C(═O)—(R⁶), wherein the C₁-C₈alkyl and C₁-C₆haloalkyl are independently substituted with 0-3 occurrences of R^{3a};
or
R³ together with the nitrogen atom to which it is attached and R² together with the carbon atom to which it is attached form a 5- or 6-membered heterocyclyl comprising 0-1 additional 0 heteroatom;
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S and phenyl, wherein the C₃-C₁₀cycloalkyl, 4- to 6-membered heterocyclyl, 5- to 10-membered heteroaryl and phenyl are substituted with 0-4 occurrences of R^{3b};
each R^{3b} is independently selected from C₁-C₆alkoxyl, halo, C₁-C₆haloalkyl, C-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁶ is selected from C₁-C₆alkyl, C₃-C₈cycloalkyl, and C₆-C₁₀aryl;
R⁷ is selected from hydrogen and C₁-C₆alkyl;
R⁸ is selected from hydrogen and C₁-C₆alkyl;
or
R⁷ and R⁸ together with the nitrogen atom to which they are attached form a 5- or 6-membered heterocyclyl comprising 0-1 additional heteroatom selected from N, O, and S;
n is 0, 1, 2 or 3, e.g., n is 0, 1 or 2;
m is 0, 1 or 2, e.g., m is 1 or 2; and
p is 0 or 1.
   Embodiment 6. The compound according to any one of the preceding Embodiments, or a pharmaceutically acceptable salt thereof, wherein
X is selected from CH and N;
R′ is hydrogen;
R¹ is hydrogen;
each R² is independently selected from unsubstituted C₁-C₆alkyl and fluoro;
R³ is selected from C₁-C₅alkyl, —SO₂R⁴ and C₁-C₆haloalkyl, wherein the C₁-C₅alkyl is substituted with 0-2 occurrences of R^{3a} and the C₁-C₆haloalkyl is substituted with 0-1 occurrence of R^{3a};
each R^{3a} is independently selected from C₃-C₈cycloalkyl, a 4- to 6-membered heterocyclyl comprising 1-2 heteroatoms independently selected from N and O, a 5- to 6-membered heteroaryl comprising 1-3 heteroatoms independently selected from N, O, and S, and phenyl, wherein the C₃-C₈cycloalkyl, 4- to 6-membered heterocyclyl, 5- to 6-membered heteroaryl and phenyl are substituted with 0-3 occurrences of R^{3b};
each R^{3b} is independently selected from halo, C₁-C₆haloalkyl, C₁-C₆haloalkoxyl, and C₁-C₆alkyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
n is 0, 1 or 2;
m is 1 or 2; and
p is 0 or 1.
   Embodiment 7. The compound according to any one of the preceding Embodiments, or a pharmaceutically acceptable salt thereof, wherein
X is selected from CH and N;
R′ is hydrogen;
R¹ is hydrogen;
each R² is independently C₁-C₆alkyl;
R³ is selected from C₁-C₅alkyl, —SO₂R⁴ and unsubstituted C₁-C₆haloalkyl, wherein the C₁-C₅alkyl is substituted with 0-2 occurrences of R^{3a};
each R^{3a} is independently selected from C₃-C₆cycloalkyl, a 4- to 6-membered heterocyclyl comprising 1 O heteroatom, a 5- to 6-membered heteroaryl comprising 1-3 heteroatoms independently selected from N, O, and S, and phenyl, wherein the C₃-C₆cycloalkyl, 4- to 6-membered heterocyclyl, 5- to 6-membered heteroaryl and phenyl are substituted with 0-2 occurrences of R^{3b};
each R^{3b} is independently selected from halo, C₁-C₆haloalkyl, C₁-C₆haloalkoxyl, and C₁-C₆alkyl;
R⁴ is selected from C₃-C₈cycloalkyl, C₁-C₆alkyl, and phenyl, e.g., R⁴ is C₁-C₆alkyl;
n is 0, 1 or 2;
m is 1 or 2; and
p is 1.
   Embodiment 8. The compound according to any one of the preceding Embodiments, or a pharmaceutically acceptable salt thereof, wherein
X is selected from CH and N;
R′ is hydrogen;
R¹ is hydrogen;
each R² is independently selected from unsubstituted C₁-C₃alkyl; R³ is selected from C₁-C₆alkyl, —SO₂R⁴ and unsubstituted C₁-C₆haloalkyl, wherein the C₁-C₆alkyl is substituted with 0-2 occurrences of R^{3a};
each R^{3a} is independently selected from C₃-C₆cycloalkyl, a 4- to 6-membered heterocyclyl comprising 1 O heteroatom, a 6-membered heteroaryl comprising 1-2 N heteroatoms, and phenyl, wherein the C₄-C₆cycloalkyl, 4- to 6-membered heterocyclyl, 6-membered heteroaryl and phenyl are substituted with 0-2 occurrences of R^{3b};
each R^{3b} is independently selected from chloro, fluoro, C₁-C₆haloalkyl, and C₁-C₆alkyl; R⁴ is C₁-C₆alkyl;
n is 0, 1 or 2;
m is 1; and
p is 1.
   Embodiment 9. The compound according to any one of the preceding Embodiments, or a pharmaceutically acceptable salt, hydrate, solvate, prodrug, stereoisomer, or tautomer thereof, of Formula (Ia″):

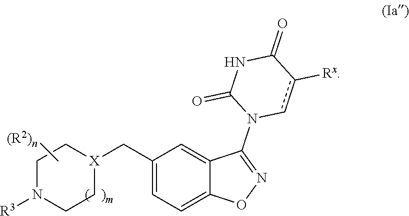

Embodiment 10. The compound according to any one of the preceding Embodiments, or a pharmaceutically acceptable salt thereof, of Formula (Ia′):

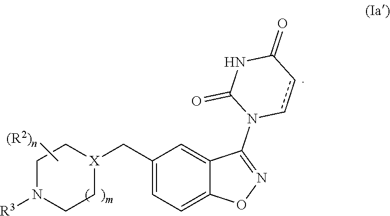

Embodiment 11. The compound according to any one of the preceding Embodiments, or a pharmaceutically acceptable salt thereof, of Formula (Ia):

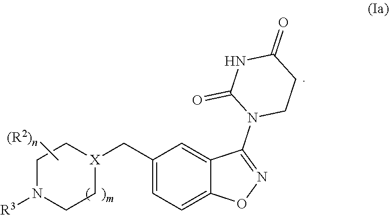

Embodiment 12. The compound according to any one of Embodiments 1 to 9, or a pharmaceutically acceptable salt thereof, of Formula (Ib″):

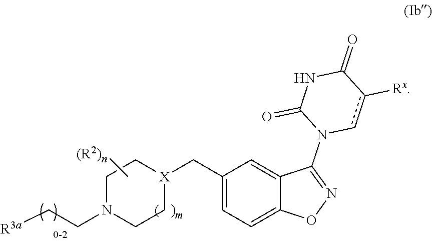

Embodiment 13. The compound according to any one of Embodiments 1 to 10 and 12, or a pharmaceutically acceptable salt thereof, of Formula (Ib′):

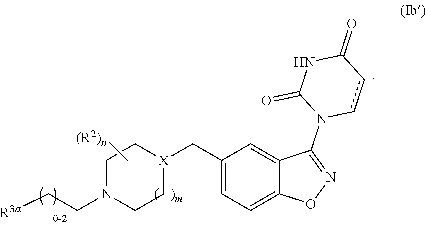

Embodiment 14. The compound according to any one of the preceding Embodiments, or a pharmaceutically acceptable salt thereof, of Formula (Ib):

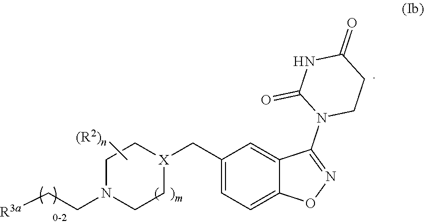

Embodiment 15. The compound according to any one of Embodiments 1 to 9 and 12, or a pharmaceutically acceptable salt thereof, of Formula (Ic″), wherein:

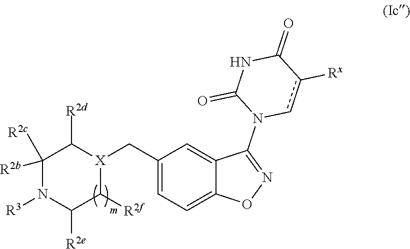

[symbol] is a single bond or a double bond;
X is selected from CH, C—F and N;
R^{x} is selected from hydrogen, C₁-C₆alkyl, halo (e.g., F, Cl), C₁-C₆alkoxyl, and C₃-C₆cycloalkyl;
R^{2b} is selected from hydrogen, C₁-C₆alkyl, C₁-C₆haloalkyl, and halo, wherein the C₁-C₆alkyl is substituted with 0-1 occurrence of R^{2a};
R^{2c} is selected from hydrogen and C₁-C₆alkyl, wherein the C₁-C₆alkyl is substituted with 0-1 occurrence of R^{2a};
or R^{2b} and R^{2c} together with the carbon atoms to which they are attached form an oxo group;
each of R^{2d} and R^{2e} is independently selected from hydrogen, C₁-C₆alkyl, C₁-C₆haloalkyl, halo, and oxo, wherein the C₁-C₆alkyl is substituted with 0-1 occurrence of R^{2a}; R^{2f} is hydrogen;
or R^{2b} and R^{2e} or R^{2b} and R^{2f} together with the carbon atoms to which they are attached form a bridging ring;
R^{2a} is selected from C₁-C₆alkoxyl and hydroxyl; and
R³ is defined according to any one of the preceding Embodiments.
   Embodiment 16. The compound according to any one of Embodiments 1 to 10, 12, 13 and 15, or a pharmaceutically acceptable salt thereof, of Formula (Ic′), wherein:

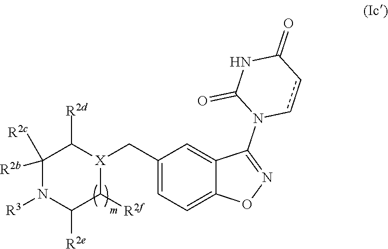

[symbol] is a single bond or a double bond;
X is selected from CH, C—F and N;
R^{2b} is selected from hydrogen, C₁-C₆alkyl, C₁-C₆haloalkyl, and halo, wherein the C₁-C₆alkyl is substituted with 0-1 occurrence of R^{2a};
R^{2C} is selected from hydrogen and C₁-C₆alkyl, wherein the C₁-C₆alkyl is substituted with 0-1 occurrence of R^{2a};
or R^{2b} and R^{2C} together with the carbon atoms to which they are attached form an oxo group;
each of R^{2d} and R^{2e} is independently selected from hydrogen, C₁-C₆alkyl, C₁-C₆haloalkyl, halo, and oxo, wherein the C₁-C₆alkyl is substituted with 0-1 occurrence of R^{2a};
R^{2f} is hydrogen;
or R^{2b} and R^{2e} or R^{2b} and R^{2f} together with the carbon atoms to which they are attached form a bridging ring;
R^{2a} is selected from C₁-C₆alkoxyl and hydroxyl; and
R³ is defined according to any one of the preceding Embodiments.
   Embodiment 17. The compound according to any one of the preceding Embodiments, or a pharmaceutically acceptable salt thereof, of Formula (Ic), wherein:

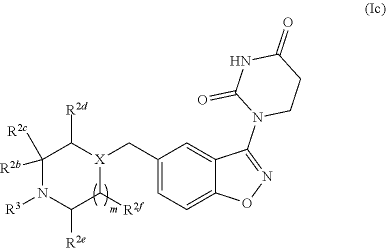

X is selected from CH, C—F and N;
R^{2b} is selected from hydrogen, C₁-C₆alkyl, C₁-C₆haloalkyl, and halo, wherein the C₁-C₆alkyl is substituted with 0-1 occurrence of R^{2a};
R^{2C} is selected from hydrogen and C₁-C₆alkyl, wherein the C₁-C₆alkyl is substituted with 0-1 occurrence of R^{2a};
or R^{2b} and R^{2C} together with the carbon atoms to which they are attached form an oxo group;
each of R^{2d} and R^{2e} is independently selected from hydrogen, C₁-C₆alkyl, C₁-C₆haloalkyl, halo, and oxo, wherein the C₁-C₆alkyl is substituted with 0-1 occurrence of R^{2a};
R^{2f} is hydrogen;
or R^{2b} and R^{2e} or R^{2b} and R^{2f} together with the carbon atoms to which they are attached form a bridging ring;
R^{2a} is selected from C₁-C₆alkoxyl and hydroxyl; and
R³ is defined according to any one of the preceding Embodiments.
   Embodiment 18. The compound according to any one of Embodiments 15 to 17, or a pharmaceutically acceptable salt thereof, wherein
X is selected from CH and N;
R^{2b} is selected from hydrogen, C₁-C₃alkyl, C₁-C₃haloalkyl, and halo;
R^{2c} is selected from hydrogen and C₁-C₃alkyl;
each of R^{2d} and R^{2e} is independently selected from hydrogen, C₁-C₃alkyl, C₁-C₃haloalkyl, and halo;
R^{2f} is hydrogen;
or R^{2b} and R^{2e} or R^{2b} and R^{2f} together with the carbon atoms to which they are attached form a C₁-C₂alkylene bridging ring;
R³ is selected from C₁-C₈alkyl, C₂-C₆alkenyl, —SO₂R⁴, C₁-C₆haloalkyl, and —C(═O)—(R⁶), wherein the C₁-C₅alkyl and C₁-C₆haloalkyl are independently substituted with 0-3 occurrences of R^{3a};
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S, C₆-C₁₀aryl, C₁-C₆alkoxyl, hydroxyl, and —C(═O)—NR⁷R⁸, wherein the C₃-C₁₀cycloalkyl, 4- to 6-membered heterocyclyl, 5- to 10-membered heteroaryl and C₆-C₁₀aryl are substituted with 0-4 occurrences of R^{3b};
each R^{3b} is independently selected from C₁-C₆alkoxyl, halo, C₁-C₆haloalkyl, C₁-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁶ is selected from C₁-C₆alkyl, C₃-C₈cycloalkyl, and C₆-C₁₀aryl, wherein the C₁-C₆alkyl is substituted with 0-1 occurrence of R^{6a} and the C₃-C₈cycloalkyl is substituted with 0-1 occurrence of R^{6b};
R^{6a} is selected from C₆-C₁₀aryl and C₃-C₈cycloalkyl;
R^{6b} is selected from halo, C₁-C₆haloalkyl, C₁-C₆haloalkoxyl, and C₁-C₆alkyl;
R⁷ is selected from hydrogen and C₁-C₆alkyl;
R⁸ is selected from hydrogen and C₁-C₆alkyl;
or
R⁷ and R⁸ together with the nitrogen atom to which they are attached form a 5- or 6-membered heterocyclyl comprising 0-1 additional heteroatom selected from N, O, and S; and
m is 1 or 2.
   Embodiment 19. The compound according to any one of Embodiments 15 to 18, or a pharmaceutically acceptable salt thereof, wherein
X is selected from CH and N;
each of R^{2b}, R^{2c}, R^{2d} and R^{2e} is independently selected from hydrogen and unsubstituted C₁-C₃alkyl;
R^{2f} is hydrogen;
R³ is selected from C₁-C₈alkyl, —SO₂R⁴, and C₁-C₆haloalkyl, wherein the C₁-C₈alkyl and C₁-C₆haloalkyl are independently substituted with 0-3 occurrences of R^{3a};
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S, and phenyl, wherein the C₃-C₁₀cycloalkyl, 4- to 6-membered heterocyclyl, 5- to 10-membered heteroaryl and phenyl are substituted with 0-4 occurrences of R^{3b};
each R^{3b} is independently selected from C₁-C₆alkoxyl, halo, C₁-C₆haloalkyl, C₁-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁷ is selected from hydrogen and C₁-C₆alkyl;
R⁸ is selected from hydrogen and C₁-C₆alkyl;
or
R⁷ and R⁸ together with the nitrogen atom to which they are attached form a 5- or 6-membered heterocyclyl comprising 0-1 additional heteroatom selected from N, O, and S; and
m is 1 or 2.
   Embodiment 20. The compound according to any one of Embodiments 15 to 19, or a pharmaceutically acceptable salt thereof, wherein
X is selected from CH and N;
each of R^{2b}, R^{2d} and R^{2e} is independently selected from hydrogen and unsubstituted C₁-C₃alkyl;
R^{2c} is hydrogen;
R^{2f} is hydrogen;
R³ is selected from C₁-C₅alkyl, —SO₂R⁴, and C₁-C₆haloalkyl, wherein the C₁-C₅alkyl is substituted with 0-2 occurrences of R^{3a} and the C₁-C₆haloalkyl is substituted with 0-1 occurrence of R^{3a};
each R^{3a} is independently selected from C₃-C₈cycloalkyl, a 4- to 6-membered heterocyclyl comprising 1-2 heteroatoms independently selected from N, O and S, a 5- to 10-membered heteroaryl comprising 1-3 heteroatoms independently selected from N, O, and S and phenyl, wherein the C₃-C₈cycloalkyl, 4- to 6-membered heterocyclyl, 5- to 10-membered heteroaryl and phenyl are substituted with 0-3 occurrences of R^{3b};
each R^{3b} is independently selected from halo, C₁-C₆haloalkyl, and C₁-C₆alkyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, e.g., R⁴ is C₁-C₆alkyl,
wherein the C₁-C₆alkyl is substituted with 0-1 occurrence of R^{4a};
R^{4a} is selected from C₃-C₈cycloalkyl, C₆-C₁₀aryl, and C₁-C₆alkoxyl; and
m is 1.
   Embodiment 21. The compound according to any one of the Embodiments 1, 4 to 9, 15 and 18 to 20, or a pharmaceutically acceptable salt thereof, of Formula (Id″), wherein:

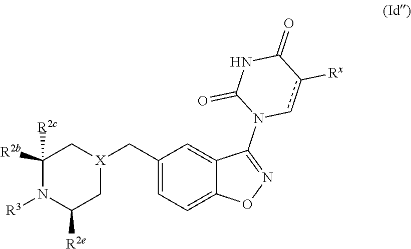

R^{2b}, R^{2c} and R^{2e} are defined according to any one of Embodiments 15 to 20.
   Embodiment 22. The compound according to any one of the Embodiments 1, 2, 4 to 10, 15, 16 and 18 to 21, or a pharmaceutically acceptable salt thereof, of Formula (Id′), wherein:

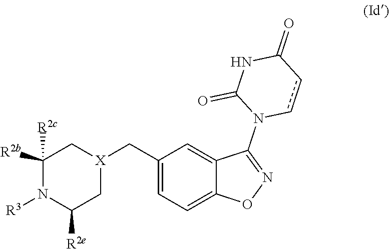

R^{2b}, R^{2c} and R^{2e} are defined according to any one of Embodiments 15 to 20.
   Embodiment 23. The compound according to any one of the Embodiments 1 to 11 and 15 to 22, or a pharmaceutically acceptable salt thereof, of Formula (Id), wherein:

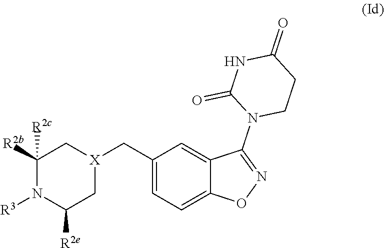

R^{2b}, R^{2c} and R^{2e} are defined according to any one of Embodiments 15 to 20.
   Embodiment 24. The compound according to any one of Embodiments 1, 4 to 9, 12, 15 and 18 to 21, or a pharmaceutically acceptable salt thereof, of Formula (Ie″), wherein:

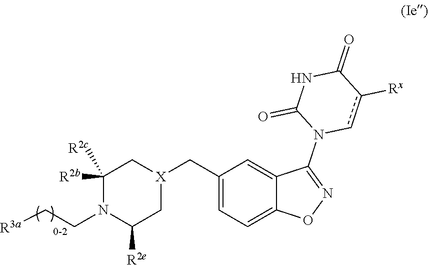

R^{2b}, R^{2c} and R^{2e} are defined according to any one of Embodiments 15 to 20.
   Embodiment 25. The compound according to any one of Embodiments 1, 2, 4 to 10, 12, 13, 15, 16, 18 to 22 and 24, or a pharmaceutically acceptable salt thereof, of Formula (Ie′), wherein:

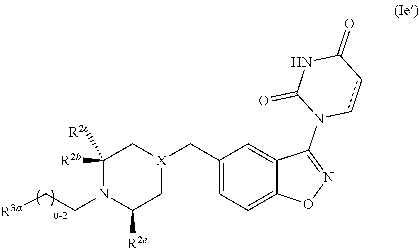

R^{2b}, R^{2c} and R^{2e} are defined according to any one of Embodiments 15 to 20.
   Embodiment 26. The compound according to any one of the preceding Embodiments, or a pharmaceutically acceptable salt thereof, of Formula (Ie), wherein:

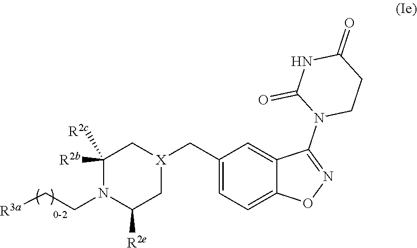

R^{2b}, R^{2c} and R^{2e} are defined according to any one of Embodiments 15 to 20.
   Embodiment 27. The compound according to any one of the preceding Embodiments, or a pharmaceutically acceptable salt thereof, wherein X is CH.
   Embodiment 28. The compound according to any one of the preceding Embodiments, or a pharmaceutically acceptable salt thereof, wherein X is N.
   Embodiment 29. The compound according to any one of Embodiments 1 to 14, or a pharmaceutically acceptable salt thereof, wherein R² is unsubstituted C₁-C₆alkyl and n is 1.
   Embodiment 30. The compound according to any one of the preceding Embodiments, wherein m is 1.
   Embodiment 31. The compound according to any one of Embodiments 1 to 11, 15 to 23 and 27 to 30, or a pharmaceutically acceptable salt thereof, wherein R³ is C₁-C₆alkyl, wherein the C₁-C₆alkyl is substituted with 1 occurrence of R^{3a}.
   Embodiment 32. The compound according to any one of Embodiments 1 to 11, 15 to 23 and 27 to 30, or a pharmaceutically acceptable salt thereof, wherein R³ is selected from methyl, ethyl, n-propyl, i-propyl, and —CH₂—(CH₂)₀₋₁—R^{3a}.
   Embodiment 33. The compound according to any one of the preceding Embodiments, or a pharmaceutically acceptable salt thereof, wherein R^{3a} is C₃-C₇cycloalkyl, wherein the C₃-C₇cycloalkyl is substituted with 0-4 occurrences of R^{3b}, wherein each R^{3b} is independently selected from chloro, fluoro, C₁-C₆haloalkyl, C₁-C₆haloalkoxyl and C₁-C₆alkyl.
   Embodiment 34. The compound according to any one of the preceding Embodiments, or a pharmaceutically acceptable salt thereof, wherein R^{3a} is C₃-C₇cycloalkyl, wherein the C₃-C₇cycloalkyl is substituted with 0-2 occurrences of fluoro.
   Embodiment 35. The compound according to any one of the preceding Embodiments, or a pharmaceutically acceptable salt thereof, wherein R^{3a} is selected from cyclopropyl, cyclobutyl, cyclohexyl, and

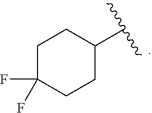 [SMILES: *C1CCC(F)(F)CC1]

Embodiment 36. The compound according to any one of Embodiments 15 to 28 and 30 to 35, or a pharmaceutically acceptable salt thereof, wherein each of R^{2b} and R^{2e} is independently selected from hydrogen and unsubstituted C₁-C₃alkyl; and R^{2c} is hydrogen.
Embodiment 37. The compound according to any one of Embodiments 15 to 28 and 30 to 36, or a pharmaceutically acceptable salt thereof, wherein each of R^{2b} and R^{2e} is independently selected from hydrogen and methyl; and R^{2c} is hydrogen.
Embodiment 38. The compound according to any one of Embodiments 15 to 28 and 30 to 36, or a pharmaceutically acceptable salt thereof, wherein
R^{2b} is unsubstituted C₁-C₃alkyl (e.g., methyl);
R^{2c} is hydrogen; and
R^{2e} is selected from hydrogen and unsubstituted C₁-C₃alkyl.
   Embodiment 39. The compound according to any one of Embodiments 15 to 28 and 30 to 38, or a pharmaceutically acceptable salt thereof, wherein R^{2b} is methyl and R^{2c}, R^{2d}, R^{2e} and R^{2f} are all hydrogen.
   Embodiment 40. The compound according to any one of Embodiments 1 to 14 and 27 to 35, or a pharmaceutically acceptable salt thereof, wherein R^{z} is unsubstituted C₁-C₃alkyl, and n is 1.
   Embodiment 41. The compound according to any one of Embodiments 1, 2, 4 to 10, 12, 13, 15, 16, 18 to 22, 24, 25 and 27 to 40, wherein [symbol] is a double bond.
   Embodiment 42. The compound according to any one of the preceding Embodiments, wherein [symbol] is a single bond.
   Embodiment 43. The compound according to Embodiment 1 or a pharmaceutically acceptable salt thereof, selected from:

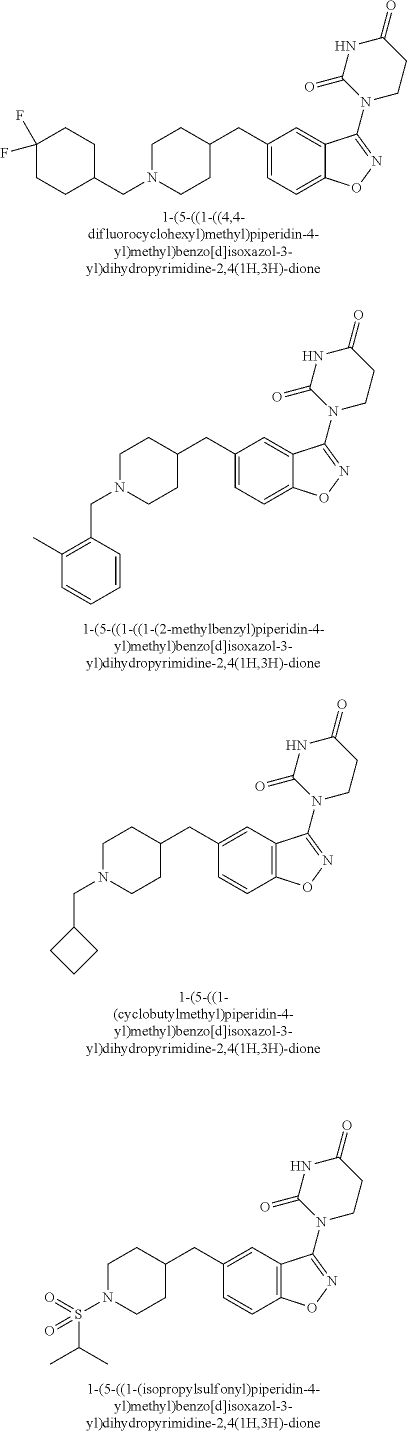 [SMILES: O=C1CCN(c2noc3ccc(CC4CCN(CC5CCC(F)(F)CC5)CC4)cc23)C(=O)N1 ; Cc1ccccc1CN1CCC(Cc2ccc3onc(N4CCC(=O)NC4=O)c3c2)CC1 ; O=C1CCN(c2noc3ccc(CC4CCN(CC5CCC5)CC4)cc23)C(=O)N1 ; CC(C)S(=O)(=O)N1CCC(Cc2ccc3onc(N4CCC(=O)NC4=O)c3c2)CC1]

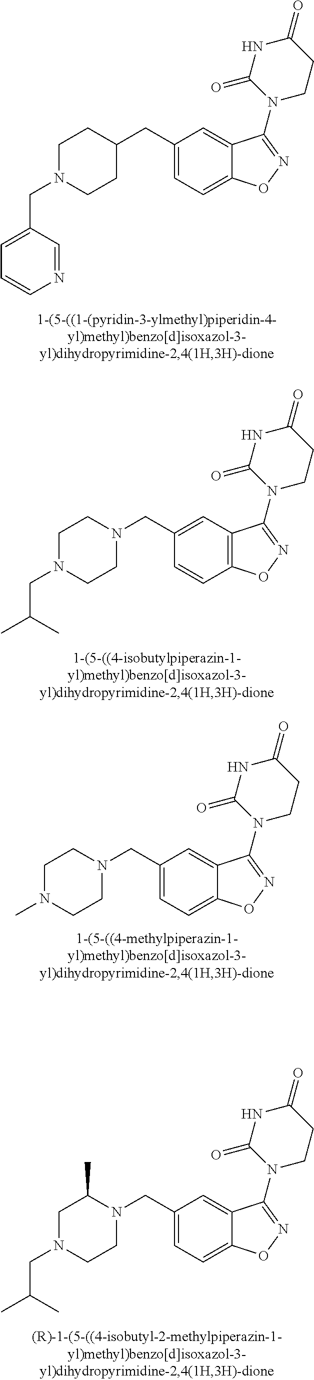 [SMILES: O=C1CCN(c2noc3ccc(CC4CCN(Cc5cccnc5)CC4)cc23)C(=O)N1 ; CC(C)CN1CCN(Cc2ccc3onc(N4CCC(=O)NC4=O)c3c2)CC1 ; CN1CCN(Cc2ccc3onc(N4CCC(=O)NC4=O)c3c2)CC1 ; CC(C)CN1CCN(Cc2ccc3onc(N4CCC(=O)NC4=O)c3c2)[C@H](C)C1]

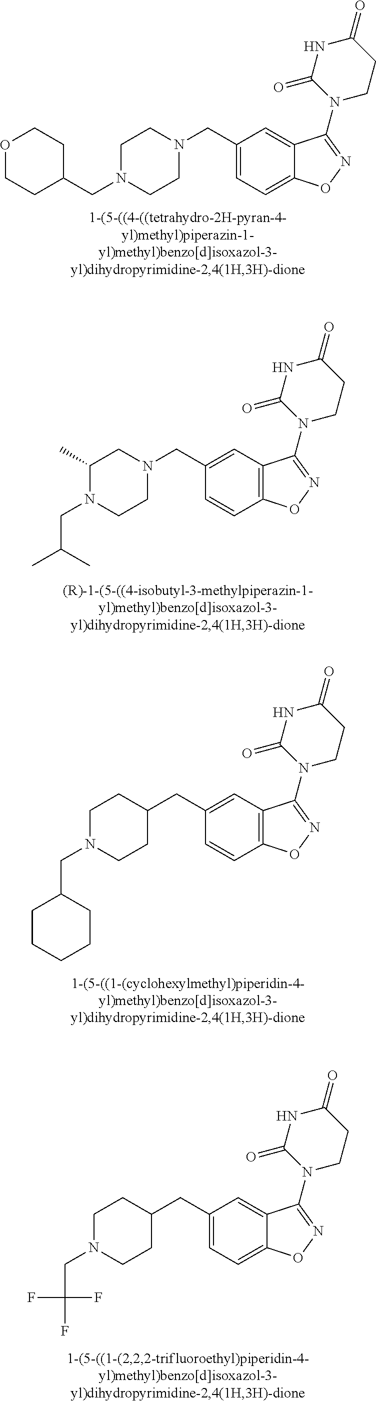 [SMILES: O=C1CCN(c2noc3ccc(CN4CCN(CC5CCOCC5)CC4)cc23)C(=O)N1 ; CC(C)CN1CCN(Cc2ccc3onc(N4CCC(=O)NC4=O)c3c2)C[C@H]1C ; O=C1CCN(c2noc3ccc(CC4CCN(CC5CCCCC5)CC4)cc23)C(=O)N1 ; O=C1CCN(c2noc3ccc(CC4CCN(CC(F)(F)F)CC4)cc23)C(=O)N1]

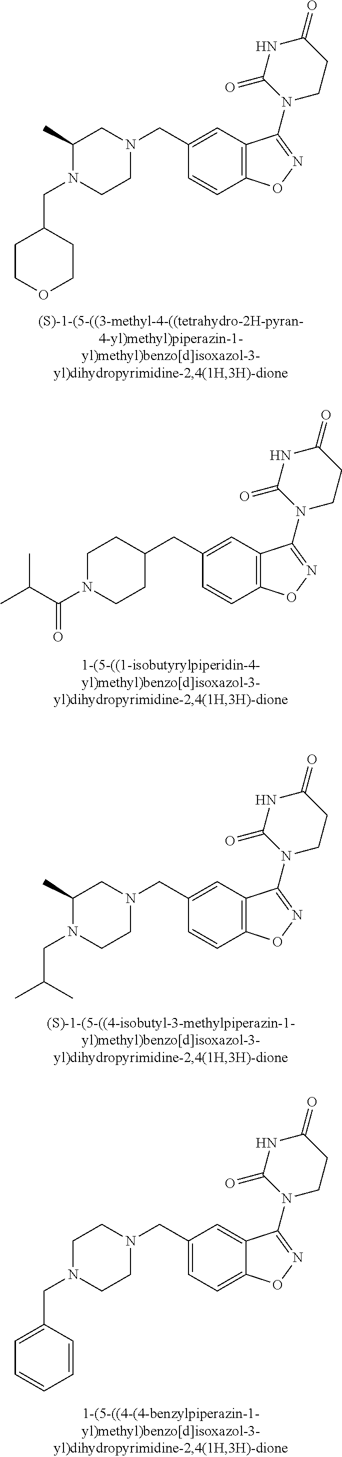 [SMILES: C[C@H]1CN(Cc2ccc3onc(N4CCC(=O)NC4=O)c3c2)CCN1CC1CCOCC1 ; CC(C)C(=O)N1CCC(Cc2ccc3onc(N4CCC(=O)NC4=O)c3c2)CC1 ; CC(C)CN1CCN(Cc2ccc3onc(N4CCC(=O)NC4=O)c3c2)C[C@@H]1C ; O=C1CCN(c2noc3ccc(CN4CCN(Cc5ccccc5)CC4)cc23)C(=O)N1]

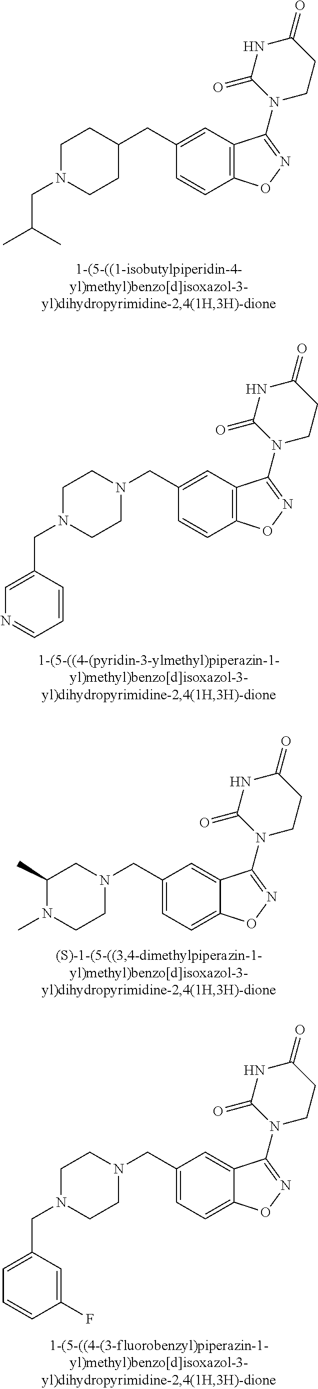 [SMILES: CC(C)CN1CCC(Cc2ccc3onc(N4CCC(=O)NC4=O)c3c2)CC1 ; O=C1CCN(c2noc3ccc(CN4CCN(Cc5cccnc5)CC4)cc23)C(=O)N1 ; C[C@H]1CN(Cc2ccc3onc(N4CCC(=O)NC4=O)c3c2)CCN1C ; O=C1CCN(c2noc3ccc(CN4CCN(Cc5cccc(F)c5)CC4)cc23)C(=O)N1]

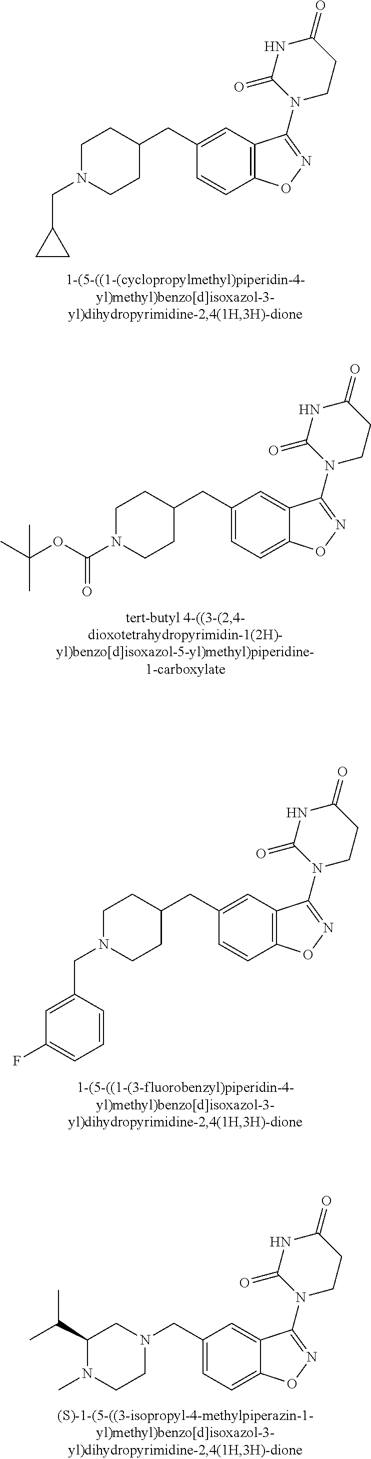 [SMILES: O=C1CCN(c2noc3ccc(CC4CCN(CC5CC5)CC4)cc23)C(=O)N1 ; CC(C)(C)OC(=O)N1CCC(Cc2ccc3onc(N4CCC(=O)NC4=O)c3c2)CC1 ; O=C1CCN(c2noc3ccc(CC4CCN(Cc5cccc(F)c5)CC4)cc23)C(=O)N1 ; CC(C)[C@H]1CN(Cc2ccc3onc(N4CCC(=O)NC4=O)c3c2)CCN1C]

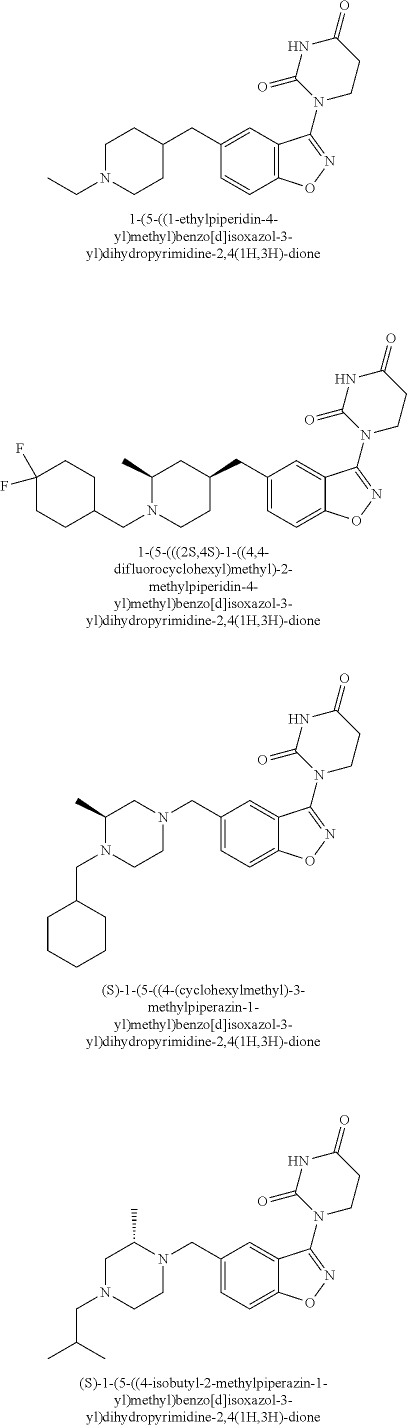 [SMILES: CCN1CCC(Cc2ccc3onc(N4CCC(=O)NC4=O)c3c2)CC1 ; C[C@H]1C[C@@H](Cc2ccc3onc(N4CCC(=O)NC4=O)c3c2)CCN1CC1CCC(F)(F)CC1 ; C[C@H]1CN(Cc2ccc3onc(N4CCC(=O)NC4=O)c3c2)CCN1CC1CCCCC1 ; CC(C)CN1CCN(Cc2ccc3onc(N4CCC(=O)NC4=O)c3c2)[C@@H](C)C1]

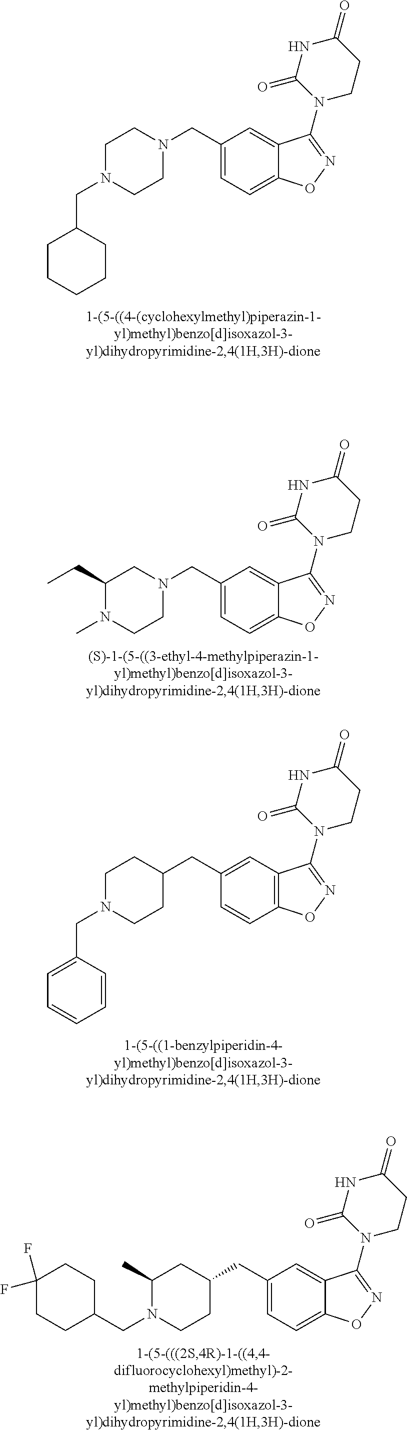 [SMILES: O=C1CCN(c2noc3ccc(CN4CCN(CC5CCCCC5)CC4)cc23)C(=O)N1 ; CC[C@H]1CN(Cc2ccc3onc(N4CCC(=O)NC4=O)c3c2)CCN1C ; O=C1CCN(c2noc3ccc(CC4CCN(Cc5ccccc5)CC4)cc23)C(=O)N1 ; C[C@H]1C[C@H](Cc2ccc3onc(N4CCC(=O)NC4=O)c3c2)CCN1CC1CCC(F)(F)CC1]

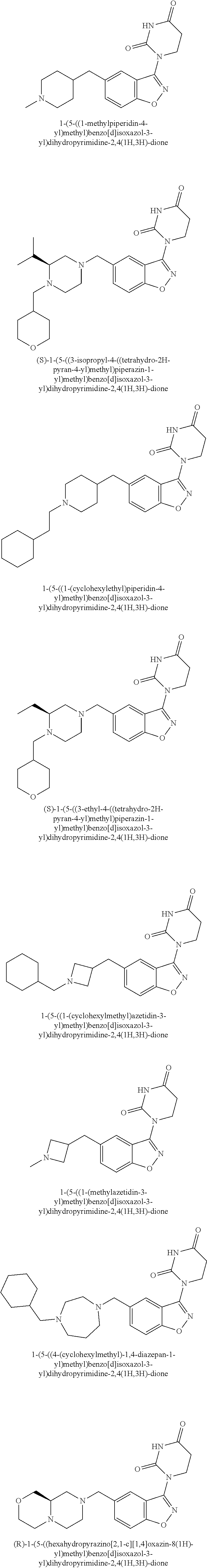

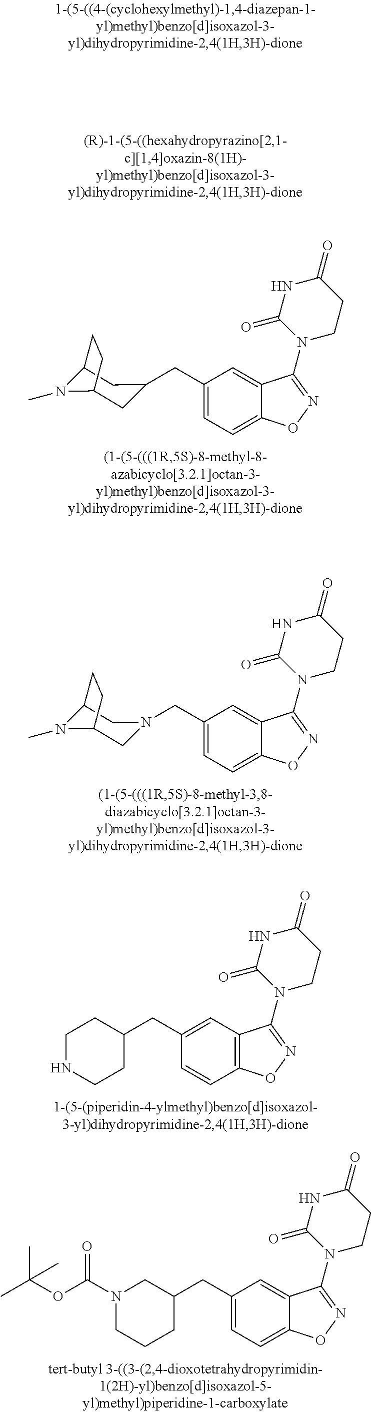 [SMILES: CN1C2CCC1CC(Cc1ccc3onc(N4CCC(=O)NC4=O)c3c1)C2 ; CN1C2CCC1CN(Cc1ccc3onc(N4CCC(=O)NC4=O)c3c1)C2 ; O=C1CCN(c2noc3ccc(CC4CCNCC4)cc23)C(=O)N1 ; CC(C)(C)OC(=O)N1CCCC(Cc2ccc3onc(N4CCC(=O)NC4=O)c3c2)C1]

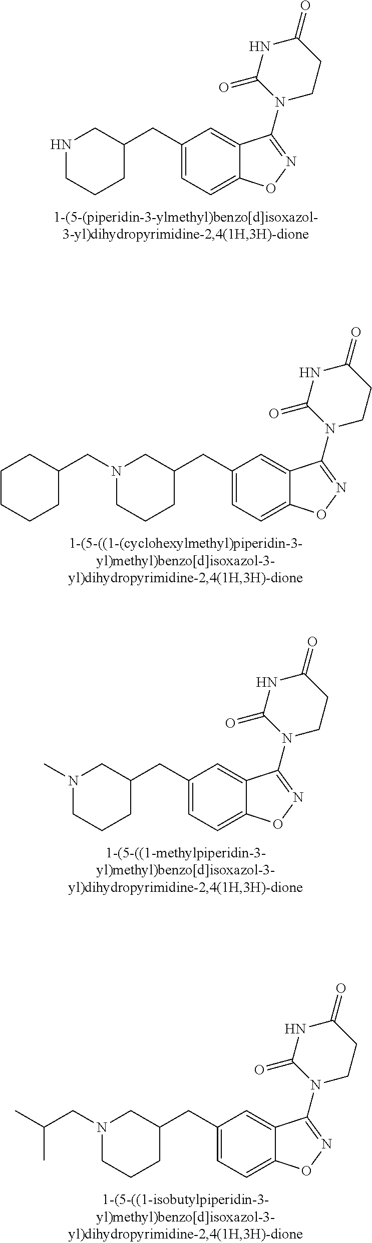 [SMILES: O=C1CCN(c2noc3ccc(CC4CCCNC4)cc23)C(=O)N1 ; O=C1CCN(c2noc3ccc(CC4CCCN(CC5CCCCC5)C4)cc23)C(=O)N1 ; CN1CCCC(Cc2ccc3onc(N4CCC(=O)NC4=O)c3c2)C1 ; CC(C)CN1CCCC(Cc2ccc3onc(N4CCC(=O)NC4=O)c3c2)C1]

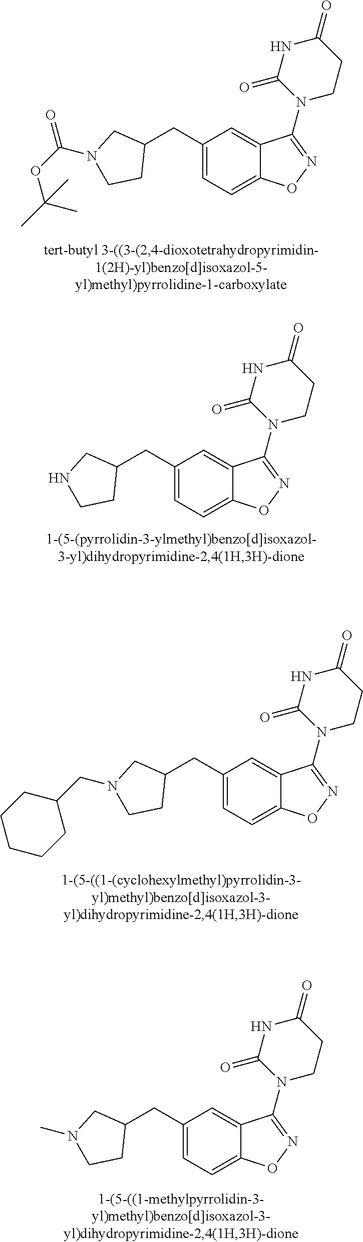 [SMILES: CC(C)(C)OC(=O)N1CCC(Cc2ccc3onc(N4CCC(=O)NC4=O)c3c2)C1 ; O=C1CCN(c2noc3ccc(CC4CCNC4)cc23)C(=O)N1 ; O=C1CCN(c2noc3ccc(CC4CCN(CC5CCCCC5)C4)cc23)C(=O)N1 ; CN1CCC(Cc2ccc3onc(N4CCC(=O)NC4=O)c3c2)C1]

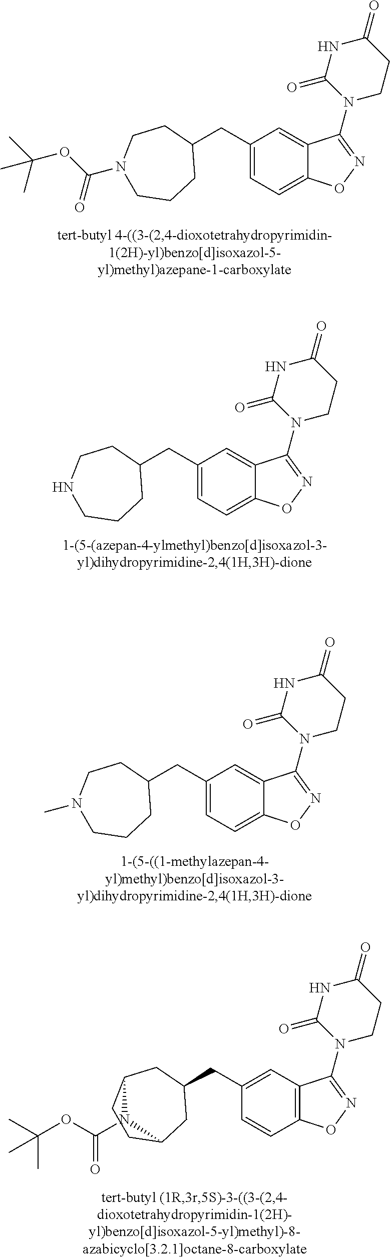 [SMILES: CC(C)(C)OC(=O)N1CCCC(Cc2ccc3onc(N4CCC(=O)NC4=O)c3c2)CC1 ; O=C1CCN(c2noc3ccc(CC4CCCNCC4)cc23)C(=O)N1 ; CN1CCCC(Cc2ccc3onc(N4CCC(=O)NC4=O)c3c2)CC1 ; CC(C)(C)OC(=O)N1[C@@H]2CC[C@H]1C[C@@H](Cc1ccc3onc(N4CCC(=O)NC4=O)c3c1)C2]

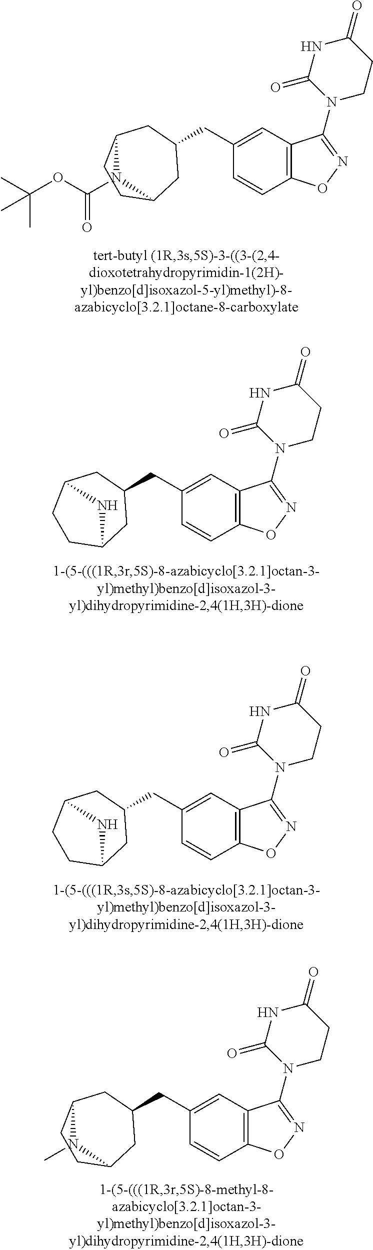 [SMILES: CC(C)(C)OC(=O)N1[C@@H]2CC[C@H]1C[C@H](Cc1ccc3onc(N4CCC(=O)NC4=O)c3c1)C2 ; O=C1CCN(c2noc3ccc(C[C@H]4C[C@H]5CC[C@@H](C4)N5)cc23)C(=O)N1 ; O=C1CCN(c2noc3ccc(C[C@@H]4C[C@H]5CC[C@@H](C4)N5)cc23)C(=O)N1 ; CN1[C@@H]2CC[C@H]1C[C@@H](Cc1ccc3onc(N4CCC(=O)NC4=O)c3c1)C2]

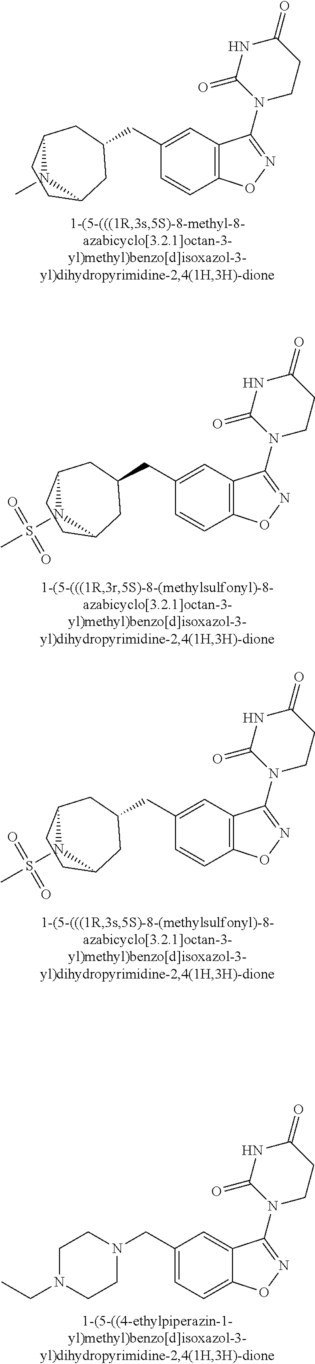 [SMILES: CN1[C@@H]2CC[C@H]1C[C@H](Cc1ccc3onc(N4CCC(=O)NC4=O)c3c1)C2 ; CS(=O)(=O)N1[C@@H]2CC[C@H]1C[C@@H](Cc1ccc3onc(N4CCC(=O)NC4=O)c3c1)C2 ; CS(=O)(=O)N1[C@@H]2CC[C@H]1C[C@H](Cc1ccc3onc(N4CCC(=O)NC4=O)c3c1)C2 ; CCN1CCN(Cc2ccc3onc(N4CCC(=O)NC4=O)c3c2)CC1]

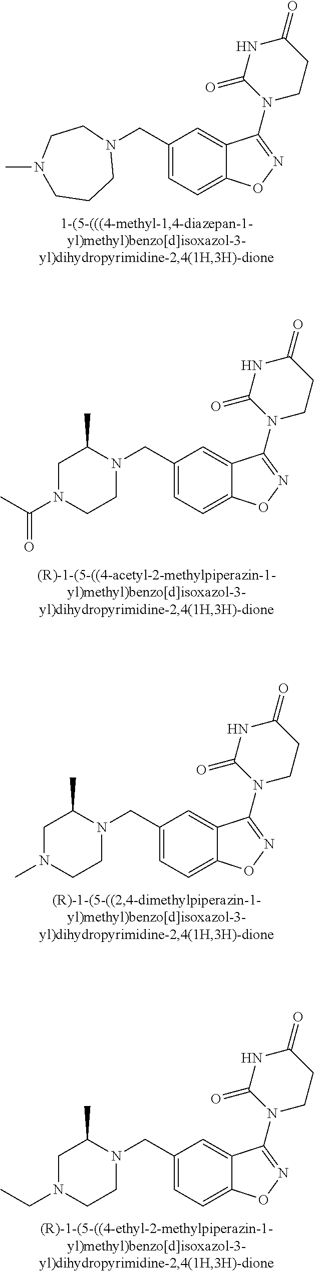 [SMILES: CN1CCCN(Cc2ccc3onc(N4CCC(=O)NC4=O)c3c2)CC1 ; CC(=O)N1CCN(Cc2ccc3onc(N4CCC(=O)NC4=O)c3c2)[C@H](C)C1 ; C[C@@H]1CN(C)CCN1Cc1ccc2onc(N3CCC(=O)NC3=O)c2c1 ; CCN1CCN(Cc2ccc3onc(N4CCC(=O)NC4=O)c3c2)[C@H](C)C1]

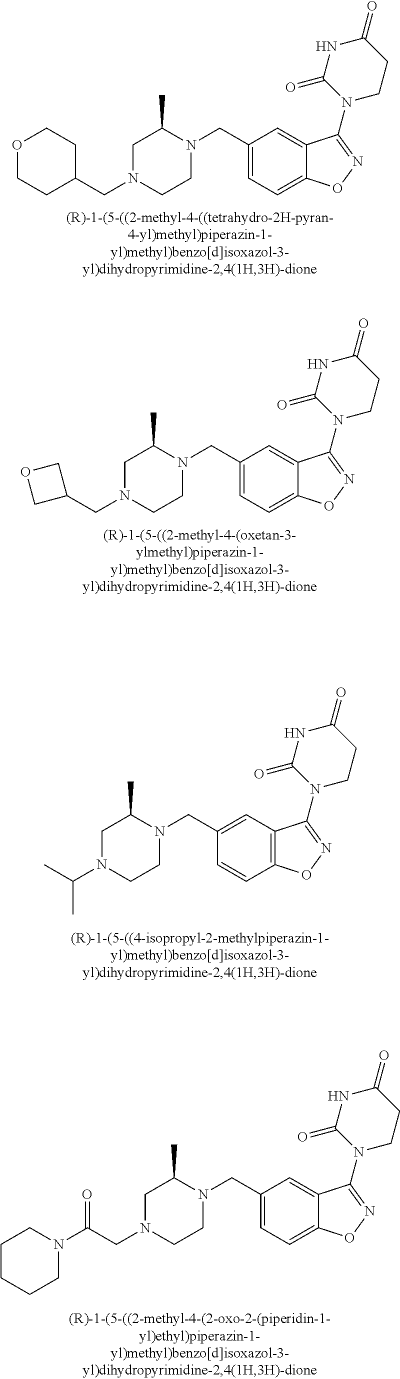 [SMILES: C[C@@H]1CN(CC2CCOCC2)CCN1Cc1ccc2onc(N3CCC(=O)NC3=O)c2c1 ; C[C@@H]1CN(CC2COC2)CCN1Cc1ccc2onc(N3CCC(=O)NC3=O)c2c1 ; CC(C)N1CCN(Cc2ccc3onc(N4CCC(=O)NC4=O)c3c2)[C@H](C)C1 ; C[C@@H]1CN(CC(=O)N2CCCCC2)CCN1Cc1ccc2onc(N3CCC(=O)NC3=O)c2c1]

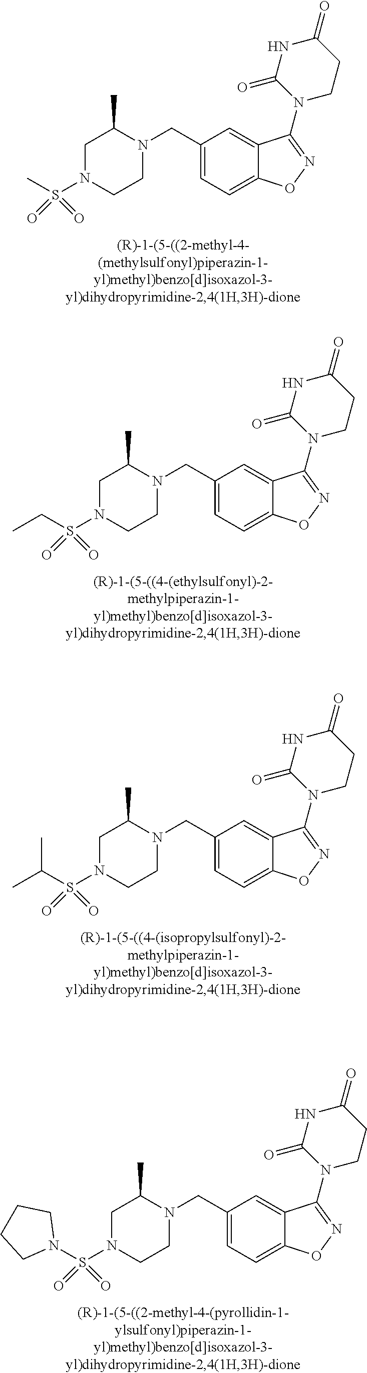 [SMILES: C[C@@H]1CN(S(C)(=O)=O)CCN1Cc1ccc2onc(N3CCC(=O)NC3=O)c2c1 ; CCS(=O)(=O)N1CCN(Cc2ccc3onc(N4CCC(=O)NC4=O)c3c2)[C@H](C)C1 ; CC(C)S(=O)(=O)N1CCN(Cc2ccc3onc(N4CCC(=O)NC4=O)c3c2)[C@H](C)C1 ; C[C@@H]1CN(S(=O)(=O)N2CCCC2)CCN1Cc1ccc2onc(N3CCC(=O)NC3=O)c2c1]

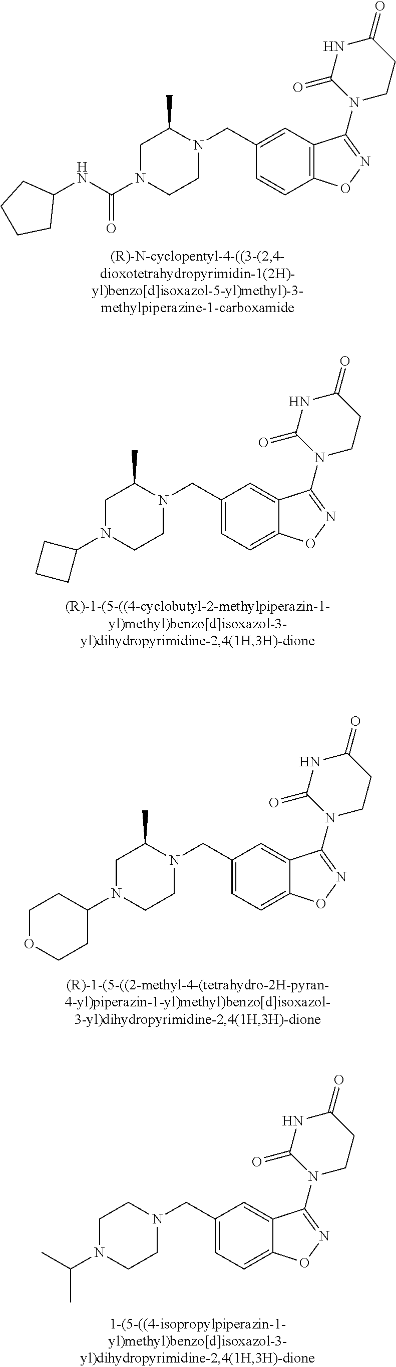 [SMILES: C[C@@H]1CN(C(=O)NC2CCCC2)CCN1Cc1ccc2onc(N3CCC(=O)NC3=O)c2c1 ; C[C@@H]1CN(C2CCC2)CCN1Cc1ccc2onc(N3CCC(=O)NC3=O)c2c1 ; C[C@@H]1CN(C2CCOCC2)CCN1Cc1ccc2onc(N3CCC(=O)NC3=O)c2c1 ; CC(C)N1CCN(Cc2ccc3onc(N4CCC(=O)NC4=O)c3c2)CC1]

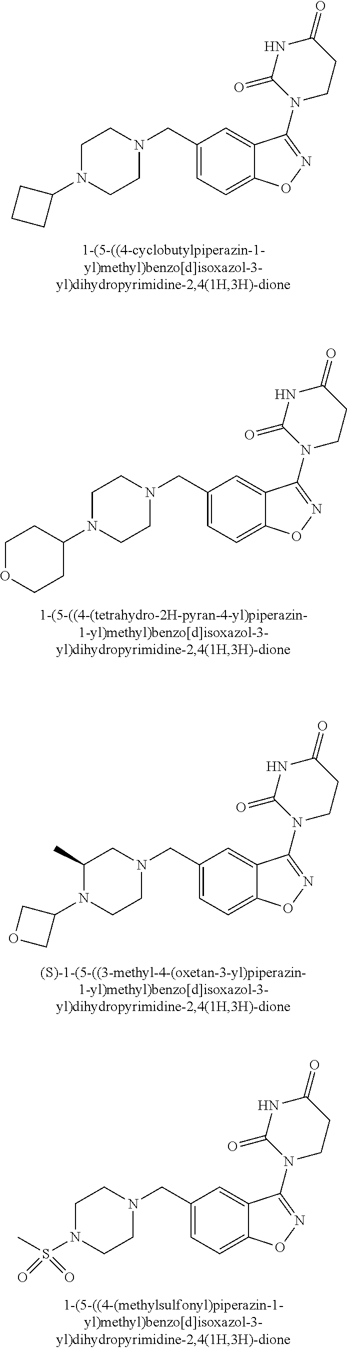 [SMILES: O=C1CCN(c2noc3ccc(CN4CCN(C5CCC5)CC4)cc23)C(=O)N1 ; O=C1CCN(c2noc3ccc(CN4CCN(C5CCOCC5)CC4)cc23)C(=O)N1 ; C[C@H]1CN(Cc2ccc3onc(N4CCC(=O)NC4=O)c3c2)CCN1C1COC1 ; CS(=O)(=O)N1CCN(Cc2ccc3onc(N4CCC(=O)NC4=O)c3c2)CC1]

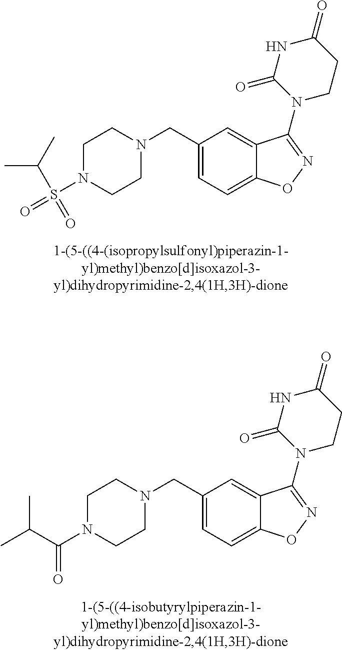 [SMILES: CC(C)S(=O)(=O)N1CCN(Cc2ccc3onc(N4CCC(=O)NC4=O)c3c2)CC1 ; CC(C)C(=O)N1CCN(Cc2ccc3onc(N4CCC(=O)NC4=O)c3c2)CC1]

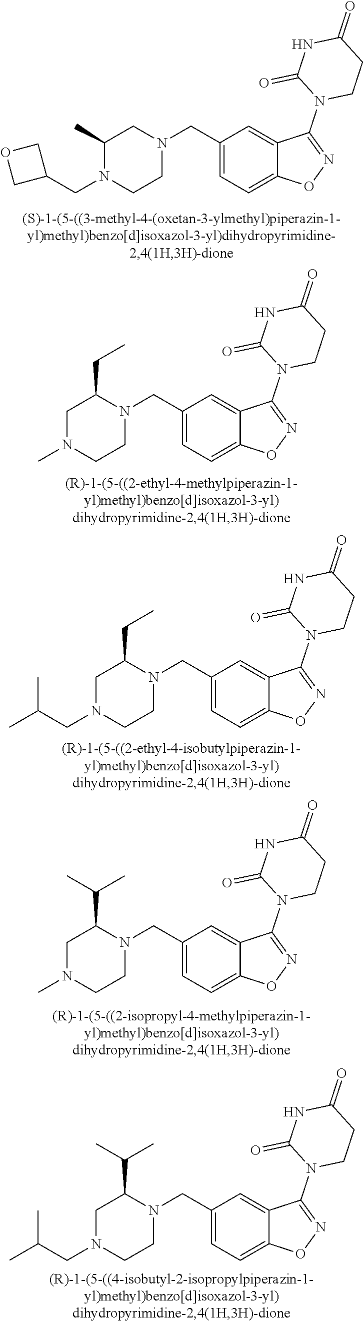 [SMILES: C[C@H]1CN(Cc2ccc3onc(N4CCC(=O)NC4=O)c3c2)CCN1CC1COC1 ; CC[C@@H]1CN(C)CCN1Cc1ccc2onc(N3CCC(=O)NC3=O)c2c1 ; CC[C@@H]1CN(CC(C)C)CCN1Cc1ccc2onc(N3CCC(=O)NC3=O)c2c1 ; CC(C)[C@@H]1CN(C)CCN1Cc1ccc2onc(N3CCC(=O)NC3=O)c2c1 ; CC(C)CN1CCN(Cc2ccc3onc(N4CCC(=O)NC4=O)c3c2)[C@H](C(C)C)C1]

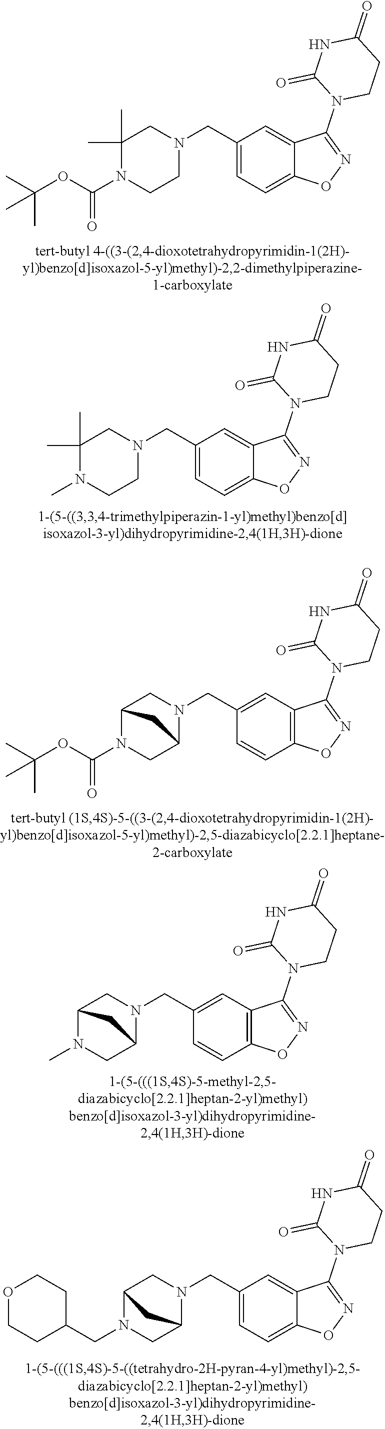 [SMILES: CC(C)(C)OC(=O)N1CCN(Cc2ccc3onc(N4CCC(=O)NC4=O)c3c2)CC1(C)C ; CN1CCN(Cc2ccc3onc(N4CCC(=O)NC4=O)c3c2)CC1(C)C ; CC(C)(C)OC(=O)N1C[C@@H]2C[C@H]1CN2Cc1ccc2onc(N3CCC(=O)NC3=O)c2c1 ; CN1C[C@@H]2C[C@H]1CN2Cc1ccc2onc(N3CCC(=O)NC3=O)c2c1 ; O=C1CCN(c2noc3ccc(CN4C[C@@H]5C[C@H]4CN5CC4CCOCC4)cc23)C(=O)N1]

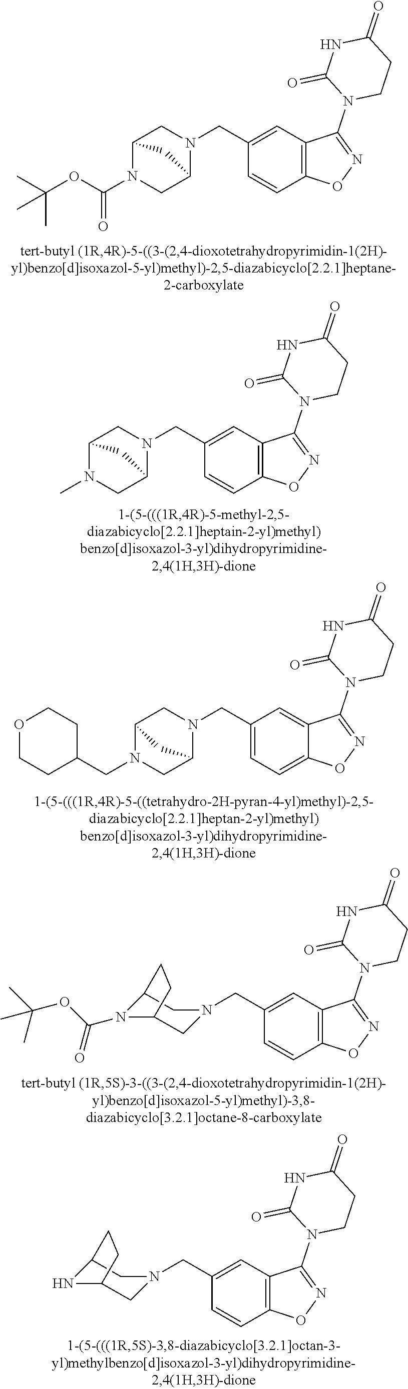 [SMILES: CC(C)(C)OC(=O)N1C[C@H]2C[C@@H]1CN2Cc1ccc2onc(N3CCC(=O)NC3=O)c2c1 ; CN1C[C@H]2C[C@@H]1CN2Cc1ccc2onc(N3CCC(=O)NC3=O)c2c1 ; O=C1CCN(c2noc3ccc(CN4C[C@H]5C[C@@H]4CN5CC4CCOCC4)cc23)C(=O)N1 ; CC(C)(C)OC(=O)N1C2CCC1CN(Cc1ccc3onc(N4CCC(=O)NC4=O)c3c1)C2 ; O=C1CCN(c2noc3ccc(CN4CC5CCC(C4)N5)cc23)C(=O)N1]

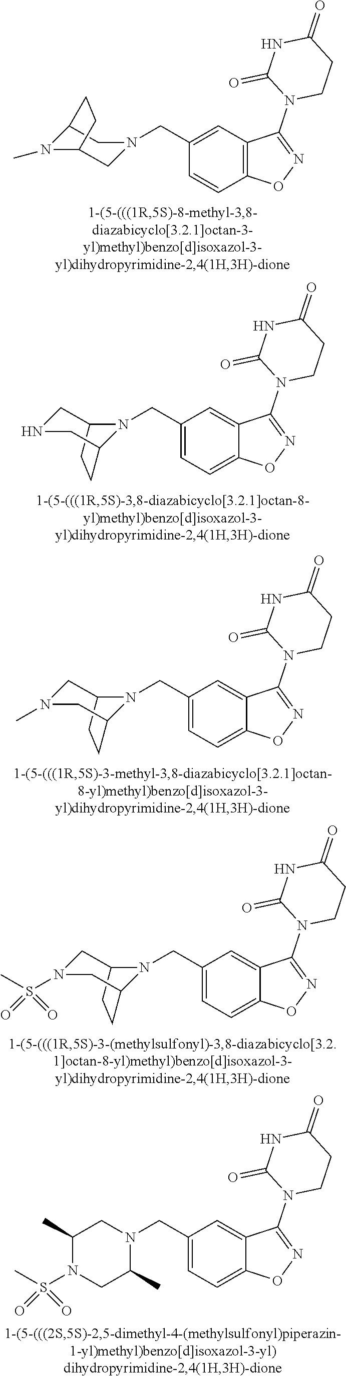 [SMILES: CN1C2CCC1CN(Cc1ccc3onc(N4CCC(=O)NC4=O)c3c1)C2 ; O=C1CCN(c2noc3ccc(CN4C5CCC4CNC5)cc23)C(=O)N1 ; CN1CC2CCC(C1)N2Cc1ccc2onc(N3CCC(=O)NC3=O)c2c1 ; CS(=O)(=O)N1CC2CCC(C1)N2Cc1ccc2onc(N3CCC(=O)NC3=O)c2c1 ; C[C@H]1CN(S(C)(=O)=O)[C@@H](C)CN1Cc1ccc2onc(N3CCC(=O)NC3=O)c2c1]

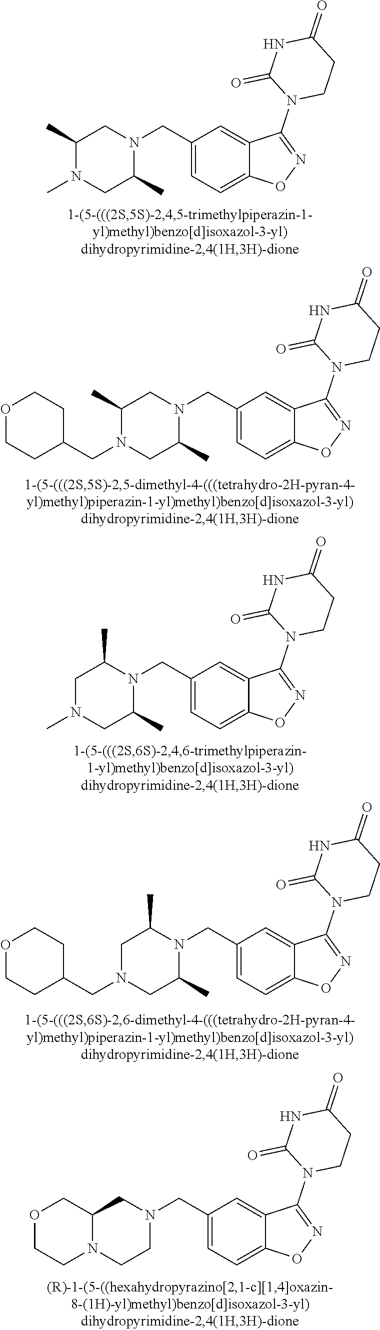 [SMILES: C[C@H]1CN(Cc2ccc3onc(N4CCC(=O)NC4=O)c3c2)[C@@H](C)CN1C ; C[C@H]1CN(CC2CCOCC2)[C@@H](C)CN1Cc1ccc2onc(N3CCC(=O)NC3=O)c2c1 ; C[C@@H]1CN(C)C[C@H](C)N1Cc1ccc2onc(N3CCC(=O)NC3=O)c2c1 ; C[C@@H]1CN(CC2CCOCC2)C[C@H](C)N1Cc1ccc2onc(N3CCC(=O)NC3=O)c2c1 ; O=C1CCN(c2noc3ccc(CN4CCN5CCOC[C@H]5C4)cc23)C(=O)N1]

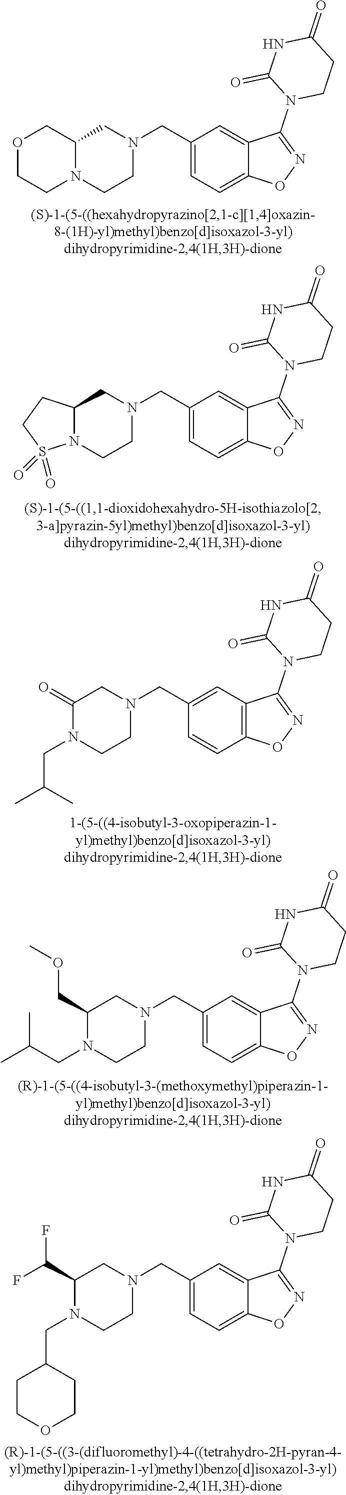 [SMILES: O=C1CCN(c2noc3ccc(CN4CCN5CCOC[C@@H]5C4)cc23)C(=O)N1 ; O=C1CCN(c2noc3ccc(CN4CCN5[C@@H](CCS5(=O)=O)C4)cc23)C(=O)N1 ; CC(C)CN1CCN(Cc2ccc3onc(N4CCC(=O)NC4=O)c3c2)CC1=O ; COC[C@H]1CN(Cc2ccc3onc(N4CCC(=O)NC4=O)c3c2)CCN1CC(C)C ; O=C1CCN(c2noc3ccc(CN4CCN(CC5CCOCC5)[C@@H](C(F)F)C4)cc23)C(=O)N1]

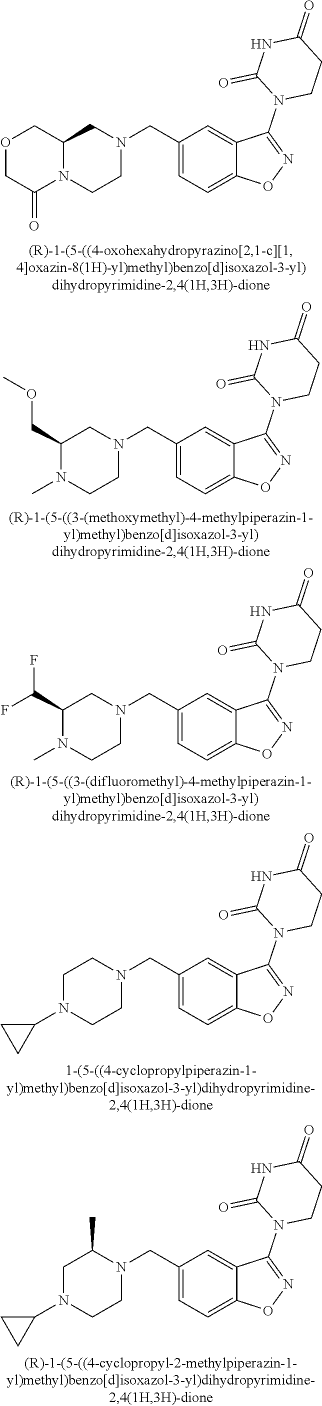 [SMILES: O=C1CCN(c2noc3ccc(CN4CCN5C(=O)COC[C@H]5C4)cc23)C(=O)N1 ; COC[C@H]1CN(Cc2ccc3onc(N4CCC(=O)NC4=O)c3c2)CCN1C ; CN1CCN(Cc2ccc3onc(N4CCC(=O)NC4=O)c3c2)C[C@@H]1C(F)F ; O=C1CCN(c2noc3ccc(CN4CCN(C5CC5)CC4)cc23)C(=O)N1 ; C[C@@H]1CN(C2CC2)CCN1Cc1ccc2onc(N3CCC(=O)NC3=O)c2c1]

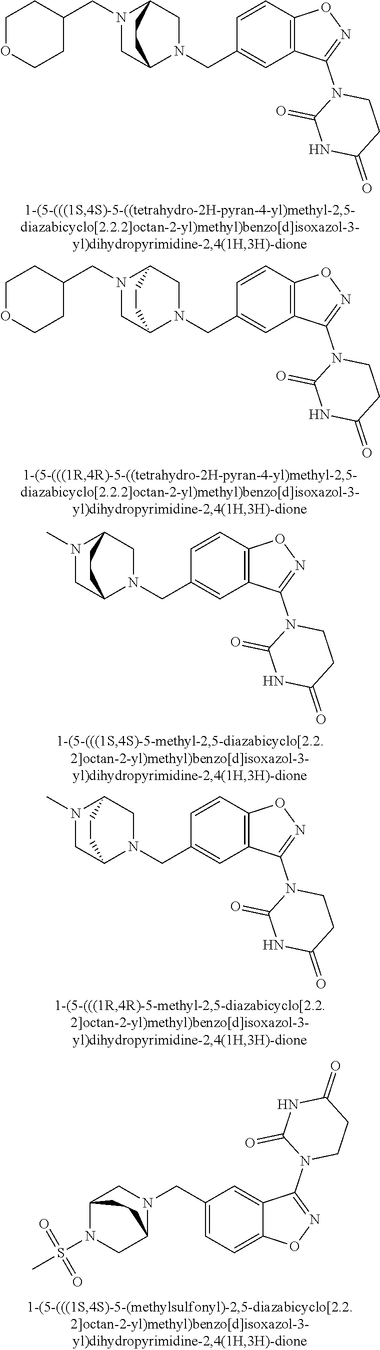 [SMILES: O=C1CCN(c2noc3ccc(CN4C[C@@H]5CC[C@H]4CN5CC4CCOCC4)cc23)C(=O)N1 ; O=C1CCN(c2noc3ccc(CN4C[C@H]5CC[C@@H]4CN5CC4CCOCC4)cc23)C(=O)N1 ; CN1C[C@@H]2CC[C@H]1CN2Cc1ccc2onc(N3CCC(=O)NC3=O)c2c1 ; CN1C[C@H]2CC[C@@H]1CN2Cc1ccc2onc(N3CCC(=O)NC3=O)c2c1 ; CS(=O)(=O)N1C[C@@H]2CC[C@H]1CN2Cc1ccc2onc(N3CCC(=O)NC3=O)c2c1]

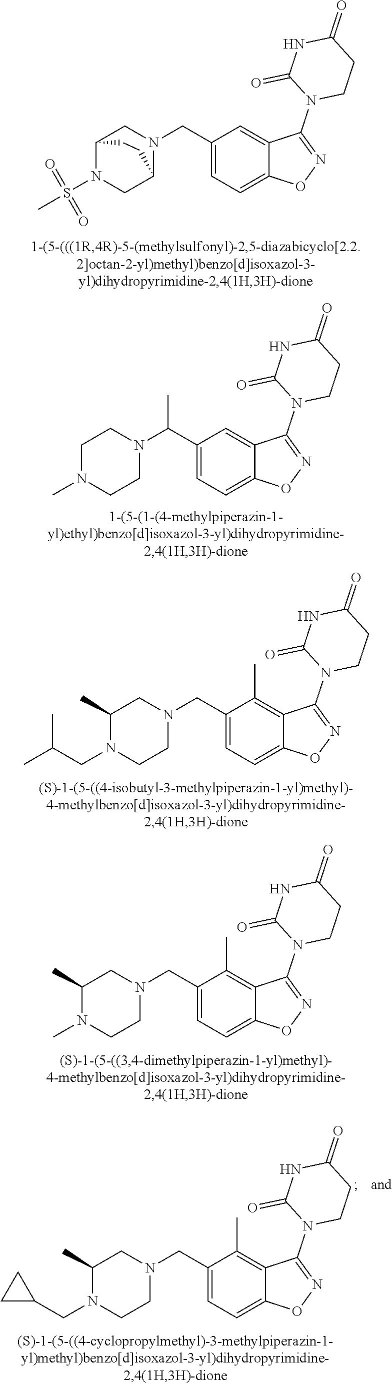 [SMILES: CS(=O)(=O)N1C[C@H]2CC[C@@H]1CN2Cc1ccc2onc(N3CCC(=O)NC3=O)c2c1 ; CC(c1ccc2onc(N3CCC(=O)NC3=O)c2c1)N1CCN(C)CC1 ; Cc1c(CN2CCN(CC(C)C)[C@@H](C)C2)ccc2onc(N3CCC(=O)NC3=O)c12 ; Cc1c(CN2CCN(C)[C@@H](C)C2)ccc2onc(N3CCC(=O)NC3=O)c12 ; Cc1c(CN2CCN(CC3CC3)[C@@H](C)C2)ccc2onc(N3CCC(=O)NC3=O)c12]

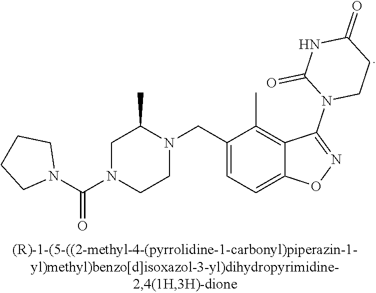 [SMILES: Cc1c(CN2CCN(C(=O)N3CCCC3)C[C@H]2C)ccc2onc(N3CCC(=O)NC3=O)c12]

Embodiment 44. The compound according to any one of the preceding Embodiments, or a pharmaceutically acceptable salt thereof, wherein the pharmaceutically acceptable salt is an acid addition salt.
Embodiment 45. A pharmaceutical composition comprising a therapeutically effective amount of a compound according to any one of Embodiments 1 to 44, or a pharmaceutically acceptable salt thereof, and a pharmaceutically acceptable carrier or excipient.
Embodiment 46. A compound according to any one of Embodiments 1 to 44, or a pharmaceutically acceptable salt thereof, for use as a medicament.
Embodiment 47. A method of treating or preventing a disease or disorder in a subject in need thereof, the method comprising administering to the subject a therapeutically effective amount of a compound according to any one of Embodiments 1 to 44, or a pharmaceutically acceptable salt, thereof.
Embodiment 48. A method of treating or preventing a disorder that is affected by the reduction of WIZ protein levels, in a subject in need thereof, the method comprising administering to the subject a therapeutically effective amount of a compound according to any one of Embodiments 1 to 44, or a pharmaceutically acceptable salt thereof.
Embodiment 49. A method of treating a disease or disorder that is affected by the modulation of WIZ protein levels comprising administering to the patient in need thereof a compound according to any one of Embodiments 1 to 44, or a pharmaceutically acceptable salt thereof.
Embodiment 50. A method of inhibiting WIZ protein expression in a subject in need thereof, the method comprising administering to the subject a therapeutically effective amount of a compound according to any one of Embodiments 1 to 44, or a pharmaceutically acceptable salt thereof.
Embodiment 51. A method of degrading WIZ protein in a subject in need thereof, the method comprising administering to the subject a therapeutically effective amount of a compound according to any one of Embodiments 1 to 44, or a pharmaceutically acceptable salt, thereof.
Embodiment 52. A method of inhibiting, reducing, or eliminating the activity of WIZ protein or WIZ protein expression, the method comprising administering to the subject a compound according to any one of Embodiments 1 to 44, or a pharmaceutically acceptable salt thereof.
Embodiment 53. A method of inducing or promoting fetal hemoglobin in a subject in need thereof, the method comprising administering to the subject a therapeutically effective amount of a compound according to any one of Embodiments 1 to 44, or a pharmaceutically acceptable salt thereof.
Embodiment 54. A method of reactivating fetal hemoglobin production or expression in a subject in need thereof, the method comprising administering to the subject a therapeutically effective amount of a compound according to any one of Embodiments 1 to 44, or a pharmaceutically acceptable salt thereof.
Embodiment 55. A method of increasing fetal hemoglobin expression in a subject in need thereof, the method comprising administering to the subject a therapeutically effective amount of a compound according to any one of Embodiments 1 to 44, or a pharmaceutically acceptable salt thereof.
Embodiment 56. A method of treating a hemoglobinopathy, e.g., a beta-hemoglobinopathy, in a subject in need thereof, the method comprising administering to the subject a therapeutically effective amount of a compound according to any one of Embodiments 1 to 44, or a pharmaceutically acceptable salt thereof.
Embodiment 57. A method of treating a sickle cell disease in a subject in need thereof, the method comprising administering to the subject a therapeutically effective amount of a compound according to any one of Embodiments 1 to 44, or a pharmaceutically acceptable salt thereof.
Embodiment 58. A method of treating beta-thalassemia in a subject in need thereof, the method comprising administering to the subject a therapeutically effective amount of a compound according to any one of Embodiments 1 to 44, or a pharmaceutically acceptable salt thereof.
Embodiment 59. A method for reducing WIZ protein levels in a subject comprising the step of administering to a subject in need thereof a therapeutically effective amount of a compound according to any one of Embodiments 1 to 44, or a pharmaceutically acceptable salt thereof.
Embodiment 60. A compound according to any one of Embodiments 1 to 44, or a pharmaceutically acceptable salt thereof, for use in treating or preventing a disease or disorder in a subject in need thereof.
Embodiment 61. A compound according to any one of Embodiments 1 to 44, or a pharmaceutically acceptable salt thereof, for use in the treatment of a disease or disorder selected from sickle cell disease and beta-thalassemia.
Embodiment 62. A compound according to any one of Embodiments 1 to 44, or a pharmaceutically acceptable salt thereof, for use in treating or preventing a disorder that is affected by the inhibition of WIZ protein levels, in a subject in need thereof.
Embodiment 63. A compound according to any one of Embodiments 1 to 44, or a pharmaceutically acceptable salt thereof, for use in treating or preventing a disorder that is affected by the reduction of WIZ protein levels, in a subject in need thereof.
Embodiment 64. A compound according to any one of Embodiments 1 to 44, or a pharmaceutically acceptable salt thereof, for use in the treatment or prevention of a disease or disorder that is affected by the degradation of WIZ protein.
Embodiment 65. A compound according to any one of Embodiments 1 to 44, or a pharmaceutically acceptable salt thereof, for use in inhibiting, reducing, or eliminating the activity of WIZ protein or WIZ protein expression in a subject in need thereof.
Embodiment 66. A compound according to any one of Embodiments 1 to 44, or a pharmaceutically acceptable salt thereof, for use in inducing or promoting fetal hemoglobin in a subject in need thereof.
Embodiment 67. A compound according to any one of Embodiments 1 to 44, or a pharmaceutically acceptable salt thereof, for use in reactivating fetal hemoglobin production or expression in a subject in need thereof.
Embodiment 68. A compound according to any one of Embodiments 1 to 44, or a pharmaceutically acceptable salt thereof, for use in increasing fetal hemoglobin expression in a subject in need thereof.
Embodiment 69. A compound according to any one of Embodiments 1 to 44, or a pharmaceutically acceptable salt thereof, for use in treating a hemoglobinopathy in a subject in need thereof.
Embodiment 70. A compound according to any one of Embodiments 1 to 44, or a pharmaceutically acceptable salt thereof, for use in treating a sickle cell disease in a subject in need thereof.
Embodiment 71. A compound according to any one of Embodiments 1 to 44, or a pharmaceutically acceptable salt thereof, for use in treating beta-thalassemia in a subject in need thereof.
Embodiment 72. A compound according to any one of Embodiments 1 to 44, or a pharmaceutically acceptable salt thereof, for use in the treatment of a disease or disorder affected by an increase in fetal hemoglobin expression.
Embodiment 73. A compound according to any one of Embodiments 1 to 44, or a pharmaceutically acceptable salt thereof, for use in the treatment of a disease or disorder affected by the inhibition, reduction, or elimination of the activity of WIZ protein or WIZ protein expression.
Embodiment 74. A compound according to any one of Embodiments 1 to 44, or a pharmaceutically acceptable salt thereof, for use in the treatment of a disease or disorder affected by the induction or promotion of fetal hemoglobin.
Embodiment 75. A compound according to any one of Embodiments 1 to 44, or a pharmaceutically acceptable salt thereof, for use in the treatment of a disease or disorder affected by the reactivation of fetal hemoglobin production or expression.
Embodiment 76. A compound according to any one of Embodiments 1 to 44, or a pharmaceutically acceptable salt thereof, for use in inhibiting WIZ protein expression in a subject in need thereof.
Embodiment 77. A compound according to any one of Embodiments 1 to 44, or a pharmaceutically acceptable salt thereof, for use in degrading WIZ protein in a subject in need thereof.
Embodiment 78. Use of a compound according to any one of Embodiments 1 to 44, or a pharmaceutically acceptable salt thereof, in the manufacture of a medicament for treating a disease or disorder that is affected by the reduction of WIZ protein levels, inhibition of WIZ protein expression or degradation of WIZ protein.
Embodiment 79. Use of a compound according to any one of Embodiments 1 to 44, or a pharmaceutically acceptable salt thereof, in the manufacture of a medicament for treating a disease or disorder that is affected by inducing or promoting fetal hemoglobin.
Embodiment 80. Use of a compound according to any one of Embodiments 1 to 44, or a pharmaceutically acceptable salt thereof, in the manufacture of a medicament for treating a disease or disorder that is affected by reactivating fetal hemoglobin production or expression.
Embodiment 81. Use of a compound according to any one of Embodiments 1 to 44, or a pharmaceutically acceptable salt thereof, in the manufacture of a medicament for treating a disease or disorder that is affected by increasing fetal hemoglobin expression.
Embodiment 82. The use of a compound according to any one of Embodiments 78 to 81, wherein the disease or disorder is selected from sickle cell disease and beta-thalassemia.
Embodiment 83. Use of a compound according to any one of Embodiments 1 to 44, or a pharmaceutically acceptable salt thereof, in the treatment of a disease or disorder that is affected by the reduction of WIZ protein levels, inhibition of WIZ protein expression or degradation of WIZ protein.
Embodiment 84. Use of a compound according to any one of Embodiments 1 to 44, or a pharmaceutically acceptable salt thereof, in the treatment of a disease or disorder that is affected by inducing fetal hemoglobin, reactivating fetal hemoglobin production or expression, or increasing fetal hemoglobin expression.
Embodiment 85. The use according to Embodiment 83 or 84, wherein the disease or disorder is selected from sickle cell disease and beta-thalassemia.
Embodiment 86. A pharmaceutical combination comprising a compound according to any one of Embodiments 1 to 44, or a pharmaceutically acceptable salt thereof, and one or more additional therapeutic agent(s).

Depending on the choice of the starting materials and procedures, the compounds can be present in the form of one of the possible isomers or as mixtures thereof, for example as pure optical isomers, or as isomer mixtures, such as racemates and diastereomeric mixtures, depending on the number of asymmetric centres. The disclosure is meant to include all such possible isomers, including racemic mixtures, enantiomerically enriched mixtures, diastereomeric mixtures and optically pure forms. Optically active (R)- and (S)-isomers may be prepared using chiral synthons or chiral reagents, or resolved using conventional techniques. If the compound contains a disubstituted or trisubstituted cycloalkyl, the cycloalkyl substituent(s) may have a cis- or trans-configuration. The disclosure includes cis and trans configurations of substituted cycloalkyl groups as well as mixtures thereof. All tautomeric forms are also intended to be included. In particular, where a heteroaryl ring containing N as a ring atom is 2-pyridone, for example, tautomers where the carbonyl is depicted as a hydroxy (e.g., 2-hydroxypyridine) are included.

### Pharmaceutically Acceptable Salts

As used herein, the terms “salt” or “salts” refers to an acid addition or base addition salt of a compound of the disclosure. “Salts” include in particular “pharmaceutically acceptable salts”. The term “pharmaceutically acceptable salts” refers to salts that retain the biological effectiveness and properties of the compounds of this disclosure and, which typically are not biologically or otherwise undesirable. The compounds of the disclosure may be capable of forming acid and/or base salts by virtue of the presence of amino and/or carboxyl groups or groups similar thereto.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, formic acid, trifluoroacetic acid, and the like.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the disclosure provides compounds in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, formate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

In another aspect, the disclosure provides compounds in sodium, potassium, ammonium, calcium, magnesium, iron, silver, zinc, copper, isopropylamine, benzathine, cholinate, diethanolamine, diethylamine, lysine, meglumine, piperazine or tromethamine salt form.

Preferably, pharmaceutically acceptable salts of compounds of formulae (I″), (I′), (I), (Ia″), (Ia′), (Ia), (Ib″), (Ib′), (Ib), (Ic″), (Ic′), (Ic), (Id″), (Id′), (Id), (Ie″), (Ie′), and (Ie) are acid addition salts.

### Isotopically Labelled Compounds

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, sulfur, fluorine, chlorine and iodine, such as ²H, ³H, ¹¹C, ¹³C, ¹⁴C, ¹⁸O, ¹⁵N, ¹⁸F, ¹⁷O, ¹⁸O, ³⁵S, ³⁶Cl, ¹²³I, ¹²⁴I, ¹²⁵I respectively. The disclosure includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F compound may be particularly desirable for PET or SPECT studies. Isotopically-labeled compounds of formulae (I″), (I′), (I), (Ia″), (Ia′), (Ia), (Ib″), (Ib′), (Ib), (Ic″), (Ic′), (Ic), (Id″), (Id′), (Id), (Ie″), (Ie′), and (Ie) can generally be prepared by conventional techniques known to those skilled in the art or by processes analogous to those described in the accompanying Examples and General Schemes (e.g., General Schemes 1 to 5) using an appropriate isotopically-labeled reagent in place of the non-labeled reagent previously employed.

In one embodiment of any aspect of the present disclosure, the hydrogens in the compound of Formula (I″), Formula (I) or Formula (I′) are present in their normal isotopic abundances. In a another embodiment, the hydrogens are isotopically enriched in deuterium (D), and in a particular embodiment of the disclosure the hydrogen(s) of the dihydrouracil (DHU) or the uracil portion in compounds of Formula (I) or Formula (I′) are enriched in D, for example,

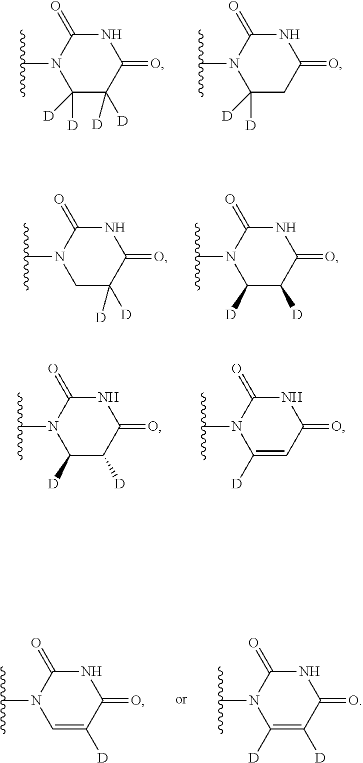

Deuterated dihydrouracil and uracil moities can be prepared as described in Hill, R. K. et al., Journal of Labelled Compounds and Radiopharmaceuticals, Vol. XXII, No. 2, p. 143-148.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formulae (I″), (I′), (I), (Ia″), (Ia′), (Ia), (Ib″), (Ib′), (Ib), (Ic″), (Ic′), (Ic), (Id″), (Id′), (Id), (Ie″), (Ie′), and (Ie). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the disclosure include those wherein the solvent of crystallization may be isotopically substituted, e.g., D₂O, d₆-acetone, d₆-DMSO.

Compounds of the disclosure, i.e. compounds of formulae (I″), (I′), (I), (Ia″), (Ia′), (Ia), (Ib″), (Ib′), (Ib), (Ic″), (Ic′), (Ic), (Id″), (Id′), (Id), (Ie″), (Ie′), and (Ie) that contain groups capable of acting as donors and/or acceptors for hydrogen bonds may be capable of forming co-crystals with suitable co-crystal formers. These co-crystals may be prepared from compounds of formulae (I″), (I′), (1), (Ia″), (Ia′), (Ia), (Ib″), (Ib′), (Ib), (Ic″), (Ic′), (Ic), (Id″), (Id′), (Id), (Ie″), (Ie′), and (Ie) by known co-crystal forming procedures. Such procedures include grinding, heating, co-subliming, co-melting, or contacting in solution compounds of formulae (I″), (I′), (I), (Ia″), (Ia′), (Ia), (Ib″), (Ib′), (Ib), (Ic″), (Ic′), (Ic), (Id″), (Id′), (Id), (Ie″), (Ie′), and (Ie) with the co-crystal former under crystallization conditions and isolating co-crystals thereby formed. Suitable co-crystal formers include those described in WO 2004/078163.

All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed.

Any asymmetric center (e.g., carbon or the like) of the compound(s) of the disclosure can be present in racemic or enantiomerically enriched, for example the (R)-, (S)- or (R,S)-configuration. In certain embodiments, for example, as a mixture of enantiomers, each asymmetric center is present in at least 10% enantiomeric excess, at least 20% enantiomeric excess, at least 30% enantiomeric excess, at least 40% enantiomeric excess, at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess. In certain embodiments, for example, in enantiomerically enriched form, each asymmetric center is present in at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess. Thus, compounds of the disclosure can be present in a racemic mixture or in enantiomerically enriched form or in an enantiopure form or as a mixture of diastereoisomers.

In the formulae of the present application the term “ [symbol] ” on a C-sp³ indicates the absolute stereochemistry, either (R) or (S). In the formulae of the present application the term “ [symbol] ” on a C-sp³ indicates the absolute stereochemistry, either (R) or (S). In the formulae of the present application the term “ [symbol] ” on a C-sp³ represents a covalent bond wherein the stereochemistry of the bond is not defined. This means that the term “ [symbol] ” on a C-sp³ comprises an (S) configuration or an (R) configuration of the respective chiral centre. Furthermore, mixtures may also be present. Therefore, mixtures of stereoisomers, e.g., mixtures of enantiomers, such as racemates, and/or mixtures of diastereoisomers are encompassed by the present disclosure.

For the avoidance of doubt, where compound structures are drawn with undefined stereochemistry with respect to any R group for example, to R² in formula (I″), (I′) or (I), as represented by a bond ( [symbol] ), this means the asymmetric center has either a (R)- or (S)-configuration, or exists as a mixture thereof and stated as such.

Accordingly, as used herein a compound of the disclosure can be in the form of one of the possible stereoisomers, rotamers, atropisomers, tautomers or mixtures thereof, for example, as substantially pure geometric (cis or trans) stereoisomers, diastereomers, optical isomers, racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the disclosure or of intermediates can be resolved into the optical isomers (enantiomers) by known methods, e.g., by separation of the diastereomeric salts thereof, obtained with an optically active acid or base, and liberating the optically active acidic or basic compound. In particular, a basic moiety may thus be employed to resolve the compounds of the disclosure into their optical antipodes, e.g., by fractional crystallization of a salt formed with an optically active acid, e.g., tartaric acid, dibenzoyl tartaric acid, diacetyl tartaric acid, di-O,O′p-toluoyl tartaric acid, mandelic acid, malic acid or camphor-10-sulfonic acid. Racemic compounds of the disclosure or racemic intermediates can also be resolved by chiral chromatography, e.g., high pressure liquid chromatography (HPLC) using a chiral adsorbent.

Furthermore, the compounds of the disclosure, including their salts, can also be obtained in the form of their hydrates, or include other solvents used for their crystallization. The compounds of the disclosure may inherently or by design form solvates with pharmaceutically acceptable solvents (including water); therefore, it is intended that the disclosure embrace both solvated and unsolvated forms. The term “solvate” refers to a molecular complex of a compound of the disclosure (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water. The presence of solvates can be identified by a person of skill in the art with tools such as NMR.

The compounds of the disclosure, including salts, hydrates and solvates thereof, may inherently or by design form polymorphs.

### Methods of Making

The compounds of the disclosure can be prepared in a number of ways well known to those skilled in the art of organic synthesis. By way of example, compounds of the present disclosure can be synthesized using the methods described below, together with synthetic methods known in the art of synthetic organic chemistry, or variations thereon as appreciated by those skilled in the art.

Generally, the compounds of formulae (I″), (I′) and (I) can be prepared according to the Schemes provided infra.

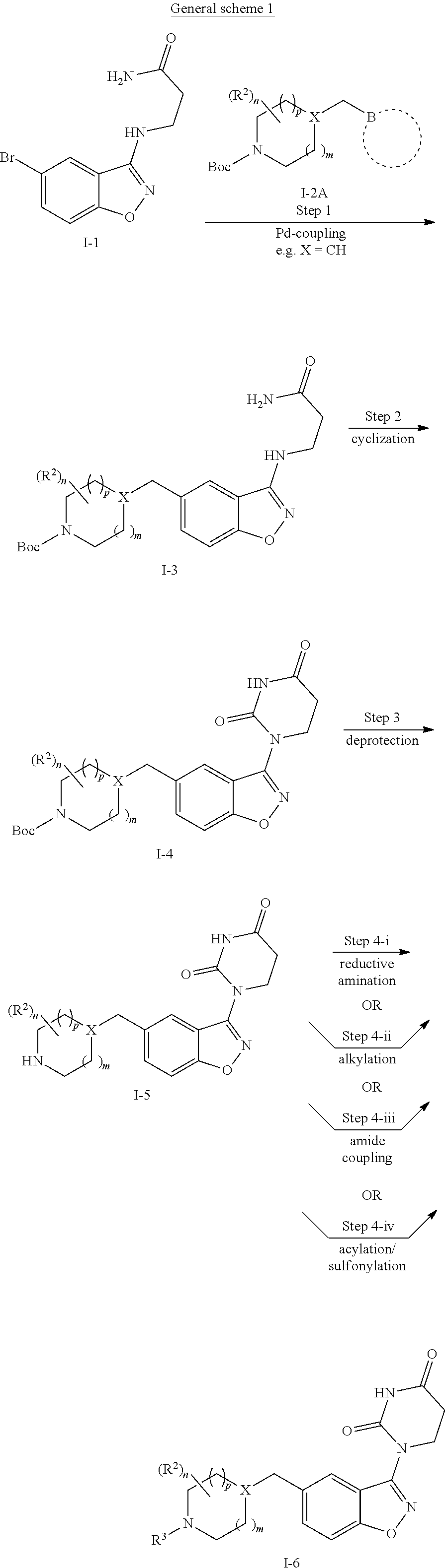

The starting materials for the above reaction scheme are commercially available or can be prepared according to methods known to one skilled in the art or by methods disclosed herein. In general, the compounds of the disclosure are prepared in the above reaction Scheme 1 as follows:

A cross-coupling reaction, such as a palladium (Pd)-catalysed coupling of I-1 with a boraneyl coupling partner of formula I-2A (prepared by hydroboration of an appropriate alkene with 9-BBN, for example) in the presence of a polar solvent, such as N,N-dimethylformamide (DMF), a suitable ligand such as dppf, and a base such as potassium carbonate (K₂CO₃) can provide the cross-coupled product I-3 in Step 1, where X is CH. Cyclization of the primary amide and amino groups of I-3, with a carbonyl equivalent, such as 1,1′-carbonyldiimidazole (CDI) in the presence of an amine or carbonate base, such as diisopropylethylamine (DIPEA) or cesium carbonate (Cs₂CO₃), and a polar solvent, such as acetonitrile to form a dihydrouracil moiety can give I-4 (step 2). Removal of the protecting group (e.g., Boc) under acidic conditions at room temperature can provide the free amine I-5 (step 3). I-5 can then be converted to I-6 via a reductive amination (Step 4-i) with an appropriate aldehyde in the presence of a borohydride reagent, such as sodium borohydride acetate; alternatively, by an alkylation reaction (Step 4-ii) with an appropriate alkyl halide, mesylate, tosylate or triflate in the presence of an amine or carbonate base and polar solvent, such as diisopropylethylamine (DIPEA) or potassium carbonate (K₂CO₃) and dimethylformamide (DMF); alternatively, by an amide coupling reaction (Step 4-iii) of the compound with an appropriate carboxylic acid, an activating agent, such as HATU, and a base such as DIPEA, when R³ forms an amide with the nitrogen to which it is attached; or alternatively, by an acylation or sulfonylation reaction (Step 4-iv) with an appropriate acyl chloride or sulfonyl chloride and a base such as DIPEA or TEA, where R³ forms an amide or sulfonamide with the nitrogen to which it is attached.

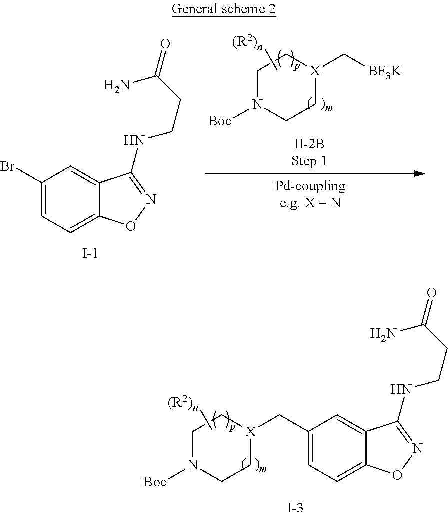

The starting materials for the above reaction scheme are commercially available or can be prepared according to methods known to one skilled in the art or by methods disclosed herein. In general, the compounds of the disclosure are prepared in the above reaction Scheme 2 as follows:

A cross-coupling reaction, such as a palladium (Pd)-catalysed coupling of I-1 with a trifluoroborate (potassium salt) coupling partner of formula II-2B in the presence of an organic solvent such as toluene, and water, a phosphine ligand such as RuPhos or Xphos, and a base such as cesium carbonate (Cs₂CO₃) can provide the cross-coupled product 1-3 in Step 1, where X is N. Compound 1-3 as prepared in this manner, can be converted to compounds of formula 1-6 by the methods of General Scheme 1, steps 2-4.

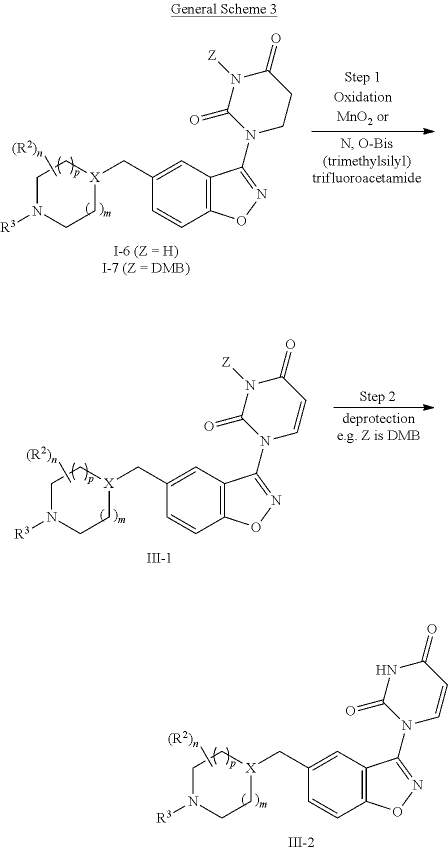

In General Scheme 3, a compound of formula 1-6 or 1-7 is subjected to oxidation conditions, e.g., MnO₂, in a suitable solvent, such as toluene (e.g., at room temperature), or in the presence of N,O-bis(trimethylsilyl)trifluoroacetamide, to produce a compound of formula III-1 (i.e., formula (I′) when Z=H), followed by an optional deprotection step when Z is a nitrogen protecting group, e.g., DMB, to give a compound of formula III-2 (i.e., formula (I′)).

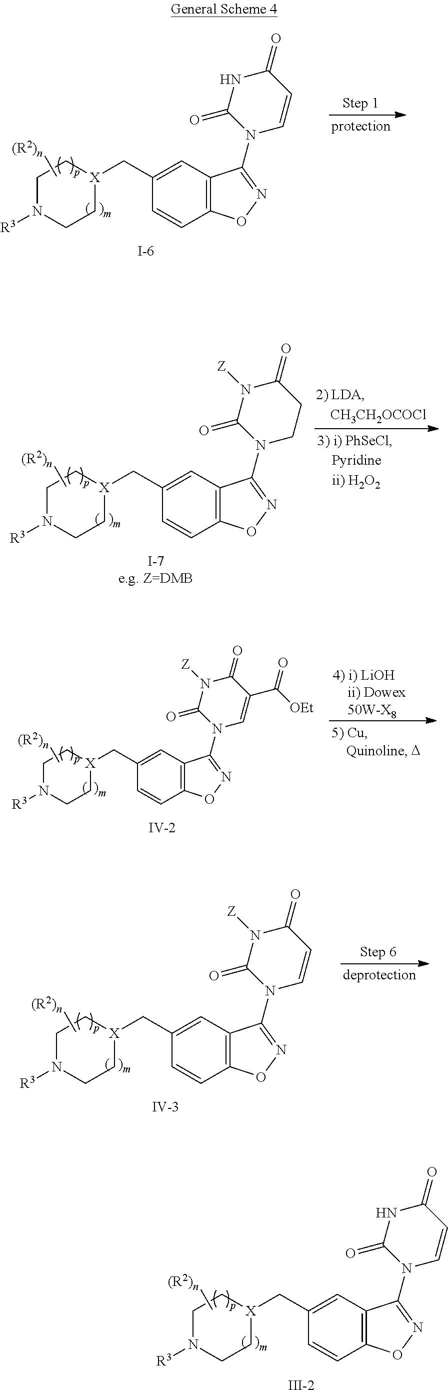

In General Scheme 4, a compound of formula 1-6 is first protected with a suitable nitrogen protecting group (represented by Z), e.g., an acid labile protecting group, e.g., DMB, followed by a Claisen condensation (step 2) and subsequent selenation/oxidation/elimination sequence (step 3) to give a compound of formula IV-2. Compound formula IV-2 undergoes hydrolysis followed by a copper-catalysed decarboxylation to give a compound of formula IV-3. Subsequent deprotection, e.g., under acidic conditions and heating, provides a compound of formula III-2 (i.e., formula (I′)).

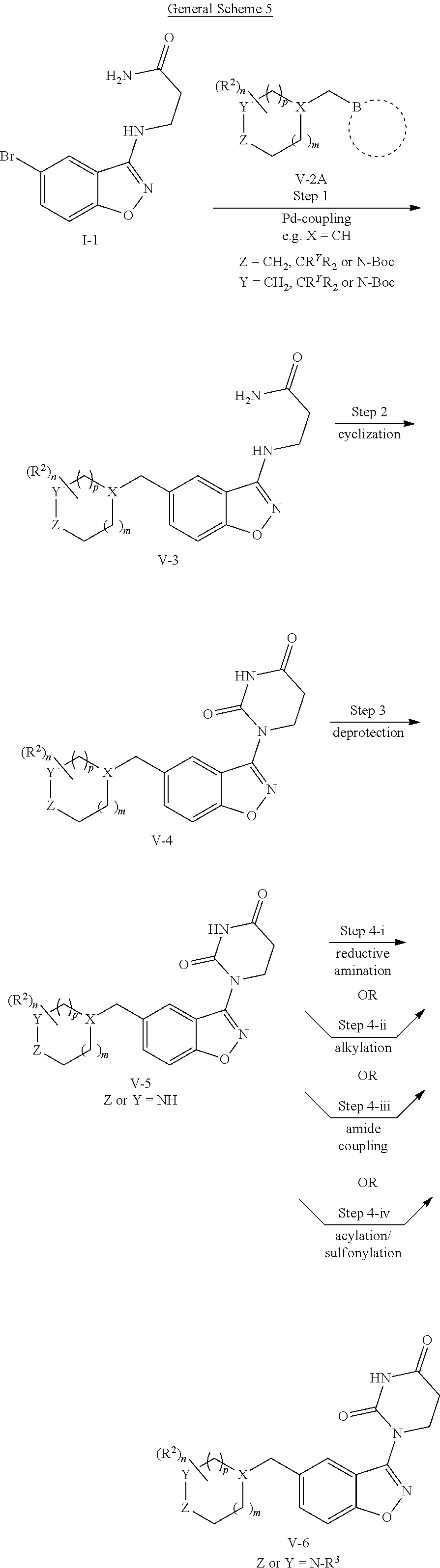

Generally, compounds of formula (I″) can be prepared in a similar manner to General Scheme 1. For intermediates V-2A, V-3, V-4 and V-5, only one of Z and Y can be N-Boc or N—H, as defined by claim 1.

For Schemes 1 to 5, X, Y, Z, R², R³, n, m and p are as defined herein, in particular according to any one of Embodiments 1 to 43.

In a further embodiment, there is provided a compound of Formula (X-1) or a salt thereof,

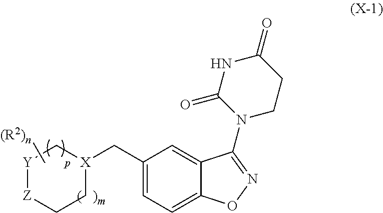

wherein:
X is selected from CH, CF, and N;
Y is selected from CH₂, CR^{Y}R² and N—R^{N};
Z is selected from CH₂, CR^{Y}R² and N—R^{N};
wherein at least one of Y and Z is N—R^{N} such that when Y is N—R^{N}, Z is selected from CH₂, and CR^{Y}R², and when Z is N—R^{N}, Y is selected from CH₂, and CR^{Y}R²,
and wherein when R² of CR^{Y}R² of Y or Z is oxo, R^{Y} is absent;
R^{Y} is selected from hydrogen and C₁-C₆alkyl;
R^{N} is selected from hydrogen and a nitrogen protecting group (PG) (e.g., tert-butyloxycarbonyl (Boc));
each R² is independently selected from C₁-C₆alkyl, C₁-C₆haloalkyl, halo, and oxo, wherein the C₁-C₆alkyl is substituted with 0-1 occurrence of R^{2a}; or 2 R² on non-adjacent carbon atoms together with the non-adjacent carbon atoms to which they are attached form a bridging ring;
R^{2a} is selected from C₁-C₆alkoxyl and hydroxyl;
n is 0, 1, 2, 3 or 4;
m is 0, 1 or 2; and
p is 0 or 1.

In an embodiment, R^{Y} is hydrogen.

In an embodiment, the nitrogen protecting group PG is an acid labile protecting group.

In an embodiment, the nitrogen protecting group PG is the Boc protecting group (tert-butyloxycarbonyl).

In a further embodiment, there is provided a compound of Formula (X) or a salt thereof,

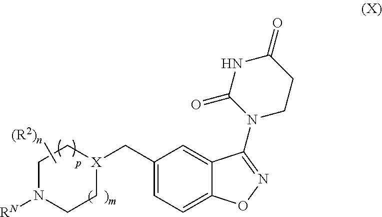

wherein:
X is selected from CH, CF, and N;
R^{N} is selected from hydrogen and a nitrogen protecting group (PG) (e.g., tert-butyloxycarbonyl (Boc));
each R² is independently selected from C₁-C₆alkyl, C₁-C₆haloalkyl, halo, and oxo, wherein the C₁-C₆alkyl is substituted with 0-1 occurrence of R^{2a}; or 2 R² on non-adjacent carbon atoms together with the non-adjacent carbon atoms to which they are attached form a bridging ring;
R^{2a} is selected from C₁-C₆alkoxyl and hydroxyl;
n is 0, 1, 2, 3 or 4;
m is 0, 1 or 2; and
p is 0 or 1.

In an embodiment, the nitrogen protecting group PG is an acid labile protecting group.

In an embodiment, the nitrogen protecting group PG is the Boc protecting group (tert-butyloxycarbonyl).

In an embodiment, the salt of a compound of formula (X-1) or (X) is selected from a HCl and TFA salt.

In a further embodiment, there is provided 3-((5-bromobenzo[d]isoxazol-3-yl)amino)propanamide or a salt thereof.

In a further aspect, the disclosure provides to a process for the preparation of a compound of formula (I″), (I′) or (I), in free form or in pharmaceutically acceptable salt form, comprising the step of:
1) coupling an aryl bromide of formula I-1 with a boraneyl coupling partner of formula I-2A, V-2A or II-2B under cross coupling conditions, to give a compound of formula 1-3) or V-3 as defined herein.

The boraneyl coupling partner of step 1 may optionally be prepared by hydroboration of a precursor alkene, e.g., with 9-BBN.

Cross coupling reaction conditions for any of the aforementioned process steps or hereinafter involve the use of a Pd catalyst in the presence of a phosphine ligand, such as Pd(OAc)₂ and RuPhos or Xphos, and a base such as cesium carbonate (Cs₂CO₃), in the presence of a suitable solvent such as toluene, water, or a mixture thereof.

In an embodiment of the process described above, there is provided the further steps of:
2) intramolecular cyclization of the amide portion in a compound of formula 1-3 or V-3 with the amine portion in a compound of formula 1-3 or V-3 to give the compound of formula 1-4 or V-4 as defined herein;
3) deprotecting a compound of formula 1-4 or V-4, e.g., under acidic conditions, to give a compound of formula 1-5 or V-5 as defined herein;
4-a) reacting a compound of formula 1-5 or V-5 under reductive amination conditions to give a compound of formula 1-6 or V-6 as defined herein; or
4-b) reacting a compound of formula 1-5 or V-5 under alkylation conditions to give a compound of formula 1-6 or V-6 as defined herein; or
4-c) reacting a compound of formula 1-5 or V-5 under amide coupling conditions to give a compound of formula 1-6 or V-6 as defined herein; or
4-d) reacting a compound of formula 1-5 or V-5 under acylation or sulfonylation conditions to give a compound of formula 1-6 or V-6 as defined herein.

Cyclization conditions for any of the aforementioned process steps or hereinafter involve the use of an organic coupling reagent, such as 1,1′-carbonyldiimidazole (CDI), in the presence of an amine or carbonate base, such as diisopropylethylamine (DIPEA) or cesium carbonate (Cs₂CO₃), and a polar solvent, such as acetonitrile.

Reductive amination conditions for any of the aforementioned process steps or hereinafter involve the use of a corresponding aldehyde, a suitable hydride reagent, such as NaBH(OAc)₃, a suitable solvent, such as DMF, the reaction conducted at room temperature (r.t.). The reaction may optionally be heated to a temperature above room temperature.

Alkylation reaction conditions for any of the aforementioned process steps or hereinafter involve the use of a corresponding alkyl halide, mesylate, tosylate or triflate in the presence of a suitable base, such as DIPEA, or a carbonate base such as K₂CO₃, a polar solvent, such as DMF, the reaction conducted at a suitable temperature, such as r.t. to 100° C., e.g., 80° C., optionally, under microwave.

Amide coupling reaction conditions for any of the aforementioned process steps or hereinafter involve the use of a corresponding carboxylic acid, an activating agent, such as HATU, a suitable base, such as DIPEA or NMM, a suitable solvent, such as DMF, the reaction conducted at a suitable temperature, such as r.t., for a suitable amount of time, for example 12 hours.

Acylation or sulfonylation reaction conditions for any of the aforementioned process steps or hereinafter involve the use of a corresponding acyl chloride or sulfonyl chloride and a base such as DIPEA or TEA, in the presence of a suitable solvent such as DCM, the reaction conducted at a suitable temperature, such as r.t..

In a further embodiment there is provided a process for the preparation of a compound of formula (I″), (I′) or (I), and sub-formulae thereof, in free form or in pharmaceutically acceptable salt form according to any of General Schemes 1 to 5.

Compounds of formulae (X-1), (X) and (I)-1, i.e., 3-((5-bromobenzo[d]isoxazol-3-yl)amino)propanamide, as defined herein are useful in the preparation of compounds of the disclosure, e.g., compounds of formulae (I″), (I′), (I), (Ia″), (Ia′), (Ia), (Ib″), (Ib′), (Ib), (Ic″), (Ic′), (Ic), (Id″), (Id′), (Id), (Ie″), (Ie′), and (Ie). Thus, in an aspect, the disclosure relates to a compound of formula (I)-1, (X-1) or (X), or a salt thereof. In another aspect, the disclosure relates to the use of a compound of formula (I)-1, (X-1) or (X), or a salt thereof, in the manufacture of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie). The disclosure further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure material.

### Pharmaceutical Compositions

In another aspect, the disclosure provides a pharmaceutical composition comprising one or more compounds of described herein or a pharmaceutically acceptable salt, hydrate, solvate, prodrug, stereoisomer, or tautomer thereof, and one or more pharmaceutically acceptable carriers. As used herein, the term “pharmaceutical composition” refers to a compound of the disclosure, or a pharmaceutically acceptable salt thereof, together with at least one pharmaceutically acceptable carrier, in a form suitable for oral or parenteral administration. As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22^{nd} Ed. Pharmaceutical Press, 2013, pp. 1049-1070).

In another aspect, the disclosure provides a pharmaceutical composition comprising a compound of the disclosure, or a pharmaceutically acceptable salt thereof, and a pharmaceutically acceptable carrier. In a further embodiment, the composition comprises at least two pharmaceutically acceptable carriers, such as those described herein. For purposes of the disclosure, unless designated otherwise, solvates and hydrates are generally considered compositions. Preferably, pharmaceutically acceptable carriers are sterile. The pharmaceutical composition can be formulated for particular routes of administration such as oral administration, parenteral administration, and rectal administration, etc. In addition, the pharmaceutical compositions of the disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol;
c) binders, e.g., magnesium aluminum silicate, starch paste, gelatin, tragacanth, methylcellulose, sodium carboxymethylcellulose and/or polyvinylpyrrolidone;
d) disintegrants, e.g., starches, agar, alginic acid or its sodium salt, or effervescent mixtures; and
e) absorbents, colorants, flavors and sweeteners.

In an embodiment, the pharmaceutical compositions are capsules comprising the active ingredient only.

Tablets may be either film coated or enteric coated according to methods known in the art.

Suitable compositions for oral administration include an effective amount of a compound of the disclosure in the form of tablets, lozenges, aqueous or oily suspensions, dispersible powders or granules, emulsion, hard or soft capsules, or syrups or elixirs, solutions or solid dispersion. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients, which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the disclosure with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The compounds of formulae (I″), (I′), (I), (Ia″), (Ia′), (Ia), (Ib″), (Ib′), (Ib), (Ic″), (Ic′), (Ic), (Id″), (Id′), (Id), (Ie″), (Ie′), and (Ie), in free form or in pharmaceutically acceptable salt form, exhibit valuable pharmacological properties, e.g., WIZ modulating properties or WIZ degrading properties or HbF inducing properties e.g., as indicated in the in vitro tests as provided in the examples, and are therefore indicated for therapy or for use as research chemicals, e.g., as tool compounds.

Additional properties of the disclosed compounds include having good potency in the biological assays described herein, favorable safety profile, and possess favorable pharmacokinetic properties.

### Diseases and Disorders

In an embodiment of the present disclosure, there is provided a compound of the disclosure, or a pharmaceutically acceptable salt thereof, which is effective in reducing WIZ protein expression levels and/or inducing fetal hemoglobin (HbF) expression.

The compounds of the disclosure can be used to treat one or more of the diseases or disorders described herein below. In one embodiment, the disease or disorder is affected by the reduction of WIZ protein expression levels and/or induction of fetal hemoglobin protein expression levels. In another embodiment, the disease or disorder is a hemoglobinopathy, e.g., beta hemoglobinopathy, including sickle cell disease (SCD) and beta-thalassemia.

### Methods of Use

All the aforementioned embodiments and embodiments hereinafter relating to the methods of reducing WIZ protein expression levels and/or inducing fetal hemoglobin (HbF) expression are equally applicable to:

A compound of the disclosure, or a pharmaceutically acceptable salt thereof, for use in a method of reducing WIZ protein expression levels and/or inducing fetal hemoglobin (HbF) expression;

A compound of the disclosure, or a pharmaceutically acceptable salt thereof, for use in the treatment of the aforementioned diseases or disorders according to the present disclosure;

Use of a compound of the disclosure, or a pharmaceutically acceptable salt thereof, in the treatment of the aforementioned diseases or disorders according to the present disclosure; and

A pharmaceutical composition comprising a compound of the disclosure, or a pharmaceutically acceptable salt thereof, for use in the treatment of the aforementioned diseases or disorders according to the present disclosure.

Having regard to their activity as WIZ modulators or degraders, compounds of formulae (I″), (I′), (I), (Ia″), (Ia′), (Ia), (Ib″), (Ib′), (Ib), (Ic″), (Ic′), (Ic), (Id″), (Id′), (Id), (Ie″), (Ie′), and (Ie), in free or pharmaceutically acceptable salt form, are useful in the treatment of conditions which may be treated by modulation of WIZ protein expression levels, reduction of WIZ protein expression levels, or induction of fetal hemoglobin (HbF), such as in a blood disorder, for example an inherited blood disorder, e.g., sickle cell disease, or beta-thalassemia. In one aspect, the disclosure provides a method of treating or preventing a disease or disorder in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a method of treating or preventing a disorder that is affected by the reduction of WIZ protein levels, in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a method of inhibiting WIZ protein expression in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a method of degrading WIZ protein in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a method of inhibiting, reducing, or eliminating the activity of WIZ protein or WIZ protein expression, the method comprising administering to the subject a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a method of inducing or promoting fetal hemoglobin in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a method of reactivating fetal hemoglobin production or expression in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a method of increasing fetal hemoglobin expression in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a method of treating a hemoglobinopathy, e.g., a beta-hemoglobinopathy, in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a method of treating a sickle cell disease in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a method of treating beta-thalassemia in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In an embodiment, the beta-thalassemia major or intermedia is the result of homozygous null or compound heterozygous mutations resulting with beta-globin deficiency and the phenotypic complications of beta-thalassemia, whether transfusion-dependent or not.

In another aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof for use in a method of treating or preventing a disease or disorder in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof for use in a method of treating or preventing a disorder that is affected by the reduction of WIZ protein levels, in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof for use in a method of inhibiting WIZ protein expression in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof for use in a method of degrading WIZ protein in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof for use in a method of inhibiting, reducing, or eliminating the activity of WIZ protein or WIZ protein expression, the method comprising administering to the subject a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof for use in a method of inducing or promoting fetal hemoglobin in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof for use in a method of reactivating fetal hemoglobin production or expression in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof for use in a method of increasing fetal hemoglobin expression in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof for use in a method of treating a hemoglobinopathy, e.g., a beta-hemoglobinopathy, in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof for use in a method of treating a sickle cell disease in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In another aspect, the disclosure provides a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof for use in a method of treating beta-thalassemia in a subject in need thereof, the method comprising administering to the subject a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof.

In an embodiment, the beta-thalassemia major or intermedia is the result of homozygous null or compound heterozygous mutations resulting with beta-globin deficiency and the phenotypic complications of beta-thalassemia, whether transfusion-dependent or not.

### Dosage

The pharmaceutical composition or combination of the disclosure can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated.

The above-cited dosage properties are demonstrable in vitro and in vivo tests using advantageously mammals, e.g., mice, rats, dogs, monkeys or isolated organs, tissues and preparations thereof. The compounds of the disclosure can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations. A therapeutically effective amount in vivo may range depending on the route of administration, between about 0.1-500 mg/kg, or between about 1-100 mg/kg.

The activity of a compound according to the disclosure can be assessed by the in vitro methods described in the Examples.

### Combination Therapy

In another aspect, the disclosure provides a pharmaceutical combination comprising a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt, hydrate, solvate, prodrug, stereoisomer, or tautomer thereof, and one or more additional therapeutic agent(s) for simultaneous, separate or sequential use in therapy. In an embodiment, the additional therapeutic agent is a myelosuppressive agent, such as hydroxyurea.

Combination therapy includes the administration of the subject compounds in further combination with other biologically active ingredients (such as, but not limited to, a second and different antineoplastic agent or a therapeutic agent that targets HbF or another cancer target) and non-drug therapies (such as, but not limited to, surgery or radiation treatment). For instance, the compounds of the application can be used in combination with other pharmaceutically active compounds, preferably compounds that are able to enhance the effect of the compounds of the application.

The compound of the disclosure may be administered either simultaneously with, or before or after, one or more other therapeutic agents. The compound of the disclosure may be administered separately, by the same or different route of administration, or together in the same pharmaceutical composition as the other agents. A therapeutic agent is, for example, a chemical compound, peptide, antibody, antibody fragment or nucleic acid, which is therapeutically active or enhances the therapeutic activity when administered to a patient in combination with a compound of the disclosure. Thus, in one embodiment, the disclosure provides a combination comprising a therapeutically effective amount of a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), or a pharmaceutically acceptable salt thereof and one or more additional therapeutically active agents.

In one embodiment, the disclosure provides a product comprising a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), and at least one other therapeutic agent as a combined preparation for simultaneous, separate or sequential use in therapy. In one embodiment, the therapy is the treatment of a disease or condition modulated by WIZ. Products provided as a combined preparation include a composition comprising the compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), and the other therapeutic agent(s) together in the same pharmaceutical composition, or the compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), and the other therapeutic agent(s) in separate form, e.g., in the form of a kit.

In one embodiment, the disclosure provides a pharmaceutical composition comprising a compound of formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie), and another therapeutic agent(s). Optionally, the pharmaceutical composition may comprise a pharmaceutically acceptable carrier, as described above.

In one embodiment, the disclosure provides a kit comprising two or more separate pharmaceutical compositions, at least one of which contains a formula (I″), (I′), (I), (Ia″), (Ia′), (Ia), (Ib″), (Ib′), (Ib), (Ic″), (Ic′), (Ic), (Id″), (Id′), (Id), (Ie″), (Ie′), or (Ie). In one embodiment, the kit comprises means for separately retaining said compositions, such as a container, divided bottle, or divided foil packet. An example of such a kit is a blister pack, as typically used for the packaging of tablets, capsules and the like.

The kit of the disclosure may be used for administering different dosage forms, for example, oral and parenteral, for administering the separate compositions at different dosage intervals, or for titrating the separate compositions against one another. To assist compliance, the kit of the disclosure typically comprises directions for administration.

In the combination therapies of the disclosure, the compound of the disclosure and the other therapeutic agent may be manufactured and/or formulated by the same or different manufacturers. Moreover, the compound of the disclosure and the other therapeutic may be brought together into a combination therapy: (i) prior to release of the combination product to physicians (e.g., in the case of a kit comprising the compound of the disclosure and the other therapeutic agent); (ii) by the physician themselves (or under the guidance of the physician) shortly before administration; (iii) in the patient themselves, e.g., during sequential administration of the compound of the disclosure and the other therapeutic agent.

### Preparation of Compounds

It is understood that in the following description, combinations of substituents and/or variables of the depicted formulae are permissible only if such combinations result in stable compounds.

It will also be appreciated by those skilled in the art that in the processes described below, the functional groups of intermediate compounds may need to be protected by suitable protecting groups. Such functional groups include hydroxy, phenol, amino and carboxylic acid. Suitable protecting groups for hydroxy or phenol include trialkylsilyl or diarylalkylsilyl (e.g., t-butyldimethylsilyl, t-butyldiphenylsilyl or trimethylsilyl), tetrahydropyranyl, benzyl, substituted benzyl, methyl, and the like. Suitable protecting groups for amino, amidino and guanidino include t-butoxycarbonyl, benzyloxycarbonyl, and the like. Suitable protecting groups for carboxylic acid include alkyl, aryl or arylalkyl esters.

Protecting groups may be added or removed in accordance with standard techniques, which are well-known to those skilled in the art and as described herein. The use of protecting groups is described in detail in J. F. W. McOmie, “Protective Groups in Organic Chemistry”, Plenum Press, London and New York 1973; T. W. Greene and P. G. M. Wuts, “Greene's Protective Groups in Organic Synthesis”, Fourth Edition, Wiley, New York 2007; P. J. Kocienski, “Protecting Groups”, Third Edition, Georg Thieme Verlag, Stuttgart and New York 2005; and in “Methoden der organischen Chemie” (Methods of Organic Chemistry), Houben Weyl, 4th edition, Volume 15/I, Georg Thieme Verlag, Stuttgart 1974.

The protecting group may also be a polymer resin, such as a Wang resin or a 2-chlorotrityl-chloride resin.

The following reaction schemes illustrate methods to make compounds of this disclosure. It is understood that one skilled in the art would be able to make these compounds by similar methods or by methods known to one skilled in the art. In general, starting components and reagents may be obtained from sources such as Sigma Aldrich, Lancaster Synthesis, Inc., Maybridge, Matrix Scientific, TCI, and Fluorochem USA, Strem, other commercial vendors, or synthesized according to sources known to those skilled in the art, or prepared as described in this disclosure.

### Analytical Methods, Materials, and Instrumentation

Unless otherwise noted, reagents and solvents were used as received from commercial suppliers. Proton nuclear magnetic resonance (NMR) spectra were obtained on either Bruker Avance spectrometer or Varian Oxford 400 MHz spectrometer unless otherwise noted. Spectra are given in ppm (δ) and coupling constants, J, are reported in Hertz. Tetramethylsilane (TMS) was used as an internal standard. Chemical shifts are reported in ppm relative to dimethyl sulfoxide (δ 2.50), methanol (δ 3.31), chloroform (δ 7.26) or other solvent as indicated in NMR spectral data. A small amount of the dry sample (2-5 mg) is dissolved in an appropriate deuterated solvent (1 mL). The chemical names were generated using ChemBioDraw Ultra v12 from CambridgeSoft.

Mass spectra (ESI-MS) were collected using a Waters System (Acquity UPLC and a Micromass ZQ mass spectrometer) or Agilent-1260 Infinity (6120 Quadrupole); all masses reported are the m/z of the protonated parent ions unless recorded otherwise. The sample was dissolved in a suitable solvent such as MeCN, DMSO, or MeOH and was injected directly into the column using an automated sample handler. The analysis is performed on Waters Acquity UPLC system (Column: Waters Acquity UPLC BEH C18 1.7 μm, 2.1×30 mm; Flow rate: 1 mL/min; 55° C. (column temperature); Solvent A: 0.05% formic acid in water, Solvent B: 0.04% formic acid in MeOH; gradient 95% Solvent A from 0 to 0.10 min; 95% Solvent A to 20% Solvent A from 0.10 to 0.50 min; 20% Solvent A to 5% Solvent A from 0.50 to 0.60 min; hold at 5% Solvent A from 0.6 min to 0.8 min; 5% Solvent A to 95% Solvent A from 0.80 to 0.90 min; and hold 95% Solvent A from 0.90 to 1.15 min.

### Abbreviations:

ACN acetonitrile
AcOH acetic acid
AIBN azobisisobutyronitrile
aq. aqueous
B₂pin₂ bis(pinacolato)diboron
9-BBN 9-borabicyclo[3.3.1]nonane
Boc₂O di-tert-butyl dicarbonate
Bn benzyl
BnBr benzyl bromide
br broad
d doublet
dd doublet of doublets
ddd doublet of doublet of doublets
ddq doublet of doublet of quartets
ddt doublet of doublet of triplets
dq doublet of quartets
dt doublet of triplets
dtbbpy 4,4′-di-tert-butyl-2,2′-dipyridyl
dtd doublet of triplet of doublets
CDI 1,1′-carbonyldiimidazole
Cs₂CO₃ cesium carbonate
DCE 1,2-dichloroethane
DCM dichloromethane
DHP dihydropyran
DIBAL-H diisobutylaluminium hydride
DIPEA (DIEA) diisopropylethylamine
DIPEA N,N-diisopropylethylamine
DMA N,N-dimethylacetamide
DMAP 4-dimethylaminopyridine
DME 1,2-dimethoxyethane
DMF N,N-dimethylformamide
DMP Dess-Martin periodinane or 1,1,1-Tris(acetyloxy)-1,1-dihydro-1,2-benziodoxol-3-(1H)-one
DMSO dimethylsulfoxide
EC₅₀ half maximal effective concentration
ELSD evaporative light scattering detector
EtOH ethanol
Et₂O diethyl ether
Et₃N triethylamine
EtOAc ethyl acetate
HATU 1-[bis(dimethylamino)methylene]-1H-1,2,3-triazolo[4,5-b]pyridinium 3-oxid hexafluorophosphate
HCl hydrogen chloride
hept heptet
HPLC high performance liquid chromatography
h or hr hour
HRMS high resolution mass spectrometry
g gram
g/min gram per minute
IC₅₀ half maximal inhibitory concentration
IPA (iPrOH) isopropyl alcohol
Ir[(dF(CF₃)ppy)₂dtbbpy]PF₆ [4,4′-Bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate
K₂CO₃ potassium carbonate
KI potassium iodide
KOAc potassium Acetate
K₃PO₄ tripotassium phosphate
LCMS liquid chromatography mass spectrometry
LDA lithium diisopropylamide
m multiplet
MeCN acetonitrile
MeOH methanol
mg milligram
MHz megahertz
min minutes
mL milliliter
mmol millimole
M molar
MS mass spectrometry
NaH sodium hydride
NaHCO₃ sodium bicarbonate
NaBH(OAc)₃ sodium triacetoxyborohydride
Na₂SO₄ sodium sulfate
NBS N-bromosuccinimide
NMM N-methylmorpholine
NMP N-methyl-2-pyrrolidone
NMR nuclear magnetic resonance
on overnight
Pd/C palladium on carbon
PdCl₂(dppf)·DCM [1,1′-bis(diphenylphosphino)ferrocene]dichloropalladium(II) complex with dichloromethane
Pd(PPh₃)₄ tetrakis(triphenylphosphine)palladium(0)
PMB para-methoxybenzyl
q quartet
qd quartet of doublets
quint quintet
quintd quintet of doublets
rbf round bottom flask
RockPhos G3 Pd [(2-di-tert-butylphosphino-3-methoxy-6-methyl-2′,4′,6′-triisopropyl-1,1′-biphenyl)-2-(2-aminobiphenyl)]palladium(II) methanesulfonate
rt or r.t. room temperature
Rt retention time
RuPhos dicyclohexyl(2′,6′-diisopropoxy-[1,1′-biphenyl]-2-yl)phosphane
s singlet
SEM 2-(trimethylsilyl)ethoxymethyl
SnBu₃ tributyltin
t triplet
td triplet of doublets
tdd triplet of doublet of doublets
TBAI tetrabutylammonium iodide
TEA (NEt₃) triethylamine
TFA trifluoroacetic acid
TfOH triflic Acid
THF tetrahydrofuran
THP tetrahydropyran
TMP 2,2,6,6-tetramethylpiperidine
Ts tosyl
tt triplet of triplets
ttd triplet of triplet of doublets
TLC thin-layer chromatography
UPLC ultra-Performance liquid Chromatography
XPhos Pd G2 chloro(2-dicyclohexylphosphino-2′,4′,6′-triisopropyl-1,1′-biphenyl)[2-(2′-amino-1,1′-biphenyl)]palladium(II)
μW or uW microwave

### Preparation of Intermediates

### Preparation of Potassium (R)-((4-(Tert-Butoxycarbonyl)-3-Methylpiperazin-1-yl)methyl)trifluoroborate

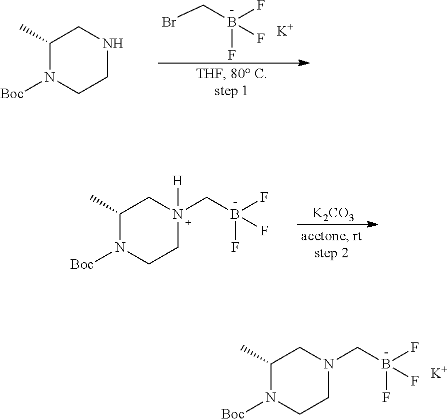

### Step 1. (((3R)-4-(tert-butoxycarbonyl)-3-methylpiperazin-1-ium-1-yl)methyl)trifluoroborate

To a solution of potassium (bromomethyl)trifluoroborate (2.00 g, 9.96 mmol) in THF (10 mL) was added tert-butyl (R)-2-methylpiperazine-1-carboxylate (2.09 g, 15.7 mmol). The reaction mixture was stirred at 80° C. for 16 h. The reaction mixture was filtered and the filter cake was washed with THF (2×10 mL), and the filter cake was collected and dried to give (((3R)-4-(tert-butoxycarbonyl)-3-methylpiperazin-1-ium-1-yl)methyl)trifluoroborate (4.3 g, crude) as a white solid. The crude was used in the next step without any other purification. ¹H NMR (400 MHz, DMSO-d6) δ 8.45-8.44 (m, 1H), 4.31-2.92 (m, 1H), 3.87-3.82 (m, 1H), 3.67-3.54 (m, 1H), 3.27-3.04 (m, 2H), 2.99-2.77 (m, 2H), 1.99 (br s, 2H), 1.83-1.70 (m, 1H), 1.50-1.37 (m, 9H), 1.21 (br d, J=7.2 Hz, 3H).

### Step 2. potassium (R)-((4-(tert-butoxycarbonyl)-3-methylpiperazin-1-yl)methyl)trifluoroborate

To a solution of (((3R)-4-(tert-butoxycarbonyl)-3-methylpiperazin-1-ium-1-yl)methyl)trifluoroborate (4.3 g, crude) in acetone (20 mL) was added K₂CO₃ (2.10 g, 15.2 mmol) and the reaction mixture was stirred at 25° C. for 16 h. The reaction mixture was filtered and the filter cake was washed with acetone (2×10 mL), and the filtrate was concentrated to give potassium (R)-((4-(tert-butoxycarbonyl)-3-methylpiperazin-1-yl)methyl)trifluoroborate (1.1 g, crude) as a white solid. The crude material was used in the next step without any other purification.

¹H NMR (400 MHz, DMSO-d6) δ 4.09 (br s, 1H), 3.79-3.60 (m, 1H), 3.51-3.21 (m, 1H), 2.98 (br s, 3H), 1.71-1.46 (m, 2H), 1.39 (s, 9H), 1.15 (d, J=7.2 Hz, 3H).

### Additional Borate Salts Prepared by the Method Above:

The borate salts in the following table were prepared by the method of potassium (R)-((4-(tert-butoxycarbonyl)-3-methylpiperazin-1-yl)methyl)trifluoroborate using the appropriate commercially

| Structure | Starting material |
|---|---|
| 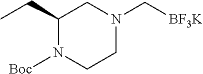 | tert-butyl (S)-2- ethylpiperazine-1- carboxylate |
| potassium (S)-((4-(tert- | |
| butoxycarbonyl)-3-ethylpiperazin- | |
| 1-yl)methyl)trifluoroborate | |
| 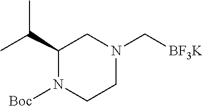 | tert-butyl (S)-2- isopropylpiperazine-1- carboxylate |
| potassium (S)-((4-(tert-butoxycarbonyl)-3- | |
| isopropylpiperazin-1- | |
| yl)methyl)trifluoroborate | |
| 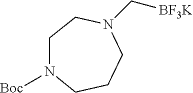 | tert-butyl 1,4-diazepane- 1-carboxylate |
| potassium ((4-(tert-butoxycarbonyl)-1,4- | |
| diazepan-1-yl)methyl)trifluoroborate | |

### Preparation of (R)-2-(difluoromethyl)-1-((tetrahydro-2H-pyran-4-yl)methyl)piperazine hydrochloride

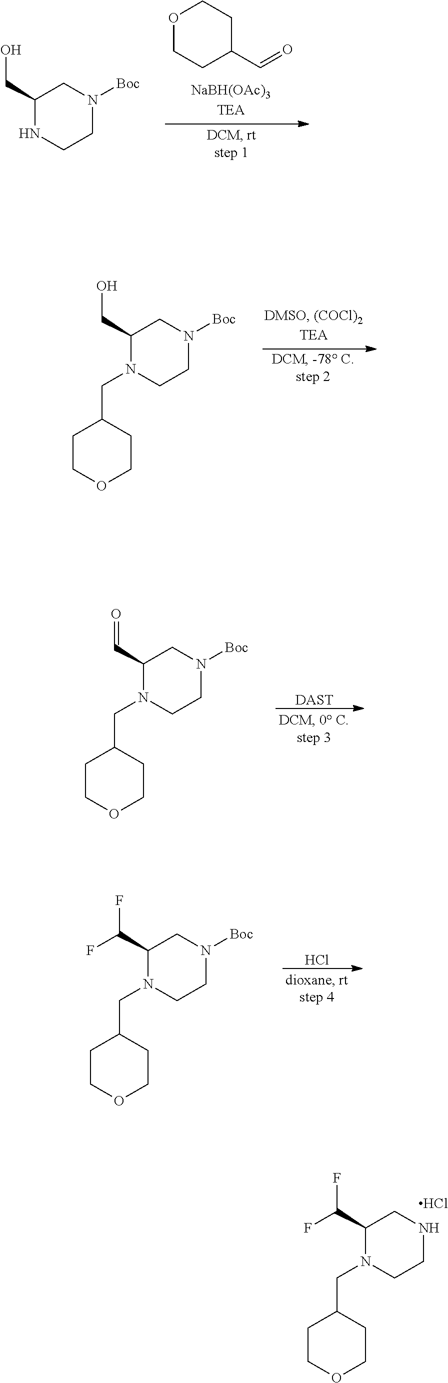

### Step 1. tert-butyl (R)-3-(hydroxymethyl)-4-((tetrahydro-2H-pyran-4-yl)methyl)piperazine-1-carboxylate

To a stirred solution of tert-butyl (R)-3-(hydroxymethyl)piperazine-1-carboxylate (3.0 g, 13.9 mmol) and tetrahydro-2H-pyran-4-carbaldehyde (2.37 g, 20.8 mmol) in DCM (25 mL) was added Et₃N (5.85 mL, 41.6 mmol). The reaction mixture was stirred for 90 min at rt. Sodium triacetoxy borohydride (5.88 g, 27.8 mmol) was then added slowly at 0° C. The reaction mixture was stirred at rt for 16 h. After completion, the reaction was diluted with DCM and water and the organic layer was dried over Na₂SO₄, filtered and concentrated. The crude compound was purified by silica gel chromotography (eluting with 20-30% EtOAc in hexanes) to afford tert-butyl (R)-3-(hydroxymethyl)-4-((tetrahydro-2H-pyran-4-yl)methyl)piperazine-1-carboxylate (3.0 g, 9.54 mmol, 69% yield). LCMS [M+H]⁺: 315.2.

### Step 2. tert-butyl (R)-3-formyl-4-((tetrahydro-2H-pyran-4-yl)methyl)piperazine-1-carboxylate

To a stirred solution of oxalyl chloride (1.62 mL, 19.1 mmol) in DCM (15 mL) at −78° C. was added DMSO (2.71 mL, 38.2 mmol) dropwise under an inert atmosphere. The reaction mixture was stirred at −78° C. for 15 min and then a solution of tert-butyl (R)-3-(hydroxymethyl)-4-((tetrahydro-2H-pyran-4-yl)methyl)piperazine-1-carboxylate (2.0 g, 6.4 mmol) in DCM (10 mL) was added dropwise at −78° C. The reaction mixture was stirred at −78° C. for 1 h and Et₃N (8.93 mL, 63.6 mmol) was added slowly. The reaction mixture was stirred at −78° C. for 1 h and allowed to warm to rt. The reaction was diluted with DCM and water and the organic layer was dried over Na₂SO₄, filtered and concentrated to afford crude tert-butyl (R)-4-(cyclohexylmethyl)-3-formylpiperazine-1-carboxylate (2.4 g, crude). LCMS [M+H]⁺: 313.2.

### Step 3. tert-butyl (R)-3-(difluoromethyl)-4-((tetrahydro-2H-pyran-4-yl)methyl)piperazine-1-carboxylate

To a stirred solution of tert-butyl (R)-4-(cyclohexylmethyl)-3-formylpiperazine-1-carboxylate (2.4 g, 7.68 mmol) in DCM (30 mL) at 0° C. was added DAST (2.0 mL, 15.4 mmol) under an inert atmosphere. The reaction mixture was stirred at 0° C. for 2 h. After completion, the reaction was quenched with saturated aqueous NaHCO₃ solution and diluted with DCM. The organic layer was dried over Na₂SO₄, filtered and concentrated. The crude compound was purified by silica gel chromotography (eluting with 20-30% EtOAc in hexanes) to afford tert-butyl (R)-3-(difluoromethyl)-4-((tetrahydro-2H-pyran-4-yl)methyl)piperazine-1-carboxylate (0.53 g, 1.58 mmol, 21% yield). LCMS [M+H]⁺: 334.9.

### Step 4. (R)-2-(difluoromethyl)-1-((tetrahydro-2H-pyran-4-yl)methyl)piperazine hydrochloride

To a stirred solution of tert-butyl (R)-3-(difluoromethyl)-4-((tetrahydro-2H-pyran-4-yl)methyl)piperazine-1-carboxylate (0.52 g, 1.55 mmol) in DCM (10 mL) at 0° C. was added a solution of HCl in dioxane (4.0 M, 6.0 mL). The reaction mixture was stirred at rt for 3 h. After completion, the mixture was concentrated and the crude compound was washed with diethyl ether to afford (R)-2-(difluoromethyl)-1-((tetrahydro-2H-pyran-4-yl)methyl)piperazine hydrochloride (0.50 g crude). LCMS [M+H]⁺: 235.1.

### Preparation of (R)-2-(difluoromethyl)-1-methylpiperazine hydrochloride

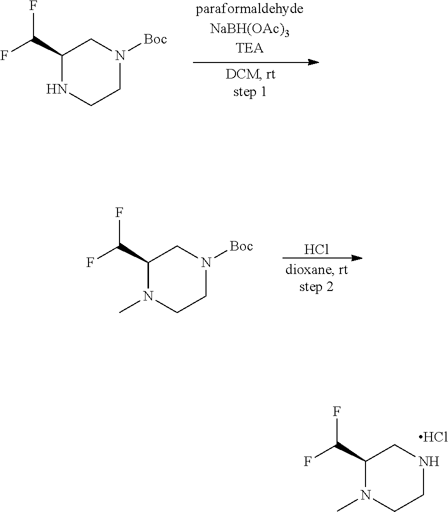

### Step 1. tert-butyl (R)-3-(difluoromethyl)-4-methylpiperazine-1-carboxylate

To a stirred solution of tert-butyl (R)-3-(difluoromethyl)piperazine-1-carboxylate [see US2019/144444, 2019, A1](0.35 g, 1.48 mmol) and paraformaldehyde (0.089 g, 2.96 mmol) in DCM (20 mL) was added Et₃N (0.41 mL, 2.96 mmol). The reaction mixture was stirred for 1 h at rt. Sodium triacetoxy borohydride (0.627 g, 2.96 mmol) was then added slowly at 0° C. The reaction mixture was stirred at rt for 14 h. After completion, the reaction was diluted with DCM and water and the organic layer was dried over Na₂SO₄, filtered and concentrated. The crude compound was purified by silica gel chromotography (eluting with 20-30% EtOAc in hexanes) to afford tert-butyl (R)-3-(difluoromethyl)-4-methylpiperazine-1-carboxylate (0.18 g, 0.71 mmol, 52% yield).

### Step 2. (R)-2-(difluoromethyl)-1-methylpiperazine hydrochloride

To a stirred solution of tert-butyl (R)-3-(difluoromethyl)-4-methylpiperazine-1-carboxylate (0.18 g, 0.71 mmol) in dioxane (2 mL) at 0° C. was added a solution of HCl in dioxane (4.0 M, 1.0 mL). The reaction mixture was stirred at rt for 3 h. After completion, the mixture was concentrated and the crude compound was washed with diethyl ether to afford (R)-2-(difluoromethyl)-1-methylpiperazine hydrochloride (0.16 g crude).

### Preparation of (R)-1-isobutyl-2-(methoxymethyl)piperazine hydrochloride

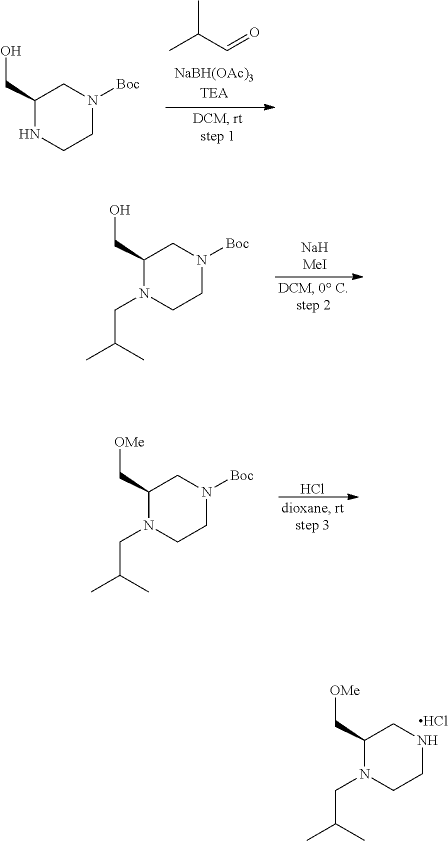

### Step 1. tert-butyl (R)-3-(hydroxymethyl)-4-isobutylpiperazine-1-carboxylate

To a stirred solution of tert-butyl (R)-3-(hydroxymethyl)piperazine-1-carboxylate (6.0 g, 27.7 mmol) and isobutyraldehyde (3.0 g, 41.6 mmol) dissolved in DCM (70 mL) was added Et₃N (11.7 mL, 83.2 mmol). The reaction mixture was stirred for 30 min at rt and then sodium triacetoxy borohydride (11.7 g, 55.5 mmol) was added in portions at 0° C. The reaction mixture was allowed to stirred at rt for 16 h. The reaction was diluted with DCM and water and the organic layer was dried over Na₂SO₄, filtered and concentrated. The crude compound was purified by silica gel chromotography (eluting with 10-20% EtOAc in hexanes) to afford tert-butyl (R)-3-(hydroxymethyl)-4-isobutylpiperazine-1-carboxylate (4.2 g, 15.5 mmol, 56% yield). LCMS [M+H]⁺: 273.3.

### Step 2. tert-butyl (R)-4-isobutyl-3-(methoxymethyl)piperazine-1-carboxylate

To a stirred solution of tert-butyl (R)-3-(hydroxymethyl)-4-isobutylpiperazine-1-carboxylate (0.60 g, 2.2 mmol) in DMF (10 mL) cooled to 0° C. was added NaH (0.13 g, 3.36 mmol) under an inert atmosphere. The reaction mixture was stirred at 0° C. for 30 min and then Mel (0.47 g, 3.36 mmol) was added at 0° C. The reaction was diluted with EtOAc and water and the organic layer was dried over Na₂SO₄, filtered and concentrated. The crude compound was purified by silica gel chromotography (eluting with 10-20% EtOAc in hexanes) to afford tert-butyl (R)-4-isobutyl-3-(methoxymethyl)piperazine-1-carboxylate (0.486 g, 1.7 mmol, 77%). LCMS [M+H]⁺: 287.1

### Step 3. (R)-1-isobutyl-2-(methoxymethyl)piperazine hydrochloride

To a stirred solution of tert-butyl (R)-4-isobutyl-3-(methoxymethyl)piperazine-1-carboxylate (0.392 g, 1.37 mmol) in DCM (7.0 mL) cooled to 0° C. was added a solution of HCl (4.0 M in dioxane, 4.0 mL). The reaction mixture was stirred at rt for 3 h and then concentrated. The crude compound was washed with diethyl ether to afford (R)-1-isobutyl-2-(methoxymethyl)piperazine hydrochloride (0.3 g, crude). LCMS [M+H]⁺: 187.1.
The borate salts in the following table were prepared by the method of potassium (R)-((4-(tert-butoxycarbonyl)-3-methylpiperazin-1-yl)methyl)trifluoroborate using the appropriate commercially available piperazine in step 1.

| Structure | Starting material |
|---|---|
| 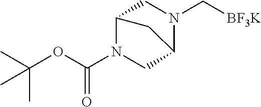 | tert-butyl (1S,4S)-2,5- diazabicyclo[2.2.1] heptane-2- carboxylate |
| potassium (((1S,4S)-5-(tert-butoxycarbonyl)- | |
| 2,5-diazabicyclo[2.2.1]heptan-2- | |
| yl)methyl)trifluoroborate | |
| 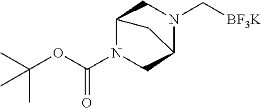 | tert-butyl (1R,4R)-2,5- diazabicyclo[2.2.1] heptane-2-carboxylate |
| potassium (((1R,4R)-5-(tert-butoxycarbonyl)- | |
| 2,5-diazabicyclo[2.2.1]heptan-2- | |
| yl)methyl)trifluoroborate | |
| 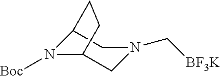 | tert-butyl (1R,5S)- 3,8- diazabicyclo[3.2.1] octane-8-carboxylate |
| potassium (((1R,5S)-8-(tert-butoxycarbonyl)- | |
| 3,8-diazabicyclo[3.2.1]octan-3- | |
| yl)methyl)trifluoroborate | |
| 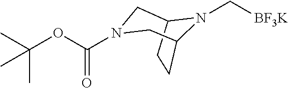 | tert-butyl (1R,5S)-3,8- diazabicyclo[3.2.1] octane-3-carboxylate |
| potassium (((1R,5S)-3-(tert-butoxycarbonyl)- | |
| 3,8-diazabicyclo[3.2.1]octan-8- | |
| yl)methyl)trifluoroborate | |
| 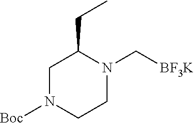 | tert-butyl (R)-3- ethylpiperazine-1- carboxylate |
| potassium (R)-((4-(tert-butoxycarbonyl)-2- | |
| ethylpiperazin-1-yl)methyl)trifluoroborate | |
| 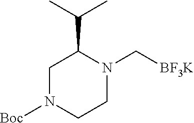 | tert-butyl (R)-3- isopropylpiperazine-1- carboxylate |
| potassium (R)-((4-(tert-butoxycarbonyl)-2- | |
| isopropylpiperazin-1-yl)methyl)trifluoroborate | |
| 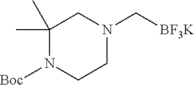 | tert-butyl 2,2- dimethylpiperazine-1- carboxylate |
| potassium ((4-(tert-butoxycarbonyl)-3,3- | |
| dimethylpiperazin-1-yl)methyl)trifluoroborate | |
| 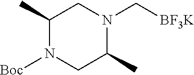 | tert-butyl (2S,5S)-2,5- dimethylpiperazine-1- carboxylate |
| potassium (((2S,5S)-4-(tert-butoxycarbonyl)- | |
| 2,5-dimethylpiperazin-1- | |
| yl)methyl)trifluoroborate | |
| 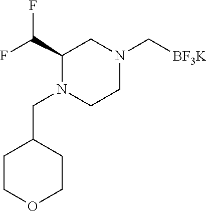 | (R)-2-(difluoromethyl)- 1-((tetrahydro-2H- pyran-4-yl)methyl) piperazine [vide infra] |
| potassium (R)-((3-(difluoromethyl)-4- | |
| ((tetrahydro-2H-pyran-4-yl)methyl)piperazin- | |
| 1-yl)methyl)trifluoroborate | |
| 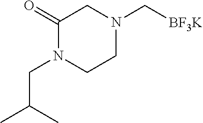 | 1-isobutylpiperazin- 2-one |
| potassium trifluoro((4-isobutyl-3- | |
| oxopiperazin-1-yl)methyl)borate | |
| 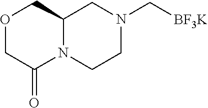 | (R)-hexahydropyrazino [2,1-c][1,4]oxazin- 4(3H)-one |
| potassium (R)-trifluoro((4- | |
| oxohexahydropyrazino[2,1-c][1,4]oxazin- | |
| 8(1H)-yl)methyl)borate | |
| 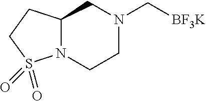 | (S)-hexahydro- 2H-isothiazolo[2,3- a]pyrazine 1,1-dioxide [see WO2007/28654, 2007, A1] |
| potassium (S)-((1,1-dioxidohexahydro-5H- | |
| isothiazolo[2,3-a]pyrazin-5- | |
| yl)methyl)trifluoroborate | |
| 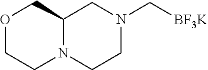 | (R)-octahydropyrazino [2,1-c][1,4]oxazine |
| potassium (R)- | |
| trifluoro((hexahydropyrazino[2,1- | |
| c][1,4]oxazin-8(1H)-yl)methyl)borate | |
| 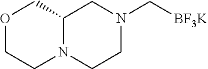 | (S)-octahydropyrazino [2,1-c][1,4]oxazine |
| potassium (S)- | |
| trifluoro((hexahydropyrazino[2,1- | |
| c][1,4]oxazin-8(1H)-yl)methyl)borate | |
| 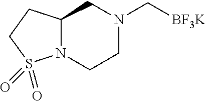 | (S)-hexahydro-2H- isothiazolo[2,3- a]pyrazine 1,1-dioxide [see US2021/47333, 2021, A1] |
| potassium (S)-((1,1-dioxidohexahydro-5H- | |
| isothiazolo[2,3-a]pyrazin-5- | |
| yl)methyl)trifluoroborate | |
| 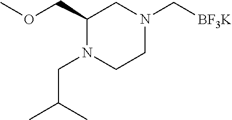 | (R)-1-isobutyl-2- (methoxymethyl) piperazine hydrochloride [vide infra] |
| potassium (R)-trifluoro((4-isobutyl-3- | |
| (methoxymethyl)piperazin-1-yl)methyl)borate | |

### Example 1. Preparation of 1-(5-((1-(cyclohexylmethyl)piperidin-4-yl)methyl)benzo[d]isoxazol-3-yl)dihydropyrimidine-2,4(1H,3H)-dione

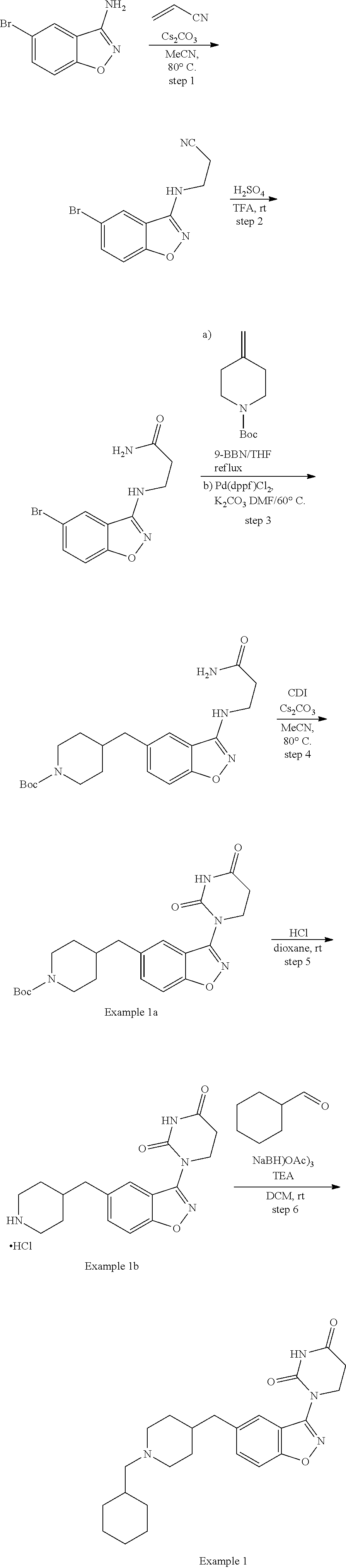

### Step 1: 3-((5-bromobenzo[d]isoxazol-3-yl)amino)propanenitrile

Cesium carbonate (2.39 g, 7.32 mmol) was added to a solution of 5-bromobenzo[d]isoxazol-3-amine (1.20 g, 5.63 mmol) and acrylonitrile (0.378 mL, 5.75 mmol) in MeCN (20 mL) at rt. The mixture was stirred at rt for 1 h and then heated at 80° C. for 2 h. The reaction mixture was cooled to rt and the orange suspension was filtered through celite, washing through with EtOAc. The filtrate was washed with brine, dried over Na₂SO₄, filtered and concentrated. The residue was purified by silica gel chromatography (eluted with EtOAc in heptane) to provide 3-((5-bromobenzo[d]isoxazol-3-yl)amino)propanenitrile as a white solid. (1.3 g, 4.9 mmol, 87% yield). LCMS [M+H]⁺: 266.1.

### Step 2: 3-((5-bromobenzo[d]isoxazol-3-yl)amino)propanamide

Sulfuric acid (4 mL, 75 mmol) was added dropwise to a solution of 3-((5-bromobenzo[d]isoxazol-3-yl)amino)propanenitrile (1.3 g, 4.9 mmol) in TFA (20 mL, 260 mmol) at rt. The mixture was stirred overnight at rt. The reaction mixture was then poured into ice water and stirred for 10 min. The precipitate that formed was collected by filtration and washed with water followed by diethyl ether three times. The solid was dried under vacuum to provide 3-((5-bromobenzo[d]isoxazol-3-yl)amino)propanamide (1.17 g, 4.12 mmol, 84% yield) as a white solid. LCMS [M+H]⁺: 284.1.

### Step 3: tert-butyl 4-((3-((3-amino-3-oxopropyl)amino)benzo[d]isoxazol-5-yl)methyl)piperidine-1-carboxylate

a) A vial containing tert-butyl 4-methylenepiperidine-1-carboxylate (400 mg, 2.03 mmol) was purged with nitrogen for 15 min and then a solution of 9-BBN (0.5M in THF, 4.07 mL, 2.03 mmol) was added. The vial was capped and the mixture was heated at 80° C. for 3.5 h and then cooled to rt.
b) The reaction mixture from part a was added by syringe to a vial containing a mixture of 3-((5-bromobenzo[d]isoxazol-3-yl)amino)propanamide (518 mg, 1.83 mmol), K₂CO₃ (350 mg, 2.53 mmol) and PdCl₂(dppf)·CH₂Cl₂ adduct (43 mg, 0.053 mmol) in DMF (10 mL). The vial was capped and the reaction mixture was heated overnight at 60° C. The reaction mixture was then cooled to rt and diluted with ethyl acetate and washed sequentially with water and brine. The organic layer was dried over Na₂SO₄, filtered and concentrated. The residue was purified by silica gel chromatography (eluted with 0-100% EtOAc in heptane) to provide tert-butyl 4-((3-((3-amino-3-oxopropyl)amino)benzo[d]isoxazol-5-yl)methyl)piperidine-1-carboxylate (572 mg, 1.42 mmol, 70% yield). LCMS [M+H]⁺: 403.1.

### Step 4: tert-butyl 4-((3-(2,4-dioxotetrahydropyrimidin-1(2H)-yl)benzo[d]isoxazol-5-yl)methyl)piperidine-1-carboxylate (Example 1a)

Cesium carbonate (607 mg, 1.86 mmol) was added to a suspension of tert-butyl 4-((3-((3-amino-3-oxopropyl)amino)benzo[d]isoxazol-5-yl)methyl)piperidine-1-carboxylate (500 mg, 1.24 mmol) and CDI (504 mg, 3.11 mmol) in acetonitrile (10 mL) at rt. The reaction mixture was then heated in a capped vial at 90° C. for 24 h. The reaction was cooled to rt and diluted with ethyl acetate and washed sequentially with water and brine. The organic layer was dried over Na₂SO₄, filtered and concentrated. The residue was purified by silica gel chromatography (eluted with 3:1 EtOAc:EtOH in heptane) to provide tert-butyl 4-((3-(2,4-dioxotetrahydropyrimidin-1(2H)-yl)benzo[d]isoxazol-5-yl)methyl)piperidine-1-carboxylate (380 mg, 0.887 mmol, 71% yield) as an off-white solid. LCMS [M+H]⁺: 429.3. ¹H NMR (500 MHz, Methanol-d4) δ 7.67 (s, 1H), 7.52 (d, J=8.6 Hz, 1H), 7.47 (d, J=8.7 Hz, 1H), 4.18 (t, J=6.6 Hz, 2H), 4.06 (d, J=13.4 Hz, 2H), 2.90 (t, J=6.6 Hz, 2H), 2.70 (d, J=7.2 Hz, 4H), 1.79 (d, J=13.0 Hz, 1H), 1.65 (d, J=13.3 Hz, 2H), 1.46 (d, J=1.5 Hz, 9H), 1.22-1.07 (m, 2H), NH proton not observed due to solvent exchange.

### Step 5: 1-(5-(piperidin-4-ylmethyl)benzo[d]isoxazol-3-yl)dihydropyrimidine-2,4(1H,3H)-dione hydrochloride (Example 1b)

A solution of HCl (4.0 M in dioxane, 4 mL, 16 mmol) was added to tert-butyl 4-((3-(2,4-dioxotetrahydropyrimidin-1 (2H)-yl)benzo[d]isoxazol-5-yl)methyl)piperidine-1-carboxylate (250 mg, 0.583 mmol) and the mixture was stirred for 2 h at rt. The reaction was then concentrated to give crude 1-(5-(piperidin-4-ylmethyl)benzo[d]isoxazol-3-yl)dihydropyrimidine-2,4(1H,3H)-dione hydrochloride (215 mg, crude) which was used without further purification. A sample was purified by reverse-phase HPLC using ACN/Water/0.1% TFA. The fractions containing the product were combined and lyophilized to afford a trifluoroacetate salt of 1-(5-(piperidin-4-ylmethyl)benzo[d]isoxazol-3-yl)dihydropyrimidine-2,4(1H,3H)-dione. LCMS [M+H]⁺: 329.3. 1H NMR (500 MHz, Methanol-d4) δ 7.70 (s, 1H), 7.55 (d, J=8.6 Hz, 1H), 7.49 (d, J=8.7 Hz, 1H), 4.19 (t, J=6.6 Hz, 2H), 3.38 (d, J=13.2 Hz, 2H), 3.00-2.87 (m, 4H), 2.77 (d, J=6.9 Hz, 2H), 2.05-1.84 (m, 3H), 1.57-1.35 (m, 2H), NH protons not observed due to solvent exchange.

### Step 6. 1-(5-((1-(cyclohexylmethyl)piperidin-4-yl)methyl)benzo[d]isoxazol-3-yl)dihydropyrimidine-2,4(1H,3H)-dione (Example 1)

Triethylamine (0.011 mL, 0.077 mmol) was added to a solution of 1-(5-(piperidin-4-ylmethyl)benzo[d]isoxazol-3-yl)dihydropyrimidine-2,4(1H,3H)-dione hydrochloride (28 mg, 0.077 mmol) and cyclohexanecarbaldehyde (17.2 mg, 0.153 mmol) in DCM (2 mL) at rt. The reaction mixture was stirred at rt for 5 min and then sodium triacetoxyborohydride (48.8 mg, 0.230 mmol) was added. The reaction was stirred at rt for 60 min and then quenched with a solution of saturated aqueous NaHCO₃. The mixture was extracted twice with DCM and the combined organic layers were washed with brine, dried over Na₂SO₄, filtered and concentrated. The residue was dissolved in DMSO, filtered through a 1 micron filter and purified by reverse-phase HPLC using ACN/Water/0.1% TFA. The fractions containing the product were combined and lyophilized to afford a trifluoroacetate salt of 1-(5-((1-(cyclohexylmethyl)piperidin-4-yl)methyl) benzo[d]isoxazol-3-yl)dihydropyrimidine-2,4(1H,3H)-dione (16.2 mg, 0.029 mmol, 38% yield). LCMS [M+H]⁺: 425.3. ¹H NMR (500 MHz, Methanol-d4) δ 7.58 (dd, J=1.8, 0.8 Hz, 1H), 7.44 (dd, J=8.6, 0.7 Hz, 1H), 7.37 (dd, J=8.7, 1.7 Hz, 1H), 4.07 (t, J=6.6 Hz, 2H), 3.48-3.38 (m, 2H), 2.87-2.73 (m, 6H), 2.65 (d, J=6.7 Hz, 2H), 1.88-1.75 (m, 3H), 1.74-1.65 (m, 5H), 1.64-1.57 (m, 1H), 1.45 (q, J=13.7 Hz, 2H), 1.32-1.18 (m, 2H), 1.13 (tdd, J=12.7, 9.4, 3.2 Hz, 1H), 1.00-0.87 (m, 2H), NH proton not observed due to solvent exchange.

The compounds in the following table were prepared by the method of Example 1, using the appropriate commercially available aldehyde in step 6.

| Example | | Mass | |
|---|---|---|---|
| No. | Structure | M + H | ¹H NMR |
| 2 | 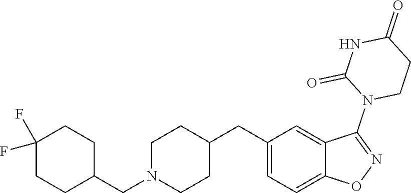 [SMILES: O=C1CCN(c2noc3ccc(CC4CCN(CC5CCC(F)(F)CC5)CC4)cc23)C(=O)N1] 1-(5-((1-((4,4- | 461.9 | (400 MHz, Methanol-d4) δ 7.66 (d, J = 1.7 Hz, 1H), 7.52 (d, J = 8.7 Hz, 1H), 7.46 (dd, J = 8.7, 1.6 Hz, 1H), 4.16 (t, J = 6.6 Hz, 2H), 3.24 (d, J = 12.2 Hz, 2H), 2.88 (t, J = 6.6 Hz, 2H), 2.72 (d, J = 6.6 Hz, 2H), 2.62 (d, J = 6.7 Hz, 2H), 2.44 (t, J = 12.0 Hz, 2H), 2.11 − 2.01 (m, 2H), 1.90 − 1.72 (m, 8H), 1.46 (q, J = 12.1, 11.7 Hz, 2H), 1.28 (d, J = 12.6 Hz, 2H), NH proton not observed due to solvent exhange. |
| | difluorocyclohexyl)methyl)piperidin-4- | | |
| | yl)methyl)benzo[d]isoxazol-3- | | |
| | yl)dihydropyrimidine-2,4(1H,3H)-dione | | |
| 3 | 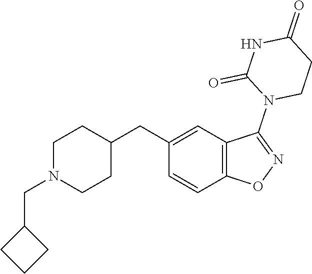 [SMILES: O=C1CCN(c2noc3ccc(CC4CCN(CC5CCC5)CC4)cc23)C(=O)N1] | 397.3 | (500 MHz, DMSO-d6) δ 10.89 (s, 1H), 7.69 − 7.65 (m, 1H), 7.62 (d, J = 1.6 Hz, 1H), 7.50 (dt, J = 8.6, 2.6 Hz, 1H), 4.07 (t, J = 6.6 Hz, 2H), 3.35 − 3.27 (m, 2H), 3.06 (dd, J = 7.0, 5.1 Hz, 2H), 2.81 (q, J = 6.4, 6.0 Hz, 4H), 2.73 − 2.59 (m, 3H), 2.15 − 1.98 (m, 2H), 1.94 − 1.83 (m, 1H), 1.77 (tt, J = 8.3, 5.8 Hz, 6H), 1.38 (q, J = 13.3 Hz, 2H). |
| | 1-(5-((1-(cyclobutylmethyl)piperidin-4- | | |
| | yl)methyl)benzo[d]isoxazol-3- | | |
| | yl)dihydropyrimidine-2,4(1H,3H)-dione | | |
| 4 | 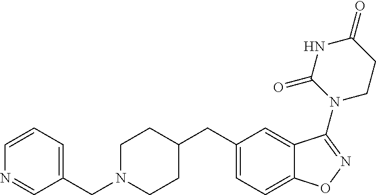 [SMILES: O=C1CCN(c2noc3ccc(CC4CCN(Cc5cccnc5)CC4)cc23)C(=O)N1] | 420.1 | (300 MHz, DMSO-d6) δ 10.84 (s, 1H), 8.42 (d, J = 4.8 Hz, 1H), 8.12 (s, 1H), 7.71 − 7.59 (m, 1H), 7.57 (d, J = 5.5 Hz, 1H), 7.43 (d, J = 8.6 Hz, 1H), 7.31 (dd, J = 7.8, 4.7 Hz, 1H), 4.03 (t, J = 6.5 Hz, 2H), 3.45 (s, 3H), 2.74 (dt, J = 10.7, 5.2 Hz, 4H), 2.60 (d, J = 6.3 Hz, 2H), 1.88 (t, J = 11.2 Hz, 2H), 1.52 (d, J = 12.7 Hz, 2H), 1.19 (d, J = 13.6 Hz, 2H). |
| | 1-(5-((1-(pyridin-3-ylmethyl)piperidin- | | |
| | 4-yl)methyl)benzo[d]isoxazol-3- | | |
| | yl)dihydropyrimidine-2,4(1H,3H)-dione | | |
| 5 | 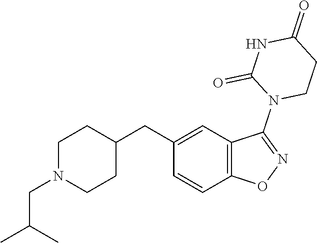 [SMILES: CC(C)CN1CCC(Cc2ccc3onc(N4CCC(=O)NC4=O)c3c2)CC1] | 385.3 | (500 MHz, DMSO-d6) δ 10.89 (s, 1H), 7.75 − 7.58 (m, 2H), 7.50 (d, J = 8.6 Hz, 1H), 4.07 (t, J= 6.5 Hz, 2H), 3.26 − 2.96 (m, 2H), 2.83 (dt, J = 25.5, 6.6 Hz, 6H), 2.69 (d, J = 6.4 Hz, 2H), 2.03 (tt, J =15.8, 8.2 Hz, 1H), 1.76 (d, J = 13.9 Hz, 3H), 1.49 (q, J = 13.2, 12.6 Hz, 2H), 0.95 (dd, J = 17.0, 6.5 Hz, 6H). |
| | 1-(5-((1-isobutylpiperidin-4- | | |
| | yl)methyl)benzo[d]isoxazol-3- | | |
| | yl)dihydropyrimidine-2,4(1H,3H)-dione | | |
| 6 | 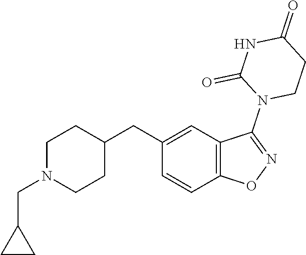 [SMILES: O=C1CCN(c2noc3ccc(CC4CCN(CC5CC5)CC4)cc23)C(=O)N1] | 383.3 | (500 MHz, DMSO-d6) δ 10.82 (s, 1H), 7.60 (d, J = 8.3 Hz, 1H), 7.56 (d, J = 1.6 Hz, 1H), 7.43 (dd, J = 8.7, 1.7 Hz, 1H), 4.00 (t, J = 6.6 Hz, 2H), 3.43 (d, J = 12.3 Hz, 2H), 3.18 (s, 1H), 2.85 (dd, J =7.3, 5.0 Hz, 2H), 2.81 − 2.77 (m, 1H), 2.73 (t, J = 6.5 Hz, 2H), 2.62 (d, J = 6.2 Hz, 2H), 1.71 (t, J = 13.5 Hz, 3H), 1.35 (q, J = 14.1, 13.7 Hz, 2H), 0.96 (ddt, J = 12.7, 8.2, 4.3 Hz, 1H), 0.61 − 0.46 (m, 2H), 0.33 − 0.17 (m, 2H). |
| | 1-(5-((1-(cyclopropylmethyl)piperidin- | | |
| | 4-yl)methyl)benzo[d]isoxazol-3- | | |
| | yl)dihydropyrimidine-2,4(1H,3H)-dione | | |
| 7 | 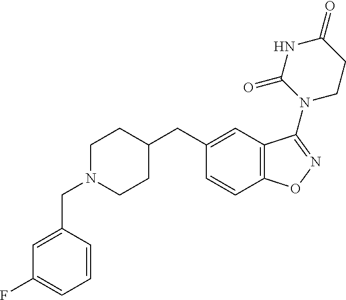 [SMILES: O=C1CCN(c2noc3ccc(CC4CCN(Cc5cccc(F)c5)CC4)cc23)C(=O)N1] | 437.3 | (500 MHz, Methanol-d4) δ 7.56 (s, 1H), 7.42 (t, J = 7.2 Hz, 2H), 7.36 (d, J = 8.7 Hz, 1H), 7.18 (dt, J = 15.5, 8.6 Hz, 3H), 4.19 (s, 2H), 4.06 (t, J = 6.2 Hz, 2H), 3.37 (d, J = 13.1 Hz, 2H), 2.87 (t, J =12.8 Hz, 2H), 2.77 (t, J = 6.5 Hz, 2H), 2.64 (d, J = 6.6 Hz, 2H), 1.83 (d, J = 13.4 Hz, 3H), 1.38 (q, J =13.6 Hz, 2H), NH proton not observed due to solvent exhange. |
| | 1-(5-((1-(3-fluorobenzyl)piperidin-4- | | |
| | yl)methyl)benzo[d]isoxazol-3- | | |
| | yl)dihydropyrimidine-2,4(1H,3H)-dione | | |
| 8 | 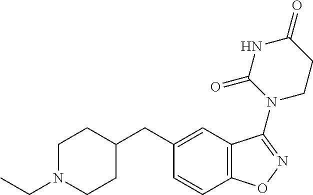 [SMILES: CCN1CCC(Cc2ccc3onc(N4CCC(=O)NC4=O)c3c2)CC1] | 357.4 | (500 MHz, DMSO-d6) δ 10.91 (d, J = 2.7 Hz, 1H), 7.71 − 7.67 (m, 1H), 7.66 − 7.63 (m, 1H), 7.54 − 7.42 (m, 1H), 4.08 (t, J = 6.6 Hz, 2H), 3.47 − 3.42 (m, 2H), 3.21 (dd, J = 12.1, 6.5 Hz, 1H), 3.06 (qd, J = 7.3, 4.9 Hz, 2H), 2.82 (q, J = 6.4, 5.6 Hz, 4H), 2.69 (d, J = 6.4 Hz, 2H), 1.86 − 1.79 (m, 2H), 1.39 (q, J = 12.5, 11.7 Hz, 2H), 1.21 (dt, J = 11.3, 6.4 Hz, 3H). |
| | 1-(5-((1-ethylpiperidin-4- | | |
| | yl)methyl)benzo[d]isoxazol-3- | | |
| | yl)dihydropyrimidine-2,4(1H,3H)-dione | | |
| 9 | 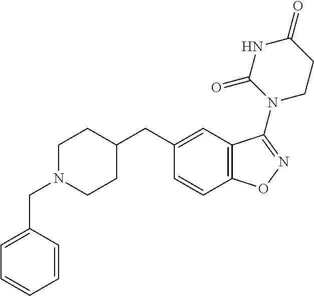 [SMILES: O=C1CCN(c2noc3ccc(CC4CCN(Cc5ccccc5)CC4)cc23)C(=O)N1] | 419.3 | (500 MHz, Methanol-d4) δ 7.68 (s, 1H), 7.57 − 7.46 (m, 7H), 4.29 (s, 2H), 4.18 (t, J = 6.3 Hz, 2H), 3.48 (d, J = 12.8 Hz, 2H), 2.98 (t, J = 12.9 Hz, 2H), 2.89 (t, J = 6.4 Hz, 2H), 2.76 (d, J = 6.5 Hz, 2H), 1.94 (d, J = 13.5 Hz, 3H), 1.49 (q, J = 13.6 Hz, 2H), NH proton not observed due to solvent exhange. |
| | 1-(5-((1-benzylpiperidin-4- | | |
| | yl)methyl)benzo[d]isoxazol-3- | | |
| | yl)dihydropyrimidine-2,4(1H,3H)-dione | | |
| 10 | 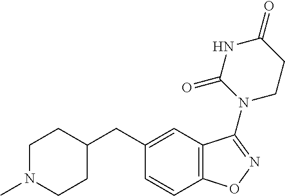 [SMILES: CN1CCC(Cc2ccc3onc(N4CCC(=O)NC4=O)c3c2)CC1] | 343.2 | (500 MHz, DMSO-d6) δ 10.90 (s, 1H), 7.76 − 7.60 (m, 2H), 7.52 (ddd, J = 8.7, 7.0, 2.5 Hz, 1H), 4.08 (t, J = 6.6 Hz, 2H), 3.44 − 3.33 (m, 2H), 2.91 − 2.79 (m, 4H), 2.71 (s, 3H), 2.68 (d, J = 6.4 Hz, 2H), 1.86 − 1.66 (m, 3H), 1.38 (q, J = 13.2 Hz, 2H). |
| | 1-(5-((1-methylpiperidin-4- | | |
| | yl)methyl)benzo[d]isoxazol-3- | | |
| | yl)dihydropyrimidine-2,4(1H,3H)-dione | | |
| 11 | 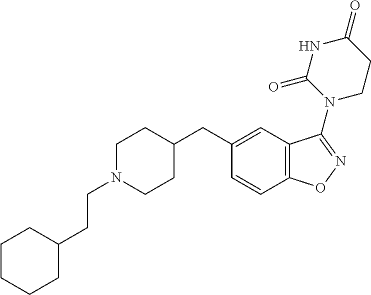 [SMILES: O=C1CCN(c2noc3ccc(CC4CCN(CCC5CCCCC5)CC4)cc23)C(=O)N1] | 439.4 | (500 MHz, Methanol-d4) δ 7.58 (s, 1H), 7.44 (d, J = 8.6 Hz, 1H), 7.37 (d, J = 8.8 Hz, 1H), 4.07 (t, J = 6.6 Hz, 2H), 3.45 (d, J = 12.5 Hz, 2H), 3.02 − 2.96 (m, 2H), 2.78 (t, J = 6.7 Hz, 4H), 2.65 (d, J= 6.6 Hz, 2H), 1.84 (d, J = 13.4 Hz, 2H), 1.61 (dd, J = 29.4, 12.2 Hz, 5H), 1.50 (q, J = 7.4 Hz, 2H), 1.38 (q, J = 13.4 Hz, 2H), 1.28 − 1.07 (m, 5H), 0.90 (q, J = 11.9 Hz, 2H), NH proton not observed due to solvent exhange. |
| | 1-(5-((1-(2-cyclohexylethyl)piperidin-4- | | |
| | yl)methyl)benzo[d]isoxazol-3- | | |
| | yl)dihydropyrimidine-2,4(1H,3H)-dione | | |
| 12 | 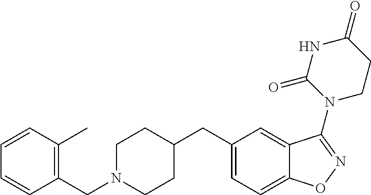 [SMILES: Cc1ccccc1CN1CCC(Cc2ccc3onc(N4CCC(=O)NC4=O)c3c2)CC1] | 433.2 | (400 MHz, DMSO-d6) δ 10.87 (s, 1H), 7.67 − 7.53 (m, 2H), 7.45 (d, J = 8.7 Hz, 1H), 7.15 (d, J = 22.3 Hz, 4H), 4.05 (t, J = 6.6 Hz, 2H), 2.82 − 2.70 (m, 4H), 2.62 (d, J = 6.3 Hz, 3H), 2.28 (s, 4H), 1.87 (t, J = 11.4 Hz, 2H), 1.53 (d, J = 12.9 Hz, 3H), 1.19 (t, J = 14.1 Hz, 2H). |
| | 1-(5-((1-(2-methylbenzyl)piperidin-4- | | |
| | yl)methyl)benzo[d]isoxazol-3- | | |
| | yl)dihydropyrimidine-2,4(1H,3H)-dione | | |

### Example 13. Preparation of 1-(5-((1-(2,2,2-trifluoroethyl)piperidin-4-yl)methyl)benzo[d]isoxazol-3-yl)dihydropyrimidine-2,4(1H,3H)-dione

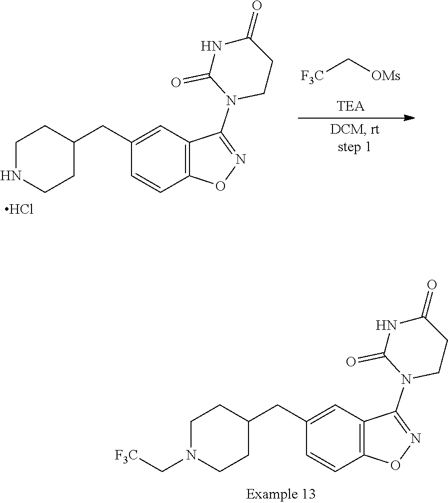 [SMILES: O=C1CCN(c2noc3ccc(CC4CCNCC4)cc23)C(=O)N1 ; CS(=O)(=O)OCC(F)(F)F ; Cl ; O=C1CCN(c2noc3ccc(CC4CCN(CC(F)(F)F)CC4)cc23)C(=O)N1]

### Step 1. 1-(5-((1-(2,2,2-trifluoroethyl)piperidin-4-yl)methyl)benzo[d]isoxazol-3-yl)dihydropyrimidine-2,4(1H,3H)-dione

2,2,2-Trifluoroethyl methanesulfonate (120 mg, 0.68 mmol) was added to a solution of 1-(5-(piperidin-4-ylmethyl)benzo[d]isoxazol-3-yl)dihydropyrimidine-2,4(1H,3H)-dione hydrochloride (150 mg, 0.45 mmol) and Et₃N (0.19 mL, 1.4 mmol) in DCM (7 mL) at rt. The mixture was stirred at rt for 36 h and then diluted with DCM and water. The organic layer was dried over Na₂SO₄, filtered and concentrated. The crude compound was purified by reverse-phase HPLC: ATLANTIS (250 mm×21.2 mm), 5.0p, A=0.1% HCOOH in water, B=MeCN, Flow: 20 mL/min. The fractions containing product were combined and lyophilized to afford 1-(5-((1-(2,2,2-trifluoroethyl)piperidin-4-yl)methyl)benzo[d]isoxazol-3-yl)dihydropyrimidine-2,4(1H,3H)-dione (46 mg, 0.11 mmol, 24% yield) as an off-white solid. LCMS [M+H]⁺: 411.2. HPLC: Rt=5.70 min. ¹H NMR (400 MHz, DMSO-d6) δ 10.86 (brs, 1H), 7.63-7.58 (m, 2H), 7.47-7.44 (m, 1H), 4.05 (t, J=6.8 Hz, 2H), 3.12-3.04 (m, 2H), 2.87-2.84 (m, 2H), 2.80-2.77 (m, 2H), 2.62-2.60 (m, 2H), 2.22 (t, J=11.2 Hz, 2H), 1.53-1.45 (m, 3H), 1.25-1.19 (m, 2H).

### Example 14. 1-(5-((1-(isopropylsulfonyl)piperidin-4-yl)methyl)benzo[d]isoxazol-3-yl)dihydropyrimidine-2,4(1H,3H)-dione

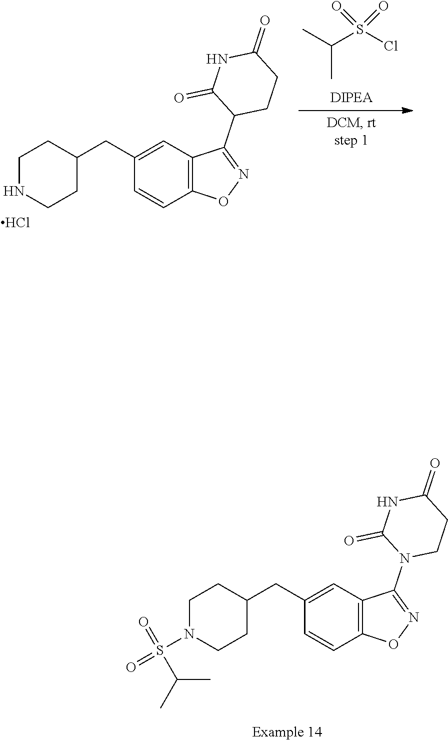

### Step 1. 1-(5-((1-(isopropylsulfonyl)piperidin-4-yl)methyl)benzo[d]isoxazol-3-yl)dihydropyrimidine-2,4(1H,3H)-dione

DIPEA (0.048 mL, 0.27 mmol) and propane-2-sulfonyl chloride (0.0074 mL, 0.066 mmol) were added to a solution of 1-(5-(piperidin-4-ylmethyl)benzo[d]isoxazol-3-yl)dihydropyrimidine-2,4(1H,3H)-dione hydrochloride (20 mg, 0.055 mmol) in DCM (1.5 mL) at rt. The mixture was stirred at rt for 2 h. Additional DIPEA (0.048 mL, 0.27 mmol) and propane-2-sulfonyl chloride (0.0074 mL, 0.066 mmol) were added and the mixture was stirred 45 min, then diluted with DCM and washed sequentially with water and brine. The organic layer was dried over sodium sulfate, filtered and concentrated. The residue was dissolved in DMSO, filtered through a 1 micron filter and purified by reverse-phase HPLC using ACN/Water/0.1% TFA. The fractions containing the product were combined and lyophilized to afford a trifluoroacetate salt of 1-(5-((1-(isopropylsulfonyl)piperidin-4-yl)methyl)benzo[d]isoxazol-3-yl)dihydropyrimidine-2,4(1H,3H)-dione (6.8 mg, 0.012 mmol, 22% yield). LCMS [M+H]⁺: 435.3. ¹H NMR (500 MHz, Methanol-d4) δ 7.56 (s, 1H), 7.41 (d, J=8.7 Hz, 1H), 7.36 (d, J=8.8 Hz, 1H), 4.06 (t, J=6.6 Hz, 2H), 3.65 (d, J=12.7 Hz, 2H), 3.15 (d, J=6.8 Hz, 1H), 2.83-2.70 (m, 4H), 2.61 (d, J=7.0 Hz, 2H), 1.62 (t, J=17.1 Hz, 3H), 1.22-1.15 (m, 8H), NH proton not observed due to solvent exchange.

### Example 15. 1-(5-(((2S,4S)-1-((4,4-difluorocyclohexyl)methyl)-2-methylpiperidin-4-yl)methyl)benzo[d]isoxazol-3-yl)dihydropyrimidine-2,4(1H,3H)-dione and Example 16. 1-(5-(((2S,4R)-1-((4,4-difluorocyclohexyl)methyl)-2-methylpiperidin-4-yl)methyl)benzo[d]isoxazol-3-yl)dihydropyrimidine-2,4(1H,3H)-dione

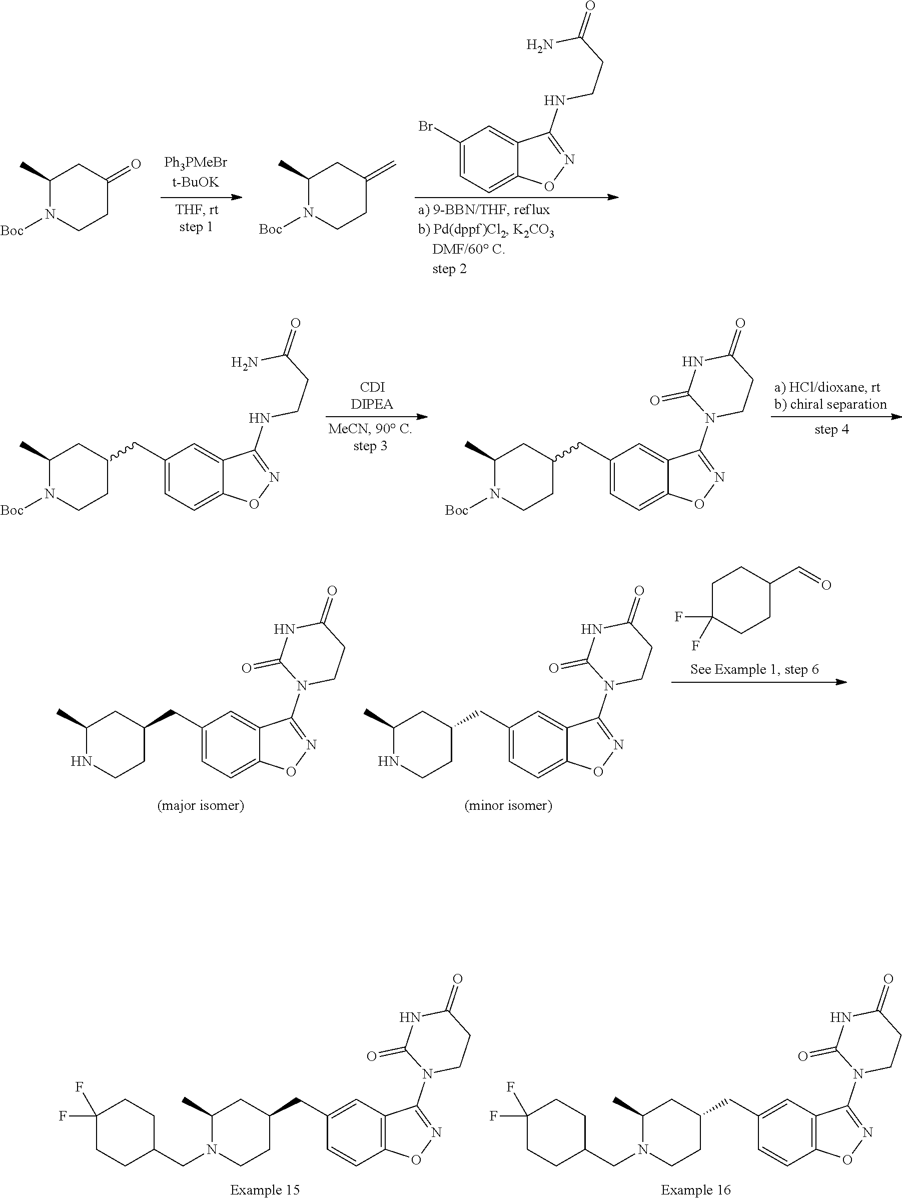

### Step 1. Step 1. tert-butyl (S)-2-methyl-4-methylenepiperidine-1-carboxylate

To dry t-BuOK (1.58 g, 14.1 mmol) in THF (20 mL) was added methyltriphenylphosphonium bromide (5.02 g, 14.07 mmol) at 0° C., then the mixture was stirred at rt for 2 h. The mixture was cooled to 0° C. and a solution of tert-butyl (S)-2-methyl-4-oxopiperidine-1-carboxylate (2 g, 9.38 mmol) in THF (5 mL) was added slowly. The reaction mixture was stirred at rt for 14 h. The reaction mixture was quenched with a solution of saturated aqueous NH₄Cl (50 mL) and extracted twice with EtOAc. The combined organic layers were concentrated to give the crude product. The crude product was purified by flash silica gel chromatography (eluted with 0-10% EtOAc/petroleum ether) to give tert-butyl (S)-2-methyl-4-methylenepiperidine-1-carboxylate (1.7 g, 8.1 mmol, 86% yield) as a yellow oil. ¹H NMR (400 MHz, CDCl₃) δ 4.85 (d, J=1.6 Hz, 1H), 4.74 (d, J=1.6 Hz, 1H), 4.51-4.48 (m, 1H), 4.04-4.01 (m, 1H), 2.89-2.82 (m, 1H), 2.42-237 (m, 1H), 2.17-2.13 (m, 2H), 2.03-2.00 (m, 1H), 1.47 (s, 9H), 1.07 (d, J=6.8 Hz, 3H).

### Step 2. tert-butyl (2S)-4-((3-((3-amino-3-oxopropyl)amino)benzo[d]isoxazol-5-yl)methyl)-2-methylpiperidine-1-carboxylate

a) A vial containing tert-butyl (S)-2-methyl-4-methylenepiperidine-1-carboxylate (0.74 g, 3.5 mmol) was purged with nitrogen for 15 min and then a solution of 9-BBN (0.5M in THF, 14.0 mL, 7.02 mmol) was added. The vial was capped and the mixture was heated at 80° C. for 3.5 h and then cooled to rt.
b) The reaction mixture from part a was added by syringe to a vial containing a mixture of 3-((5-bromobenzo[d]isoxazol-3-yl)amino)propanamide (1.0 g, 3.5 mmol), K₂CO₃ (726 mg, 5.26 mmol) and PdCl₂(dppf)·CH₂Cl₂ adduct (57 mg, 0.070 mmol) in DMF (5 mL) and water (2.5 mL). The vial was capped and the reaction mixture was heated at 90° C. for 14 h. The reaction mixture was then cooled to rt and diluted with ethyl acetate and washed sequentially with water and brine. The organic layer was dried over Na₂SO₄, filtered and concentrated. The residue was purified by silica gel chromatography (eluted with 5% MeOH in DCM) to provide tert-butyl (2S)-4-((3-((3-amino-3-oxopropyl)amino)benzo[d]isoxazol-5-yl)methyl)-2-methylpiperidine-1-carboxylate (1.0 g, 2.4 mmol, 69% yield) as a mixture of cis (major) and trans (minor) isomers. LCMS [M+H]⁺: 417.6.

### Step 3. tert-butyl (2S)-4-((3-(2,4-dioxotetrahydropyrimidin-1(2H)-yl)benzo[d]isoxazol-5-yl)methyl)-2-methylpiperidine-1-carboxylate

CDI (1.39 g, 8.62 mmol) and DIPEA (1.5 mL, 8.62 mmol) were added to a solution of tert-butyl (2S)-4-((3-((3-amino-3-oxopropyl)amino)benzo[d]isoxazol-5-yl)methyl)-2-methylpiperidine-1-carboxylate (900 mg, 2.15 mmol) in acetonitrile (20 mL) at rt. The reaction mixture was then heated at 90° C. for 8 h. The reaction was cooled to rt and concentrated. The residue was purified first by silica gel chromatography (eluted with 70% EtOAc in heptane) and then re-purified by reverse-phase HPLC: Mobile Phase: A=0.1% HCOOH in WATER, B=CH3CN
Column: JUPITER Phenomenex (250 mm×21.2 mm), 4.0p. The fractions containing product were concentrated to obtain tert-butyl (2S)-4-((3-(2,4-dioxotetrahydropyrimidin-1(2H)-yl)benzo[d]isoxazol-5-yl)methyl)-2-methylpiperidine-1-carboxylate (280 mg, 0.633, 29% yield). LCMS [M+H]⁺: 443.1.

### Step 4. 1-(5-(((2S,4S)-2-methylpiperidin-4-yl)methyl)benzo[d]isoxazol-3-yl)dihydropyrimidine-2,4(1H,3H)-dione and 1-(5-(((2S,4R)-2-methylpiperidin-4-yl)methyl)benzo[d]isoxazol-3-yl)dihydropyrimidine-2,4(1H,3H)-dione

A solution of HCl (4.0 M in dioxane, 3 mL) was added dropwise to a solution of tert-butyl (2S)-4-((3-(2,4-dioxotetrahydropyrimidin-1(2H)-yl)benzo[d]isoxazol-5-yl)methyl)-2-methylpiperidine-1-carboxylate (0.25 g, 0.56 mmol) and the mixture was stirred for 4 h at rt. The reaction was then concentrated and the residue was basified by the addition of a solution of saturated aqueous NaHCO₃. The mixture was extracted with DCM and the organic layer was washed with brine, dried over Na₂SO₄, filtered and concentrated to afford the crude product as a mixture of cis (major) and trans (minor) isomers. The isomers were separated by chiral HPLC: COLUMN: CHIRALPAK IH, 250 MM×20 MM×5 MICRON, MOBILE PHASE: HEXANE (A) 0.1% DEA IN EtOH:MeOH, 1:1, (B), FLOW; 15 ML, ISOCRATIC:75(A):25(B), DILUENT: ETHANOL-8 ML, INJECTION VOLUME-0.5 ML, RUN TIME—28 min. The collected fractions were concentrated under reduced pressure to afford:
Compound 1: 1-(5-(((2S,4S)-2-methylpiperidin-4-yl)methyl)benzo[d]isoxazol-3-yl)dihydropyrimidine-2,4(1H,3H)-dione (70 mg, 0.20 mmol, 35%); LCMS [M+H]⁺: 343.1; HPLC: (6.03 min).
Compound 2: 1-(5-(((2S,4R)-2-methylpiperidin-4-yl)methyl)benzo[d]isoxazol-3-yl)dihydropyrimidine-2,4(1H,3H)-dione (30 mg, 0.087 mmol, 15%); LCMS [M+H]⁺: 343.2; HPLC: (7.20 min).

### Step 5

The isomers separated in the previous step were treated separately to the conditions of Example 1, step 6 using 4,4-difluorocyclohexane-1-carbaldehyde to give the respective products.

### Example 15. 1-(5-(((2S,4S)-1-((4,4-difluorocyclohexyl)methyl)-2-methylpiperidin-4-yl)methyl)benzo[d]isoxazol-3-yl)dihydropyrimidine-2,4(1H,3H)-dione

LCMS [M+H]⁺: 475.1. ¹H NMR (400 MHz, Methanol-d4) δ 7.68 (d, J=1.6 Hz, 1H), 7.53 (d, J=8.7 Hz, 1H), 7.47 (dd, J=8.6, 1.7 Hz, 1H), 4.17 (t, J=6.6 Hz, 2H), 3.53 (d, J=12.7 Hz, 1H), 3.21-3.11 (m, 1H), 3.05 (s, 1H), 2.88 (t, J=6.6 Hz, 2H), 2.73 (d, J=7.0 Hz, 4H), 2.07 (s, 2H), 1.87 (dt, J=28.3, 14.9 Hz, 8H), 1.55-1.27 (m, 7H), NH proton not observed due to solvent exchange.

### Example 16. 1-(5-(((2S,4R)-1-((4,4-difluorocyclohexyl)methyl)-2-methylpiperidin-4-yl)methyl)benzo[d]isoxazol-3-yl)dihydropyrimidine-2,4(1H,3H)-dione

LCMS [M+H]⁺: 475.1. ¹H NMR (400 MHz, Methanol-d4) δ 7.68 (s, 1H), 7.54 (d, J=8.5 Hz, 1H), 7.51-7.44 (m, 1H), 4.17 (t, J=6.7 Hz, 2H), 3.74 (s, 1H), 3.24 (s, 2H), 3.02 (d, J=5.8 Hz, 2H), 2.88 (t, J=6.6 Hz, 2H), 2.76 (d, J=7.2 Hz, 2H), 2.13 (d, J=32.9 Hz, 3H), 1.97-1.73 (m, 8H), 1.63 (d, J=20.5 Hz, 1H), 1.45-1.28 (m, 5H), NH proton not observed due to solvent exchange.

### Example 17. tert-butyl 3-((3-(2,4-dioxotetrahydropyrimidin-1(2H)-yl)benzo[d]isoxazol-5-yl)methyl)azetidine-1-carboxylate

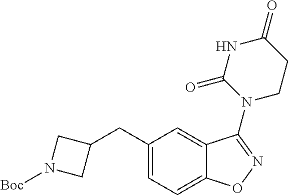 [SMILES: CC(C)(C)OC(=O)N1CC(Cc2ccc3onc(N4CCC(=O)NC4=O)c3c2)C1]

Prepared from tert-butyl 3-methyleneazetidine-1-carboxylate by the method of Example 1, steps 3-4. LCMS [M+H]⁺: 401.0. ¹H NMR (400 MHz, Methanol-d4) δ 7.69 (s, 1H), 7.49 (q, J=8.8 Hz, 2H), 4.16 (td, J=6.7, 1.4 Hz, 2H), 3.97 (t, J=8.3 Hz, 2H), 3.66 (dd, J=8.6, 5.3 Hz, 2H), 3.02 (d, J=7.8 Hz, 2H), 2.97-2.82 (m, 3H), 1.42 (s, 9H), NH proton not observed due to solvent exchange.

### Example 18. 1-(5-(azetidin-3-ylmethyl)benzo[d]isoxazol-3-yl)dihydropyrimidine-2,4(1H,3H)-dione

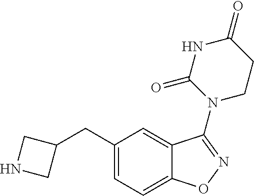 [SMILES: O=C1CCN(c2noc3ccc(CC4CNC4)cc23)C(=O)N1]

Prepared from Example 17 by the method of Example 1, step 5. LCMS [M+H]⁺: 301.2. ¹H NMR (400 MHz, Methanol-d4) δ 7.77 (s, 1H), 7.59 (d, J=8.7 Hz, 1H), 7.53 (dd, J=8.7, 1.6 Hz, 1H), 4.18 (t, J=6.6 Hz, 2H), 3.70 (dd, J=11.8, 3.7 Hz, 1H), 3.58 (dd, J=11.8, 4.5 Hz, 1H), 3.17 (dd, J=13.1, 7.7 Hz, 1H), 3.03 (dd, J=13.1, 5.9 Hz, 1H), 2.91 (dt, J=16.0, 6.4 Hz, 4H), 2.43 (s, 1H), NH protons not observed due to solvent exchange.

### Example 19. 1-(5-((1-(cyclohexylmethyl)azetidin-3-yl)methyl)benzo[d]isoxazol-3-yl)dihydropyrimidine-2,4(1H,3H)-dione

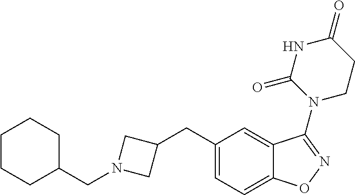 [SMILES: O=C1CCN(c2noc3ccc(CC4CN(CC5CCCCC5)C4)cc23)C(=O)N1]

Prepared from Example 18 by the method of Example 1, step 6. LCMS [M+H]⁺: 397.3. ¹H NMR (400 MHz, Methanol-d4) δ 7.72 (s, 1H), 7.55 (d, J=8.6 Hz, 1H), 7.48 (d, J=8.9 Hz, 1H), 4.16 (t, J=7.2 Hz, 4H), 3.95 (t, J=9.1 Hz, 2H), 3.22 (p, J=7.8 Hz, 1H), 3.09 (d, J=7.6 Hz, 2H), 3.02 (d, J=7.0 Hz, 2H), 2.88 (t, J=6.6 Hz, 2H), 1.81-1.58 (m, 6H), 1.25 (dt, J=29.2, 12.4 Hz, 3H), 1.00 (q, J=12.0, 11.3 Hz, 2H), NH proton not observed due to solvent exchange.

### Example 20. 1-(5-((1-methylazetidin-3-yl)methyl)benzo[d]isoxazol-3-yl)dihydropyrimidine-2,4(1H,3H)-dione

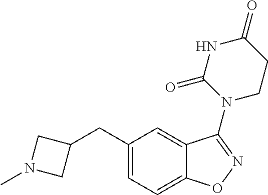 [SMILES: CN1CC(Cc2ccc3onc(N4CCC(=O)NC4=O)c3c2)C1]

Prepared from Example 18 by the method of Example 1, step 6 using acetaldehyde. LCMS [M+H]⁺: 315.4. ¹H NMR (400 MHz, Methanol-d4) δ 7.72 (s, 1H), 7.56 (d, J=8.6 Hz, 1H), 7.48 (dd, J=8.9, 1.6 Hz, 1H), 4.17 (t, J=6.7 Hz, 2H), 4.11 (t, J=9.4 Hz, 2H), 3.93 (t, J=8.6 Hz, 2H), 3.26-3.15 (m, 1H), 3.09 (d, J=7.7 Hz, 2H), 2.93-2.83 (m, 5H), NH proton not observed due to solvent exchange.

### Example 21. 1-(5-((4-(cyclohexylmethyl)piperazin-1-yl)methyl)benzo[d]isoxazol-3-yl)dihydropyrimidine-2,4(1H,3H)-dione

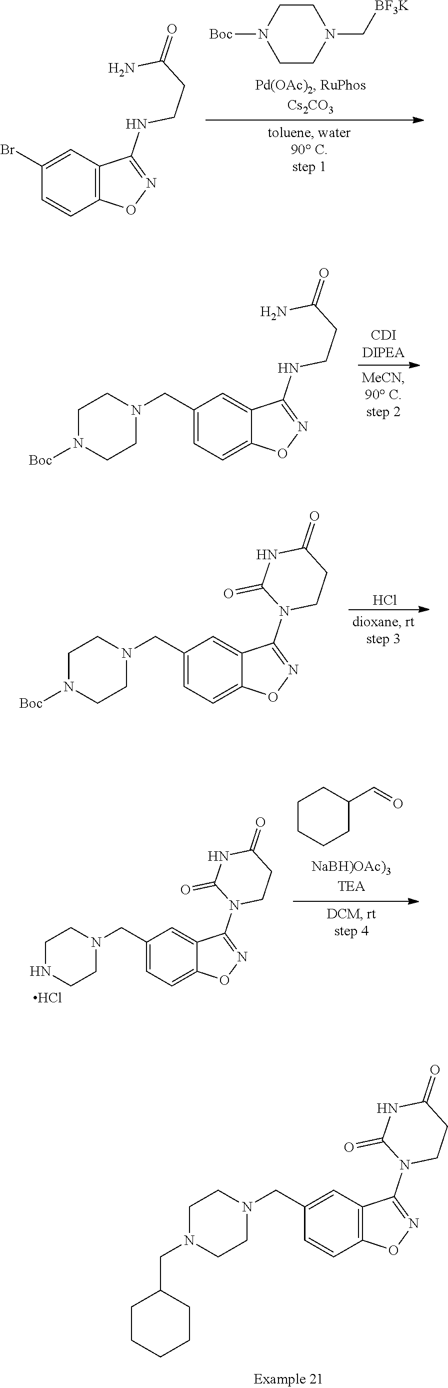

### Step 1. tert-butyl 4-((3-((3-amino-3-oxopropyl)amino)benzo[d]isoxazol-5-yl)methyl)piperazine-1-carboxylate

To a suspension of 3-((5-bromobenzo[d]isoxazol-3-yl)amino)propanamide (125 mg, 0.44 mmol) in toluene (4 mL) and water (0.4 mL) at room temperature was added Cs₂CO₃ (573 mg, 1.76 mmol), potassium {[4-(tert-butoxycarbonyl)-1-piperazinyl]methyl}(trifluoro)borate (269 mg, 0.88 mmol) and RuPhos (41 mg, 0.088 mmol), followed by Pd(OAc)₂ (9.9 mg, 0.044 mmol). The mixture was stirred at 90° C. for 3 h, then cooled to rt and partitioned between EtOAc and water. The organic layer was separated, washed with brine, dried over sodium sulfate, filtered and concentrated. The residue was purified by silica gel chromatography (eluting with 0-100% 3:1 EtOAc:EtOH in heptane) to give tert-butyl 4-((3-((3-amino-3-oxopropyl)amino)benzo[d]isoxazol-5-yl)methyl)piperazine-1-carboxylate (100 mg, 0.248 mmol, 56% yield). LCMS [M+H]⁺: 404.4. ¹H NMR (500 MHz, Methanol-d4) δ 7.70 (d, J=1.6 Hz, 1H), 7.56 (dd, J=8.6, 1.7 Hz, 1H), 7.37 (d, J=8.6 Hz, 1H), 3.69-3.59 (m, 4H), 3.45 (s, 4H), 2.65 (t, J=6.8 Hz, 2H), 2.44 (t, J=5.2 Hz, 4H), 1.47 (s, 9H), NH protons not observed due to solvent exchange.

### Step 2. tert-butyl 4-((3-(2,4-dioxotetrahydropyrimidin-1(2H)-yl)benzo[d]isoxazol-5-yl)methyl)piperazine-1-carboxylate

DIPEA (189 mg, 1.47 mmol) and CDI (317 mg, 1.96 mmol) was added to a solution of tert-butyl 4-((3-((3-amino-3-oxopropyl)amino)benzo[d]isoxazol-5-yl)methyl)piperazine-1-carboxylate (200 mg, 0.49 mmol) in acetonitrile (20 mL) at rt. The reaction mixture was then heated at 90° C. for 8 h. The reaction was cooled to rt and concentrated. The residue was purified by silica gel chromatography (eluted with 70% EtOAc in heptane) to provide tert-butyl 4-((3-(2,4-dioxotetrahydropyrimidin-1(2H)-yl)benzo[d]isoxazol-5-yl)methyl)piperazine-1-carboxylate (120 mg, 0.279 mmol, 57% yield). LCMS [M+H]⁺: 430.2.

### Step 3. 1-(5-(piperazin-1-ylmethyl)benzo[d]isoxazol-3-yl)dihydropyrimidine-2,4(1H,3H)-dione hydrochloride

A solution of HCl (4.0 M in dioxane, 2 mL, 8 mmol) was added to a solution of tert-butyl 4-((3-(2,4-dioxotetrahydropyrimidin-1(2H)-yl)benzo[d]isoxazol-5-yl)methyl)piperazine-1-carboxylate (120 mg, 0.279 mmol) in DCM (5 mL) and the mixture was stirred at rt for 1 h. The reaction was then concentrated to give 1-(5-(piperazin-1-ylmethyl)benzo[d]isoxazol-3-yl)dihydropyrimidine-2,4(1H,3H)-dione hydrochloride (130 mg, crude) which was used without further purification. LCMS [M+H]⁺: 330.1.

### Step 4. 1-(5-((4-(cyclohexylmethyl)piperazin-1-yl)methyl)benzo[d]isoxazol-3-yl)dihydropyrimidine-2,4(1H,3H)-dione

Triethylamine (164 mg, 1.63 mmol) was added to a solution of 1-(5-(piperazin-1-ylmethyl)benzo[d]isoxazol-3-yl)dihydropyrimidine-2,4(1H,3H)-dione hydrochloride (180 mg, 0.546 mmol) and cyclohexanecarbaldehyde (122 mg, 1.09 mmol) in DCM (10 mL) at rt. The reaction mixture was stirred at rt for 10 min and then sodium triacetoxyborohydride (347 mg, 1.63 mmol) was added. The reaction was stirred at rt for 2 h and then diluted with DCM and water. The organic layer was dried over Na₂SO₄, filtered and concentrated. The residue was dissolved in DMSO, filtered through a 1 micron filter and purified by reverse-phase HPLC using ACN/Water/0.1% formic acid. The fractions containing the product were combined and lyophilized to afford a formate salt of 1-(5-((4-(cyclohexylmethyl)piperazin-1-yl)methyl)benzo[d]isoxazol-3-yl)dihydropyrimidine-2,4(1H,3H)-dione (80 mg, 0.188 mmol, 34% yield). LCMS [M+H]⁺: 426.3. ¹H NMR (400 MHz, DMSO-d6) δ 10.89 (s, 1H), 7.72 (s, 1H), 7.66 (d, J=8.6 Hz, 1H), 7.58 (d, J=8.7 Hz, 1H), 4.06 (t, J=6.6 Hz, 2H), 3.56 (6, 2H), 2.79 (t, J=6.6 Hz, 2H), 2.36 (m, 8H), 2.09 (d, J=7.3 Hz, 2H), 1.65 (q, J=14.2, 13.0 Hz, 5H), 1.45 (ddd, J=11.0, 7.5, 3.6 Hz, 1H), 1.16 (h, J=13.0 Hz, 3H), 0.80 (q, J=11.5 Hz, 2H).
The compounds in the following table were prepared by the method of Example 21, using the appropriate commercially available aldehyde in step 4.

| Example | | Mass | |
|---|---|---|---|
| No. | Structure | M + H | ¹H NMR |
| 22 | 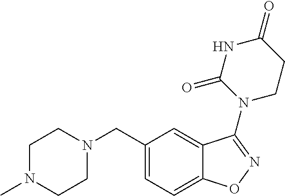 [SMILES: CN1CCN(Cc2ccc3onc(N4CCC(=O)NC4=O)c3c2)CC1] | 344.3 | (300 MHz, Methanol-d4) δ 7.82 (s, 1H), 7.72 − 7.44 (m, 2H), 4.16 (t, J = 6.6 Hz, 2H), 3.69 (s, 2H), 3.30 (s, 4H), 2.95 − 2.79 (m, 6H), 2.54 (s, 3H), NH proton not observed due to solvent exhange. |
| | 1-(5-((4-methylpiperazin-1- | | |
| | yl)methyl)benzo[d]isoxazol-3- | | |
| | yl)dihydropyrimidine-2,4(1H,3H)-dione | | |
| 23 | 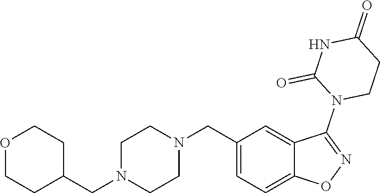 [SMILES: O=C1CCN(c2noc3ccc(CN4CCN(CC5CCOCC5)CC4)cc23)C(=O)N1] | 428.2 | (300 MHz, Methanol-d4) δ 7.85 (s, 1H), 7.74 − 7.40 (m, 2H), 4.16 (t, J = 6.7 Hz, 2H), 3.92 (s, 2H), 3.76 (s, 2H), 3.40 (t, J = 11.9 Hz, 2H), 3.30 (s, 3H), 2.87 (t, J = 6.8 Hz, 2H), 2.73 (d, J = 23.1 Hz, 5H), 2.48 (d, J = 7.1 Hz, 2H), 1.89 (s, 1H), 1.67 (d, J = 13.1 Hz, 2H), 1.26 (q, J = 13.8, 12.4 Hz, 2H), NH proton not observed due to solvent exhange. |
| | 1-(5-((4-((tetrahydro-2H-pyran-4- | | |
| | yl)methyl)piperazin-1- | | |
| | yl)methyl)benzo[d]isoxazol-3- | | |
| | yl)dihydropyrimidine-2,4(1H,3H)-dione | | |
| 24 | 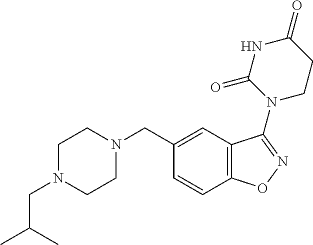 [SMILES: CC(C)CN1CCN(Cc2ccc3onc(N4CCC(=O)NC4=O)c3c2)CC1] | 386.4 | (500 MHz, DMSO-d6) δ 10.94 (s, 1H), 7.93 − 7.57 (m, 3H), 4.09 (t, J = 6.6 Hz, 2H), 3.73 (s, 2H), 2.82 (t, J = 6.6 Hz, 8H), 2.56 (s, 1H), 1.98 (s, 1H), 0.92 (d, J = 6.4 Hz, 6H). (3 protons obscured by solvent peak) |
| | 1-(5-((4-isobutylpiperazin-1- | | |
| | yl)methyl)benzo[d]isoxazol-3- | | |
| | yl)dihydropyrimidine-2,4(1H,3H)-dione | | |
| 25 | 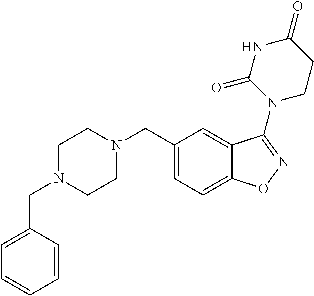 [SMILES: O=C1CCN(c2noc3ccc(CN4CCN(Cc5ccccc5)CC4)cc23)C(=O)N1] | 420.1 | (300 MHz, DMSO-d6) δ 10.86 (s, 1H), 7.69 (s, 1H), 7.63 (d, J = 8.7 Hz, 1H), 7.55 (d, J = 8.7 Hz, 1H), 7.36 − 7.08 (m, 5H), 4.03 (t, J = 6.6 Hz, 2H), 3.54 (s, 2H), 3.43 (s, 2H), 2.77 (t, J = 6.5 Hz, 2H), 2.70 (s, 1H), 2.36 (s, 6H), 2.25 (s, 1H). |
| | 1-(5-((4-benzylpiperazin-1- | | |
| | yl)methyl)benzo[d]isoxazol-3- | | |
| | yl)dihydropyrimidine-2,4(1H,3H)-dione | | |
| 26 | 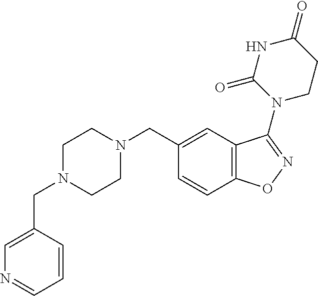 [SMILES: O=C1CCN(c2noc3ccc(CN4CCN(Cc5cccnc5)CC4)cc23)C(=O)N1] | 421.4 | (500 MHz, DMSO-d6) δ 11.07 − 10.81 (m, 1H), 8.56 (s, 2H), 7.81 (m, 4H), 7.43 (s, 1H), 4.22 (s, 3H), 4.10 (t, J = 6.6 Hz, 3H), 3.52 (m, 5H), 3.03 (m, 2H), 2.82 (t, J = 6.6 Hz, 3H). |
| | 1-(5-((4-(pyridin-3-ylmethyl)piperazin- | | |
| | 1-yl)methyl)benzo[d]isoxazol-3- | | |
| | yl)dihydropyrimidine-2,4(1H,3H)-dione | | |
| 27 | 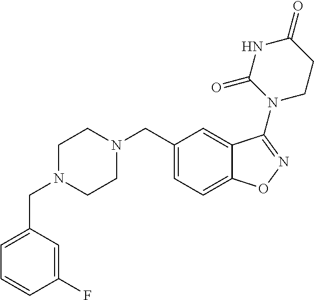 [SMILES: O=C1CCN(c2noc3ccc(CN4CCN(Cc5cccc(F)c5)CC4)cc23)C(=O)N1] | 438.1 | (400 MHz, DMSO-d6) δ 10.89 (s, 1H), 7.72 (s, 1H), 7.66 (d, J = 8.6 Hz, 1H), 7.58 (d, J = 8.9 Hz, 1H), 7.35 (q, J = 7.4 Hz, 1H), 7.18 − 6.97 (m, 3H), 4.06 (t, J = 6.5 Hz, 2H), 3.58 (s, 2H), 3.48 (s, 2H), 2.79 (t, J = 6.6 Hz, 2H), 2.36 (d, J = 28.1 Hz, 8H). |
| | 1-(5-((4-(3-fluorobenzyl)piperazin-1- | | |
| | yl)methyl)benzo[d]isoxazol-3- | | |
| | yl)dihydropyrimidine-2,4(1H,3H)-dione | | |

The compounds in the following table were prepared by the method of Example 21, using potassium (S)-((4-(tert-butoxycarbonyl)-3-methylpiperazin-1-yl)methyl)trifluoroborate [see ChemMedChem, 2016, 11, 2640-2648] in step 1 and the appropriate commercially available aldehyde in step 4.

| Example | | Mass | |
|---|---|---|---|
| No | Structure | M + H | ¹H NMR |
| 28 | 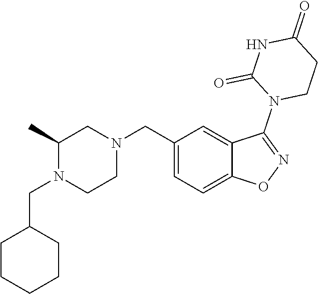 [SMILES: C[C@H]1CN(Cc2ccc3onc(N4CCC(=O)NC4=O)c3c2)CCN1CC1CCCCC1] | 440.2 | (400 MHz, Methanol-d4) δ 7.84 (d, J = 1.5 Hz, 1H), 7.64 (dd, J = 8.8, 1.7 Hz, 1H), 7.58 (d, J = 8.7 Hz, 1H), 4.17 (t, J = 6.6 Hz, 2H), 3.80 − 3.68 (m, 2H), 3.45 (d, J = 12.5 Hz, 1H), 3.14 − 2.83 (m, 6H), 2.78 − 2.35 (m, 4H), 1.78 (td, J = 28.1, 24.6, 11.8 Hz, 6H), 1.44 − 1.18 (m, 6H), 1.13 − 0.96 (m, 2H). NH proton not observed due to solvent exhange. |
| | (S)-1-(5-((4-(cyclohexylmethyl)-3- | | |
| | methylpiperazin-1- | | |
| | yl)methyl)benzo[d]isoxazol-3- | | |
| | yl)dihydropyrimidine-2,4(1H,3H)-dione | | |
| 29 | 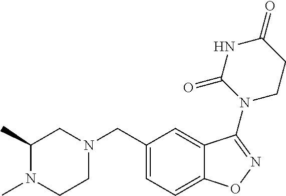 [SMILES: C[C@H]1CN(Cc2ccc3onc(N4CCC(=O)NC4=O)c3c2)CCN1C] | 358.3 | (400 MHz, Methanol-d4) δ 7.84 (s, 1H), 7.70 − 7.54 (m, 2H), 4.18 (t, J = 6.6 Hz, 2H), 3.73 (d, J = 2.4 Hz, 2H), 3.41 − 3.35 (m, 1H), 3.27 − 2.95 (m, 4H), 2.89 (t, J = 6.7 Hz, 2H), 2.80 (s, 3H), 2.49 (s, 1H), 2.27 (s, 1H), 1.31 (d, J = 6.4 Hz, 3H), NH proton not observed due to solvent exhange. |
| | (S)-1-(5-((3,4-dimethylpiperazin-1- | | |
| | yl)methyl)benzo[d]isoxazol-3- | | |
| | yl)dihydropyrimidine-2,4(1H,3H)-dione | | |
| 30 | 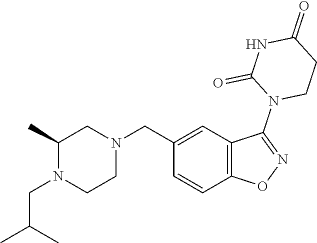 [SMILES: CC(C)CN1CCN(Cc2ccc3onc(N4CCC(=O)NC4=O)c3c2)C[C@@H]1C] | 400.2 | (500 MHz, DMSO-d6) δ 10.89 (s, 1H), 7.75 − 7.70 (m, 1H), 7.67 (d, J = 8.6 Hz, 1H), 7.59 (dd,J = 8.7, 1.7 Hz, 1H), 4.07 (t, J = 6.6 Hz, 2H), 3.58 − 3.43 (m, 2H), 2.80 (t, J = 6.6 Hz, 2H), 2.74 (d, J =11.3 Hz, 1H), 2.56 (d, J = 10.8 Hz, 2H), 2.33 (dd, J = 12.3, 9.3 Hz, 2H), 2.13 (dt, J = 37.2, 10.2 Hz, 2H), 2.00 − 1.77 (m, 2H), 1.69 (td, J = 15.0, 13.8, 6.2 Hz, 1H), 0.91 (d, J = 6.2 Hz, 3H), 0.86 − 0.77 (m, 6H). |
| | (S)-1-(5-((4-isobutyl-3- | | |
| | methylpiperazin-1- | | |
| | yl)methyl)benzo[d]isoxazol-3- | | |
| | yl)dihydropyrimidine-2,4(1H,3H)-dione | | |
| 31 | 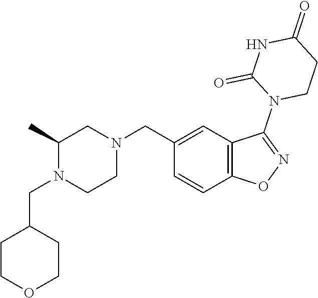 [SMILES: C[C@H]1CN(Cc2ccc3onc(N4CCC(=O)NC4=O)c3c2)CCN1CC1CCOCC1] | 442.3 | (400 MHz, DMSO-d6) δ 10.93 (s, 1H), 7.86 (s, 1H), 7.77 (d, J = 8.6 Hz, 1H), 7.68 (d, J = 8.8 Hz, 1H), 4.09 (t, J = 6.6 Hz, 2H), 4.05 − 3.76 (m, 4H), 3.63 (s,1H), 3.29 (tdd, J = 11.5, 9.2, 2.1 Hz, 3H), 3.09 (s, 4H), 2.81 (t, J = 6.6 Hz, 3H), 2.55 (s, 1H), 1.97 (s, 1H), 1.78 − 1.64 (m, 1H), 1.54 (s, 1H), 1.24 (s, 6H). |
| | (S)-1-(5-((3-methyl-4-((tetrahydro-2H- | | |
| | pyran-4-yl)methyl)piperazin-1- | | |
| | yl)methyl)benzo[d]isoxazol-3- | | |
| | yl)dihydropyrimidine-2,4(1H,3H)-dione | | |

The compounds in the following table were prepared by the method of Example 21, using potassium (S)-((4-(tert-butoxycarbonyl)-3-methylpiperazin-1-yl)methyl)trifluoroborate [see ChemMedChem, 2016, 11, 2640-2648] in step 1 and the appropriate commercially available aldehyde in step 4.

### Example 32. (S)-1-(5-((4-isobutyl-2-methylpiperazin-1-yl)methyl)benzo[d]isoxazol-3-yl)dihydropyrimidine-2,4(1H,3H)-dione

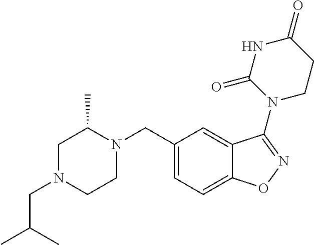 [SMILES: CC(C)CN1CCN(Cc2ccc3onc(N4CCC(=O)NC4=O)c3c2)[C@@H](C)C1]

Prepared by the method of Example 21, using potassium (S)-((4-(tert-butoxycarbonyl)-2-methylpiperazin-1-yl)methyl)trifluoroborate [see J. Med. Chem. 2012, 55, 7796-7816] in step 1 and isobutyraldehyde in step 4. LCMS [M+H]⁺: 400.0. ¹H NMR (400 MHz, Methanol-d4) δ 7.80 (s, 1H), 7.60 (dd, J=8.7, 1.7 Hz, 1H), 7.52 (d, J=8.7 Hz, 1H), 4.15 (q, J=6.7 Hz, 3H), 3.32 (d, J=3.1 Hz, 1H), 2.86 (t, J=6.6 Hz, 2H), 2.77-2.61 (m, 3H), 2.51 (s, 1H), 2.22 (t, J=10.5 Hz, 1H), 2.13-1.98 (m, 3H), 1.91 (t, J=10.4 Hz, 1H), 1.77 (dt, J=13.7, 6.9 Hz, 1H), 1.18 (d, J=6.2 Hz, 3H), 0.87 (d, J=6.6 Hz, 6H), NH proton not observed due to solvent exchange.

### Example 33. (R)-1-(5-((4-isobutyl-2-methylpiperazin-1-yl)methyl)benzo[d]isoxazol-3-yl)dihydropyrimidine-2,4(1H,3H)-dione

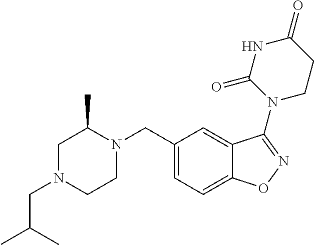 [SMILES: CC(C)CN1CCN(Cc2ccc3onc(N4CCC(=O)NC4=O)c3c2)[C@H](C)C1]

Prepared by the method of Example 21, using potassium (R)-((4-(tert-butoxycarbonyl)-2-methylpiperazin-1-yl)methyl)trifluoroborate [see J. Med. Chem. 2012, 55, 7796-7816] in step 1 and isobutyraldehyde in step 4. LCMS [M+H]⁺: 400.2. ¹H NMR (400 MHz, Methanol-d4) δ 7.82 (d, J=1.5 Hz, 1H), 7.61 (dd, J=8.5, 1.7 Hz, 1H), 7.54 (d, J=8.7 Hz, 1H), 4.23 (d, J=13.2 Hz, 1H), 4.15 (t, J=6.6 Hz, 2H), 3.37 (d, J=13.2 Hz, 1H), 3.01-2.81 (m, 4H), 2.75 (dd, J=9.4, 3.4 Hz, 1H), 2.65 (d, J=4.8 Hz, 1H), 2.41-2.24 (m, 4H), 2.18 (t, J=10.7 Hz, 1H), 1.85 (dq, J=13.5, 6.7 Hz, 1H), 1.23 (d, J=6.4 Hz, 3H), 0.91 (d, J=6.6 Hz, 6H), NH proton not observed due to solvent exchange

### Example 34. (R)-1-(5-((4-isobutyl-3-methylpiperazin-1-yl)methyl)benzo[d]isoxazol-3-yl)dihydropyrimidine-2,4(1H,3H)-dione

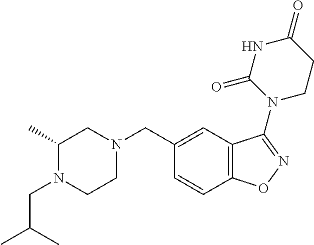 [SMILES: CC(C)CN1CCN(Cc2ccc3onc(N4CCC(=O)NC4=O)c3c2)C[C@H]1C]

Prepared by the method of Example 21, using potassium (R)-((4-(tert-butoxycarbonyl)-3-methylpiperazin-1-yl)methyl)trifluoroborate in step 1 and isobutyraldehyde in step 4. LCMS [M+H]⁺: 400.3. ¹H NMR (400 MHz, Methanol-d4) δ 7.82 (s, 1H), 7.63 (d, J=8.7 Hz, 1H), 7.55 (d, J=8.6 Hz, 1H), 4.17 (t, J=6.7 Hz, 2H), 3.62 (t, J=4.5 Hz, 2H), 2.90 (q, J=8.3, 6.6 Hz, 3H), 2.84-2.60 (m, 2H), 2.48 (dd, J=26.8, 15.9 Hz, 2H), 2.28 (d, J=7.3 Hz, 2H), 2.17-1.92 (m, 2H), 1.80 (s, 1H), 1.02 (d, J=6.2 Hz, 3H), 0.91 (t, J=6.5 Hz, 6H), NH proton not observed due to solvent exchange.

### Example 35. (S)-1-(5-((3-ethyl-4-methylpiperazin-1-yl)methyl)benzo[d]isoxazol-3-yl)dihydropyrimidine-2,4(1H,3H)-dione

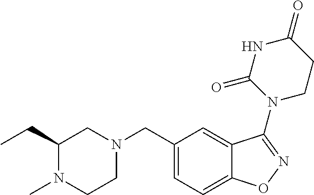 [SMILES: CC[C@H]1CN(Cc2ccc3onc(N4CCC(=O)NC4=O)c3c2)CCN1C]

Prepared by the method of Example 21, using potassium (S)-((4-(tert-butoxycarbonyl)-3-ethylpiperazin-1-yl)methyl)trifluoroborate in step 1 and acetaldehyde in step 4. LCMS [M+H]⁺: 372.3. ¹H NMR (400 MHz, Methanol-d4) δ 7.84 (d, J=1.6 Hz, 1H), 7.64 (dd, J=8.6, 1.8 Hz, 1H), 7.57 (d, J=8.6 Hz, 1H), 4.17 (t, J=6.7 Hz, 2H), 3.75 (d, J=13.0 Hz, 1H), 3.67 (d, J=13.0 Hz, 1H), 3.18 (d, J=11.9 Hz, 1H), 2.99 (d, J=12.3 Hz, 1H), 2.88 (t, J=6.6 Hz, 4H), 2.73 (s, 1H), 2.64 (s, 3H), 2.43 (s, 1H), 2.19 (d, J=12.0 Hz, 1H), 1.84 (ddd, J=14.1, 7.5, 3.5 Hz, 1H), 1.53 (s, 1H), 0.92 (t, J=7.4 Hz, 3H), NH proton not observed due to solvent exchange.

### Example 36. (S)-1-(5-((3-ethyl-4-((tetrahydro-2H-pyran-4-yl)methyl)piperazin-1-yl)methyl)benzo[d]isoxazol-3-yl)dihydropyrimidine-2,4(1H,3H)-dione

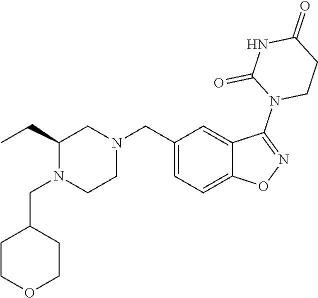 [SMILES: CC[C@H]1CN(Cc2ccc3onc(N4CCC(=O)NC4=O)c3c2)CCN1CC1CCOCC1]

Prepared by the method of Example 21, using potassium (S)-((4-(tert-butoxycarbonyl)-3-ethylpiperazin-1-yl)methyl)trifluoroborate in step 1 and tetrahydro-2H-pyran-4-carbaldehyde in step 4. LCMS [M+H]⁺: 456.3. ¹H NMR (400 MHz, Methanol-d4) δ 7.85 (d, J=1.6 Hz, 1H), 7.65 (dd, J=8.7, 1.7 Hz, 1H), 7.58 (d, J=8.7 Hz, 1H), 4.17 (t, J=6.6 Hz, 2H), 4.00-3.88 (m, 2H), 3.81 (d, J=13.1 Hz, 1H), 3.71 (d, J=13.1 Hz, 1H), 3.50-3.35 (m, 2H), 3.23 (d, J=13.0 Hz, 1H), 2.87 (t, J=6.6 Hz, 4H), 2.73 (s, 2H), 2.49 (d, J=48.5 Hz, 3H), 1.89 (s, 1H), 1.81-1.53 (m, 4H), 1.29 (pd, J=11.9, 4.5 Hz, 3H), 0.88 (t, J=7.4 Hz, 3H), NH proton not observed due to solvent exchange.

### Example 37. (S)-1-(5-((3-isopropyl-4-methylpiperazin-1-yl)methyl)benzo[d]isoxazol-3-yl)dihydropyrimidine-2,4(1H,3H)-dione

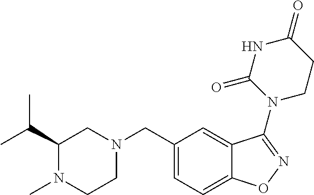 [SMILES: CC(C)[C@H]1CN(Cc2ccc3onc(N4CCC(=O)NC4=O)c3c2)CCN1C]

Prepared by the method of Example 21, using potassium (S)-((4-(tert-butoxycarbonyl)-3-isopropylpiperazin-1-yl)methyl)trifluoroborate in step 1 and acetaldehyde in step 4. LCMS [M+H]⁺: 386.3. ¹H NMR (400 MHz, Methanol-d4) δ 7.84 (d, J=1.6 Hz, 1H), 7.67-7.46 (m, 2H), 4.17 (t, J=6.6 Hz, 2H), 3.82 (d, J=13.2 Hz, 1H), 3.72 (d, J=13.1 Hz, 1H), 3.34 (d, J=3.1 Hz, 1H), 3.10 (dt, J=12.4, 2.6 Hz, 2H), 2.98 (d, J=12.3 Hz, 2H), 2.87 (t, J=6.6 Hz, 2H), 2.54-2.40 (m, 1H), 2.39-2.23 (m, 2H), 1.29 (s, 3H), 0.97 (dd, J=14.8, 7.0 Hz, 6H), NH proton not observed due to solvent exchange.

### Example 38. (S)-1-(5-((3-isopropyl-4-((tetrahydro-2H-pyran-4-yl)methyl)piperazin-1-yl)methyl)benzo[d]isoxazol-3-yl)dihydropyrimidine-2,4(1H,3H)-dione

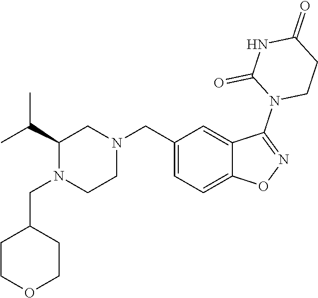 [SMILES: CC(C)[C@H]1CN(Cc2ccc3onc(N4CCC(=O)NC4=O)c3c2)CCN1CC1CCOCC1]

Prepared by the method of Example 21, using potassium (S)-((4-(tert-butoxycarbonyl)-3-isopropylpiperazin-1-yl)methyl)trifluoroborate in step 1 and tetrahydro-2H-pyran-4-carbaldehyde in step 4. LCMS [M+H]⁺: 470.4. ¹H NMR (400 MHz, Methanol-d4) δ 7.87 (d, J=1.6 Hz, 1H), 7.65 (dd, J=8.6, 1.7 Hz, 1H), 7.58 (d, J=8.9 Hz, 1H), 4.17 (t, J=6.6 Hz, 2H), 3.99-3.81 (m, 3H), 3.74 (d, J=13.1 Hz, 1H), 3.51-3.34 (m, 3H), 3.13 (d, J=9.1 Hz, 1H), 2.98-2.82 (m, 4H), 2.77 (s, 1H), 2.49-2.09 (m, 4H), 1.91-1.72 (m, 2H), 1.60 (d, J=13.1 Hz, 1H), 1.24 (ddd, J=34.5, 12.4, 8.1 Hz, 3H), 0.89 (dd, J=18.7, 6.7 Hz, 6H), NH proton not observed due to solvent exchange.

### Example 39. tert-butyl 4-((3-(2,4-dioxotetrahydropyrimidin-1(2H)-yl)benzo[d]isoxazol-5-yl)methyl)-1,4-diazepane-1-carboxylate

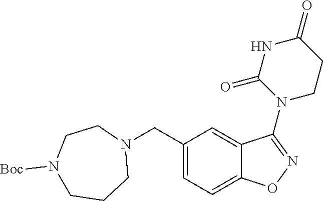 [SMILES: CC(C)(C)OC(=O)N1CCCN(Cc2ccc3onc(N4CCC(=O)NC4=O)c3c2)CC1]

Prepared by the method of Example 21, steps 1-2 using potassium ((4-(tert-butoxycarbonyl)-1,4-diazepan-1-yl)methyl)trifluoroborate in step 1. LCMS [M+H]⁺: 444.0. ¹H NMR (400 MHz, Methanol-d4) δ 7.82 (s, 1H), 7.65 (d, J=8.8 Hz, 1H), 7.54 (d, J=8.8 Hz, 1H), 4.17 (t, J=6.6 Hz, 2H), 3.76 (s, 2H), 3.52-3.43 (m, 4H), 2.88 (t, J=6.6 Hz, 2H), 2.73-2.60 (m, 4H), 1.82 (s, 2H), 1.46 (d, J=4.4 Hz, 9H), NH proton not observed due to solvent exchange.

### Example 40. 1-(5-((1,4-diazepan-1-yl)methyl)benzo[d]isoxazol-3-yl)dihydropyrimidine-2,4(1H,3H)-dione

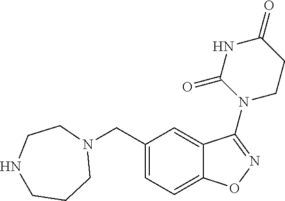 [SMILES: O=C1CCN(c2noc3ccc(CN4CCCNCC4)cc23)C(=O)N1]

Prepared from Example 39 by the method of Example 21, step 3. LCMS [M+H]⁺: 343.9. ¹H NMR (400 MHz, Methanol-d4) δ 7.97 (s, 1H), 7.70 (d, J=8.8 Hz, 1H), 7.44 (d, J=8.8 Hz, 1H), 4.18 (m, 4H), 3.40 (m, 2H), 3.36 (m, 2H), 3.28 (m, 2H), 3.12 (m, 2H), 2.88 (t, J=6.6 Hz, 2H), 2.11 (m, 2H), NH proton not observed due to solvent exchange.

### Example 41. 1-(5-((4-(cyclohexylmethyl)-1,4-diazepan-1-yl)methyl)benzo[d]isoxazol-3-yl)dihydropyrimidine-2,4(1H,3H)-dione

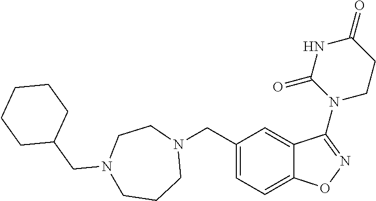 [SMILES: O=C1CCN(c2noc3ccc(CN4CCCN(CC5CCCCC5)CC4)cc23)C(=O)N1]

Prepared from Example 40 by the method of Example 21, step 4. LCMS [M+H]⁺: 440.2. ¹H NMR (400 MHz, Methanol-d4) δ 7.85 (s, 1H), 7.65 (dd, J=9.0, 1.7 Hz, 1H), 7.56 (d, J=8.8 Hz, 1H), 4.18 (t, J=6.6 Hz, 2H), 3.82 (s, 2H), 3.21-3.02 (m, 4H), 2.93-2.66 (m, 7H), 1.97 (s, 2H), 1.85-1.61 (m, 5H), 1.38-1.19 (m, 4H), 1.06-0.88 (m, 3H), NH proton not observed due to solvent exchange.

### Example 42. Preparation of tert-butyl 3-((3-(2,4-dioxotetrahydropyrimidin-1 (2H)-yl)benzo[d]isoxazol-5-yl)methyl)piperidine-1-carboxylate

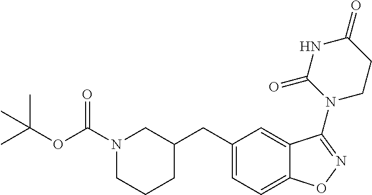 [SMILES: CC(C)(C)OC(=O)N1CCCC(Cc2ccc3onc(N4CCC(=O)NC4=O)c3c2)C1]

Prepared using the method of Example 1a wherein tert-butyl 3-methylenepiperidine-1-carboxylate was used in place of tert-butyl 4-methylenepiperidine-1-carboxylate in step 3a. LCMS [M+H-tBu]⁺: 373.1. ¹H NMR (400 MHz, cd3od) δ 7.67 (s, 1H), 7.51 (d, J=8.7 Hz, 1H), 7.47 (dd, J=8.7, 1.7 Hz, 1H), 4.16 (t, J=6.6 Hz, 2H), 3.84 (br s, 2H), 2.88 (m, 3H), 2.66 (m, 3H), 1.85-1.62 (m, 3H), 1.38 (m, 11H). NH not observed due to solvent exchange.

### Example 43. Preparation of 1-(5-(piperidin-3-ylmethyl)benzo[d]isoxazol-3-yl)dihydropyrimidine-2,4(1H,3H)-dione

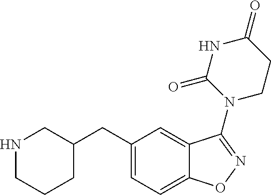 [SMILES: O=C1CCN(c2noc3ccc(CC4CCCNC4)cc23)C(=O)N1]

Prepared using the method of Example 1b wherein tert-butyl 3-methylenepiperidine-1-carboxylate was used in place of tert-butyl 4-methylenepiperidine-1-carboxylate in step 3a. LCMS [M+H]⁺: 329.9. ¹H NMR (400 MHz, cd3od) δ 8.42 (s, 1H), 7.70 (s, 1H), 7.55 (d, J=8.6 Hz, 1H), 7.48 (d, J=8.6 Hz, 1H), 4.16 (d, J=5.7 Hz, 2H), 3.34 (d, J=4.5 Hz, 1H), 3.25 (dd, J=12.6, 3.7 Hz, 1H), 2.96-2.63 (m, 6H), 2.13-1.98 (m, 1H), 1.89 (t, J=17.1 Hz, 2H), 1.69 (tdd, J=14.1, 8.8, 3.9 Hz, 1H), 1.39-1.24 (m, 1H). NH not observed due to solvent exchange.

### Example 44. Preparation of 1-(5-((1-(cyclohexylmethyl)piperidin-3-yl)methyl)benzo[d]isoxazol-3-yl)dihydropyrimidine-2,4(1H,3H)-dione

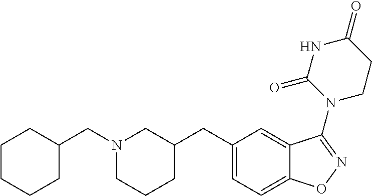 [SMILES: O=C1CCN(c2noc3ccc(CC4CCCN(CC5CCCCC5)C4)cc23)C(=O)N1]

Prepared using the method of Example 1 wherein tert-butyl 3-methylenepiperidine-1-carboxylate was used in place of tert-butyl 4-methylenepiperidine-1-carboxylate in step 3a. LCMS [M+H]⁺: 425.3. ¹H NMR (400 MHz, cd3od) δ 8.38 (s, 1H), 7.70 (s, 1H), 7.56 (d, J=8.6 Hz, 1H), 7.49 (dd, J=8.7, 1.7 Hz, 1H), 4.17 (t, J=6.6 Hz, 2H), 3.51 (d, J=12.6 Hz, 1H), 3.42-3.34 (m, 1H), 2.97-2.62 (m, 8H), 2.16 (s, 1H), 1.99-1.63 (m, 9H), 1.38-1.14 (m, 4H), 1.00 (q, J=12.1 Hz, 2H).

### Example 45. Preparation of 1-(5-((1-methylpiperidin-3-yl)methyl)benzo[d]isoxazol-3-yl)dihydropyrimidine-2,4(1H,3H)-dione

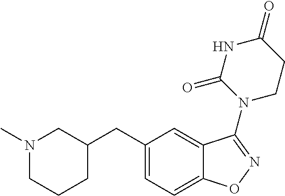 [SMILES: CN1CCCC(Cc2ccc3onc(N4CCC(=O)NC4=O)c3c2)C1]

Prepared using the method of Example 1 wherein tert-butyl 3-methylenepiperidine-1-carboxylate was used in place of tert-butyl 4-methylenepiperidine-1-carboxylate in step 3a and paraformaldehyde was used in place of cyclohexanecarbaldehyde in step 6. LCMS [M+H]⁺: 342.9. ¹H NMR (400 MHz, cd3od) δ 8.43 (s, 1H), 7.66 (d, J=1.7 Hz, 1H), 7.52 (d, J=8.7 Hz, 1H), 7.45 (dd, J=8.8, 1.8 Hz, 1H), 4.14 (t, J=6.7 Hz, 2H), 3.39 (d, J=12.2 Hz, 1H), 3.32 (t, J=2.2 Hz, 1H), 2.90-2.60 (m, 9H), 2.09 (ddt, J=15.7, 7.9, 3.9 Hz, 1H), 1.91 (dq, J=13.6, 3.4 Hz, 1H), 1.81 (d, J=13.4 Hz, 1H), 1.77-1.63 (m, 1H), 1.22 (qd, J=12.6, 3.8 Hz, 1H).

### Example 46. Preparation of 1-(5-((1-isobutylpiperidin-3-yl)methyl)benzo[d]isoxazol-3-yl)dihydropyrimidine-2,4(1H,3H)-dione

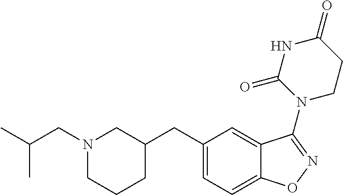 [SMILES: CC(C)CN1CCCC(Cc2ccc3onc(N4CCC(=O)NC4=O)c3c2)C1]

Prepared using the method of Example 1 wherein tert-butyl 3-methylenepiperidine-1-carboxylate was used in place of tert-butyl 4-methylenepiperidine-1-carboxylate in step 3a and isobutyraldehyde was used in place of cyclohexanecarbaldehyde in step 6. LCMS [M+H]⁺: 384.9. ¹H NMR (400 MHz, cd3od) δ 8.45 (s, 1H), 7.70 (s, 1H), 7.56 (d, J=8.7 Hz, 1H), 7.49 (d, J=8.7 Hz, 1H), 4.17 (t, J=6.7 Hz, 2H), 3.52 (d, J=12.4 Hz, 1H), 3.39 (d, J=11.9 Hz, 1H), 2.96-2.63 (m, 8H), 2.24-2.08 (m, 2H), 1.86 (td, J=30.3, 14.1 Hz, 3H), 1.29 (t, J=12.9 Hz, 1H), 1.00 (dd, J=6.7, 3.7 Hz, 6H).

### Example 47. Preparation of tert-butyl 3-((3-(2,4-dioxotetrahydropyrimidin-1 (2H)-yl)benzo[d]isoxazol-5-yl)methyl)pyrrolidine-1-carboxylate

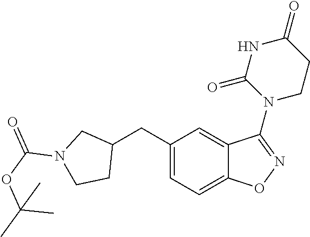 [SMILES: CC(C)(C)OC(=O)N1CCC(Cc2ccc3onc(N4CCC(=O)NC4=O)c3c2)C1]

Prepared using the method of Example 1a wherein tert-butyl 3-methylenepyrrolidine-1-carboxylate was used in place of tert-butyl 4-methylenepiperidine-1-carboxylate in step 3a. LCMS [M+H]⁺: 415.3. ¹H NMR (400 MHz, cd3od) δ 8.52 (s, 1H), 7.68 (s, 1H), 7.54-7.44 (m, 2H), 4.14 (t, J=6.6 Hz, 2H), 3.41 (dt, J=16.8, 10.0 Hz, 2H), 3.24 (d, J=8.1 Hz, 1H), 2.97 (d, J=13.0 Hz, 1H), 2.86 (t, J=6.6 Hz, 2H), 2.81 (d, J=6.8 Hz, 2H), 2.48 (s, 1H), 1.93 (s, 1H), 1.70-1.56 (m, 1H), 1.42 (s, 9H).

### Example 48. Preparation of 1-(5-(pyrrolidin-3-ylmethyl)benzo[d]isoxazol-3-yl)dihydropyrimidine-2,4(1H,3H)-dione

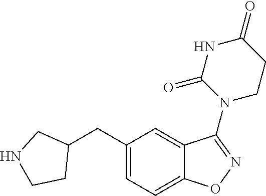 [SMILES: O=C1CCN(c2noc3ccc(CC4CCNC4)cc23)C(=O)N1]

Prepared using the method of Example 1b wherein tert-butyl 3-methylenepyrrolidine-1-carboxylate was used in place of tert-butyl 4-methylenepiperidine-1-carboxylate in step 3a. LCMS [M+H]⁺: 314.8. ¹H NMR (300 MHz, cd3od) δ 8.53 (s, 1H), 7.74 (s, 1H), 7.59-7.47 (m, 2H), 4.17 (t, J=6.6 Hz, 2H), 2.99-2.83 (m, 6H), 2.75-2.61 (m, 2H), 2.21-2.04 (m, 2H), 1.75 (dd, J=13.1, 8.6 Hz, 1H). NH not observed due to solvent exchange.

### Example 49. Preparation of 1-(5-((1-(cyclohexylmethyl)pyrrolidin-3-yl)methyl)benzo[d]isoxazol-3-yl)dihydropyrimidine-2,4(1H,3H)-dione

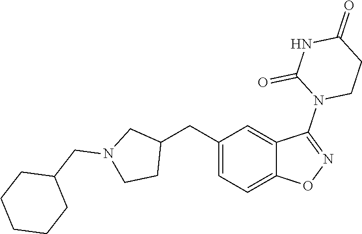 [SMILES: O=C1CCN(c2noc3ccc(CC4CCN(CC5CCCCC5)C4)cc23)C(=O)N1]

Prepared using the method of Example 1 wherein tert-butyl 3-methylenepyrrolidine-1-carboxylate was used in place of tert-butyl 4-methylenepiperidine-1-carboxylate in step 3a. LCMS [M+H]⁺: 411.3. ¹H NMR (300 MHz, cd3od) δ 7.74 (s, 1H), 7.56 (d, J=8.7 Hz, 1H), 7.53-7.48 (m, 1H), 4.17 (t, J=6.7 Hz, 2H), 3.77-3.58 (m, 2H), 3.22 (s, 1H), 3.04 (dd, J=6.8, 4.1 Hz, 2H), 2.98-2.80 (m, 5H), 2.79-2.63 (m, 1H), 2.26-2.07 (m, 1H), 1.95 (d, J=15.9 Hz, 1H), 1.78 (d, J=12.0 Hz, 6H), 1.27 (dt, J=23.6, 12.4 Hz, 3H), 1.02 (d, J=12.3 Hz, 2H). NH not observed due to solvent exchange.

### Example 50. Preparation of 1-(5-((1-methylpyrrolidin-3-yl)methyl)benzo[d]isoxazol-3-yl)dihydropyrimidine-2,4(1H,3H)-dione

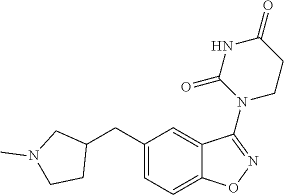 [SMILES: CN1CCC(Cc2ccc3onc(N4CCC(=O)NC4=O)c3c2)C1]

Prepared using the method of Example 1 wherein tert-butyl 3-methylenepyrrolidine-1-carboxylate was used in place of tert-butyl 4-methylenepiperidine-1-carboxylate in step 3a and paraformaldehyde was used in place of cyclohexanecarbaldehyde in step 6. LCMS [M+H]⁺: 329.2. ¹H NMR (400 MHz, cd3od) δ 7.74 (s, 1H), 7.56 (d, J=8.6 Hz, 1H), 7.54-7.49 (m, 1H), 4.17 (t, J=6.6 Hz, 2H), 3.48 (s, 4H), 3.10 (d, J=23.9 Hz, 1H), 2.95-2.85 (m, 6H), 2.81 (d, J=7.7 Hz, 1H), 2.26-2.14 (m, 1H), 1.92-1.81 (m, 1H). NH not observed due to solvent exchange.

### Example 51. Preparation of tert-butyl 4-((3-(2,4-dioxotetrahydropyrimidin-1 (2H)-yl)benzo[d]isoxazol-5-yl)methyl)azepane-1-carboxylate

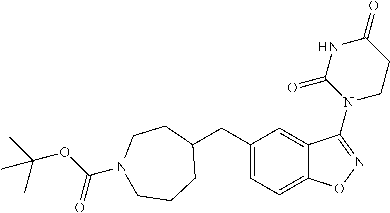 [SMILES: CC(C)(C)OC(=O)N1CCCC(Cc2ccc3onc(N4CCC(=O)NC4=O)c3c2)CC1]

Prepared using the method of Example 1a wherein tert-butyl 4-methyleneazepane-1-carboxylate was used in place of tert-butyl 4-methylenepiperidine-1-carboxylate in step 3a. LCMS [M+H]⁺: 443.2. ¹H NMR (400 MHz, cd3od) δ 7.64 (s, 1H), 7.49 (d, J=8.9 Hz, 1H), 7.44 (d, J=8.7 Hz, 1H), 4.15 (t, J=6.6 Hz, 2H), 3.53 (dp, J=14.9, 4.7 Hz, 1H), 3.45-3.33 (m, 2H), 3.15 (qd, J=10.3, 5.1 Hz, 1H), 2.88 (t, J=6.6 Hz, 2H), 2.67 (dt, J=5.1, 2.5 Hz, 2H), 1.78 (dt, J=18.0, 7.6 Hz, 4H), 1.59-1.48 (m, 1H), 1.44 (dd, J=7.8, 2.0 Hz, 9H), 1.34 (ddt, J=18.7, 13.8, 6.9 Hz, 1H), 1.27-1.13 (m, 1H). NH not observed due to solvent exchange.

### Example 52. Preparation of 1-(5-(azepan-4-ylmethyl)benzo[d]isoxazol-3-yl)dihydropyrimidine-2,4(1H,3H)-dione

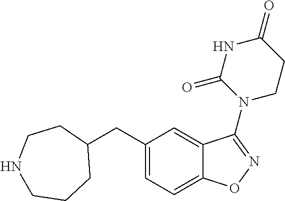 [SMILES: O=C1CCN(c2noc3ccc(CC4CCCNCC4)cc23)C(=O)N1]

Prepared using the method of Example 1b wherein tert-butyl 4-methyleneazepane-1-carboxylate was used in place of tert-butyl 4-methylenepiperidine-1-carboxylate in step 3a. LCMS [M+H]⁺: 343.2. ¹H NMR (400 MHz, cd3od) δ 8.50 (s, 1H), 7.67 (s, 1H), 7.53 (d, J=8.6 Hz, 1H), 7.47 (dd, J=8.6, 1.6 Hz, 1H), 4.17 (t, J=6.6 Hz, 2H), 3.30-3.22 (m, 2H), 3.11 (dddd, J=29.3, 13.5, 10.3, 2.4 Hz, 2H), 2.88 (t, J=6.7 Hz, 2H), 2.73 (h, J=6.8 Hz, 2H), 1.97 (dt, J=16.5, 5.5 Hz, 4H), 1.85-1.71 (m, 1H), 1.66-1.52 (m, 1H), 1.35 (dt, J=25.0, 12.2 Hz, 2H).

### Example 53. Preparation of 1-(5-((1-methylazepan-4-yl)methyl)benzo[d]isoxazol-3-yl)dihydropyrimidine-2,4(1H,3H)-dione

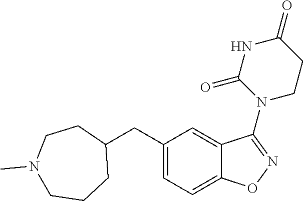 [SMILES: CN1CCCC(Cc2ccc3onc(N4CCC(=O)NC4=O)c3c2)CC1]

Prepared using the method of Example 1 wherein tert-butyl 4-methyleneazepane-1-carboxylate was used in place of tert-butyl 4-methylenepiperidine-1-carboxylate in step 3a and paraformaldehyde was used in place of cyclohexanecarbaldehyde in step 6. LCMS [M+H]⁺: 357.2. ¹H NMR (400 MHz, cd3od) δ 8.39 (s, 1H), 7.66 (d, J=6.2 Hz, 1H), 7.49 (dt, J=18.7, 5.8 Hz, 2H), 4.15 (p, J=6.6 Hz, 2H), 3.40-3.12 (m, 5H), 2.86 (dt, J=10.1, 4.8 Hz, 4H), 2.71 (dq, J=18.6, 10.0 Hz, 2H), 1.97 (d, J=44.1 Hz, 5H), 1.69 (q, J=12.1 Hz, 1H), 1.41 (q, J=10.0 Hz, 1H).

### Examples 54 and 55. Preparation of tert-butyl (1R,5S)-3-((3-(2,4-dioxotetrahydropyrimidin-1(2H)-yl)benzo[d]isoxazol-5-yl)methyl)-8-azabicyclo[3.2.1]octane-8-carboxylate

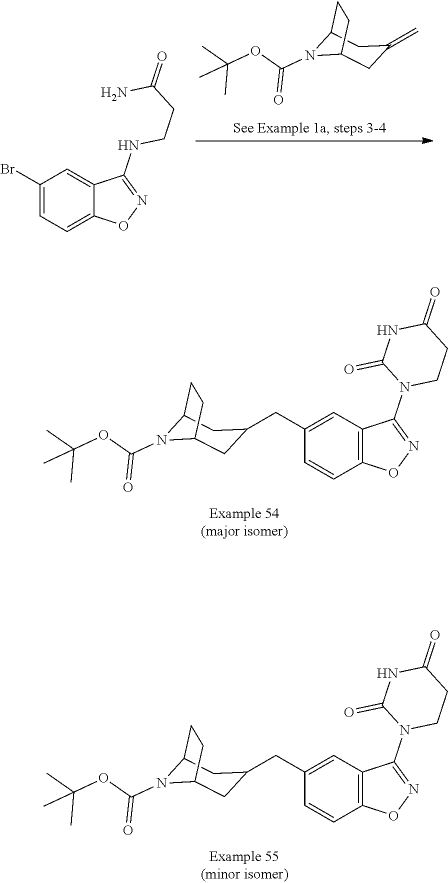

Prepared from 3-((5-bromobenzo[d]isoxazol-3-yl)amino)propanamide using the method of Example 1a wherein tert-butyl (1R,5S)-3-methylene-8-azabicyclo[3.2.1]octane-8-carboxylate was used in place of tert-butyl 4-methylenepiperidine-1-carboxylate in step 3a. The isomers were separated by chiral HPLC: COLUMN: LUX CELLULOSE-4, 250 MM×21.2 MM×5 MICRON, MOBILE PHASE: HEXANE (A) 0.1% HCOOH IN EtOH:MeOH, 1:1, (B), FLOW; 15 ML, ISOCRATIC:60(A):40(B). The collected fractions were concentrated under reduced pressure to afford:
Example 54 (major product): tert-butyl (1R,5S)-3-((3-(2,4-dioxotetrahydropyrimidin-1(2H)-yl)benzo[d]isoxazol-5-yl)methyl)-8-azabicyclo[3.2.1]octane-8-carboxylate (20 mg, 0.044 mmol); LCMS [M+H]⁺: 455.2; chiral HPLC: (12.5 min). ¹H NMR (400 MHz, MeOD) δ 8.55 (s, 1H), 7.64 (dd, J=1.7, 0.8 Hz, 1H), 7.49 (dd, J=8.7, 0.7 Hz, 1H), 7.44 (dd, J=8.7, 1.7 Hz, 1H), 4.16 (dd, J=8.4, 5.0 Hz, 4H), 2.89 (t, J=6.7 Hz, 2H), 2.61 (d, J=7.0 Hz, 2H), 2.18 (dp, J=12.1, 6.1 Hz, 1H), 1.89 (s, 2H), 1.66 (d, J=7.6 Hz, 2H), 1.54 (d, J=13.1 Hz, 2H), 1.45 (s, 11H).
Example 55 (minor product): tert-butyl (1R,5S)-3-((3-(2,4-dioxotetrahydropyrimidin-1(2H)-yl)benzo[d]isoxazol-5-yl)methyl)-8-azabicyclo[3.2.1]octane-8-carboxylate (10 mg, 0.022 mmol); LCMS [M+H-Boc]⁺: 355.2; chiral HPLC: (14.1 min). ¹H NMR (400 MHz, MeOD) δ 7.66 (d, J=1.7 Hz, 1H), 7.50 (d, J=8.7 Hz, 1H), 7.44 (dd, J=8.7, 1.7 Hz, 1H), 4.16 (dd, J=9.9, 3.5 Hz, 4H), 2.88 (t, J=6.8 Hz, 4H), 2.15-1.80 (m, 5H), 1.44 (s, 9H), 1.34 (ddd, J=19.1, 10.5, 3.0 Hz, 4H). NH not observed due to solvent exchange.

### Example 56. Preparation of 1-(5-(((1R,5S)-8-azabicyclo[3.2.1]octan-3-yl)methyl)benzo[d]isoxazol-3-yl)dihydropyrimidine-2,4(1H,3H)-dione

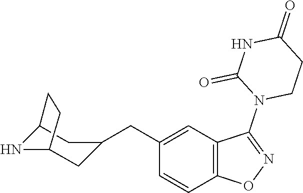 [SMILES: O=C1CCN(c2noc3ccc(CC4CC5CCC(C4)N5)cc23)C(=O)N1]

Prepared from Example 5 using the method of Example 1 b, step 5 wherein tert-butyl (1R,5S)-3-((3-(2,4-dioxotetrahydropyrimidin-1(2H)-yl)benzo[d]isoxazol-5-yl)methyl)-8-azabicyclo[3.2.1]octane-8-carboxylate was used in place of tert-butyl 4-((3-(2,4-dioxotetrahydropyrimidin-1(2H)-yl)benzo[d]isoxazol-5-yl)methyl)piperidine-1-carboxylate. LCMS [M+H]⁺: 355.0. ¹H NMR (300 MHz, cd3od) δ 7.68 (s, 1H), 7.53 (d, J=8.7 Hz, 1H), 7.50-7.42 (m, 1H), 4.17 (t, J=6.7 Hz, 2H), 3.99 (d, J=5.1 Hz, 2H), 2.88 (t, J=6.7 Hz, 2H), 2.71 (d, J=7.0 Hz, 2H), 2.27-2.14 (m, 1H), 2.11-1.89 (m, 4H), 1.82-1.69 (m, 2H), 1.58 (t, J=13.2 Hz, 2H), 1.31 (d, J=12.1 Hz, 1H). NH protons not observed due to solvent exchange.

### Example 57. Preparation of 1-(5-(((1R,5S)-8-azabicyclo[3.2.1]octan-3-yl)methyl)benzo[d]isoxazol-3-yl)dihydropyrimidine-2,4(1H,3H)-dione

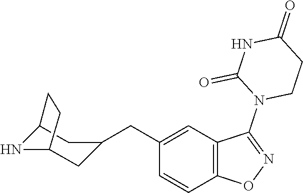 [SMILES: O=C1CCN(c2noc3ccc(CC4CC5CCC(C4)N5)cc23)C(=O)N1]

Prepared from Example 55 using the method of Example 1 b, step 5 wherein tert-butyl (1R,5S)-3-((3-(2,4-dioxotetrahydropyrimidin-1(2H)-yl)benzo[d]isoxazol-5-yl)methyl)-8-azabicyclo[3.2.1]octane-8-carboxylate was used in place of tert-butyl 4-((3-(2,4-dioxotetrahydropyrimidin-1(2H)-yl)benzo[d]isoxazol-5-yl)methyl)piperidine-1-carboxylate. LCMS [M+H]⁺: 355.2. ¹H NMR (400 MHz, MeOD) δ 7.72 (d, J=1.7 Hz, 1H), 7.55 (d, J=8.6 Hz, 1H), 7.50 (dd, J=8.7, 1.7 Hz, 1H), 4.17 (t, J=6.7 Hz, 2H), 3.99 (s, 2H), 2.99 (d, J=7.9 Hz, 2H), 2.88 (t, J=6.6 Hz, 2H), 2.29-2.12 (m, 7H), 1.76 (d, J=14.4 Hz, 2H). NH protons not observed due to solvent exchange.

### Example 58. Preparation of 1-(5-(((1R,5S)-8-methyl-8-azabicyclo[3.2.1]octan-3-yl)methyl)benzo[d]isoxazol-3-yl)dihydropyrimidine-2,4(1H,3H)-dione

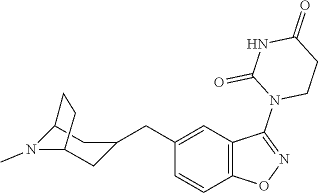 [SMILES: CN1C2CCC1CC(Cc1ccc3onc(N4CCC(=O)NC4=O)c3c1)C2]

Prepared from Example 56 using the method of Example 1, step 6 wherein 1-(5-(((1R,5S)-8-azabicyclo[3.2.1]octan-3-yl)methyl)benzo[d]isoxazol-3-yl)dihydropyrimidine-2,4(1H,3H)-dione was used in place of tert-butyl 4-((3-(2,4-dioxotetrahydropyrimidin-1(2H)-yl)benzo[d]isoxazol-5-yl)methyl)piperidine-1-carboxylate and paraformaldehyde was used in place of cyclohexanecarbaldehyde. LCMS [M+H]⁺: 369.2. ¹H NMR (300 MHz, cd3od) δ 8.57 (s, 1H), 7.68 (s, 1H), 7.53 (d, J=8.6 Hz, 1H), 7.50-7.43 (m, 1H), 4.17 (t, J=6.6 Hz, 2H), 3.80 (s, 2H), 2.88 (t, J=6.6 Hz, 2H), 2.71 (d, J=6.2 Hz, 5H), 2.23 (s, 3H), 1.95 (d, J=8.6 Hz, 2H), 1.69 (dd, J=28.5, 15.9 Hz, 4H).

### Example 59. Preparation of 1-(5-(((1R,5S)-8-methyl-8-azabicyclo[3.2.1]octan-3-yl)methyl)benzo[d]isoxazol-3-yl)dihydropyrimidine-2,4(1H,3H)-dione

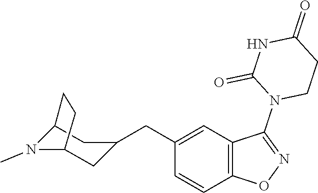 [SMILES: CN1C2CCC1CC(Cc1ccc3onc(N4CCC(=O)NC4=O)c3c1)C2]

Prepared from Example 57 using the method of Example 1, step 6 wherein 1-(5-(((1R,5S)-8-azabicyclo[3.2.1]octan-3-yl)methyl)benzo[d]isoxazol-3-yl)dihydropyrimidine-2,4(1H,3H)-dione was used in place of tert-butyl 4-((3-(2,4-dioxotetrahydropyrimidin-1(2H)-yl)benzo[d]isoxazol-5-yl)methyl)piperidine-1-carboxylate and paraformaldehyde was used in place of cyclohexanecarbaldehyde. LCMS [M+H]⁺: 369.2. ¹H NMR (400 MHz, MeOD) δ 8.54 (s, 1H), 7.74-7.69 (m, 1H), 7.54 (dd, J=8.7, 0.8 Hz, 1H), 7.49 (dd, J=8.7, 1.7 Hz, 1H), 4.16 (t, J=6.7 Hz, 2H), 3.75 (s, 2H), 3.00 (d, J=7.1 Hz, 2H), 2.87 (t, J=6.7 Hz, 2H), 2.72 (s, 3H), 2.41-2.32 (m, 2H), 2.28-2.15 (m, 5H), 1.80 (d, J=14.0 Hz, 2H).

### Example 60. Preparation of 1-(5-(((1R,5S)-8-(methylsulfonyl)-8-azabicyclo[3.2.1]octan-3-yl)methyl)benzo[d]isoxazol-3-yl)dihydropyrimidine-2,4(1H,3H)-dione

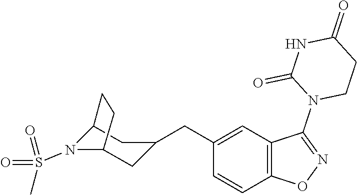 [SMILES: CS(=O)(=O)N1C2CCC1CC(Cc1ccc3onc(N4CCC(=O)NC4=O)c3c1)C2]

Prepared from Example 56 using the method of Example 14 wherein 1-(5-(((1R,5S)-8-azabicyclo[3.2.1]octan-3-yl)methyl)benzo[d]isoxazol-3-yl)dihydropyrimidine-2,4(1H,3H)-dione was used in place of 3-(5-(piperidin-4-ylmethyl)benzo[d]isoxazol-3-yl)piperidine-2,6-dione and methanesulfonyl chloride was used in place of propane-2-sulfonyl chloride. LCMS [M+H]⁺: 433.2. ¹H NMR (400 MHz, DMSO) δ 10.88 (s, 1H), 7.66-7.56 (m, 2H), 7.49 (dd, J=8.6, 1.8 Hz, 1H), 4.11-4.01 (m, 4H), 2.89 (s, 3H), 2.79 (t, J=6.6 Hz, 2H), 2.60 (d, J=7.0 Hz, 2H), 2.04-1.86 (m, 3H), 1.63-1.49 (m, 4H), 1.37 (dd, J=12.9, 10.4 Hz, 2H).

### Example 61. Preparation of 1-(5-(((1R,5S)-8-(methylsulfonyl)-8-azabicyclo[3.2.1]octan-3-yl)methyl)benzo[d]isoxazol-3-yl)dihydropyrimidine-2,4(1H,3H)-dione

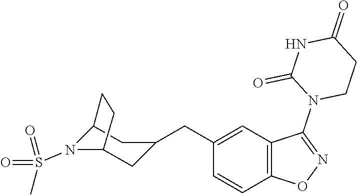 [SMILES: CS(=O)(=O)N1C2CCC1CC(Cc1ccc3onc(N4CCC(=O)NC4=O)c3c1)C2]

Prepared from Example 57 using the method of Example 14 wherein 1-(5-(((1R,5S)-8-azabicyclo[3.2.1]octan-3-yl)methyl)benzo[d]isoxazol-3-yl)dihydropyrimidine-2,4(1H,3H)-dione was used in place of 3-(5-(piperidin-4-ylmethyl)benzo[d]isoxazol-3-yl)piperidine-2,6-dione and methanesulfonyl chloride was used in place of propane-2-sulfonyl chloride. LCMS [M+H]⁺: 433.1. ¹H NMR (400 MHz, DMSO) δ 10.87 (s, 1H), 7.66-7.62 (m, 2H), 7.51 (dd, J=8.8, 1.6 Hz, 1H), 4.07 (dd, J=12.6, 6.0 Hz, 4H), 2.91 (s, 3H), 2.80 (q, J=7.0 Hz, 4H), 2.09-1.94 (m, 5H), 1.85 (t, J=7.1 Hz, 2H), 1.42 (d, J=12.4 Hz, 2H).

### Example 62. Preparation of 1-(5-((4-ethylpiperazin-1-yl)methyl)benzo[d]isoxazol-3-yl)dihydropyrimidine-2,4(1H,3H)-dione

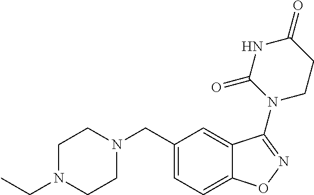 [SMILES: CCN1CCN(Cc2ccc3onc(N4CCC(=O)NC4=O)c3c2)CC1]

Prepared by the method of Example 21 wherein acetaldehyde was used in place of cyclohexanecarbaldehyde in step 4. LCMS [M+H]⁺: 358.2. ¹H NMR (400 MHz, MeOD) δ 8.53 (s, 1H), 7.84 (d, J=1.6 Hz, 1H), 7.64 (dd, J=8.7, 1.7 Hz, 1H), 7.57 (d, J=8.7 Hz, 1H), 4.17 (t, J=6.6 Hz, 2H), 3.73 (s, 2H), 3.08 (s, 4H), 2.99 (q, J=7.3 Hz, 2H), 2.88 (t, J=6.6 Hz, 2H), 2.71 (s, 4H), 1.27 (t, J=7.3 Hz, 3H).

### Example 63. Preparation of 1-(5-((4-methyl-1,4-diazepan-1-yl)methyl)benzo[d]isoxazol-3-yl)dihydropyrimidine-2,4(1H,3H)-dione

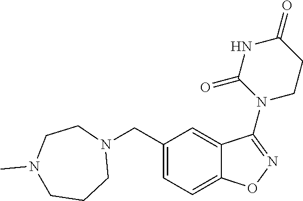 [SMILES: CN1CCCN(Cc2ccc3onc(N4CCC(=O)NC4=O)c3c2)CC1]

Prepared from Example 40 by the method of Example 21, step 4 wherein paraformaldehyde was used in place of cyclohexanecarbaldehyde. LCMS [M+H]⁺: 357.9. ¹H NMR (400 MHz, cd3od) δ 8.10 (d, J=1.8 Hz, 1H), 7.76 (dd, J=8.8, 1.8 Hz, 1H), 7.73-7.68 (m, 1H), 4.48 (s, 2H), 4.19 (dd, J=7.7, 5.9 Hz, 2H), 3.69 (d, J=16.2 Hz, 4H), 3.51 (s, 2H), 3.44 (d, J=5.5 Hz, 2H), 2.96 (d, J=1.8 Hz, 3H), 2.88 (td, J=6.7, 1.7 Hz, 2H), 2.28 (s, 2H). NH not observed due to solvent exchange.

### Example 64. (R)-1-(5-((4-acetyl-2-methylpiperazin-1-yl)methyl)benzo[d]isoxazol-3-yl)dihydropyrimidine-2,4(1H,3H)-dione

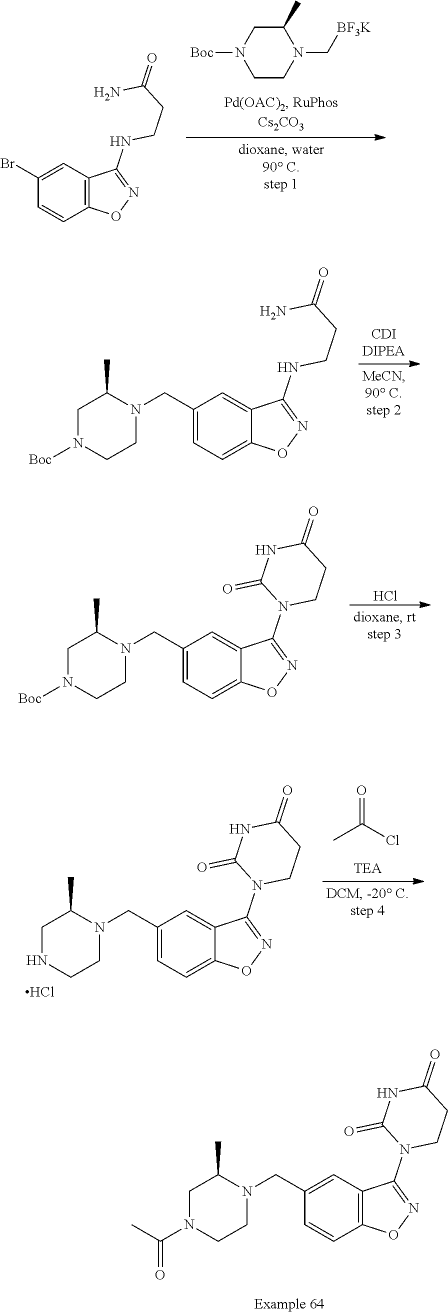

### Step 1. tert-butyl (R)-4-((3-((3-amino-3-oxopropyl)amino)benzo[d]isoxazol-5-yl)methyl)-3-methylpiperazine-1-carboxylate

To a suspension of 3-((5-bromobenzo[d]isoxazol-3-yl)amino)propanamide (500 mg, 1.75 mmol) in dioxane (15 mL) and water (1.5 mL) at room temperature was added Cs₂CO₃ (1.14 g, 3.5 mmol), potassium (R)-((4-(tert-butoxycarbonyl)-2-methylpiperazin-1-yl)methyl)trifluoroborate [see J. Med. Chem. 2012, 55, 7796-7816](1.12 g, 3.5 mmol) and RuPhos (81 mg, 0.18 mmol), followed by Pd(OAc)₂ (39 mg, 0.18 mmol). The mixture was stirred at 90° C. for 3 h, then cooled to rt and partitioned between EtOAc and water. The organic layer was separated, washed with brine, dried over sodium sulfate, filtered and concentrated. The residue was purified by silica gel chromatography (eluting with 100% EtOAc in hexane) to give tert-butyl (R)-4-((3-((3-amino-3-oxopropyl)amino)benzo[d]isoxazol-5-yl)methyl)-3-methylpiperazine-1-carboxylate (400 mg, 0.95 mmol, 54% yield). LCMS [M+H]⁺: 418.0.

### Step 2. tert-butyl (R)-4-((3-(2,4-dioxotetrahydropyrimidin-1(2H)-yl)benzo[d]isoxazol-5-yl)methyl)-3-methylpiperazine-1-carboxylate

DIPEA (495 mg, 3.83 mmol) and CDI (621 mg, 3.83 mmol) was added to a solution of tert-butyl (R)-4-((3-((3-amino-3-oxopropyl)amino)benzo[d]isoxazol-5-yl)methyl)-3-methylpiperazine-1-carboxylate (400 mg, 0.95 mmol) in acetonitrile (20 mL) at rt. The reaction mixture was then heated at 90° C. for 8 h. The reaction was cooled to rt and diluted with EtOAc and washed with water. The organic layer was separated, dried over sodium sulfate, filtered and concentrated to give crude tert-butyl (R)-4-((3-(2,4-dioxotetrahydropyrimidin-1(2H)-yl)benzo[d]isoxazol-5-yl)methyl)-3-methylpiperazine-1-carboxylate (300 mg, crude) which was used without further purification. LCMS [M+H]⁺: 444.1.

### Step 3. (R)-1-(5-((2-methylpiperazin-1-yl)methyl)benzo[d]isoxazol-3-yl)dihydropyrimidine-2,4(1H,3H)-dione hydrochloride

A solution of HCl (4.0 M in dioxane, 5 mL) was added to a solution of tert-butyl (R)-4-((3-(2,4-dioxotetrahydropyrimidin-1(2H)-yl)benzo[d]isoxazol-5-yl)methyl)-3-methylpiperazine-1-carboxylate (300 mg, 0.67 mmol) in DCM (5 mL) and the mixture was stirred at rt for 2 h. The reaction was then concentrated to give (R)-1-(5-((2-methylpiperazin-1-yl)methyl)benzo[d]isoxazol-3-yl)dihydropyrimidine-2,4(1H,3H)-dione hydrochloride (130 mg, crude) which was used without further purification. LCMS [M+H]⁺: 344.2.

### Step 4. (R)-1-(5-((4-acetyl-2-methylpiperazin-1-yl)methyl)benzo[d]isoxazol-3-yl)dihydropyrimidine-2,4(1H,3H)-dione

Triethylamine (115 mg, 1.05 mmol) was added to a solution of (R)-1-(5-((2-methylpiperazin-1-yl)methyl)benzo[d]isoxazol-3-yl)dihydropyrimidine-2,4(1H,3H)-dione hydrochloride (120 mg, 0.349 mmol) in DCM (2 mL) at −20° C. Acetyl chloride (40 mg, 0.52 mmol) was added and the mixture was stirred at −20° C. for 20 min. The reaction was quenched with water and then extracted with EtOAc. The organic layer was dried over Na₂SO₄, filtered and concentrated. The residue was dissolved in DMSO, filtered through a 1 micron filter and purified by reverse-phase HPLC using ACN/Water/0.1% formic acid. The fractions containing the product were combined and lyophilized to afford (R)-1-(5-((4-acetyl-2-methylpiperazin-1-yl)methyl)benzo[d]isoxazol-3-yl)dihydropyrimidine-2,4(1H,3H)-dione (20 mg, 0.051 mmol, 15% yield). LCMS [M+H]⁺: 386.2 ¹H NMR (400 MHz, MeOD) δ 7.85 (s, 1H), 7.65 (dt, J=8.7, 1.3 Hz, 1H), 7.56 (d, J=8.7 Hz, 1H), 4.16 (td, J=6.1, 3.2 Hz, 3H), 4.07-3.87 (m, 1H), 3.76-3.60 (m, 1H), 3.46 (dd, J=13.4, 4.5 Hz, 1H), 3.17 (td, J=13.2, 9.1 Hz, 1H), 2.97 (dd, J=13.2, 8.5 Hz, 1H), 2.88 (t, J=6.7 Hz, 2H), 2.82-2.70 (m, 1H), 2.60 (dtd, J=30.9, 6.0, 3.0 Hz, 1H), 2.31-2.13 (m, 1H), 2.08 (d, J=8.4 Hz, 3H), 1.22 (dd, J=10.1, 6.2 Hz, 3H). NH not observed due to solvent exchange.

### Example 65. (R)-1-(5-((2,4-dimethylpiperazin-1-yl)methyl)benzo[d]isoxazol-3-yl)dihydropyrimidine-2,4(1H,3H)-dione

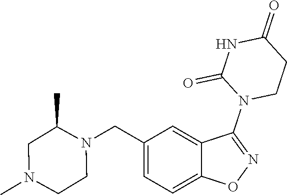 [SMILES: C[C@@H]1CN(C)CCN1Cc1ccc2onc(N3CCC(=O)NC3=O)c2c1]

Prepared by the method of Example 21, step 4 wherein (R)-1-(5-((2-methylpiperazin-1-yl)methyl)benzo[d]isoxazol-3-yl)dihydropyrimidine-2,4(1H,3H)-dione hydrochloride was used in place of 1-(5-(piperazin-1-ylmethyl)benzo[d]isoxazol-3-yl)dihydropyrimidine-2,4(1H,3H)-dione hydrochloride and paraformaldehyde was used in place of cyclohexanecarbaldehyde. LCMS [M+H]⁺: 357.9. ¹H NMR (400 MHz, cd3od) δ 8.41 (s, 1H), 7.82 (s, 1H), 7.65-7.58 (m, 1H), 7.55 (d, J=8.7 Hz, 1H), 4.26 (d, J=13.3 Hz, 1H), 4.15 (t, J=6.6 Hz, 2H), 3.35 (d, J=20.3 Hz, 2H), 3.19 (dt, J=30.7, 14.4 Hz, 3H), 2.92-2.75 (m, 4H), 2.71 (s, 3H), 2.38 (d, J=12.9 Hz, 1H), 1.27 (d, J=5.7 Hz, 3H).

### Example 66. (R)-1-(5-((4-ethyl-2-methylpiperazin-1-yl)methyl)benzo[d]isoxazol-3-yl)dihydropyrimidine-2,4(1H,3H)-dione

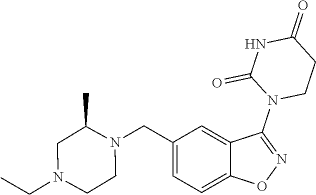 [SMILES: CCN1CCN(Cc2ccc3onc(N4CCC(=O)NC4=O)c3c2)[C@H](C)C1]

Prepared by the method of Example 21, step 4 wherein (R)-1-(5-((2-methylpiperazin-1-yl)methyl)benzo[d]isoxazol-3-yl)dihydropyrimidine-2,4(1H,3H)-dione hydrochloride was used in place of 1-(5-(piperazin-1-ylmethyl)benzo[d]isoxazol-3-yl)dihydropyrimidine-2,4(1H,3H)-dione hydrochloride and acetaldehyde was used in place of cyclohexanecarbaldehyde. LCMS [M+H]⁺: 372.0. ¹H NMR (400 MHz, MeOD) δ 8.51 (s, 1H), 7.84 (d, J=1.7 Hz, 1H), 7.63 (dd, J=8.7, 1.7 Hz, 1H), 7.56 (dd, J=8.7, 0.7 Hz, 1H), 4.26 (d, J=13.4 Hz, 1H), 4.17 (t, J=6.7 Hz, 2H), 3.20-3.06 (m, 2H), 2.92-2.73 (m, 5H), 2.70-2.26 (m, 5H), 1.27 (d, J=6.2 Hz, 3H), 1.21 (t, J=7.3 Hz, 3H).

### Example 67. (R)-1-(5-((2-methyl-4-((tetrahydro-2H-pyran-4-yl)methyl)piperazin-1-yl)methyl)benzo[d]isoxazol-3-yl)dihydropyrimidine-2,4(1H,3H)-dione

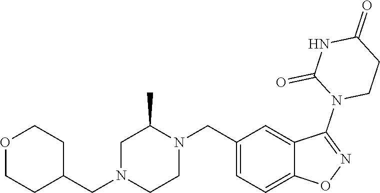 [SMILES: C[C@@H]1CN(CC2CCOCC2)CCN1Cc1ccc2onc(N3CCC(=O)NC3=O)c2c1]

Prepared by the method of Example 21, step 4 wherein (R)-1-(5-((2-methylpiperazin-1-yl)methyl)benzo[d]isoxazol-3-yl)dihydropyrimidine-2,4(1H,3H)-dione hydrochloride was used in place of 1-(5-(piperazin-1-ylmethyl)benzo[d]isoxazol-3-yl)dihydropyrimidine-2,4(1H,3H)-dione hydrochloride and tetrahydro-2H-pyran-4-carbaldehyde was used in place of cyclohexanecarbaldehyde. LCMS [M+H]⁺: 442.3. ¹H NMR (400 MHz, MeOD) δ 8.31 (s, 1H), 7.91 (d, J=1.7 Hz, 1H), 7.67 (dd, J=8.7, 1.7 Hz, 1H), 7.62 (d, J=8.7 Hz, 1H), 4.42 (d, J=13.4 Hz, 1H), 4.18 (t, J=6.6 Hz, 2H), 3.97-3.88 (m, 2H), 3.63 (d, J=13.4 Hz, 1H), 3.42 (tt, J=11.9, 2.1 Hz, 2H), 3.24-3.11 (m, 2H), 2.98 (d, J=11.5 Hz, 2H), 2.88 (t, J=6.7 Hz, 2H), 2.73-2.52 (m, 4H), 1.96 (ddd, J=13.0, 6.6, 2.8 Hz, 1H), 1.68 (dt, J=12.3, 2.8 Hz, 2H), 1.37 (d, J=6.3 Hz, 3H), 1.29 (tt, J=12.0, 6.0 Hz, 3H).

### Example 68. (R)-1-(5-((2-methyl-4-(oxetan-3-ylmethyl)piperazin-1-yl)methyl)benzo[d]isoxazol-3-yl)dihydropyrimidine-2,4(1H,3H)-dione

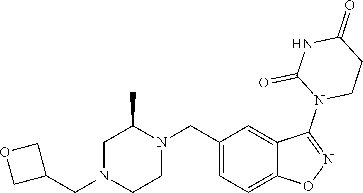 [SMILES: C[C@@H]1CN(CC2COC2)CCN1Cc1ccc2onc(N3CCC(=O)NC3=O)c2c1]

Prepared by the method of Example 21, step 4 wherein (R)-1-(5-((2-methylpiperazin-1-yl)methyl)benzo[d]isoxazol-3-yl)dihydropyrimidine-2,4(1H,3H)-dione hydrochloride was used in place of 1-(5-(piperazin-1-ylmethyl)benzo[d]isoxazol-3-yl)dihydropyrimidine-2,4(1H,3H)-dione hydrochloride and oxetane-3-carbaldehyde was used in place of cyclohexanecarbaldehyde. LCMS [M+H]⁺: 414.3. ¹H NMR (400 MHz, MeOD) δ 7.86 (d, J=1.7 Hz, 1H), 7.64 (dd, J=8.7, 1.7 Hz, 1H), 7.58 (dd, J=8.7, 0.7 Hz, 1H), 4.79 (ddt, J=7.7, 6.1, 3.9 Hz, 3H), 4.41 (td, J=6.2, 3.9 Hz, 2H), 4.31 (d, J=13.3 Hz, 1H), 4.17 (t, J=6.6 Hz, 2H), 3.52-3.44 (m, 1H), 2.93-2.67 (m, 8H), 2.38 (q, J=9.3 Hz, 2H), 2.28-2.19 (m, 1H), 1.28 (t, J=5.1 Hz, 3H). NH not observed due to solvent exchange.

### Example 69. (R)-1-(5-((4-isopropyl-2-methylpiperazin-1-yl)methyl)benzo[d]isoxazol-3-yl)dihydropyrimidine-2,4(1H,3H)-dione

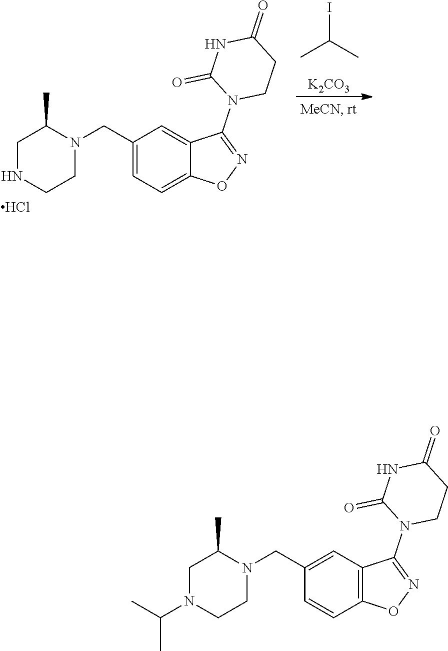

Potassium carbonate (120 mg, 0.872 mmol) and isopropyl iodide (75 mg, 0.44 mmol) were added to a solution of (R)-1-(5-((2-methylpiperazin-1-yl)methyl)benzo[d]isoxazol-3-yl)dihydropyrimidine-2,4(1H,3H)-dione hydrochloride (150 mg, 0.436 mmol) in MeCN (5 mL) at rt. The mixture was stirred at rt for 2 h. The reaction was quenched with water and then extracted with EtOAc. The organic layer was dried over Na₂SO₄, filtered and concentrated. The residue was dissolved in DMSO, filtered through a 1 micron filter and purified by reverse-phase HPLC using ACN/Water/0.1% formic acid. The fractions containing the product were combined and lyophilized to afford (R)-1-(5-((4-isopropyl-2-methylpiperazin-1-yl)methyl)benzo[d]isoxazol-3-yl)dihydropyrimidine-2,4(1H,3H)-dione (20 mg, 0.051 mmol, 12% yield). LCMS [M+H]⁺: 386.2 ¹H NMR (400 MHz, MeOD) δ 7.84 (d, J=1.7 Hz, 1H), 7.64 (dd, J=8.7, 1.7 Hz, 1H), 7.57 (dd, J=8.7, 0.7 Hz, 1H), 4.29 (d, J=13.3 Hz, 1H), 4.17 (t, J=6.7 Hz, 2H), 3.25 (d, J=20.3 Hz, 4H), 2.97-2.85 (m, 4H), 2.75 (s, 2H), 2.38 (t, J=12.3 Hz, 1H), 1.30 (dt, J=6.7, 2.0 Hz, 9H). NH not observed due to solvent exchange.

### Example 70. (R)-1-(5-((2-methyl-4-(2-oxo-2-(piperidin-1-yl)ethyl)piperazin-1-yl)methyl)benzo[d]isoxazol-3-yl)dihydropyrimidine-2,4(1H,3H)-dione

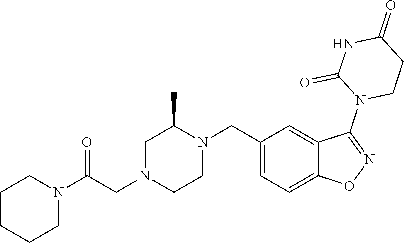 [SMILES: C[C@@H]1CN(CC(=O)N2CCCCC2)CCN1Cc1ccc2onc(N3CCC(=O)NC3=O)c2c1]

Prepared by the method of Example 69 wherein 2-bromo-1-(piperidin-1-yl)ethan-1-one was used in place of isopropyl iodide. LCMS [M+H]⁺: 469.2. ¹H NMR (400 MHz, MeOD) δ 8.29 (s, 1H), 7.92 (d, J=1.6 Hz, 1H), 7.67 (dd, J=8.8, 1.7 Hz, 1H), 7.62 (d, J=8.7 Hz, 1H), 4.42 (d, J=13.3 Hz, 1H), 4.18 (t, J=6.6 Hz, 2H), 3.66 (d, J=24.7 Hz, 3H), 3.54 (t, J=5.6 Hz, 2H), 3.44 (t, J=5.5 Hz, 2H), 3.16-2.94 (m, 4H), 2.89 (t, J=6.6 Hz, 2H), 2.64 (dt, J=32.3, 10.4 Hz, 3H), 1.72-1.51 (m, 6H), 1.36 (d, J=6.3 Hz, 3H).

### Example 71. (R)-1-(5-((2-methyl-4-(methylsulfonyl)piperazin-1-yl)methyl)benzo[d]isoxazol-3-yl)dihydropyrimidine-2,4(1H,3H)-dione

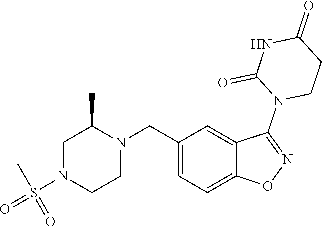 [SMILES: C[C@@H]1CN(S(C)(=O)=O)CCN1Cc1ccc2onc(N3CCC(=O)NC3=O)c2c1]

Prepared by the method of Example 64 wherein methanesulfonyl chloride was used in place of acetyl chloride in step 4. LCMS [M+H]⁺: 422.2. ¹H NMR (400 MHz, MeOD) δ 7.83 (d, J=1.6 Hz, 1H), 7.64 (dd, J=8.7, 1.7 Hz, 1H), 7.55 (d, J=8.9 Hz, 1H), 4.20-4.10 (m, 3H), 3.44-3.35 (m, 3H), 2.98 (ddd, J=11.8, 9.2, 3.0 Hz, 1H), 2.89 (t, J=6.7 Hz, 2H), 2.84-2.77 (m, 5H), 2.72-2.59 (m, 1H), 2.30 (ddd, J=12.1, 9.2, 3.2 Hz, 1H), 1.23 (d, J=6.2 Hz, 3H). NH not observed due to solvent exchange.

### Example 72. (R)-1-(5-((4-(ethylsulfonyl)-2-methylpiperazin-1-yl)methyl)benzo[d]isoxazol-3-yl)dihydropyrimidine-2,4(1H,3H)-dione

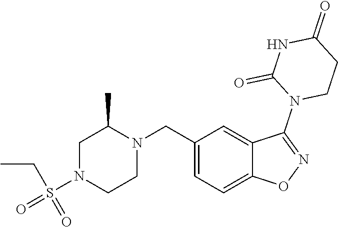 [SMILES: CCS(=O)(=O)N1CCN(Cc2ccc3onc(N4CCC(=O)NC4=O)c3c2)[C@H](C)C1]

Prepared by the method of Example 64 wherein ethanesulfonyl chloride was used in place of acetyl chloride in step 4. LCMS [M+H]⁺: 436.2. ¹H NMR (400 MHz, DMSO) δ 10.89 (s, 1H), 7.75 (d, J=1.6 Hz, 1H), 7.67 (dd, J=8.7, 0.7 Hz, 1H), 7.61 (dd, J=8.7, 1.7 Hz, 1H), 4.10-3.97 (m, 3H), 3.37 (s, 1H), 3.24 (d, J=12.2 Hz, 1H), 3.08-3.00 (m, 2H), 2.95 (t, J=8.9 Hz, 1H), 2.83-2.75 (m, 3H), 2.22-2.13 (m, 1H), 1.20 (q, J=8.0 Hz, 6H), 1.12 (d, J=6.2 Hz, 3H).

### Example 73. (R)-1-(5-((4-(isopropylsulfonyl)-2-methylpiperazin-1-yl)methyl)benzo[d]isoxazol-3-yl)dihydropyrimidine-2,4(1H,3H)-dione

64 wherein propane-2-sulfonyl chloride was used in place of acetyl chloride in step 4. LCMS [M+H]⁺: 450.2. ¹H NMR (400 MHz, DMSO) δ 10.88 (s, 1H), 7.77-7.73 (m, 1H), 7.67 (dd, J=8.7, 0.7 Hz, 1H), 7.61 (dd, J=8.7, 1.6 Hz, 1H), 4.10-3.99 (m, 3H), 3.39 (d, J=11.4 Hz, 4H), 3.04 (t, J=9.3 Hz, 1H), 2.87 (dd, J=12.1, 7.9 Hz, 1H), 2.80 (t, J=6.6 Hz, 2H), 2.64-2.58 (m, 1H), 2.19-2.10 (m, 1H), 2.00 (q, J=6.9 Hz, 1H), 1.20 (dd, J=6.8, 2.0 Hz, 6H), 1.11 (d, J=6.3 Hz, 3H).

### Example 74. (R)-1-(5-((2-methyl-4-(pyrrolidin-1-ylsulfonyl)piperazin-1-yl)methyl)benzo[d]isoxazol-3-yl)dihydropyrimidine-2,4(1H,3H)-dione

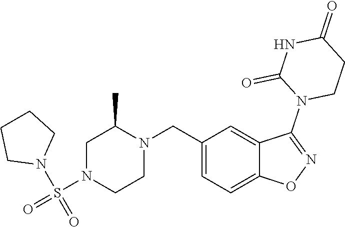 [SMILES: C[C@@H]1CN(S(=O)(=O)N2CCCC2)CCN1Cc1ccc2onc(N3CCC(=O)NC3=O)c2c1]

Prepared by the method of Example 64 wherein pyrrolidine-1-sulfonyl chloride was used in place of acetyl chloride in step 4 and step 4 was carried out at rt. LCMS [M+H]⁺: 477.3. ¹H NMR (400 MHz, MeOD) δ 8.34 (s, 1H), 7.83 (t, J=1.1 Hz, 1H), 7.63 (dd, J=8.7, 1.7 Hz, 1H), 7.55 (dd, J=8.7, 0.7 Hz, 1H), 4.21-4.07 (m, 3H), 3.44-3.36 (m, 2H), 3.28 (d, J=6.7 Hz, 5H), 3.01 (ddd, J=12.1, 9.4, 3.0 Hz, 1H), 2.89 (t, J=6.7 Hz, 2H), 2.86-2.81 (m, 1H), 2.77 (ddd, J=11.8, 4.8, 3.0 Hz, 1H), 2.63 (tt, J=9.3, 6.2 Hz, 1H), 2.27 (ddd, J=12.3, 9.4, 3.2 Hz, 1H), 1.94-1.87 (m, 4H), 1.22 (d, J=6.2 Hz, 3H).

### Example 75. (R)-1-(5-((2-methyl-4-(pyrrolidine-1-carbonyl)piperazin-1-yl)methyl)benzo[d]isoxazol-3-yl)dihydropyrimidine-2,4(1H,3H)-dione

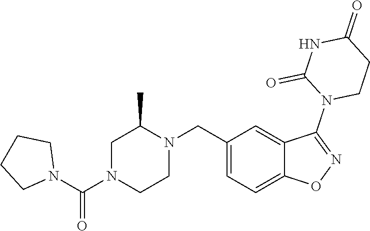 [SMILES: C[C@@H]1CN(C(=O)N2CCCC2)CCN1Cc1ccc2onc(N3CCC(=O)NC3=O)c2c1]

Prepared by the method of Example 64 wherein pyrrolidine-1-carbonyl chloride was used in place of acetyl chloride in step 4 and step 4 was carried out at rt. LCMS [M+H]⁺: 441.3. ¹H NMR (400 MHz, MeOD) δ 8.32 (s, 1H), 7.88 (d, J=1.6 Hz, 1H), 7.66 (dd, J=8.8, 1.7 Hz, 1H), 7.58 (d, J=8.7 Hz, 1H), 4.29 (d, J=13.3 Hz, 1H), 4.17 (t, J=6.7 Hz, 2H), 3.63-3.46 (m, 3H), 3.36 (q, J=5.2 Hz, 4H), 3.06 (ddd, J=13.2, 10.1, 2.9 Hz, 1H), 2.96-2.85 (m, 3H), 2.82 (ddd, J=12.0, 4.2, 2.9 Hz, 1H), 2.71 (ddd, J=9.2, 6.2, 3.1 Hz, 1H), 2.36 (ddd, J=11.9, 10.1, 3.2 Hz, 1H), 1.84 (td, J=7.9, 4.5 Hz, 4H), 1.28 (d, J=6.2 Hz, 3H).

### Example 76. (R)—N-cyclopentyl-4-((3-(2,4-dioxotetrahydropyrimidin-1(2H)-yl)benzo[d]isoxazol-5-yl)methyl)-3-methylpiperazine-1-carboxamide

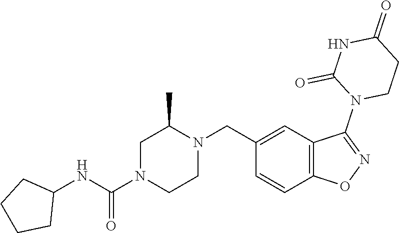 [SMILES: C[C@@H]1CN(C(=O)NC2CCCC2)CCN1Cc1ccc2onc(N3CCC(=O)NC3=O)c2c1]

Prepared by the method of Example 64 wherein isocyanatocyclopentane was used in place of acetyl chloride in step 4 and step 4 was carried out at rt. LCMS [M+H]⁺: 455.1. ¹H NMR (400 MHz, MeOD) δ 8.30 (s, 1H), 7.87 (d, J=1.6 Hz, 1H), 7.66 (dd, J=8.7, 1.7 Hz, 1H), 7.58 (d, J=8.7 Hz, 1H), 4.25 (d, J=13.3 Hz, 1H), 4.17 (t, J=6.7 Hz, 2H), 3.99 (p, J=7.3 Hz, 1H), 3.76 (ddd, J=13.2, 3.3, 1.7 Hz, 1H), 3.72-3.62 (m, 1H), 3.49 (d, J=13.3 Hz, 1H), 3.07 (ddd, J=13.2, 10.0, 3.1 Hz, 1H), 2.90 (dt, J=13.4, 7.7 Hz, 3H), 2.77 (ddd, J=11.9, 4.3, 3.1 Hz, 1H), 2.61 (ddd, J=9.2, 6.2, 3.2 Hz, 1H), 2.26 (ddd, J=11.9, 10.0, 3.3 Hz, 1H), 1.97-1.86 (m, 2H), 1.76-1.65 (m, 2H), 1.62-1.52 (m, 2H), 1.42 (dddd, J=14.1, 8.8, 7.5, 4.2 Hz, 2H), 1.25 (d, J=6.3 Hz, 3H). NH proton not observed due to solvent exchange.

### Example 77. (R)-1-(5-((4-cyclobutyl-2-methylpiperazin-1-yl)methyl)benzo[d]isoxazol-3-yl)dihydropyrimidine-2,4(1H,3H)-dione

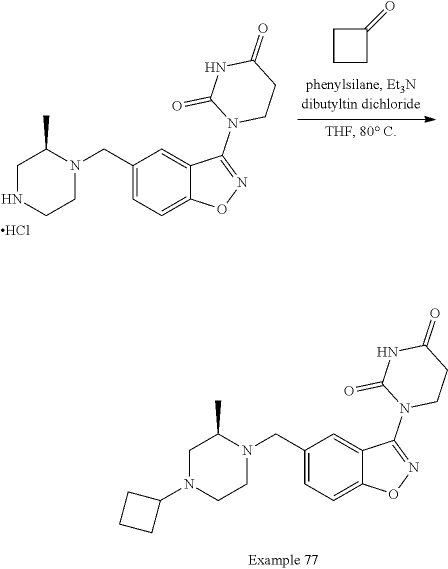 [SMILES: C[C@@H]1CNCCN1Cc1ccc2onc(N3CCC(=O)NC3=O)c2c1 ; O=C1CCC1 ; Cl ; C[C@@H]1CN(C2CCC2)CCN1Cc1ccc2onc(N3CCC(=O)NC3=O)c2c1]

To a solution of (R)-1-(5-((2-methylpiperazin-1-yl)methyl)benzo[d]isoxazol-3-yl)dihydropyrimidine-2,4(1H,3H)-dione hydrochloride (80 mg, 0.21 mmol) in THF (10 mL) was added cyclobutanone (45 mg, 0.63 mmol), dibutyltin dichloride (128 mg, 0.42 mmol), and triethylamine (0.1 mL, 0.63 mmol). The mixture was stirred at 80° C. for 3 h and then cooled to 0° C. and phenylsilane (68 mg, 0.63 mmol) was added. The reaction was stirred in a capped vial at 80° C. for 3 h. The reaction was cooled to rt, diluted with DCM and washed sequentially with water and brine. The organic layer was dried over Na₂SO₄, filtered and concentrated. The crude material was purified by reverse phase HPLC using ACN/water/0.1% formic acid. The fractions containing the product were combined, frozen and lyophilized to afford (R)-1-(5-((4-cyclobutyl-2-methylpiperazin-1-yl)methyl)benzo[d]isoxazol-3-yl)dihydropyrimidine-2,4(1H,3H)-dione as a white solid (18 mg, 0.04 mmole, 21% yield). LCMS [M+H]⁺: 398.3. ¹H NMR (400 MHz, MeOD) δ 7.84 (d, J=1.6 Hz, 1H), 7.63 (dd, J=8.8, 1.7 Hz, 1H), 7.59-7.53 (m, 1H), 4.28 (d, J=13.4 Hz, 1H), 4.17 (t, J=6.7 Hz, 2H), 3.38 (d, J=13.4 Hz, 1H), 3.07 (dd, J=19.5, 11.8 Hz, 2H), 2.93-2.80 (m, 3H), 2.69 (d, J=3.7 Hz, 1H), 2.47 (t, J=11.6 Hz, 1H), 2.40-2.27 (m, 2H), 2.27-2.00 (m, 3H), 1.87-1.65 (m, 3H), 1.42 (p, J=7.4 Hz, 1H), 1.27 (d, J=6.2 Hz, 3H). NH not observed due to solvent exchange.

### Example 78. (R)-1-(5-((2-methyl-4-(tetrahydro-2H-pyran-4-yl)piperazin-1-yl)methyl)benzo[d]isoxazol-3-yl)dihydropyrimidine-2,4(1H,3H)-dione

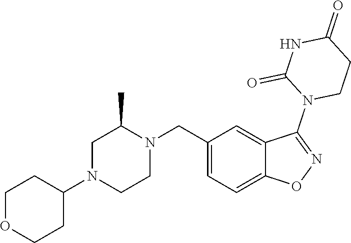 [SMILES: C[C@@H]1CN(C2CCOCC2)CCN1Cc1ccc2onc(N3CCC(=O)NC3=O)c2c1]

Prepared by the method of Example 77 wherein tetrahydro-4H-pyran-4-one was used in place of cyclobutanone. LCMS [M+H]⁺: 428.1. ¹H NMR (400 MHz, MeOD) δ 8.39 (s, 1H), 7.87 (d, J=1.6 Hz, 1H), 7.65 (dd, J=8.7, 1.7 Hz, 1H), 7.59 (d, J=8.7 Hz, 1H), 4.34 (d, J=13.3 Hz, 1H), 4.18 (t, J=6.6 Hz, 2H), 4.03 (d, J=11.2 Hz, 2H), 3.49-3.36 (m, 3H), 3.11-3.02 (m, 1H), 2.96 (dt, J=12.9, 3.0 Hz, 1H), 2.91-2.84 (m, 2H), 2.84-2.73 (m, 2H), 2.66 (s, 1H), 2.44 (t, J=12.0 Hz, 1H), 1.97 (t, J=11.6 Hz, 2H), 1.64 (ddd, J=12.3, 7.8, 4.6 Hz, 2H), 1.36-1.31 (m, 5H).

### Example 79. 1-(5-((4-isopropylpiperazin-1-yl)methyl)benzo[d]isoxazol-3-yl)dihydropyrimidine-2,4(1H,3H)-dione

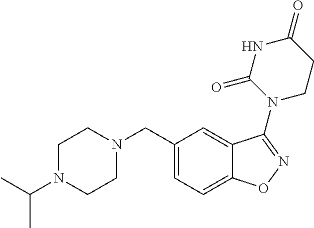 [SMILES: CC(C)N1CCN(Cc2ccc3onc(N4CCC(=O)NC4=O)c3c2)CC1]

Prepared by the method of Example 77 wherein 1-(5-(piperazin-1-ylmethyl)benzo[d]isoxazol-3-yl)dihydropyrimidine-2,4(1H,3H)-dione hydrochloride was used in place of (R)-1-(5-((2-methylpiperazin-1-yl)methyl)benzo[d]isoxazol-3-yl)dihydropyrimidine-2,4(1H,3H)-dione hydrochloride and acetone was used in place of cyclobutanone. LCMS [M+H]⁺: 372.2. ¹H NMR (400 MHz, MeOD) δ 8.53 (s, 1H), 7.83 (dd, J=1.6, 0.8 Hz, 1H), 7.64 (dd, J=8.7, 1.7 Hz, 1H), 7.56 (dd, J=8.7, 0.7 Hz, 1H), 4.17 (t, J=6.7 Hz, 2H), 3.69 (s, 2H), 3.05 (t, J=6.6 Hz, 1H), 2.89 (q, J=9.5 Hz, 6H), 2.78-2.54 (m, 4H), 1.21 (d, J=6.6 Hz, 6H).

### Example 80. 1-(5-((4-cyclobutylpiperazin-1-yl)methyl)benzo[d]isoxazol-3-yl)dihydropyrimidine-2,4(1H,3H)-dione

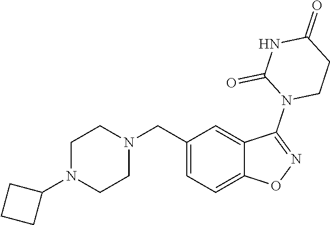 [SMILES: O=C1CCN(c2noc3ccc(CN4CCN(C5CCC5)CC4)cc23)C(=O)N1]

Prepared by the method of Example 77 wherein 1-(5-(piperazin-1-ylmethyl)benzo[d]isoxazol-3-yl)dihydropyrimidine-2,4(1H,3H)-dione hydrochloride was used in place of (R)-1-(5-((2-methylpiperazin-1-yl)methyl)benzo[d]isoxazol-3-yl)dihydropyrimidine-2,4(1H,3H)-dione hydrochloride. LCMS [M+H]⁺: 384.0. ¹H NMR (400 MHz, MeOD) δ 8.40 (s, 1H), 7.85 (d, J=1.5 Hz, 1H), 7.64 (dd, J=8.7, 1.7 Hz, 1H), 7.57 (d, J=8.7 Hz, 1H), 4.17 (t, J=6.7 Hz, 2H), 3.77 (s, 2H), 3.57 (p, J=8.2 Hz, 1H), 3.08 (s, 2H), 2.88 (t, J=6.7 Hz, 2H), 2.76 (s, 2H), 2.33-2.14 (m, 4H), 1.92-1.78 (m, 2H), 1.63 (dd, J=37.5, 8.0 Hz, 1H), 1.51-1.25 (m, 2H), 1.00-0.87 (m, 1H).

### Example 81. 1-(5-((4-(tetrahydro-2H-pyran-4-yl)piperazin-1-yl)methyl)benzo[d]isoxazol-3-yl)dihydropyrimidine-2,4(1H,3H)-dione

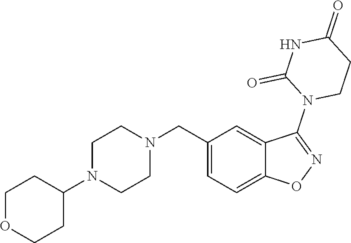 [SMILES: O=C1CCN(c2noc3ccc(CN4CCN(C5CCOCC5)CC4)cc23)C(=O)N1]

Prepared by the method of Example 77 wherein 1-(5-(piperazin-1-ylmethyl)benzo[d]isoxazol-3-yl)dihydropyrimidine-2,4(1H,3H)-dione hydrochloride was used in place of (R)-1-(5-((2-methylpiperazin-1-yl)methyl)benzo[d]isoxazol-3-yl)dihydropyrimidine-2,4(1H,3H)-dione hydrochloride and tetrahydro-4H-pyran-4-one was used in place of cyclobutanone. LCMS [M+H]⁺: 414.2. ¹H NMR (400 MHz, MeOD) δ 8.42 (s, 1H), 7.86 (d, J=1.6 Hz, 1H), 7.65 (dd, J=8.7, 1.7 Hz, 1H), 7.58 (d, J=8.6 Hz, 1H), 4.17 (t, J=6.6 Hz, 2H), 4.03 (dd, J=11.6, 4.4 Hz, 2H), 3.77 (s, 2H), 3.41 (td, J=12.0, 1.7 Hz, 2H), 3.07 (q, J=15.9 Hz, 5H), 2.88 (t, J=6.6 Hz, 2H), 2.75 (s, 4H), 2.00-1.92 (m, 2H), 1.64 (qd, J=12.1, 4.6 Hz, 2H).

### Example 82. (S)-1-(5-((3-methyl-4-(oxetan-3-yl)piperazin-1-yl)methyl)benzo[d]isoxazol-3-yl)dihydropyrimidine-2,4(1H,3H)-dione

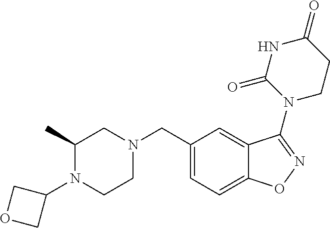 [SMILES: C[C@H]1CN(Cc2ccc3onc(N4CCC(=O)NC4=O)c3c2)CCN1C1COC1]

Prepared by the method of Example 77 wherein (S)-1-(5-((3-methylpiperazin-1-yl)methyl)benzo[d]isoxazol-3-yl)dihydropyrimidine-2,4(1H,3H)-dione hydrochloride was used in place of (R)-1-(5-((2-methylpiperazin-1-yl)methyl)benzo[d]isoxazol-3-yl)dihydropyrimidine-2,4(1H,3H)-dione hydrochloride and oxetan-3-one was used in place of cyclobutanone. LCMS [M+H]⁺: 400.0. ¹H NMR (400 MHz, MeOD) δ 8.39 (s, 1H), 7.90 (d, J=1.6 Hz, 1H), 7.66 (dd, J=8.8, 1.7 Hz, 1H), 7.60 (d, J=8.7 Hz, 1H), 4.68 (dd, J=12.6, 6.8 Hz, 3H), 4.60 (t, J=6.8 Hz, 1H), 4.17 (t, J=6.6 Hz, 2H), 3.91-3.80 (m, 3H), 2.96 (d, J=12.0 Hz, 1H), 2.92-2.75 (m, 4H), 2.64-2.47 (m, 2H), 2.31 (dt, J=20.6, 10.5 Hz, 2H), 0.92 (d, J=6.3 Hz, 3H).

### Example 83. 1-(5-((4-(methylsulfonyl)piperazin-1-yl)methyl)benzo[d]isoxazol-3-yl)dihydropyrimidine-2,4(1H,3H)-dione

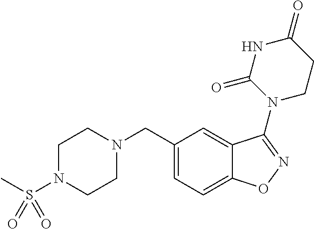 [SMILES: CS(=O)(=O)N1CCN(Cc2ccc3onc(N4CCC(=O)NC4=O)c3c2)CC1]

Prepared by the method of Example 64, step 4 wherein 1-(5-(piperazin-1-ylmethyl)benzo[d]isoxazol-3-yl)dihydropyrimidine-2,4(1H,3H)-dione hydrochloride was used in place of (R)-1-(5-((2-methylpiperazin-1-yl)methyl)benzo[d]isoxazol-3-yl)dihydropyrimidine-2,4(1H,3H)-dione hydrochloride and methanesulfonyl chloride was used in place of acetyl chloride. LCMS [M+H]⁺: 408.1. ¹H NMR (400 MHz, cd3od) δ 8.24 (s, 1H), 7.83 (s, 1H), 7.64 (dd, J=8.7, 1.8 Hz, 1H), 7.56 (d, J=8.9 Hz, 1H), 4.17 (t, J=6.7 Hz, 2H), 3.70 (s, 2H), 3.23 (t, J=5.0 Hz, 4H), 2.91-2.85 (m, 2H), 2.83 (d, J=1.5 Hz, 3H), 2.59 (t, J=4.9 Hz, 4H).

### Example 84. 1-(5-((4-(isopropylsulfonyl)piperazin-1-yl)methyl)benzo[d]isoxazol-3-yl)dihydropyrimidine-2,4(1H,3H)-dione

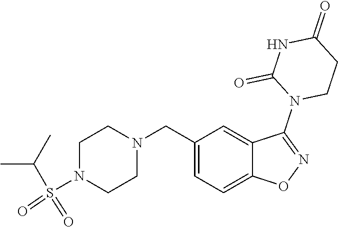 [SMILES: CC(C)S(=O)(=O)N1CCN(Cc2ccc3onc(N4CCC(=O)NC4=O)c3c2)CC1]

Prepared by the method of Example 64, step 4 wherein 1-(5-(piperazin-1-ylmethyl)benzo[d]isoxazol-3-yl)dihydropyrimidine-2,4(1H,3H)-dione hydrochloride was used in place of (R)-1-(5-((2-methylpiperazin-1-yl)methyl)benzo[d]isoxazol-3-yl)dihydropyrimidine-2,4(1H,3H)-dione hydrochloride and propane-2-sulfonyl chloride was used in place of acetyl chloride. LCMS [M+H]⁺: 436.2. ¹H NMR (400 MHz, cd3od) δ 8.11 (s, 1H), 7.86 (s, 1H), 7.65 (d, J=8.7 Hz, 1H), 7.58 (d, J=8.6 Hz, 1H), 4.17 (t, J=6.6 Hz, 2H), 3.77 (s, 2H), 3.38 (t, J=5.0 Hz, 5H), 2.88 (t, J=6.7 Hz, 2H), 2.62 (t, J=4.9 Hz, 4H), 1.30 (d, J=6.9 Hz, 6H).

### Example 85. 1-(5-((4-isobutyrylpiperazin-1-yl)methyl)benzo[d]isoxazol-3-yl)dihydropyrimidine-2,4(1H,3H)-dione

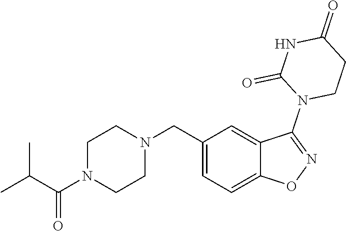 [SMILES: CC(C)C(=O)N1CCN(Cc2ccc3onc(N4CCC(=O)NC4=O)c3c2)CC1]

Prepared by the method of Example 64, step 4 wherein 1-(5-(piperazin-1-ylmethyl)benzo[d]isoxazol-3-yl)dihydropyrimidine-2,4(1H,3H)-dione hydrochloride was used in place of (R)-1-(5-((2-methylpiperazin-1-yl)methyl)benzo[d]isoxazol-3-yl)dihydropyrimidine-2,4(1H,3H)-dione hydrochloride and isobutyryl chloride was used in place of acetyl chloride. LCMS [M+H]⁺: 400.0. ¹H NMR (400 MHz, cd3od) δ 8.09 (s, 1H), 7.75 (m, 1H), 7.69 (m, 1H), 4.94 (br s, 4H), 4.51 (s, 2H), 4.17 (t, J=6.6 Hz, 2H), 3.35 (m, 4H), 2.95 (m, 1H), 2.87 (t, J=6.7 Hz, 2H), 1.09 (t, J=6.6 Hz, 6H). NH not observed due to solvent exchange.

### Example 86. (S)-1-(5-((3-methyl-4-(oxetan-3-ylmethyl)piperazin-1-yl)methyl)benzo[d]isoxazol-3-yl)dihydropyrimidine-2,4(1H,3H)-dione

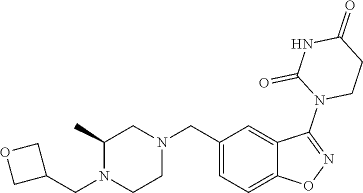 [SMILES: C[C@H]1CN(Cc2ccc3onc(N4CCC(=O)NC4=O)c3c2)CCN1CC1COC1]

Prepared by the method of Example 21, using potassium (S)-((4-(tert-butoxycarbonyl)-3-methylpiperazin-1-yl)methyl)trifluoroborate in step 1 and oxetane-3-carbaldehyde in step 4. LCMS [M+H]⁺: 414.0. ¹H NMR (400 MHz, cd3od) δ 8.42 (s, 1H), 7.85 (s, 1H), 7.64 (dd, J=8.6, 1.7 Hz, 1H), 7.58 (d, J=8.6 Hz, 1H), 4.80 (dt, J=9.7, 6.7 Hz, 2H), 4.44 (dt, J=8.5, 6.0 Hz, 2H), 4.17 (t, J=6.6 Hz, 2H), 3.74 (d, J=4.1 Hz, 2H), 3.44-3.33 (m, 2H), 3.03-2.93 (m, 1H), 2.87 (q, J=7.1 Hz, 5H), 2.58 (t, J=11.1 Hz, 1H), 2.46 (d, J=11.7 Hz, 1H), 2.23 (s, 1H), 1.36-1.27 (m, 1H), 1.20 (d, J=6.2 Hz, 3H).

### Example 87. (R)-1-(5-((2-ethyl-4-methylpiperazin-1-yl)methyl)benzo[d]isoxazol-3-yl)dihydropyrimidine-2,4(1H,3H)-dione

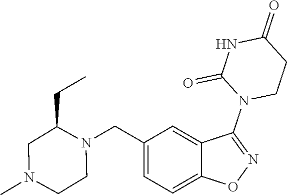 [SMILES: CC[C@@H]1CN(C)CCN1Cc1ccc2onc(N3CCC(=O)NC3=O)c2c1]

Prepared by the method of Example 21, using potassium (R)-((4-(tert-butoxycarbonyl)-2-ethylpiperazin-1-yl)methyl)trifluoroborate in step 1 and paraformaldehyde in step 4. LCMS [M+H]⁺: 372.2. ¹H NMR (400 MHz, cd3od) δ 8.49 (s, 1H), 7.84 (s, 1H), 7.63 (dd, J=8.8, 1.7 Hz, 1H), 7.57 (d, J=8.8 Hz, 1H), 4.25 (d, J=13.5 Hz, 1H), 4.17 (t, J=6.6 Hz, 2H), 3.37 (d, J=14.4 Hz, 1H), 3.22 (d, J=11.9 Hz, 1H), 3.09 (d, J=11.9 Hz, 1H), 2.92-2.85 (m, 3H), 2.77 (s, 2H), 2.68 (s, 3H), 2.61 (s, 1H), 2.38 (t, J=11.9 Hz, 1H), 1.89-1.68 (m, 2H), 1.01 (t, J=7.4 Hz, 3H).

### Example 88. (R)-1-(5-((2-ethyl-4-isobutylpiperazin-1-yl)methyl)benzo[d]isoxazol-3-yl)dihydropyrimidine-2,4(1H,3H)-dione

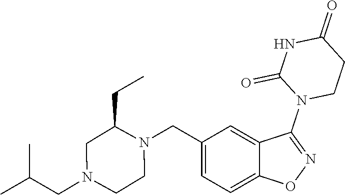 [SMILES: CC[C@@H]1CN(CC(C)C)CCN1Cc1ccc2onc(N3CCC(=O)NC3=O)c2c1]

Prepared by the method of Example 21, using potassium (R)-((4-(tert-butoxycarbonyl)-2-ethylpiperazin-1-yl)methyl)trifluoroborate in step 1 and isobutyraldehyde in step 4. LCMS [M+H]⁺: 414.1. ¹H NMR (400 MHz, cd3od) δ 8.42 (s, 1H), 7.88 (d, J=1.6 Hz, 1H), 7.65 (dd, J=8.8, 1.7 Hz, 1H), 7.59 (d, J=8.7 Hz, 1H), 4.33 (d, J=13.3 Hz, 1H), 4.18 (t, J=6.7 Hz, 2H), 3.49 (d, J=13.0 Hz, 1H), 3.24 (dd, J=24.8, 10.2 Hz, 2H), 2.94 (dt, J=13.1, 3.3 Hz, 1H), 2.88 (t, J=6.7 Hz, 2H), 2.85-2.69 (m, 5H), 2.53 (t, J=12.0 Hz, 1H), 2.05 (dt, J=13.6, 6.9 Hz, 1H), 1.82 (dt, J=14.2, 7.7 Hz, 2H), 1.02 (dd, J=16.2, 7.0 Hz, 9H).

### Example 89. (R)-1-(5-((2-isopropyl-4-methylpiperazin-1-yl)methyl)benzo[d]isoxazol-3-yl)dihydropyrimidine-2,4(1H,3H)-dione

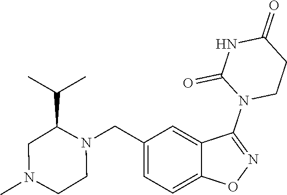 [SMILES: CC(C)[C@@H]1CN(C)CCN1Cc1ccc2onc(N3CCC(=O)NC3=O)c2c1]

Prepared by the method of Example 21, using potassium (R)-((4-(tert-butoxycarbonyl)-2-isopropylpiperazin-1-yl)methyl)trifluoroborate in step 1 and paraformaldehyde in step 4. LCMS [M+H]⁺: 386.3. ¹H NMR (400 MHz, cd3od) δ 7.83 (s, 1H), 7.64 (dd, J=8.6, 1.8 Hz, 1H), 7.56 (d, J=8.7 Hz, 1H), 4.29 (d, J=13.4 Hz, 1H), 4.17 (t, J=6.7 Hz, 2H), 3.25 (d, J=13.5 Hz, 1H), 3.10 (d, J=11.1 Hz, 1H), 2.96 (d, J=11.7 Hz, 1H), 2.93-2.80 (m, 4H), 2.56 (s, 3H), 2.53-2.39 (m, 3H), 2.29 (t, J=12.0 Hz, 1H), 1.02 (dd, J=8.2, 6.5 Hz, 6H). NH not observed due to solvent exchange.

### Example 90. (R)-1-(5-((4-isobutyl-2-isopropylpiperazin-1-yl)methyl)benzo[d]isoxazol-3-yl)dihydropyrimidine-2,4(1H,3H)-dione

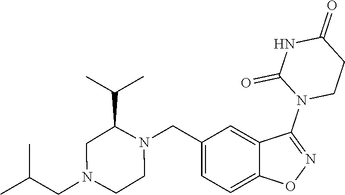 [SMILES: CC(C)CN1CCN(Cc2ccc3onc(N4CCC(=O)NC4=O)c3c2)[C@H](C(C)C)C1]

Prepared by the method of Example 21, using potassium (R)-((4-(tert-butoxycarbonyl)-2-isopropylpiperazin-1-yl)methyl)trifluoroborate in step 1 and isobutyraldehyde in step 4. LCMS [M+H]⁺: 428.1. ¹H NMR (400 MHz, cd3od) δ 8.41 (s, 1H), 7.85 (s, 1H), 7.64 (dd, J=8.9, 1.8 Hz, 1H), 7.58 (d, J=8.8 Hz, 1H), 4.34 (d, J=13.4 Hz, 1H), 4.17 (t, J=6.7 Hz, 2H), 3.36 (d, J=12.9 Hz, 3H), 2.98-2.80 (m, 7H), 2.68 (t, J=7.1 Hz, 1H), 2.48 (dd, J=15.3, 9.3 Hz, 2H), 2.11 (dt, J=13.6, 6.8 Hz, 1H), 1.10-0.97 (m, 12H).

### Example 91. tert-butyl 4-((3-(2,4-dioxotetrahydropyrimidin-1(2H)-yl)benzo[d]isoxazol-5-yl)methyl)-2,2-dimethylpiperazine-1-carboxylate

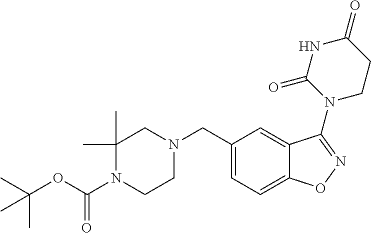 [SMILES: CC(C)(C)OC(=O)N1CCN(Cc2ccc3onc(N4CCC(=O)NC4=O)c3c2)CC1(C)C]

Prepared by the method of Example 21, steps 1-2 using potassium ((4-(tert-butoxycarbonyl)-3,3-dimethylpiperazin-1-yl)methyl)trifluoroborate in step 1. LCMS [M+H]⁺: 458.2. ¹H NMR (400 MHz, cd3od) δ 7.82 (s, 1H), 7.66 (dd, J=8.8, 1.7 Hz, 1H), 7.55 (d, J=8.7 Hz, 1H), 4.17 (t, J=6.7 Hz, 2H), 3.59 (s, 2H), 3.47-3.40 (m, 2H), 2.88 (t, J=6.6 Hz, 2H), 2.46 (t, J=5.5 Hz, 2H), 2.21 (s, 2H), 1.45 (s, 9H), 1.36 (s, 6H). NH not observed due to solvent exchange.

### Example 92. 1-(5-((3,3,4-trimethylpiperazin-1-yl)methyl)benzo[d]isoxazol-3-yl)dihydropyrimidine-2,4(1H,3H)-dione

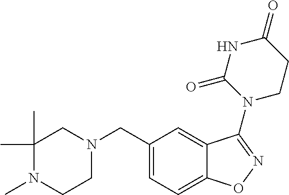 [SMILES: CN1CCN(Cc2ccc3onc(N4CCC(=O)NC4=O)c3c2)CC1(C)C]

Prepared by the method of Example 21, using potassium ((4-(tert-butoxycarbonyl)-3,3-dimethylpiperazin-1-yl)methyl)trifluoroborate in step 1 and paraformaldehyde in step 4. LCMS [M+H]⁺: 372.2. ¹H NMR (400 MHz, cd3od) δ 8.55 (s, 1H), 7.82 (s, 1H), 7.64 (dd, J=8.8, 1.8 Hz, 1H), 7.56 (dd, J=9.0, 1.7 Hz, 1H), 4.17 (td, J=6.7, 1.8 Hz, 2H), 3.65 (s, 2H), 3.12 (s, 2H), 2.88 (td, J=6.7, 1.8 Hz, 2H), 2.63 (s, 3H), 2.58-2.23 (m, 4H), 1.30 (s, 6H).

### Example 93. tert-butyl (1S,4S)-5-((3-(2,4-dioxotetrahydropyrimidin-1(2H)-yl)benzo[d]isoxazol-5-yl)methyl)-2,5-diazabicyclo[2.2.1]heptane-2-carboxylate

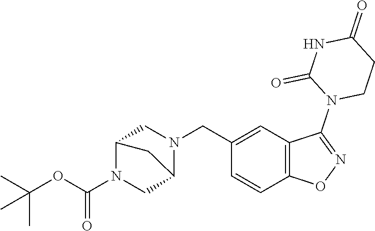 [SMILES: CC(C)(C)OC(=O)N1C[C@@H]2C[C@H]1CN2Cc1ccc2onc(N3CCC(=O)NC3=O)c2c1]

Prepared by the method of Example 21, steps 1-2 using potassium (((1S,4S)-5-(tert-butoxycarbonyl)-2,5-diazabicyclo[2.2.1]heptan-2-yl)methyl)trifluoroborate in step 1. LCMS [M+H]⁺: 442.0. ¹H NMR (400 MHz, cd3od) δ 7.90 (s, 1H), 7.68 (d, J=8.7 Hz, 1H), 7.58 (d, J=8.8 Hz, 1H), 4.37 (s, 1H), 4.17 (t, J=6.6 Hz, 2H), 4.00 (t, J=10.8 Hz, 2H), 3.71 (s, 1H), 3.54 (d, J=10.6 Hz, 1H), 3.28-3.19 (m, 1H), 2.97 (d, J=10.5 Hz, 1H), 2.88 (t, J=6.6 Hz, 2H), 2.81 (t, J=12.4 Hz, 1H), 2.02 (d, J=10.5 Hz, 1H), 1.83 (t, J=9.4 Hz, 1H), 1.46 (s, 9H). NH not observed due to solvent exchange.

### Example 94. 1-(5-((3,3,4-trimethylpiperazin-1-yl)methyl)benzo[d]isoxazol-3-yl)dihydropyrimidine-2,4(1H,3H)-dione

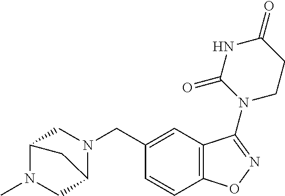 [SMILES: CN1C[C@@H]2C[C@H]1CN2Cc1ccc2onc(N3CCC(=O)NC3=O)c2c1]

Prepared by the method of Example 21, using potassium (((1S,4S)-5-(tert-butoxycarbonyl)-2,5-diazabicyclo[2.2.1]heptan-2-yl)methyl)trifluoroborate in step 1 and paraformaldehyde in step 4. LCMS [M+H]⁺: 356.2. ¹H NMR (400 MHz, DMSO) δ 8.42 (s, 1H), 7.76 (s, 1H), 7.67-7.57 (m, 2H), 4.06 (t, J=6.6 Hz, 2H), 3.75 (q, J=13.6 Hz, 2H), 3.20 (d, J=30.0 Hz, 2H), 2.82-2.73 (m, 2H), 2.70-2.57 (m, 4H), 2.28 (s, 3H), 1.63 (s, 2H).

### Example 95. 1-(5-(((1S,4S)-5-((tetrahydro-2H-pyran-4-yl)methyl)-2,5-diazabicyclo[2.2.1]heptan-2-yl)methyl)benzo[d]isoxazol-3-yl)dihydropyrimidine-2,4(1H,3H)-dione

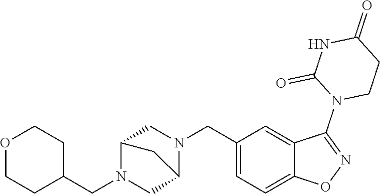 [SMILES: O=C1CCN(c2noc3ccc(CN4C[C@@H]5C[C@H]4CN5CC4CCOCC4)cc23)C(=O)N1]

Prepared by the method of Example 21, using potassium (((1S,4S)-5-(tert-butoxycarbonyl)-2,5-diazabicyclo[2.2.1]heptan-2-yl)methyl)trifluoroborate in step 1 and tetrahydro-2H-pyran-4-carbaldehyde in step 4. LCMS [M+H]⁺: 440.2. ¹H NMR (400 MHz, MeOD) δ 8.43 (s, 1H), 7.93 (s, 1H), 7.70 (dd, J=9.1, 1.5 Hz, 1H), 7.60 (d, J=8.7 Hz, 1H), 4.18 (t, J=6.6 Hz, 2H), 4.10 (d, J=12.1 Hz, 2H), 3.96 (t, J=10.6 Hz, 3H), 3.77 (s, 1H), 3.43 (t, J=11.8 Hz, 3H), 3.22-3.13 (m, 2H), 3.05-2.84 (m, 4H), 2.21 (d, J=11.5 Hz, 1H), 2.08 (d, J=11.7 Hz, 1H), 1.94 (s, 1H), 1.72 (t, J=15.1 Hz, 2H), 1.35 (ddt, J=20.0, 14.0, 6.6 Hz, 3H).

### Example 96. tert-butyl (1R,4R)-5-((3-(2,4-dioxotetrahydropyrimidin-1(2H)-yl)benzo[d]isoxazol-5-yl)methyl)-2,5-diazabicyclo[2.2.1]heptane-2-carboxylate

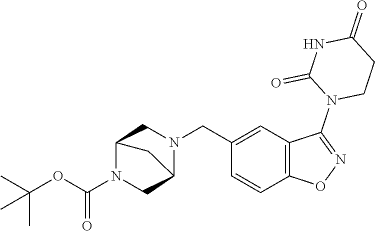 [SMILES: CC(C)(C)OC(=O)N1C[C@H]2C[C@@H]1CN2Cc1ccc2onc(N3CCC(=O)NC3=O)c2c1]

Prepared by the method of Example 21, steps 1-2 using potassium (((1R,4R)-5-(tert-butoxycarbonyl)-2,5-diazabicyclo[2.2.1]heptan-2-yl)methyl)trifluoroborate in step 1. LCMS [M+H]⁺: 442.1. ¹H NMR (400 MHz, cd3od) δ 7.88 (s, 1H), 7.68 (d, J=8.7 Hz, 1H), 7.57 (d, J=8.6 Hz, 1H), 4.34 (s, 1H), 4.17 (t, J=6.7 Hz, 2H), 3.93 (d, J=4.3 Hz, 2H), 3.64 (s, 1H), 3.54 (d, J=10.6 Hz, 1H), 3.22 (t, J=12.3 Hz, 1H), 2.89 (q, J=7.5 Hz, 3H), 2.76 (dd, J=23.4, 10.1 Hz, 1H), 1.98 (d, J=10.4 Hz, 1H), 1.79 (t, J=10.3 Hz, 1H), 1.47 (s, 9H). NH not observed due to solvent exchange.

### Example 97. 1-(5-(((1R,4R)-5-methyl-2,5-diazabicyclo[2.2.1]heptan-2-yl)methyl)benzo[d]isoxazol-3-yl)dihydropyrimidine-2,4(1H,3H)-dione

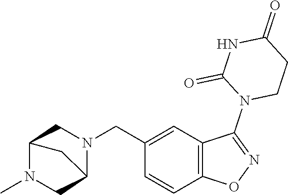 [SMILES: CN1C[C@H]2C[C@@H]1CN2Cc1ccc2onc(N3CCC(=O)NC3=O)c2c1]

Prepared by the method of Example 21, using potassium (((1R,4R)-5-(tert-butoxycarbonyl)-2,5-diazabicyclo[2.2.1]heptan-2-yl)methyl)trifluoroborate in step 1 and paraformaldehyde in step 4. LCMS [M+H]⁺: 356.2. ¹H NMR (400 MHz, MeOD) δ 8.27 (s, 1H), 7.86 (s, 1H), 7.67 (d, J=8.6 Hz, 1H), 7.56 (d, J=8.6 Hz, 1H), 4.17 (t, J=6.5 Hz, 2H), 4.11 (br s, 1H), 3.95 (m, 1H), 3.86 (m, 1H), 3.69 (m, 2H), 3.10 (d, J=11.0 Hz, 1H), 3.00 (3, 1H), 2.88 (m, 6H), 2.21 (m, 1H), 2.10 (m, 1H).

### Example 98. 1-(5-(((1R,4R)-5-((tetrahydro-2H-pyran-4-yl)methyl)-2,5-diazabicyclo[2.2.1]heptan-2-yl)methyl)benzo[d]isoxazol-3-yl)dihydropyrimidine-2,4(1H,3H)-dione

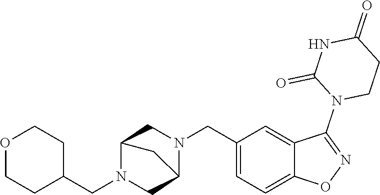 [SMILES: O=C1CCN(c2noc3ccc(CN4C[C@H]5C[C@@H]4CN5CC4CCOCC4)cc23)C(=O)N1]

Prepared by the method of Example 21, using potassium (((1R,4R)-5-(tert-butoxycarbonyl)-2,5-diazabicyclo[2.2.1]heptan-2-yl)methyl)trifluoroborate in step 1 and tetrahydro-2H-pyran-4-carbaldehyde in step 4. LCMS [M+H]⁺: 440.1. ¹H NMR (400 MHz, CD₃OD) δ 8.36 (s, 1H), 7.93 (s, 1H), 7.70 (dd, J=8.7, 1.7 Hz, 1H), 7.59 (d, J=8.7 Hz, 1H), 4.17 (t, J=6.6 Hz, 2H), 4.13-4.07 (m, 2H), 4.00-3.90 (m, 3H), 3.77 (s, 1H), 3.50-3.34 (m, 4H), 3.20 (td, J=7.9, 4.1 Hz, 2H), 3.05-2.85 (m, 4H), 2.23-2.06 (m, 2H), 2.02-1.91 (m, 1H), 1.70 (dtt, J=18.0, 14.4, 3.0 Hz, 2H), 1.34 (dtd, J=17.1, 11.7, 5.0 Hz, 2H).

### Example 99. tert-butyl (1R,5S)-3-((3-(2,4-dioxotetrahydropyrimidin-1(2H)-yl)benzo[d]isoxazol-5-yl)methyl)-3,8-diazabicyclo[3.2.1]octane-8-carboxylate

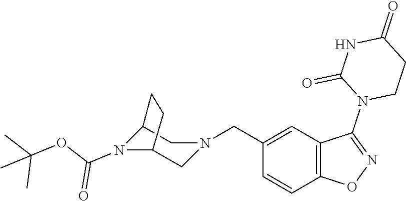 [SMILES: CC(C)(C)OC(=O)N1C2CCC1CN(Cc1ccc3onc(N4CCC(=O)NC4=O)c3c1)C2]

Prepared by the method of Example 21, steps 1-2 using potassium (((1R,5S)-8-(tert-butoxycarbonyl)-3,8-diazabicyclo[3.2.1]octan-3-yl)methyl)trifluoroborate in step 1. LCMS [M+H]⁺: 456.1. ¹H NMR (400 MHz, CD₃OD) δ 7.82 (s, 1H), 7.62 (dd, J=8.8, 1.7 Hz, 1H), 7.53 (d, J=8.7 Hz, 1H), 4.20-4.07 (m, 4H), 3.59 (s, 2H), 2.88 (t, J=6.7 Hz, 2H), 2.66 (dd, J=10.9, 2.6 Hz, 2H), 2.27 (s, 2H), 1.96 (d, J=7.0 Hz, 2H), 1.82 (d, J=7.9 Hz, 2H), 1.46 (s, 9H). NH not observed due to solvent exchange.

### Example 100. 1-(5-(((1R,5S)-3,8-diazabicyclo[3.2.1]octan-3-yl)methyl)benzo[d]isoxazol-3-yl)dihydropyrimidine-2,4(1H,3H)-dione

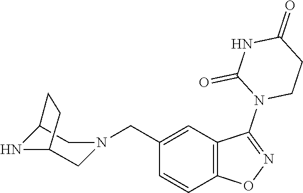 [SMILES: O=C1CCN(c2noc3ccc(CN4CC5CCC(C4)N5)cc23)C(=O)N1]

Prepared by the method of Example 21, steps 1-3 using potassium (((1R,5S)-8-(tert-butoxycarbonyl)-3,8-diazabicyclo[3.2.1]octan-3-yl)methyl)trifluoroborate in step 1. LCMS [M+H]⁺: 356.2. ¹H NMR (400 MHz, MeOD) δ 8.54 (s, 1H), 7.86 (d, J=1.6 Hz, 1H), 7.62 (dd, J=8.7, 1.7 Hz, 1H), 7.56 (d, J=8.7 Hz, 1H), 4.17 (t, J=6.6 Hz, 2H), 3.96 (dq, J=4.5, 2.2 Hz, 2H), 3.70 (s, 2H), 2.91-2.80 (m, 4H), 2.52 (d, J=12.3 Hz, 2H), 2.23-2.14 (m, 2H), 2.04-1.93 (m, 2H). NH not observed due to solvent exchange.

### Example 101. 1-(5-(((1R,5S)-8-methyl-3,8-diazabicyclo[3.2.1]octan-3-yl)methyl)benzo[d]isoxazol-3-yl)dihydropyrimidine-2,4(1H,3H)-dione

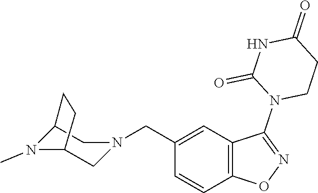 [SMILES: CN1C2CCC1CN(Cc1ccc3onc(N4CCC(=O)NC4=O)c3c1)C2]

Prepared by the method of Example 21, using potassium (((1R,5S)-8-(tert-butoxycarbonyl)-3,8-diazabicyclo[3.2.1]octan-3-yl)methyl)trifluoroborate in step 1 and paraformaldehyde in step 4. LCMS [M+H]⁺: 370.0. ¹H NMR (400 MHz, MeOD) δ 8.53 (s, 1H), 7.86 (dd, J=1.7, 0.8 Hz, 1H), 7.62 (dd, J=8.7, 1.7 Hz, 1H), 7.57 (dd, J=8.7, 0.7 Hz, 1H), 4.18 (t, J=6.6 Hz, 2H), 3.81 (s, 2H), 3.72 (s, 2H), 2.92-2.84 (m, 4H), 2.76 (s, 3H), 2.58 (d, J=12.4 Hz, 2H), 2.25-2.14 (m, 4H).

### Example 102. 1-(5-(((1R,5S)-3,8-diazabicyclo[3.2.1]octan-8-yl)methyl)benzo[d]isoxazol-3-yl)dihydropyrimidine-2,4(1H,3H)-dione

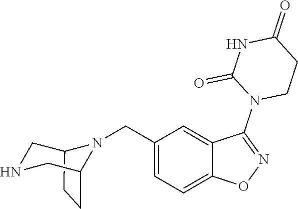 [SMILES: O=C1CCN(c2noc3ccc(CN4C5CCC4CNC5)cc23)C(=O)N1]

Prepared by the method of Example 21, steps 1-3 using potassium (((1R,5S)-3-(tert-butoxycarbonyl)-3,8-diazabicyclo[3.2.1]octan-8-yl)methyl)trifluoroborate in step 1. LCMS [M+H]⁺: 356.2. ¹H NMR (400 MHz, METHANOL-d4) b ppm 7.87 (s, 1H), 7.70 (m, 1H), 7.57 (d, J=8.7 Hz, 1H), 4.17 (t, J=6.6 Hz, 2H), 3.65 (m, 2H), 3.39 (br s, 2H), 3.21 (br d, J=13.6 Hz, 2H), 3.08 (br d, J=11.0 Hz, 2H), 2.88 (t, J=6.6 Hz, 2H), 2.31 (m, 2H), 1.89 (m, 2H). NH protons not observed due to solvent exchange.

### Example 103. 1-(5-(((1R,5S)-3-methyl-3,8-diazabicyclo[3.2.1]octan-8-yl)methyl)benzo[d]isoxazol-3-yl)dihydropyrimidine-2,4(1H,3H)-dione

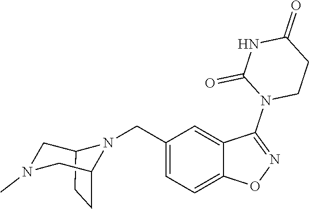 [SMILES: CN1CC2CCC(C1)N2Cc1ccc2onc(N3CCC(=O)NC3=O)c2c1]

Prepared by the method of Example 21, using potassium (((1R,5S)-3-(tert-butoxycarbonyl)-3,8-diazabicyclo[3.2.1]octan-8-yl)methyl)trifluoroborate in step 1 and paraformaldehyde in step 4. LCMS [M+H]⁺: 370.3. ¹H NMR (400 MHz, MeOD) δ 8.47 (s, 1H), 7.97 (d, J=1.6 Hz, 1H), 7.73 (ddd, J=7.9, 6.1, 1.7 Hz, 1H), 7.63 (d, J=8.7 Hz, 1H), 4.18 (td, J=6.6, 4.7 Hz, 2H), 3.95 (s, 1H), 3.57 (s, 1H), 3.16-3.08 (m, 1H), 2.92-2.83 (m, 3H), 2.66 (s, 3H), 2.36-2.26 (m, 1H), 2.04 (t, J=7.2 Hz, 1H), 1.61 (dt, J=44.4, 7.8 Hz, 2H), 1.35-1.25 (m, 2H), 0.93 (t, J=7.4 Hz, 2H).

### Example 104. 1-(5-(((1R,5S)-3-(methylsulfonyl)-3,8-diazabicyclo[3.2.1]octan-8-yl)methyl)benzo[d]isoxazol-3-yl)dihydropyrimidine-2,4(1H,3H)-dione

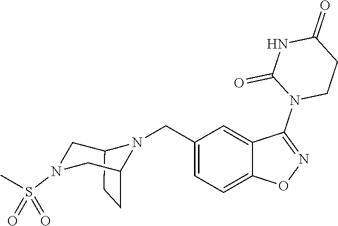 [SMILES: CS(=O)(=O)N1CC2CCC(C1)N2Cc1ccc2onc(N3CCC(=O)NC3=O)c2c1]

Prepared by the method of Example 64, step 4 wherein 1-(5-(((1R,5S)-3,8-diazabicyclo[3.2.1]octan-8-yl)methyl)benzo[d]isoxazol-3-yl)dihydropyrimidine-2,4(1H,3H)-dione hydrochloride was used in place of (R)-1-(5-((2-methylpiperazin-1-yl)methyl)benzo[d]isoxazol-3-yl)dihydropyrimidine-2,4(1H,3H)-dione hydrochloride and methanesulfonyl chloride was used in place of acetyl chloride. LCMS [M+H]⁺: 434.0. ¹H NMR (400 MHz, DMSO-d6) b ppm 1.61-1.69 (m, 2H), 1.94-2.03 (m, 2H), 2.80 (t, J=6.60 Hz, 2H), 2.85 (s, 3H), 2.91 (d, J=10.12 Hz, 2H), 3.15-3.19 (m, 2H) 3.23 (br s, 2H), 3.25-3.29 (m, 1H), 4.07 (t, J=6.60 Hz, 2H), 7.68 (s, 2H), 7.80 (s, 1H), 8.19 (s, 1H), 10.89 (s, 1H).

### Example 105. 1-(5-(((2S,5S)-2,5-dimethyl-4-(methylsulfonyl)piperazin-1-yl)methyl)benzo[d]isoxazol-3-yl)dihydropyrimidine-2,4(1H,3H)-dione PGP-154

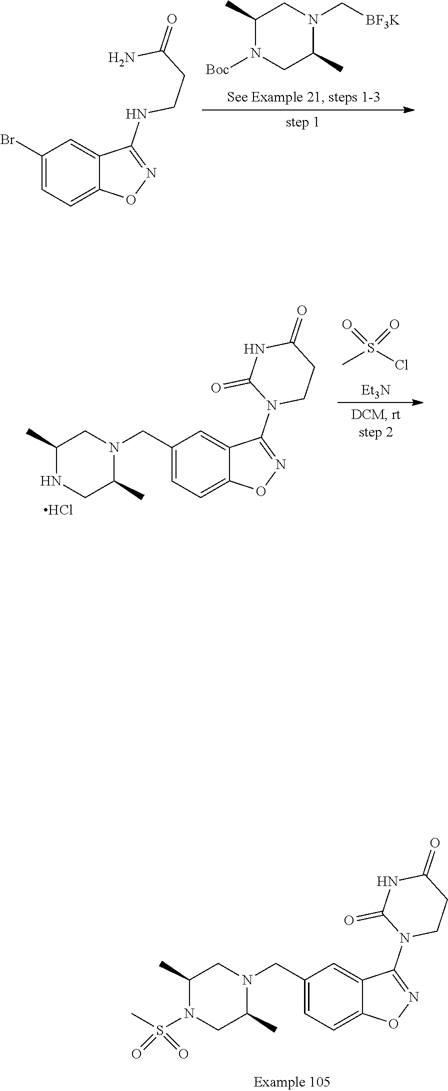

### Step 1. 1-(5-(((2S,5S)-2,5-dimethylpiperazin-1-yl)methyl)benzo[d]isoxazol-3-yl)dihydropyrimidine-2,4(1H,3H)-dione hydrochloride

Prepared by the method of Example 21, steps 1-3 wherein potassium (((2S,5S)-4-(tert-butoxycarbonyl)-2,5-dimethylpiperazin-1-yl)methyl)trifluoroborate was used in place of potassium ((4-(tert-butoxycarbonyl)piperazin-1-yl)methyl)trifluoroborate in step 1. LCMS [M+H]⁺: 357.8.

### Step 2. 1-(5-(((2S,5S)-2,5-dimethyl-4-(methylsulfonyl)piperazin-1-yl)methyl)benzo[d]isoxazol-3-yl)dihydropyrimidine-2,4(1H,3H)-dione

Triethylamine (61 mg, 0.56 mmol) and methanesulfonyl chloride (30 mg, 0.27 mmol) were added to a solution of 1-(5-(((2S,5S)-2,5-dimethylpiperazin-1-yl)methyl)benzo[d]isoxazol-3-yl)dihydropyrimidine-2,4(1H,3H)-dione hydrochloride (80 mg, 0.22 mmol) in DCM (2 mL) at rt. The mixture was stirred at rt for 2 h, then diluted with DCM and washed sequentially with water and brine. The organic layer was dried over sodium sulfate, filtered and concentrated. The residue was dissolved in DMSO, filtered through a 1 micron filter and purified by reverse-phase HPLC using ACN/Water/0.1% HCOOH. The fractions containing the product were combined and lyophilized to afford 1-(5-(((2S,5S)-2,5-dimethyl-4-(methylsulfonyl)piperazin-1-yl)methyl)benzo[d]isoxazol-3-yl)dihydropyrimidine-2,4(1H,3H)-dione (16 mg, 0.045 mmol, 19% yield). LCMS [M+H]⁺: 436.0. ¹H NMR (400 MHz, DMSO) δ 10.88 (s, 1H), 7.74 (s, 1H), 7.68 (d, J=8.6 Hz, 1H), 7.61 (dd, J=8.8, 1.7 Hz, 1H), 4.18 (d, J=13.7 Hz, 1H), 4.11-4.02 (m, 2H), 3.84 (d, J=7.5 Hz, 1H), 3.39 (dd, J=12.8, 3.4 Hz, 1H), 3.19-3.10 (m, 1H), 2.92 (s, 3H), 2.90-2.75 (m, 3H), 2.50 (m, 1H), 2.40-2.32 (m, 1H), 2.18 (dd, J=11.6, 3.7 Hz, 1H), 1.15 (dd, J=9.2, 6.3 Hz, 6H).

### Example 106. 1-(5-(((2S,5S)-2,4,5-trimethylpiperazin-1-yl)methyl)benzo[d]isoxazol-3-yl)dihydropyrimidine-2,4(1H,3H)-dione

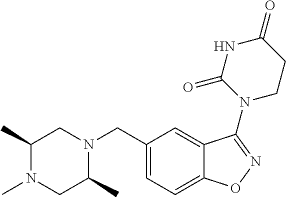 [SMILES: C[C@H]1CN(Cc2ccc3onc(N4CCC(=O)NC4=O)c3c2)[C@@H](C)CN1C]

Prepared by the method of Example 21, wherein potassium (((2S,5S)-4-(tert-butoxycarbonyl)-2,5-dimethylpiperazin-1-yl)methyl)trifluoroborate was used in place of potassium ((4-(tert-butoxycarbonyl)piperazin-1-yl)methyl)trifluoroborate in step 1 and paraformaldehyde was used in place of cyclohexanecarbaldehyde in step 4. LCMS [M+H]⁺: 372.2. ¹H NMR (400 MHz, MeOD) δ 8.54 (s, 1H), 7.83 (d, J=1.6 Hz, 1H), 7.65 (dd, J=8.7, 1.7 Hz, 1H), 7.56 (d, J=8.7 Hz, 1H), 4.17 (t, J=6.7 Hz, 2H), 4.01 (d, J=13.4 Hz, 1H), 3.54-3.47 (m, 1H), 3.09-2.92 (m, 4H), 2.88 (t, J=6.6 Hz, 2H), 2.70-2.60 (m, 4H), 2.54 (dd, J=12.9, 3.3 Hz, 1H), 1.24 (dd, J=6.5, 3.0 Hz, 6H).

### Example 107. 1-(5-(((2S,5S)-2,5-dimethyl-4-((tetrahydro-2H-pyran-4-yl)methyl)piperazin-1-yl)methyl)benzo[d]isoxazol-3-yl)dihydropyrimidine-2,4(1H,3H)-dione

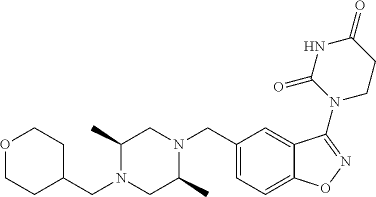 [SMILES: C[C@H]1CN(CC2CCOCC2)[C@@H](C)CN1Cc1ccc2onc(N3CCC(=O)NC3=O)c2c1]

Prepared by the method of Example 21, wherein potassium (((2S,5S)-4-(tert-butoxycarbonyl)-2,5-dimethylpiperazin-1-yl)methyl)trifluoroborate was used in place of potassium ((4-(tert-butoxycarbonyl)piperazin-1-yl)methyl)trifluoroborate in step 1 and tetrahydro-2H-pyran-4-carbaldehyde was used in place of cyclohexanecarbaldehyde in step 4. LCMS [M+H]⁺: 456.3. ¹H NMR (400 MHz, MeOD) δ 8.50 (s, 1H), 7.85 (d, J=1.5 Hz, 1H), 7.66 (dd, J=8.8, 1.7 Hz, 1H), 7.57 (dd, J=8.7, 0.7 Hz, 1H), 4.58 (s, 1H), 4.17 (t, J=6.7 Hz, 2H), 4.11 (d, J=13.2 Hz, 1H), 3.99-3.87 (m, 2H), 3.53-3.35 (m, 4H), 2.99-2.78 (m, 4H), 2.77-2.35 (m, 4H), 1.85 (s, 1H), 1.73 (d, J=13.3 Hz, 1H), 1.64 (d, J=13.4 Hz, 1H), 1.37-1.07 (m, 8H).

### Example 108. 1-(5-(((2R,6S)-2,4,6-trimethylpiperazin-1-yl)methyl)benzo[d]isoxazol-3-yl)dihydropyrimidine-2,4(1H,3H)-dione

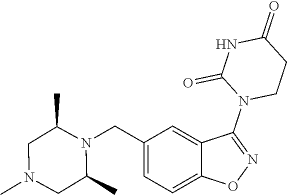 [SMILES: C[C@@H]1CN(C)C[C@H](C)N1Cc1ccc2onc(N3CCC(=O)NC3=O)c2c1]

Prepared by the method of Example 21, wherein potassium (((2S,6R)-4-(tert-butoxycarbonyl)-2,6-dimethylpiperazin-1-yl)methyl)trifluoroborate was used in place of potassium ((4-(tert-butoxycarbonyl)piperazin-1-yl)methyl)trifluoroborate in step 1 and paraformaldehyde was used in place of cyclohexanecarbaldehyde in step 4. LCMS [M+H]⁺: 371.9. ¹H NMR (400 MHz, MeOD) δ 7.94 (d, J=1.7 Hz, 1H), 7.67 (dd, J=8.8, 1.7 Hz, 1H), 7.55 (d, J=8.8 Hz, 1H), 4.18 (t, J=6.7 Hz, 2H), 4.05 (s, 2H), 3.61 (q, J=7.1 Hz, 1H), 3.38 (s, 1H), 3.00-2.80 (m, 9H), 1.17 (d, J=5.8 Hz, 6H). NH not observed due to solvent exchange.

### Example 109. 1-(5-(((2R,6S)-2,6-dimethyl-4-((tetrahydro-2H-pyran-4-yl)methyl)piperazin-1-yl)methyl)benzo[d]isoxazol-3-yl)dihydropyrimidine-2,4(1H,3H)-dione

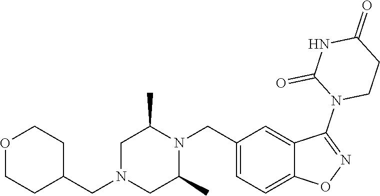 [SMILES: C[C@@H]1CN(CC2CCOCC2)C[C@H](C)N1Cc1ccc2onc(N3CCC(=O)NC3=O)c2c1]

Prepared by the method of Example 21, wherein potassium (((2S,6R)-4-(tert-butoxycarbonyl)-2,6-dimethylpiperazin-1-yl)methyl)trifluoroborate was used in place of potassium ((4-(tert-butoxycarbonyl)piperazin-1-yl)methyl)trifluoroborate in step 1 and tetrahydro-2H-pyran-4-carbaldehyde was used in place of cyclohexanecarbaldehyde in step 4. LCMS [M+H]⁺: 456.1. ¹H NMR (400 MHz, MeOD) δ 8.39 (s, 1H), 7.97 (s, 1H), 7.71-7.64 (m, 1H), 7.57 (d, J=8.8 Hz, 1H), 4.18 (t, J=6.6 Hz, 2H), 4.14 (s, 1H), 3.92 (dd, J=11.7, 4.3 Hz, 2H), 3.51-3.37 (m, 3H), 3.11 (d, J=17.5 Hz, 3H), 2.89 (t, J=6.7 Hz, 4H), 2.53 (d, J=9.8 Hz, 2H), 2.33 (s, 2H), 2.07-1.88 (m, 2H), 1.66 (d, J=13.3 Hz, 2H), 1.21 (d, J=6.2 Hz, 6H).

### Example 110. (R)-1-(5-((hexahydropyrazino[2,1-c][1,4]oxazin-8(1H)-yl)methyl)benzo[d]isoxazol-3-yl)dihydropyrimidine-2,4(1H,3H)-dione

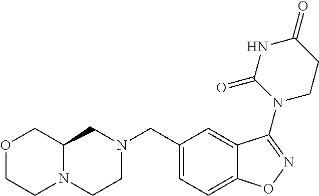 [SMILES: O=C1CCN(c2noc3ccc(CN4CCN5CCOC[C@H]5C4)cc23)C(=O)N1]

Prepared by the method of Example 21, steps 1-2 wherein potassium (R)-trifluoro((hexahydropyrazino[2,1-c][1,4]oxazin-8(1H)-yl)methyl)borate was used in place of potassium {[4-(tert-butoxycarbonyl)-1-piperazinyl]methyl}(trifluoro)borate in step 1. LCMS [M+H]⁺: 386.0. ¹H NMR (400 MHz, MeOD) δ 8.23 (s, 1H), 7.85 (t, J=1.2 Hz, 1H), 7.64 (dd, J=8.7, 1.7 Hz, 1H), 7.58 (d, J=8.7 Hz, 1H), 4.17 (t, J=6.7 Hz, 2H), 3.88-3.59 (m, 5H), 3.22 (t, J=10.8 Hz, 1H), 2.96 (d, J=8.1 Hz, 1H), 2.93-2.81 (m, 3H), 2.77 (ddd, J=12.2, 7.3, 5.0 Hz, 2H), 2.51-2.37 (m, 4H), 1.99 (t, J=11.0 Hz, 1H).

### Example 111. (S)-1-(5-((hexahydropyrazino[2,1-c][1,4]oxazin-8(1H)-yl)methyl)benzo[d]isoxazol-3-yl)dihydropyrimidine-2,4(1H,3H)-dione

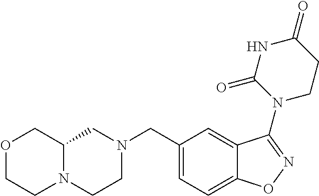 [SMILES: O=C1CCN(c2noc3ccc(CN4CCN5CCOC[C@@H]5C4)cc23)C(=O)N1]

Prepared by the method of Example 21, steps 1-2 wherein potassium (S)-trifluoro((hexahydropyrazino[2,1-c][1,4]oxazin-8(1H)-yl)methyl)borate was used in place of potassium {[4-(tert-butoxycarbonyl)-1-piperazinyl]methyl}(trifluoro)borate in step 1. LCMS [M+H]⁺: 386.1. ¹H NMR (400 MHz, MeOD) δ 7.82 (dd, J=1.6, 0.7 Hz, 1H), 7.63 (dd, J=8.7, 1.7 Hz, 1H), 7.56 (dd, J=8.7, 0.7 Hz, 1H), 4.17 (t, J=6.7 Hz, 2H), 3.86-3.78 (m, 1H), 3.69-3.58 (m, 4H), 3.20 (dd, J=11.2, 10.3 Hz, 1H), 2.92-2.85 (m, 3H), 2.81-2.75 (m, 1H), 2.69 (dq, J=11.1, 2.2 Hz, 2H), 2.42-2.31 (m, 4H), 1.86 (t, J=10.8 Hz, 1H). NH not observed due to solvent exchange.

### Example 112. (S)-1-(5-((1,1-dioxidohexahydro-5H-isothiazolo[2,3-a]pyrazin-5-yl)methyl)benzo[d]isoxazol-3-yl)dihydropyrimidine-2,4(1H,3H)-dione

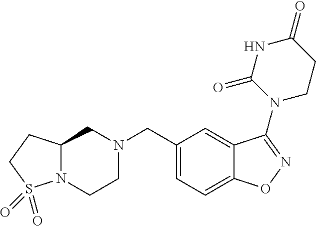 [SMILES: O=C1CCN(c2noc3ccc(CN4CCN5[C@@H](CCS5(=O)=O)C4)cc23)C(=O)N1]

Prepared by the method of Example 21, steps 1-2 wherein potassium (S)-((1,1-dioxidohexahydro-5H-isothiazolo[2,3-a]pyrazin-5-yl)methyl)trifluoroborate was used in place of potassium {[4-(tert-butoxycarbonyl)-1-piperazinyl]methyl}(trifluoro)borate in step 1. LCMS [M+H]⁺: 420.0. ¹H NMR (400 MHz, MeOD) δ 7.84 (dd, J=1.7, 0.8 Hz, 1H), 7.64 (dd, J=8.7, 1.7 Hz, 1H), 7.58-7.52 (m, 1H), 4.17 (t, J=6.7 Hz, 2H), 3.72 (q, J=13.1 Hz, 2H), 3.27-3.10 (m, 4H), 3.03 (d, J=11.1 Hz, 1H), 2.95 (d, J=11.6 Hz, 1H), 2.92-2.83 (m, 3H), 2.33 (dddd, J=12.6, 9.2, 5.8, 3.5 Hz, 1H), 2.23 (td, J=11.4, 3.4 Hz, 1H), 2.05-1.92 (m, 2H). NH not observed due to solvent exchange.

### Example 113. 1-(5-((4-isobutyl-3-oxopiperazin-1-yl)methyl)benzo[d]isoxazol-3-yl)dihydropyrimidine-2,4(1H,3H)-dione

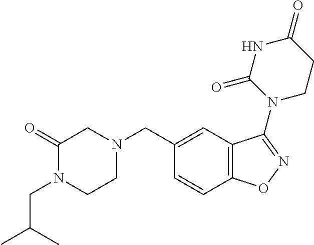 [SMILES: CC(C)CN1CCN(Cc2ccc3onc(N4CCC(=O)NC4=O)c3c2)CC1=O]

Prepared by the method of Example 21, steps 1-2 wherein potassium trifluoro((4-isobutyl-3-oxopiperazin-1-yl)methyl)borate was used in place of potassium {[4-(tert-butoxycarbonyl)-1-piperazinyl]methyl}(trifluoro)borate in step 1. LCMS [M+H]⁺: 400.3. ¹H NMR (400 MHz, MeOD) δ 7.85 (dd, J=1.7, 0.8 Hz, 1H), 7.65 (dd, J=8.7, 1.7 Hz, 1H), 7.57 (dd, J=8.7, 0.7 Hz, 1H), 4.17 (t, J=6.6 Hz, 2H), 3.71 (s, 2H), 3.39-3.33 (m, 2H), 3.21 (d, J=7.7 Hz, 2H), 3.15 (s, 2H), 2.88 (t, J=6.6 Hz, 2H), 2.77-2.68 (m, 2H), 2.06-1.95 (m, 1H), 0.90 (d, J=6.7 Hz, 6H). NH not observed due to solvent exchange.

### Example 114. (R)-1-(5-((4-isobutyl-3-(methoxymethyl)piperazin-1-yl)methyl)benzo[d]isoxazol-3-yl)dihydropyrimidine-2,4(1H,3H)-dione

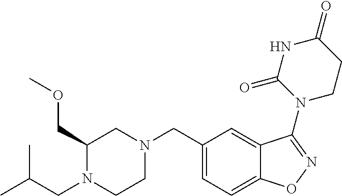 [SMILES: COC[C@H]1CN(Cc2ccc3onc(N4CCC(=O)NC4=O)c3c2)CCN1CC(C)C]

Prepared by the method of Example 21, steps 1-2 wherein potassium (R)-trifluoro((4-isobutyl-3-(methoxymethyl)piperazin-1-yl)methyl)borate was used in place of potassium {[4-(tert-butoxycarbonyl)-1-piperazinyl]methyl}(trifluoro)borate in step 1. LCMS [M+H]⁺: 430.3. ¹H NMR (400 MHz, MeOD) δ 8.47 (s, 1H), 7.86 (d, J=1.6 Hz, 1H), 7.65 (dd, J=8.7, 1.7 Hz, 1H), 7.59 (d, J=8.7 Hz, 1H), 4.18 (t, J=6.7 Hz, 2H), 3.81-3.71 (m, 2H), 3.60 (qd, J=10.7, 4.4 Hz, 2H), 3.36 (s, 3H), 3.16-3.09 (m, 1H), 2.99-2.76 (m, 7H), 2.64-2.48 (m, 3H), 2.03-1.90 (m, 1H), 1.04-0.95 (m, 6H).

### Example 115. (R)-1-(5-((3-(difluoromethyl)-4-((tetrahydro-2H-pyran-4-yl)methyl)piperazin-1-yl)methyl)benzo[d]isoxazol-3-yl)dihydropyrimidine-2,4(1H,3H)-dione

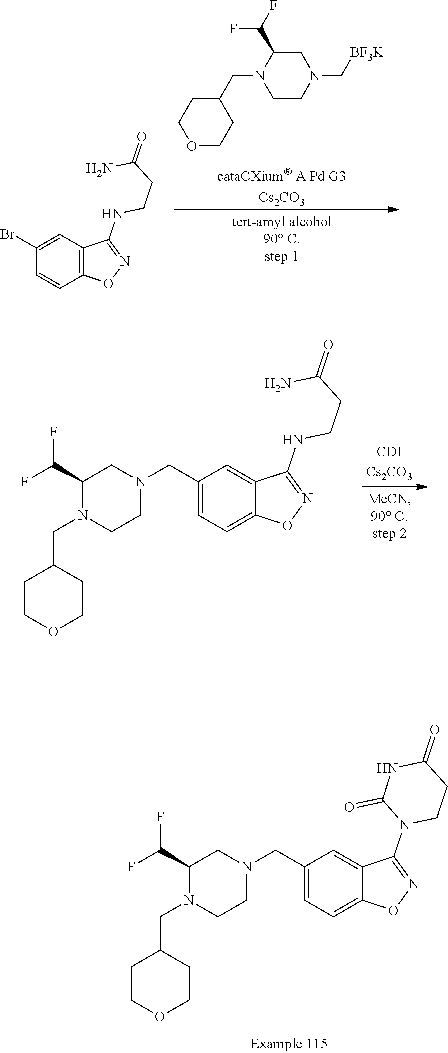

### Step 1. (R)-3-((5-((3-(difluoromethyl)-4-((tetrahydro-2H-pyran-4-yl)methyl)piperazin-1-yl)methyl)benzo[d]isoxazol-3-yl)amino)propanamide

A solution of 3-((5-bromobenzo[d]isoxazol-3-yl)amino)propanamide (200 mg, 0.70 mmol) and potassium (R)-((3-(difluoromethyl)-4-((tetrahydro-2H-pyran-4-yl)methyl)piperazin-1-yl)methyl)trifluoroborate (0.62 g, 1.75 mmol) in tert-amyl alcohol (6 mL) and aqueous cesium carbonate (1.5 M, 1.5 mL) was degassed by bubbling argon through the mixture for 10 min. cataCXium-A-Pd-G3 (20 mg, 0.028 mmol) was added the mixture degassed with argon for another minute. The mixture was stirred at 90° C. for 16 h, then cooled to rt and partitioned between EtOAc and water. The organic layer was separated, washed with brine, dried over sodium sulfate, filtered and concentrated. The residue was purified by silica gel chromatography (eluting with 7-8% MeOH in DCM) to give (R)-3-((5-((3-(difluoromethyl)-4-((tetrahydro-2H-pyran-4-yl)methyl)piperazin-1-yl)methyl)benzo[d]isoxazol-3-yl)amino)propanamide (100 mg, 0.22 mmol, 32% yield). LCMS [M+H]⁺: 452.2.

### Step 2. (R)-1-(5-((3-(difluoromethyl)-4-((tetrahydro-2H-pyran-4-yl)methyl)piperazin-1-yl)methyl)benzo[d]isoxazol-3-yl)dihydropyrimidine-2,4(1H,3H)-dione

Cesium carbonate (210 mg, 0.66 mmol) and CDI (107 mg, 0.66 mmol) were added to a solution of (R)-3-((5-((3-(difluoromethyl)-4-((tetrahydro-2H-pyran-4-yl)methyl)piperazin-1-yl)methyl)benzo[d]isoxazol-3-yl)amino)propanamide (100 mg, 0.22 mmol) in acetonitrile (12 mL) at rt. The reaction mixture was then heated at 90° C. for 24 h. The reaction was cooled to rt and diluted with EtOAc and washed with water. The organic layer was separated, dried over sodium sulfate, filtered and concentrated. The residue was dissolved in DMSO, filtered through a 1 micron filter and purified by reverse-phase HPLC using ACN/Water/0.1% formic acid. The fractions containing the product were combined and lyophilized to afford (R)-1-(5-((3-(difluoromethyl)-4-((tetrahydro-2H-pyran-4-yl)methyl)piperazin-1-yl)methyl)benzo[d]isoxazol-3-yl)dihydropyrimidine-2,4(1H,3H)-dione (14 mg, 0.029 mmol, 13% yield). LCMS [M+H]⁺: 478.2 ¹H NMR (400 MHz, MeOD) δ 8.48 (s, 1H), 7.80 (d, J=1.6 Hz, 1H), 7.62 (dd, J=8.7, 1.7 Hz, 1H), 7.54 (d, J=8.7 Hz, 1H), 6.24 (td, J=56.0, 5.6 Hz, 1H), 4.17 (t, J=6.6 Hz, 2H), 3.95-3.85 (m, 2H), 3.67-3.53 (m, 2H), 3.39 (t, J=12.0 Hz, 2H), 2.99-2.92 (m, 1H), 2.88 (t, J=6.6 Hz, 2H), 2.76 (dd, J=9.6, 5.1 Hz, 1H), 2.65-2.48 (m, 5H), 2.42 (dt, J=12.8, 7.2 Hz, 2H), 1.71 (dt, J=25.9, 10.4 Hz, 3H), 1.19 (dt, J=14.5, 11.1 Hz, 2H).

### Example 116. (R)-1-(5-((4-oxohexahydropyrazino[2,1-c][1,4]oxazin-8(1H)-yl)methyl)benzo[d]isoxazol-3-yl)dihydropyrimidine-2,4(1H,3H)-dione

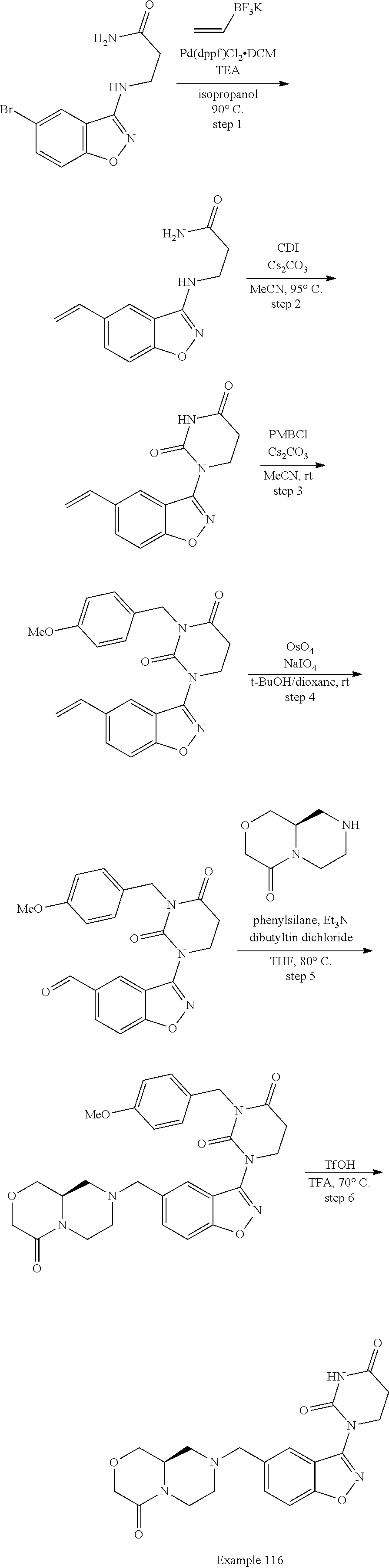

### Step 1. 3-((5-vinylbenzo[d]isoxazol-3-yl)amino)propanamide

A solution of 3-((5-bromobenzo[d]isoxazol-3-yl)amino)propanamide (4 g, 14 mmol), potassium vinyltrifluoroborate (3.7 g, 28 mmol) and TEA (5.8 mL, 42 mmol) in tert-butanol (60 mL) was degassed by bubbling argon through the mixture for 10 min. Pd(dppf)Cl₂·DCM (571 mg, 0.16 mmol) was added and the mixture was stirred at 90° C. for 14 h. The reaction was cooled to rt and concentrated. The crude material was partitioned between DCM and water. The organic layer was separated, washed with brine, dried over sodium sulfate, filtered and concentrated. The residue was purified by silica gel chromatography (eluting with 90% EtOAc in hexane) to give 3-((5-vinylbenzo[d]isoxazol-3-yl)amino)propanamide (1.1 g, 4.8 mmol, 34% yield). LCMS [M+H]⁺: 232.1.

### Step 2. 1-(5-vinvlbenzo[d]isoxazol-3-yl)dihydropyrimidine-2,4(1H,3H)-dione

Cesium carbonate (5.7 g, 17.2 mmol) and CDI (2.7 g, 17.2 mmol) were added to a solution of 3-((5-vinylbenzo[d]isoxazol-3-yl)amino)propanamide (1.0 g, 4.3 mmol) in acetonitrile (50 mL) at rt. The reaction mixture was then heated at 95° C. for 6 h. The reaction was cooled to rt and diluted with EtOAc and washed with water. The organic layer was separated, dried over sodium sulfate, filtered and concentrated. The residue was purified by silica gel chromatography (eluting with 90% EtOAc in hexane) to give 1-(5-vinylbenzo[d]isoxazol-3-yl)dihydropyrimidine-2,4(1H,3H)-dione (0.8 g, 3.1 mmol, 72% yield). LCMS [M+H]⁺: 257.8.

### Step 3. 3-(4-methoxybenzyl)-1-(5-vinvlbenzo[d]isoxazol-3-yl)dihydropyrimidine-2,4(1H,3H)-dione

Cesium carbonate (2.5 g, 7.8 mmol) and PMBCl (1.1 mL, 7.8 mmol) were added to a solution of 1-(5-vinylbenzo[d]isoxazol-3-yl)dihydropyrimidine-2,4(1H,3H)-dione (1.0 g, 3.9 mmol) in acetonitrile (20 mL) at rt. The reaction mixture was stirred at rt for 4 h. The reaction was diluted with EtOAc and washed with water. The organic layer was separated, dried over sodium sulfate, filtered and concentrated. The residue was purified by silica gel chromatography (eluting with 20-30% EtOAc in hexane) to give 3-(4-methoxybenzyl)-1-(5-vinylbenzo[d]isoxazol-3-yl)dihydropyrimidine-2,4(1H,3H)-dione (0.6 g, 1.6 mmol, 41% yield). LCMS [M+H]⁺: 377.9.
Step 4. 3-(3-(4-methoxybenzyl)-2,4-dioxotetrahydropyrimidin-1(2H)-yl)benzo[d]isoxazole-5-carbaldehyde Osmium tetroxide (2.5 wt % in t-BuOH, 0.79 mL, 0.079 mmol) was added to a solution of 3-(4-methoxybenzyl)-1-(5-vinylbenzo[d]isoxazol-3-yl)dihydropyrimidine-2,4(1H,3H)-dione (0.6 g, 1.6 mmol) in t-BuOH (4 mL) and dioxane (4 mL) at 0° C. The reaction mixture was stirred at rt for 2 h. The mixture was then cooled to 0° C. and a solution of sodium periodate (1.69 g, 7.95 mmol) in water (5 mL) was added. The mixture was stirred at rt for 2 h. The reaction was diluted with EtOAc and washed with water. The organic layer was separated, dried over sodium sulfate, filtered and concentrated to give crude 3-(3-(4-methoxybenzyl)-2,4-dioxotetrahydropyrimidin-1(2H)-yl)benzo[d]isoxazole-5-carbaldehyde (0.25 g, crude) which was used without further purification. LCMS [M+H]⁺: 380.2.

### Step 5. (R)-3-(4-methoxybenzyl)-1-(5-((4-oxohexahydropyrazino[2,1-c][1,4]oxazin-8(1H)-yl)methyl)benzo[d]isoxazol-3-yl)dihydropyrimidine-2,4(1H,3H)-dione

To a solution of 3-(3-(4-methoxybenzyl)-2,4-dioxotetrahydropyrimidin-1(2H)-yl)benzo[d]isoxazole-5-carbaldehyde (200 mg, 0.52 mmol) in THF (5 mL) was added (R)-hexahydropyrazino[2,1-c][1,4]oxazin-4(3H)-one (82 mg, 0.52 mmol), dibutyltin dichloride (158 mg, 1.02 mmol), and triethylamine (0.3 mL, 1.58 mmol). The mixture was stirred at 80° C. for 1 h and phenylsilane (111 mg, 0.52 mmol) was added. The reaction was stirred at 80° C. for 1 h. The reaction was cooled to rt, diluted with water and extracted with EtOAc. The organic layer was dried over Na₂SO₄, filtered and concentrated to give crude (R)-3-(4-methoxybenzyl)-1-(5-((4-oxohexahydropyrazino[2,1-c][1,4]oxazin-8(1H)-yl)methyl)benzo[d]isoxazol-3-yl)dihydropyrimidine-2,4(1H,3H)-dione (130 mg, crude) which was used without further purification. LCMS [M+H]⁺: 520.3.

### Step 6. (R)-1-(5-((4-oxohexahydropyrazino[2,1-c][1,4]oxazin-8(1H)-yl)methyl)benzo[d]isoxazol-3-yl)dihydropyrimidine-2,4(1H,3H)-dione

A solution of 10% TfOH in TFA (2 mL) was added to (R)-3-(4-methoxybenzyl)-1-(5-((4-oxohexahydropyrazino[2,1-c][1,4]oxazin-8(1H)-yl)methyl)benzo[d]isoxazol-3-yl)dihydropyrimidine-2,4(1H,3H)-dione (130 mg, 0.25 mmol) at rt. The reaction mixture was then stirred at 70° C. for 3 h. The reaction was then concentrated. The residue was dissolved in DMSO, filtered through a 1 micron filter and purified by reverse-phase HPLC using ACN/Water/0.1% formic acid. The fractions containing the product were combined and lyophilized to afford (R)-1-(5-((4-oxohexahydropyrazino[2,1-c][1,4]oxazin-8(1H)-yl)methyl)benzo[d]isoxazol-3-yl)dihydropyrimidine-2,4(1H,3H)-dione (10 mg, 0.025 mmol, 10% yield). LCMS [M+H]⁺: 400.1 ¹H NMR (400 MHz, MeOD) δ 8.28 (s, 1H), 7.83 (dd, J=1.7, 0.8 Hz, 1H), 7.65 (dd, J=8.7, 1.7 Hz, 1H), 7.56 (dd, J=8.7, 0.7 Hz, 1H), 4.47 (ddd, J=13.2, 3.3, 1.8 Hz, 1H), 4.17 (t, J=6.6 Hz, 2H), 4.09 (d, J=1.6 Hz, 2H), 3.97 (dd, J=11.9, 4.6 Hz, 1H), 3.75-3.62 (m, 4H), 3.60-3.48 (m, 2H), 2.98-2.78 (m, 3H), 2.11 (td, J=11.8, 3.3 Hz, 1H), 1.97 (t, J=11.1 Hz, 1H).

### Example 117. (R)-1-(5-((3-(methoxymethyl)-4-methylpiperazin-1-yl)methyl)benzo[d]isoxazol-3-yl)dihydropyrimidine-2,4(1H,3H)-dione

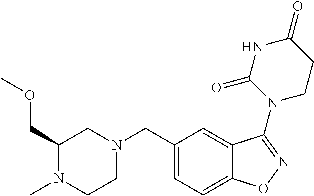 [SMILES: COC[C@H]1CN(Cc2ccc3onc(N4CCC(=O)NC4=O)c3c2)CCN1C]

Prepared by the method of Example 116, steps 5-6 wherein (R)-2-(methoxymethyl)-1-methylpiperazine [see US2014/323463, 2014, A1] was used in place of (R)-hexahydropyrazino[2,1-c][1,4]oxazin-4(3H)-one in step 5. LCMS [M+H]⁺: 388.0. ¹H NMR (400 MHz, MeOD) δ 8.15 (t, J=1.2 Hz, 1H), 7.81-7.73 (m, 2H), 4.63 (s, 2H), 4.20 (t, J=6.6 Hz, 2H), 3.97 (d, J=11.8 Hz, 1H), 3.82 (d, J=13.3 Hz, 3H), 3.69 (s, 1H), 3.61-3.50 (m, 4H), 3.48 (s, 3H), 3.06 (s, 3H), 2.90 (t, J=6.7 Hz, 2H). NH not observed due to solvent exchange.

### Example 118. (R)-1-(5-((3-(difluoromethyl)-4-methylpiperazin-1-yl)methyl)benzo[d]isoxazol-3-yl)dihydropyrimidine-2,4(1H,3H)-dione

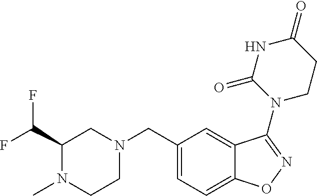 [SMILES: CN1CCN(Cc2ccc3onc(N4CCC(=O)NC4=O)c3c2)C[C@@H]1C(F)F]

Prepared by the method of Example 116, steps 5-6 wherein (R)-2-(difluoromethyl)-1-methylpiperazine was used in place of (R)-hexahydropyrazino[2,1-c][1,4]oxazin-4(3H)-one in step 5. LCMS [M+H]⁺: 394.1. ¹H NMR (400 MHz, MeOD) δ 8.29 (s, 1H), 7.82 (d, J=1.8 Hz, 1H), 7.63 (dd, J=8.7, 1.7 Hz, 1H), 7.55 (d, J=8.7 Hz, 1H), 6.05 (td, J=55.0, 3.4 Hz, 1H), 4.17 (t, J=6.6 Hz, 2H), 3.73-3.60 (m, 2H), 2.86 (dt, J=11.3, 7.4 Hz, 4H), 2.73 (d, J=11.4 Hz, 1H), 2.58 (d, J=10.9 Hz, 1H), 2.50-2.38 (m, 4H), 2.38-2.21 (m, 2H).

### Example 119. 1-(5-((4-cyclopropylpiperazin-1-yl)methyl)benzo[d]isoxazol-3-yl)dihydropyrimidine-2,4(1H,3H)-dione

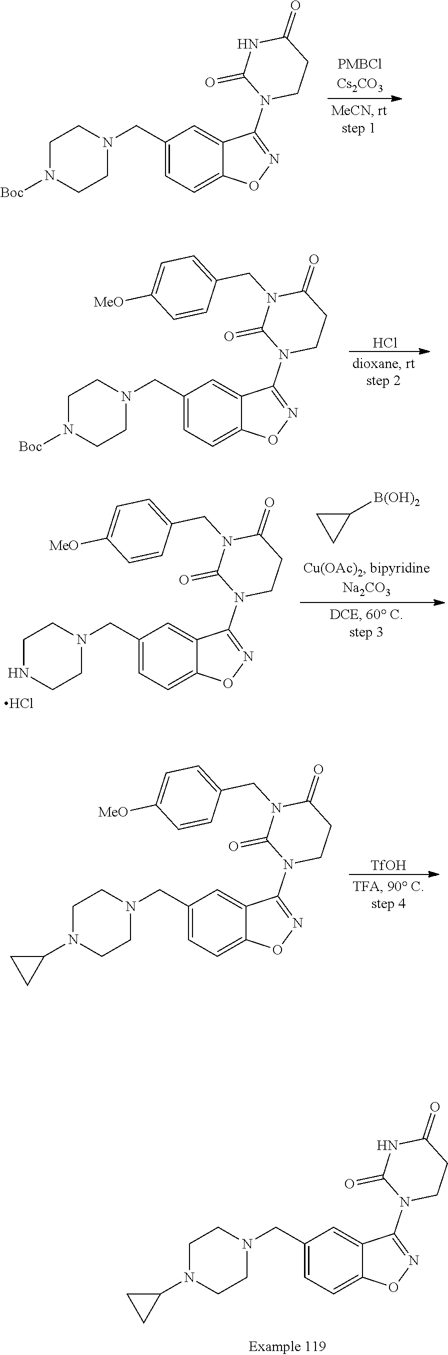

### Step 1. tert-butyl 4-((3-(3-(4-methoxybenzyl)-2,4-dioxotetrahydropyrimidin-1(2H)-yl)benzo[d]isoxazol-5-yl)methyl)piperazine-1-carboxylate

Cesium carbonate (0.303 g, 0.93 mmol) and PMBCl (83 mg, 0.53 mmol) were added to a solution of tert-butyl 4-((3-(2,4-dioxotetrahydropyrimidin-1(2H)-yl)benzo[d]isoxazol-5-yl)methyl)piperazine-1-carboxylate (0.3 g, 0.46 mmol) in acetonitrile (5 mL) at rt. The reaction mixture was stirred at rt for 2 h. The reaction was diluted with EtOAc and washed with water. The organic layer was separated, dried over sodium sulfate, filtered and concentrated. The residue was purified by silica gel chromatography (eluting with 90% EtOAc in hexane) to give tert-butyl 4-((3-(3-(4-methoxybenzyl)-2,4-dioxotetrahydropyrimidin-1(2H)-yl)benzo[d]isoxazol-5-yl)methyl)piperazine-1-carboxylate (0.12 g, 0.21 mmol, 47% yield). LCMS [M+H]⁺: 550.5.

### Step 2. 3-(4-methoxybenzyl)-1-(5-(piperazin-1-ylmethyl)benzo[d]isoxazol-3-yl)dihydropyrimidine-2,4(1H,3H)-dione hydrochloride

A solution of HCl (4.0 M in dioxane, 2 mL) was added to tert-butyl 4-((3-(3-(4-methoxybenzyl)-2,4-dioxotetrahydropyrimidin-1(2H)-yl)benzo[d]isoxazol-5-yl)methyl)piperazine-1-carboxylate (100 mg, 0.21 mmol) and the mixture was stirred at rt for 2 h. The reaction was then concentrated to give 3-(4-methoxybenzyl)-1-(5-(piperazin-1-ylmethyl)benzo[d]isoxazol-3-yl)dihydropyrimidine-2,4(1H,3H)-dione hydrochloride (110 mg, crude) which was used without further purification. LCMS [M+H]⁺: 450.2.

### Step 3. 1-(5-((4-cyclopropylpiperazin-1-yl)methyl)benzo[d]isoxazol-3-yl)-3-(4-methoxybenzyl)dihydropyrimidine-2,4(1H,3H)-dione

To a solution of 3-(4-methoxybenzyl)-1-(5-(piperazin-1-ylmethyl)benzo[d]isoxazol-3-yl)dihydropyrimidine-2,4(1H,3H)-dione hydrochloride (100 mg, 0.20 mmol) and cyclopropylboronic acid (35 mg, 0.41 mmol) in DCE (3 mL) was added Na₂CO₃ (43 mg, 0.41 mmol), bipyridine (32 mg, 0.20 mmol), and Cu(OAc)₂ (37 mg, 0.20 mmol). The mixture was stirred at 60° C. for 16 h. The reaction was cooled to rt, diluted with DCM and washed with water. The organic layer was dried over Na₂SO₄, filtered and concentrated to give crude 1-(5-((4-cyclopropylpiperazin-1-yl)methyl)benzo[d]isoxazol-3-yl)-3-(4-methoxybenzyl)dihydropyrimidine-2,4(1H,3H)-dione (150 mg, crude) which was used without further purification. LCMS [M+H]⁺: 490.3.

### Step 4. 1-(5-((4-cyclopropylpiperazin-1-yl)methyl)benzo[d]isoxazol-3-yl)dihydropyrimidine-2,4(1H,3H)-dione

A solution of 10% TfOH in TFA (5 mL) was added to 1-(5-((4-cyclopropylpiperazin-1-yl)methyl)benzo[d]isoxazol-3-yl)-3-(4-methoxybenzyl)dihydropyrimidine-2,4(1H,3H)-dione (150 mg, 0.30 mmol) at rt. The reaction mixture was then heated at 90° C. for 16 h. The reaction was then cooled to rt and concentrated. The residue was dissolved in DMSO, filtered through a 1 micron filter and purified by reverse-phase HPLC using ACN/Water/0.1% formic acid. The fractions containing the product were combined and lyophilized to afford 1-(5-((4-cyclopropylpiperazin-1-yl)methyl)benzo[d]isoxazol-3-yl)dihydropyrimidine-2,4(1H,3H)-dione (11 mg, 0.02 mmol, 9% yield). LCMS [M+H]⁺: 370.0 ¹H NMR (400 MHz, MeOD) δ 8.16 (d, J=1.4 Hz, 1H), 7.83-7.70 (m, 2H), 4.63 (s, 2H), 4.20 (t, J=6.6 Hz, 2H), 3.80-3.52 (m, 8H), 3.00 (s, 1H), 2.90 (t, J=6.7 Hz, 2H), 1.03 (d, J=4.3 Hz, 4H). NH not observed due to solvent exchange.

### Example 120. (R)-1-(5-((4-cyclopropyl-2-methylpiperazin-1-yl)methyl)benzo[d]isoxazol-3-yl)dihydropyrimidine-2,4(1H,3H)-dione

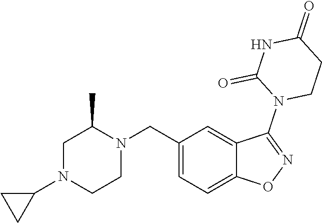 [SMILES: C[C@@H]1CN(C2CC2)CCN1Cc1ccc2onc(N3CCC(=O)NC3=O)c2c1]

Prepared by the method of Example 119, steps 5-6 wherein tert-butyl (R)-4-((3-(2,4-dioxotetrahydropyrimidin-1(2H)-yl)benzo[d]isoxazol-5-yl)methyl)-3-methylpiperazine-1-carboxylate was used in place of tert-butyl 4-((3-(2,4-dioxotetrahydropyrimidin-1(2H)-yl)benzo[d]isoxazol-5-yl)methyl)piperazine-1-carboxylate in step 1. LCMS [M+H]⁺: 384.0. ¹H NMR (400 MHz, MeOD) δ 8.06 (s, 1H), 7.72 (d, J=1.3 Hz, 2H), 4.74 (s, 1H), 4.20 (t, J=6.6 Hz, 2H), 4.14 (s, 1H), 3.72-3.53 (m, 1H), 3.22 (d, J=10.6 Hz, 4H), 2.89 (t, J=6.6 Hz, 4H), 2.21 (t, J=7.6 Hz, 1H), 1.53 (d, J=6.4 Hz, 3H), 0.76-0.58 (m, 4H). NH not observed due to solvent exchange.

### Examples 121 and 122. 1-(5-((5-((tetrahydro-2H-pyran-4-yl)methyl)-2,5-diazabicyclo[2.2.2]octan-2-yl)methyl)benzo[d]isoxazol-3-yl)dihydropyrimidine-2,4(1H,3H)-dione

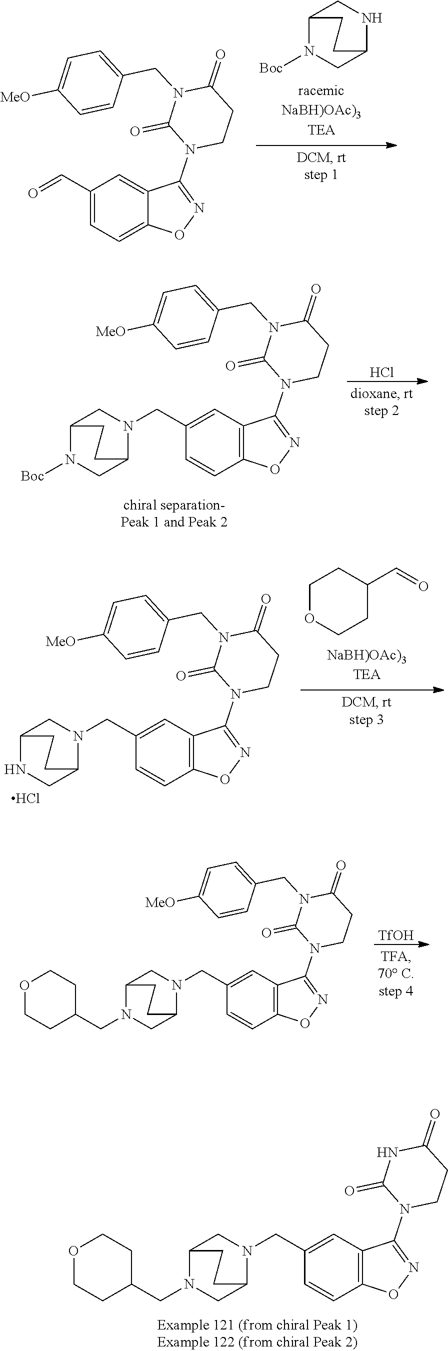

### Step 1. tert-butyl 5-((3-(3-(4-methoxybenzyl)-2,4-dioxotetrahydropyrimidin-1(2H)-yl)benzo[d]isoxazol-5-yl)methyl)-2,5-diazabicyclo[2.2.2]octane-2-carboxylate

Triethylamine (0.29 mL, 2.1 mmol) was added to a solution of 3-(3-(4-methoxybenzyl)-2,4-dioxotetrahydropyrimidin-1(2H)-yl)benzo[d]isoxazole-5-carbaldehyde (400 mg, 1.05 mmol) and tert-butyl 2,5-diazabicyclo[2.2.2]octane-2-carboxylate (223 mg, 1.05 mmol) in DCM (5 mL) at rt. The reaction mixture was stirred at rt for 1 h and then sodium triacetoxyborohydride (444 mg, 2.1 mmol) was added. The reaction was stirred at rt for 14 h and then quenched with a solution of saturated aqueous NaHCO₃. The mixture was extracted twice with DCM and the combined organic layers were washed with brine, dried over Na₂SO₄, filtered and concentrated. The residue was purified by silica gel chromatography (eluting with 20-30% EtOAc in hexane) to give racemic tert-butyl 5-((3-(3-(4-methoxybenzyl)-2,4-dioxotetrahydropyrimidin-1(2H)-yl)benzo[d]isoxazol-5-yl)methyl)-2,5-diazabicyclo[2.2.2]octane-2-carboxylate (0.25 g, 0.43 mmol, 41% yield). LCMS [M+H]⁺: 576.3.
The isomers were separated by chiral HPLC: COLUMN: CHIRALPAK IG, 250 MM×21 MM×5 MICRON, MOBILE PHASE: HEXANE (A) 0.1% HCOOH IN EtOH:MeOH, 1:1, (B), FLOW; 15 ML, ISOCRATIC:60(A):40(B). The collected fractions were concentrated under reduced pressure to afford:
Chiral Peak 1: tert-butyl 5-((3-(3-(4-methoxybenzyl)-2,4-dioxotetrahydropyrimidin-1(2H)-yl)benzo[d]isoxazol-5-yl)methyl)-2,5-diazabicyclo[2.2.2]octane-2-carboxylate (80 mg); Chiral HPLC: (8.64 min).
Chiral Peak 2: tert-butyl 5-((3-(3-(4-methoxybenzyl)-2,4-dioxotetrahydropyrimidin-1(2H)-yl)benzo[d]isoxazol-5-yl)methyl)-2,5-diazabicyclo[2.2.2]octane-2-carboxylate (80 mg); Chiral HPLC: (9.08 min).

### Step 2. 1-(5-((2,5-diazabicyclo[2.2.2]octan-2-yl)methyl)benzo[d]isoxazol-3-yl)-3-(4-methoxybenzyl)dihydropyrimidine-2,4(1H,3H)-dione hydrochloride

A solution of HCl (4.0 M in dioxane, 1 mL) was added to a solution of tert-butyl 5-((3-(3-(4-methoxybenzyl)-2,4-dioxotetrahydropyrimidin-1(2H)-yl)benzo[d]isoxazol-5-yl)methyl)-2,5-diazabicyclo[2.2.2]octane-2-carboxylate (80 mg, 0.017 mmol, Chiral Peak 1) in dioxane (2 mL) and the mixture was stirred at rt for 3 h. The reaction was then concentrated to give 1-(5-((2,5-diazabicyclo[2.2.2]octan-2-yl)methyl)benzo[d]isoxazol-3-yl)-3-(4-methoxybenzyl)dihydropyrimidine-2,4(1H,3H)-dione hydrochloride (60 mg, crude) which was used without further purification. LCMS [M+H]⁺: 476.1.

### Step 3. 3-(4-methoxybenzyl)-1-(5-((5-((tetrahydro-2H-pyran-4-yl)methyl)-2,5-diazabicyclo[2.2.2]octan-2-yl)methyl)benzo[d]isoxazol-3-yl)dihydropyrimidine-2,4(1H,3H)-dione

Triethylamine (0.028 mL, 0.20 mmol) was added to a solution of 1-(5-((2,5-diazabicyclo[2.2.2]octan-2-yl)methyl)benzo[d]isoxazol-3-yl)-3-(4-methoxybenzyl)dihydropyrimidine-2,4(1H,3H)-dione hydrochloride (50 mg, 0.1 mmol) and tetrahydro-2H-pyran-4-carbaldehyde (11 mg, 0.10 mmol) in DCM (5 mL) at rt. The reaction mixture was stirred at rt for 1 h and then sodium triacetoxyborohydride (42 mg, 0.20 mmol) was added. The reaction was stirred at rt for 4 h and then quenched with a solution of saturated aqueous NaHCO₃. The mixture was extracted twice with DCM and the combined organic layers were washed with brine, dried over Na₂SO₄, filtered and concentrated to give crude 3-(4-methoxybenzyl)-1-(5-((5-((tetrahydro-2H-pyran-4-yl)methyl)-2,5-diazabicyclo[2.2.2]octan-2-yl)methyl)benzo[d]isoxazol-3-yl)dihydropyrimidine-2,4(1H,3H)-dione (0.07 g, crude) which was used without further purification. LCMS [M+H]⁺: 574.3.

### Step 4. 1-(5-((5-((tetrahydro-2H-pyran-4-yl)methyl)-2,5-diazabicyclo[2.2.2]octan-2-yl)methyl)benzo[d]isoxazol-3-yl)dihydropyrimidine-2,4(1H,3H)-dione (Example 121)

A solution of 10% TfOH in TFA (5 mL) was added to 3-(4-methoxybenzyl)-1-(5-((5-((tetrahydro-2H-pyran-4-yl)methyl)-2,5-diazabicyclo[2.2.2]octan-2-yl)methyl)benzo[d]isoxazol-3-yl)dihydropyrimidine-2,4(1H,3H)-dione (70 mg) at rt. The reaction mixture was then heated at 70° C. for 2 h. The reaction was then cooled to rt and concentrated. The residue was dissolved in DMSO, filtered through a 1 micron filter and purified by reverse-phase HPLC using ACN/Water/0.1% formic acid. The fractions containing the product were combined and lyophilized to afford 1-(5-((5-((tetrahydro-2H-pyran-4-yl)methyl)-2,5-diazabicyclo[2.2.2]octan-2-yl)methyl)benzo[d]isoxazol-3-yl)dihydropyrimidine-2,4(1H,3H)-dione (20 mg, 0.044 mmol, 40% yield). LCMS [M+H]⁺: 454.0 ¹H NMR (400 MHz, MeOD) δ 8.28 (s, 1H), 7.94 (d, J=1.7 Hz, 1H), 7.70 (dd, J=8.7, 1.7 Hz, 1H), 7.60 (d, J=8.7 Hz, 1H), 4.18 (t, J=6.6 Hz, 2H), 4.05 (s, 2H), 3.95 (dd, J=10.7, 5.2 Hz, 2H), 3.56 (d, J=11.9 Hz, 1H), 3.51-3.34 (m, 4H), 3.21 (s, 1H), 3.15-2.98 (m, 3H), 2.95-2.83 (m, 3H), 2.17 (d, J=14.3 Hz, 2H), 2.07-1.88 (m, 2H), 1.85-1.66 (m, 3H), 1.43-1.27 (m, 2H).

### Example 122. 1-(5-((5-((tetrahydro-2H-pyran-4-yl)methyl)-2,5-diazabicyclo[2.2.2]octan-2-yl)methyl)benzo[d]isoxazol-3-yl)dihydropyrimidine-2,4(1H,3H)-dione

Prepared by the method of Example 121, from chiral Peak 2 in step 1. LCMS [M+H]⁺: 454.1. ¹H NMR (400 MHz, MeOD) δ 8.07 (s, 1H), 8.02 (s, 1H), 7.77-7.71 (m, 1H), 7.65 (d, J=8.7 Hz, 1H), 4.24 (s, 1H), 4.19 (t, J=6.6 Hz, 2H), 3.97 (d, J=11.5 Hz, 2H), 3.75-3.36 (m, 6H), 3.13 (dt, J=3.4, 1.7 Hz, 3H), 2.89 (t, J=6.6 Hz, 2H), 2.36-2.17 (m, 2H), 2.10-1.83 (m, 3H), 1.73 (d, J=13.1 Hz, 2H), 1.46-1.25 (m, 4H).

### Example 123. 1-(5-((5-methyl-2,5-diazabicyclo[2.2.2]octan-2-yl)methyl)benzo[d]isoxazol-3-yl)dihydropyrimidine-2,4(1H,3H)-dione

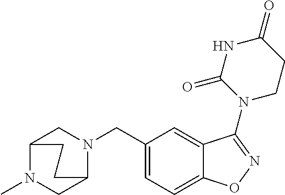 [SMILES: CN1CC2CCC1CN2Cc1ccc2onc(N3CCC(=O)NC3=O)c2c1]

Prepared by the method of Example 121, from chiral Peak 1 in step 1 wherein paraformaldehyde was used in place of tetrahydro-2H-pyran-4-carbaldehyde in step 3. LCMS [M+H]⁺: 370.2. ¹H NMR (400 MHz, MeOD) δ 8.53 (s, 1H), 7.88 (d, J=1.5 Hz, 1H), 7.67 (dd, J=8.8, 1.7 Hz, 1H), 7.56 (d, J=8.6 Hz, 1H), 4.17 (t, J=6.6 Hz, 2H), 3.89 (s, 2H), 3.56-3.48 (m, 1H), 3.37 (s, 1H), 3.28-3.18 (m, 2H), 2.98-2.84 (m, 6H), 2.79 (dd, J=11.8, 1.7 Hz, 1H), 2.26-2.08 (m, 2H), 1.97 (dd, J=13.6, 10.3 Hz, 1H), 1.78-1.65 (m, 1H).

### Example 124. 1-(5-((5-methyl-2,5-diazabicyclo[2.2.2]octan-2-yl)methyl)benzo[d]isoxazol-3-yl)dihydropyrimidine-2,4(1H,3H)-dione

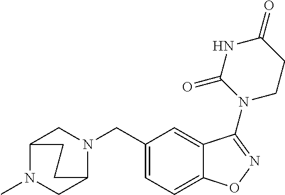 [SMILES: CN1CC2CCC1CN2Cc1ccc2onc(N3CCC(=O)NC3=O)c2c1]

Prepared by the method of Example 121, from chiral Peak 2 in step 1 wherein paraformaldehyde was used in place of tetrahydro-2H-pyran-4-carbaldehyde in step 3. LCMS [M+H]⁺: 370.1. ¹H NMR (400 MHz, MeOD) δ 8.23 (d, J=1.7 Hz, 1H), 7.86 (dd, J=8.8, 1.8 Hz, 1H), 7.78-7.72 (m, 1H), 4.80-4.67 (m, 2H), 4.20 (t, J=6.6 Hz, 2H), 4.00 (d, J=4.1 Hz, 1H), 3.91 (s, 1H), 3.83 (s, 2H), 3.72-3.59 (m, 2H), 3.12 (s, 3H), 2.91 (t, J=6.6 Hz, 2H), 2.41 (s, 2H), 2.05 (d, J=22.3 Hz, 2H). NH not observed due to solvent exchange.

### Example 125. 1-(5-((5-(methylsulfonyl)-2,5-diazabicyclo[2.2.2]octan-2-yl)methyl)benzo[d]isoxazol-3-yl)dihydropyrimidine-2,4(1H,3H)-dione

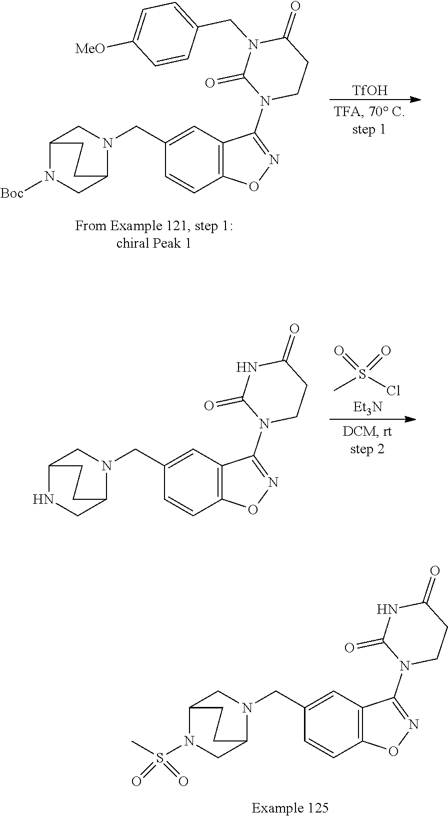

### Step 1. 1-(5-((2,5-diazabicyclo[2.2.2]octan-2-yl)methyl)benzo[d]isoxazol-3-yl)dihydropyrimidine-2,4(1H,3H)-dione

A solution of 10% TfOH in TFA (5 mL) was added to tert-butyl 5-((3-(3-(4-methoxybenzyl)-2,4-dioxotetrahydropyrimidin-1(2H)-yl)benzo[d]isoxazol-5-yl)methyl)-2,5-diazabicyclo[2.2.2]octane-2-carboxylate (70 mg, chiral Peak 1) at rt. The reaction mixture was then heated at 70° C. for 2 h. The reaction was then cooled to rt and concentrated. The crude was dissolved in 10% MeOH in DCM and basified with amberlyst-A21 (freebase) resin and filtered. The filtrate was concentrated to give crude 1-(5-((2,5-diazabicyclo[2.2.2]octan-2-yl)methyl)benzo[d]isoxazol-3-yl)dihydropyrimidine-2,4(1H,3H)-dione (70 mg, crude) which was used without further purification. LCMS [M+H]⁺: 356.1.

### Step 2. 1-(5-((5-(methylsulfonyl)-2,5-diazabicyclo[2.2.2]octan-2-yl)methyl)benzo[d]isoxazol-3-yl)dihydropyrimidine-2,4(1H,3H)-dione

Triethylamine (0.11 mL, 0.78 mmol) and methanesulfonyl chloride (0.024 mL, 0.31 mmol) were added to a solution of 1-(5-((2,5-diazabicyclo[2.2.2]octan-2-yl)methyl)benzo[d]isoxazol-3-yl)dihydropyrimidine-2,4(1H,3H)-dione (70 mg) in DCM (10 mL) at rt. The mixture was stirred at rt for 2 h, then diluted with DCM and washed sequentially with water and brine. The organic layer was dried over sodium sulfate, filtered and concentrated. The residue was dissolved in DMSO, filtered through a 1 micron filter and purified by reverse-phase HPLC using ACN/Water/0.1% HCOOH. The fractions containing the product were combined and lyophilized to afford 1-(5-((5-(methylsulfonyl)-2,5-diazabicyclo[2.2.2]octan-2-yl)methyl)benzo[d]isoxazol-3-yl)dihydropyrimidine-2,4(1H,3H)-dione (15 mg, 0.031 mmol, 16% yield). LCMS [M+H]⁺: 434.1. ¹H NMR (400 MHz, DMSO) δ 10.88 (s, 1H), 7.78 (s, 1H), 7.67 (d, J=8.6 Hz, 1H), 7.63 (dd, J=8.7, 1.6 Hz, 1H), 4.06 (t, J=6.6 Hz, 2H), 3.88-3.77 (m, 2H), 3.70-3.58 (m, 2H), 3.23 (dd, J=10.0, 2.1 Hz, 1H), 2.96 (s, 3H), 2.90 (ddt, J=12.2, 5.8, 2.9 Hz, 2H), 2.80 (t, J=6.6 Hz, 2H), 2.73 (dd, J=10.6, 2.1 Hz, 1H), 2.05-1.82 (m, 2H), 1.79-1.56 (m, 2H).

### Example 126. 1-(5-((5-(methylsulfonyl)-2,5-diazabicyclo[2.2.2]octan-2-yl)methyl)benzo[d]isoxazol-3-yl)dihydropyrimidine-2,4(1H,3H)-dione

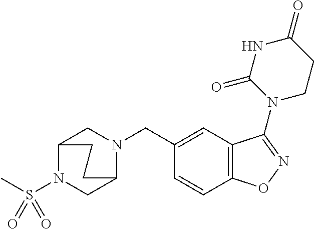 [SMILES: CS(=O)(=O)N1CC2CCC1CN2Cc1ccc2onc(N3CCC(=O)NC3=O)c2c1]

Prepared by the method of Example 125, from chiral Peak 2 (Example 121, step 1) in step 1. LCMS [M+H]⁺: 434.1. ¹H NMR (400 MHz, MeOD) δ 8.29 (s, 1H), 7.90 (s, 1H), 7.67 (dd, J=8.7, 1.7 Hz, 1H), 7.56 (d, J=8.7 Hz, 1H), 4.17 (t, J=6.6 Hz, 2H), 3.97 (d, J=3.1 Hz, 2H), 3.84-3.75 (m, 2H), 3.39-3.33 (m, 1H), 3.15 (dd, J=10.8, 2.7 Hz, 1H), 3.08-3.02 (m, 1H), 2.97 (s, 3H), 2.90 (dt, J=13.3, 4.3 Hz, 3H), 2.16 (s, 1H), 2.08-1.98 (m, 1H), 1.87 (dddd, J=13.5, 11.2, 4.6, 2.4 Hz, 1H), 1.79-1.70 (m, 1H).

### Example 127. 1-(5-(1-(4-methylpiperazin-1-yl)ethyl)benzo[d]isoxazol-3-yl)dihydropyrimidine-2,4(1H,3H)-dione

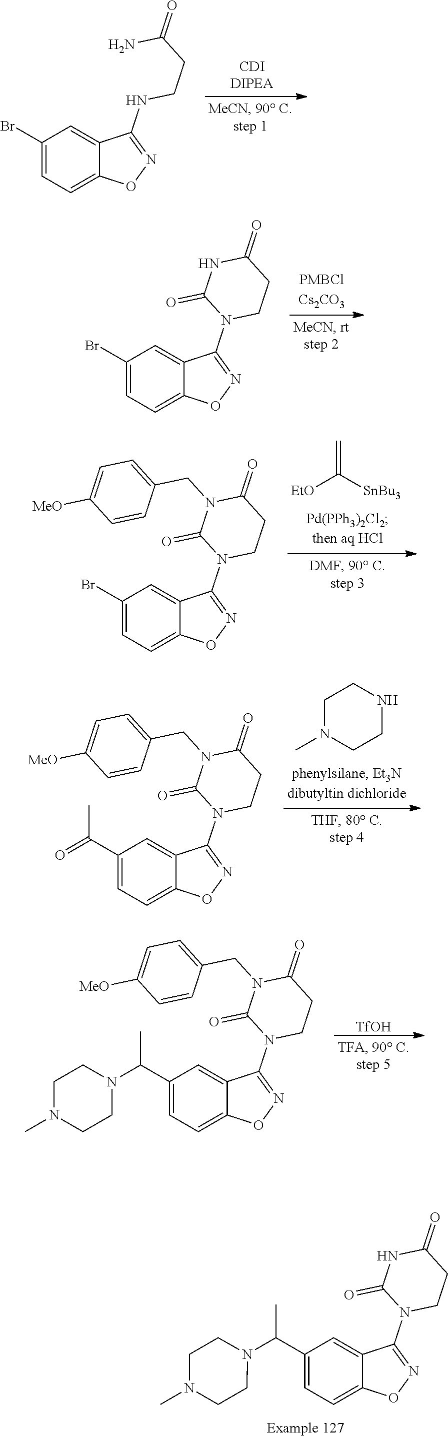

### Step 1. 1-(5-bromobenzo[d]isoxazol-3-yl)dihydropyrimidine-2,4(1H,3H)-dione

Cesium carbonate (4.59 g, 14.1 mmol) and CDI (2.28 g, 14.1 mmol) were added to a solution of 3-((5-bromobenzo[d]isoxazol-3-yl)amino)propanamide (1.0 g, 3.5 mmol) in acetonitrile (43 mL) at rt. The reaction mixture was then heated at 90° C. for 16 h. The reaction was cooled to rt and concentrated. The reaction mixture was filtered through celite which was washed with 10% MeOH in DCM. The filtrate was concentrated and the crude material was purified by silica gel chromatography (eluting with 10% MeOH in DCM) to give 1-(5-bromobenzo[d]isoxazol-3-yl)dihydropyrimidine-2,4(1H,3H)-dione (0.8 g, 2.6 mmol, 74% yield).

### Step 2. 1-(5-bromobenzo[d]isoxazol-3-yl)-3-(4-methoxybenzyl)dihydropyrimidine-2,4(1H,3H)-dione

Cesium carbonate (2.51 g, 7.74 mmol) and PMBCl (0.52 g, 3.35 mmol) were added to a solution of 1-(5-bromobenzo[d]isoxazol-3-yl)dihydropyrimidine-2,4(1H,3H)-dione (0.80 g, 2.6 mmol) in acetonitrile (18 mL) at rt. The reaction mixture was stirred at rt for 4 h. The reaction was diluted with EtOAc and washed with water. The organic layer was separated, dried over sodium sulfate, filtered and concentrated. The residue was purified by silica gel chromatography (eluting with 20-30% EtOAc in hexane) to give 1-(5-bromobenzo[d]isoxazol-3-yl)-3-(4-methoxybenzyl)dihydropyrimidine-2,4(1H,3H)-dione (1.2 g, 2.7 mmol, 100% yield). LCMS [M+H]⁺: 429.9.

### Step 3. 1-(5-acetylbenzo[d]isoxazol-3-yl)-3-(4-methoxybenzyl)dihydropyrimidine-2,4(1H,3H)-dione

Tributyl(1-ethoxyvinyl)stannane (604 mg, 1.67 mmol) and Pd(PPh₃)₂Cl₂ (98 mg, 0.14 mmol) were added to a solution of 1-(5-bromobenzo[d]isoxazol-3-yl)-3-(4-methoxybenzyl)dihydropyrimidine-2,4(1H,3H)-dione (600 mg, 1.39 mmol) in DMF (8 mL) at rt. The mixture was stirred at 90° C. for 3 h, then cooled to rt and acidified with aqueous 1 N HCl solution. The mixture was partitioned between EtOAc and water. The organic layer was separated, washed with brine, dried over sodium sulfate, filtered and concentrated. The crude product was purified by flash silica gel chromatography (eluted with 30% EtOAc/hexane) to give 1-(5-acetylbenzo[d]isoxazol-3-yl)-3-(4-methoxybenzyl)dihydropyrimidine-2,4(1H,3H)-dione (0.18 g, 0.46 mmol, 33% yield) as a yellow solid. LCMS [M+H]⁺: 393.9.

### Step 4. 3-(4-methoxybenzyl)-1-(5-(1-(4-methylpiperazin-1-yl)ethyl)benzo[d]isoxazol-3-yl)dihydropyrimidine-2,4(1H,3H)-dione

To a solution of 1-(5-acetylbenzo[d]isoxazol-3-yl)-3-(4-methoxybenzyl)dihydropyrimidine-2,4(1H,3H)-dione (180 mg, 0.48 mmol) in THF (5 mL) was added 1-methylpiperazine (138 mg, 1.37 mmol), dibutyltin dichloride (289 mg, 0.95 mmol), and triethylamine (0.2 mL). The mixture was stirred at 80° C. for 1 h and phenylsilane (0.2) was added. The reaction was stirred at 80° C. for 12 h. The reaction was cooled to rt, diluted with water and extracted with EtOAc. The organic layer was dried over Na₂SO₄, filtered and concentrated. The crude product was purified by flash silica gel chromatography (eluted with 5% MeOH/DCM) to give 3-(4-methoxybenzyl)-1-(5-(1-(4-methylpiperazin-1-yl)ethyl)benzo[d]isoxazol-3-yl)dihydropyrimidine-2,4(1H,3H)-dione (0.12 g, 0.25 mmol, 55% yield) as a yellow oil. LCMS [M+H]⁺: 478.1.

### Step 5. 1-(5-(1-(4-methylpiperazin-1-yl)ethyl)benzo[d]isoxazol-3-yl)dihydropyrimidine-2,4(1H,3H)-dione

A solution of 10% TfOH in TFA (2.2 mL) was added to 3-(4-methoxybenzyl)-1-(5-(1-(4-methylpiperazin-1-yl)ethyl)benzo[d]isoxazol-3-yl)dihydropyrimidine-2,4(1H,3H)-dione (120 mg, 0.25 mmol) at rt. The reaction mixture was then heated at 90° C. for 2 h. The reaction was then cooled to rt and concentrated. The residue was dissolved in DMSO, filtered through a 1 micron filter and purified by reverse-phase HPLC using ACN/Water/0.1% formic acid. The fractions containing the product were combined and lyophilized to afford 1-(5-(1-(4-methylpiperazin-1-yl)ethyl)benzo[d]isoxazol-3-yl)dihydropyrimidine-2,4(1H,3H)-dione (18 mg, 0.05 mmol, 20% yield). LCMS [M+H]⁺: 358.0 ¹H NMR (400 MHz, MeOD) δ 8.52 (s, 1H), 7.81 (d, J=1.7 Hz, 1H), 7.65 (dd, J=8.8, 1.7 Hz, 1H), 7.56 (d, J=8.7 Hz, 1H), 4.17 (t, J=6.7 Hz, 2H), 3.60 (q, J=6.7 Hz, 1H), 2.89 (t, J=6.6 Hz, 2H), 2.64 (d, J=77.3 Hz, 8H), 2.45 (s, 3H), 1.43 (d, J=6.7 Hz, 3H).

### Example 128

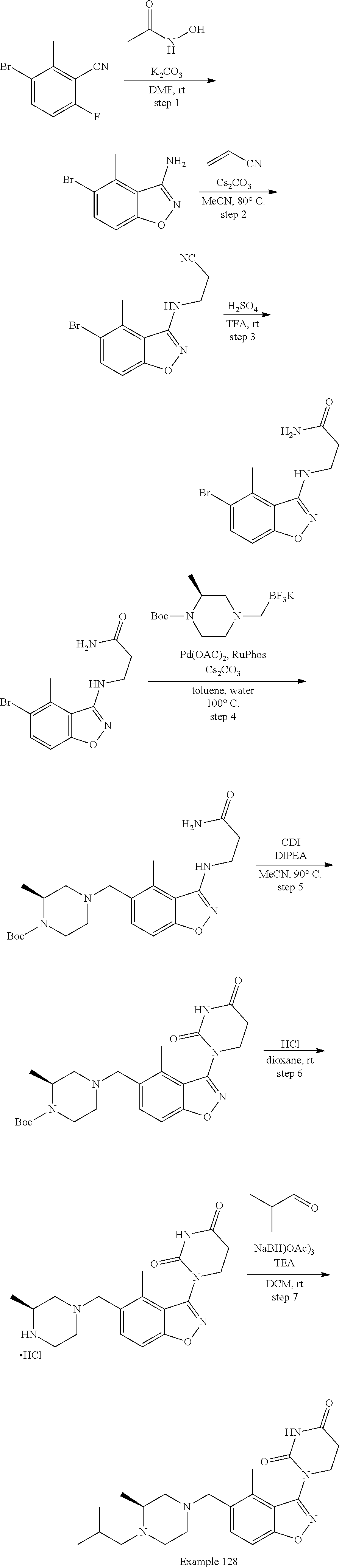

### Step 1: 5-bromo-4-methylbenzo[d]isoxazol-3-amine

Potassium carbonate (0.77 g, 5.6 mmol) and acetohydroxamic acid (0.42 g, 5.6 mmol) were added to a solution of 3-bromo-6-fluoro-2-methylbenzonitrile (0.40 g, 1.9 mmol) in DMF (4 mL) at rt. The mixture was stirred at rt overnight. The reaction mixture was partitioned between EtOAc and water and the layers were separated. The organic layer was washed with brine, dried over Na₂SO₄, filtered and concentrated to provide crude 5-bromo-4-methylbenzo[d]isoxazol-3-amine (0.40 g, 1.8 mmol, 94% yield) which was used without further purification. LCMS [M+H]⁺: 227.0.

### Step 2: 3-((5-bromo-4-methylbenzo[d]isoxazol-3-yl)amino)propanenitrile

Cesium carbonate (0.75 g, 2.3 mmol) was added to a solution of 5-bromo-4-methylbenzo[d]isoxazol-3-amine (0.40 g, 1.8 mmol) and acrylonitrile (0.12 mL, 1.8 mmol) in MeCN (8 mL) at rt. The mixture was heated at 80° C. for 3 h. The reaction mixture was cooled to rt and the orange suspension was filtered through celite, washing through with EtOAc. The filtrate was washed with brine, dried over Na₂SO₄, filtered and concentrated to provide crude 3-((5-bromo-4-methylbenzo[d]isoxazol-3-yl)amino)propanenitrile (0.48 g, 1.7 mmol, 97% yield) which was used without further purification. LCMS [M+H]⁺: 280.0.

### Step 3: 3-((5-bromo-4-methylbenzo[d]isoxazol-3-yl)amino)propanamide

Sulfuric acid (1 mL) was added dropwise to a solution of 3-((5-bromo-4-methylbenzo[d]isoxazol-3-yl)amino)propanenitrile (0.48 g, 1.7 mmol) in TFA (5 mL) at rt. The mixture was stirred overnight at rt. The reaction mixture was then poured into ice water and stirred for 10 min. The precipitate that formed was collected by filtration and washed with water followed by diethyl ether three times. The solid was dried under vacuum to provide 3-((5-bromo-4-methylbenzo[d]isoxazol-3-yl)amino)propanamide (0.42 g, 1.4 mmol, 82% yield) as an off-white solid. LCMS [M+H]⁺: 298.0.

### Step 4. tert-butyl (S)-4-((3-((3-amino-3-oxopropyl)amino)-4-methylbenzo[d]isoxazol-5-yl)methyl)-2-methylpiperazine-1-carboxylate

To a suspension of 3-((5-bromo-4-methylbenzo[d]isoxazol-3-yl)amino)propanamide (250 mg, 0.84 mmol) in toluene (3 mL) and water (0.3 mL) at room temperature was added Cs₂CO₃ (820 mg, 2.52 mmol), potassium (S)-((4-(tert-butoxycarbonyl)-3-methylpiperazin-1-yl)methyl)trifluoroborate (1.34 g, 4.19 mmol) and RuPhos (78 mg, 0.17 mmol), followed by Pd(OAc)₂ (19 mg, 0.084 mmol). The mixture was stirred at 100° C. for 4 h, then cooled to rt and partitioned between EtOAc and water. The organic layer was separated, washed with brine, dried over sodium sulfate, filtered and concentrated. The residue was purified by silica gel chromatography (eluting with 10-100% EtOAc in heptane) to give tert-butyl (S)-4-((3-((3-amino-3-oxopropyl)amino)-4-methylbenzo[d]isoxazol-5-yl)methyl)-2-methylpiperazine-1-carboxylate (220 mg, 0.51 mmol, 61% yield). LCMS [M+H]⁺: 432.5.

### Step 5. tert-butyl (S)-4-((3-(2,4-dioxotetrahydropyrimidin-1(2H)-yl)-4-methylbenzo[d]isoxazol-5-yl)methyl)-2-methylpiperazine-1-carboxylate

Cesium carbonate (82 mg, 0.25 mmol) and CDI (68 mg, 0.42 mmol) was added to a solution of tert-butyl (S)-4-((3-((3-amino-3-oxopropyl)amino)-4-methylbenzo[d]isoxazol-5-yl)methyl)-2-methylpiperazine-1-carboxylate (90 mg, 0.21 mmol) in acetonitrile (2 mL) at rt. The reaction mixture was then heated at 90° C. for 16 h. The reaction was cooled to rt and concentrated. The reaction mixture was partitioned between EtOAc and water and the layers were separated. The organic layer was washed with brine, dried over Na₂SO₄, filtered and concentrated to provide crude tert-butyl (S)-4-((3-(2,4-dioxotetrahydropyrimidin-1(2H)-yl)-4-methylbenzo[d]isoxazol-5-yl)methyl)-2-methylpiperazine-1-carboxylate (100 mg, crude) which was used without further purification. LCMS [M+H]⁺: 458.2.

### Step 6. (S)-1-(4-methyl-5-((3-methylpiperazin-1-yl)methyl)benzo[d]isoxazol-3-yl)dihydropyrimidine-2,4(1H,3H)-dione hydrochloride

A solution of HCl (4.0 M in dioxane, 2 mL, 8 mmol) was added to a solution of tert-butyl (S)-4-((3-(2,4-dioxotetrahydropyrimidin-1(2H)-yl)-4-methylbenzo[d]isoxazol-5-yl)methyl)-2-methylpiperazine-1-carboxylate (100 mg, 0.22 mmol) and the mixture was stirred at rt for 2 h. The reaction was then concentrated to give (S)-1-(4-methyl-5-((3-methylpiperazin-1-yl)methyl)benzo[d]isoxazol-3-yl)dihydropyrimidine-2,4(1H,3H)-dione hydrochloride (100 mg, crude) which was used without further purification. LCMS [M+H]⁺: 358.2.

### Step 7. (S)-1-(5-((4-isobutyl-3-methylpiperazin-1-yl)methyl)-4-methylbenzo[d]isoxazol-3-yl)dihydropyrimidine-2,4(1H,3H)-dione

Triethylamine (8 mg, 0.076 mmol) was added to a suspension of (S)-1-(4-methyl-5-((3-methylpiperazin-1-yl)methyl)benzo[d]isoxazol-3-yl)dihydropyrimidine-2,4(1H,3H)-dione hydrochloride (20 mg, 0.051 mmol) and isobutyraldehyde (11 mg, 0.15 mmol) in DCM (2 mL) at rt. The reaction mixture was stirred at rt for 10 min and then sodium triacetoxyborohydride (43 mg, 0.20 mmol) was added. The reaction was stirred at rt for 2 h and then diluted with DCM and water. The organic layer was dried over Na₂SO₄, filtered and concentrated. The residue was dissolved in DMSO, filtered through a 1 micron filter and purified by reverse-phase HPLC using ACN/Water/0.1% formic acid. The fractions containing the product were combined and lyophilized to afford a formate salt of (S)-1-(5-((4-isobutyl-3-methylpiperazin-1-yl)methyl)-4-methylbenzo[d]isoxazol-3-yl)dihydropyrimidine-2,4(1H,3H)-dione (14 mg, 0.027 mmol, 53% yield). LCMS [M+H]⁺: 414.2. ¹H NMR (500 MHz, DMSO) δ 10.91 (s, 1H), 7.61 (d, J=7.1 Hz, 2H), 4.08 (t, J=11.8 Hz, 2H), 3.84 (dt, J=11.6, 5.3 Hz, 1H), 3.80-3.45 (m, 3H), 3.36-3.06 (m, 3H), 2.94 (ddd, J=17.3, 11.3, 6.4 Hz, 4H), 2.74 (dt, J=17.0, 4.6 Hz, 1H), 2.36 (s, 3H), 2.00 (s, 1H), 1.25 (s, 3H), 0.95 (dd, J=10.7, 6.5 Hz, 7H).

### Example 129. (S)-1-(5-((3,4-dimethylpiperazin-1-yl)methyl)-4-methylbenzo[d]isoxazol-3-yl)dihydropyrimidine-2,4(1H,3H)-dione

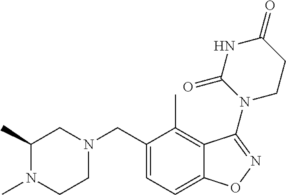 [SMILES: Cc1c(CN2CCN(C)[C@@H](C)C2)ccc2onc(N3CCC(=O)NC3=O)c12]

Prepared by the method of Example 128, wherein paraformaldehyde was used in place of isobutyraldehyde in step 7. LCMS [M+H]⁺: 372.4. ¹H NMR (500 MHz, DMSO) δ 10.92 (s, 1H), 7.61 (q, J=8.6 Hz, 2H), 4.97 (s, 2H), 4.10 (td, J=11.7, 4.6 Hz, 1H), 3.93-3.84 (m, 1H), 3.73 (s, 2H), 3.45 (s, 1H), 3.30-2.89 (m, 4H), 2.87-2.62 (m, 4H), 2.49 (s, 3H), 2.27 (s, 1H), 1.23 (d, J=6.4 Hz, 3H).

### Example 130. (S)-1-(5-((4-(cyclopropylmethyl)-3-methylpiperazin-1-yl)methyl)-4-methylbenzo[d]isoxazol-3-yl)dihydropyrimidine-2,4(1H,3H)-dione

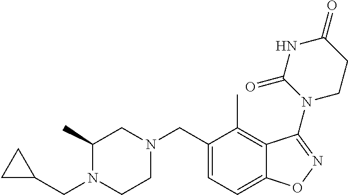 [SMILES: Cc1c(CN2CCN(CC3CC3)[C@@H](C)C2)ccc2onc(N3CCC(=O)NC3=O)c12]

Prepared by the method of Example 128, wherein cyclopropanecarbaldehyde was used in place of isobutyraldehyde in step 7. LCMS [M+H]⁺: 412.2. ¹H NMR (500 MHz, DMSO) δ 10.93 (s, 1H), 7.62 (q, J=8.7 Hz, 2H), 4.09 (dt, J=9.8, 6.7 Hz, 2H), 3.86 (dt, J=11.6, 5.3 Hz, 1H), 3.72 (d, J=30.6 Hz, 3H), 3.31 (s, 1H), 3.17 (d, J=18.7 Hz, 2H), 3.06-2.83 (m, 4H), 2.75 (dt, J=17.0, 4.5 Hz, 1H), 2.49 (s, 3H), 2.36 (d, J=20.0 Hz, 1H), 1.24 (d, J=6.4 Hz, 3H), 1.04 (s, 1H), 0.64 (s, 2H), 0.38 (d, J=27.5 Hz, 2H).

### Biological Data

### Abbreviations

BSA bovine serum albumin
Cas9 CRISPR associated protein 9
CRISPR Clustered regularly interspaced short palindromic repeats
crRNA CRISPR RNA
DMEM Dulbecco's modified eagle media
DMSO Dimethyl sulfoxide
DTT Dithiothreitol
EDTA ethylenediaminetetraacetic acid
eGFP enhanced green fluorescent protein
FACS fluorescence-activated cell sorting
FBS fetal bovine serum
FITC fluorescein
FIt3L Fms-related tyrosine kinase 3 ligand, FIt3L
HbF Fetal hemoglobin
HEPES (4-(2-hydroxyethyl)-1-piperazineethanesulfonic acid)
IMDM Iscove's modified Dulbecco's medium
KCl potassium chloride
mPB mobilized peripheral blood
PBS phosphate buffered saline
rhEPO recombinant human erythropoietin
rhIL-3 recombinant human interleukin-3
rhIL-6 recombinant human interleukin-6
rhSCF recombinant human stem cell factor
rhTPO recombinant human thrombopoietin
RNP ribonucleoprotein
shRNA short hairpin RNA
tracrRNA trans-activating crRNA
WIZ Widely-Interspaced Zinc Finger Containing Protein

### Materials and Methods

### Example 131: Quantification of WIZ Protein Levels in HiBit Tag Fusion Protein Assay

The HiBit system from Promega was used to develop high-throughput and quantitative assays to measure changes in WIZ protein levels in response to compounds. The HiBit tag was derived from a split Nanoluciferase and has the following protein sequence: VSGWRLFKKIS (SEQ ID NO: 1). The complementary fragment of Nanoluciferase (known as LgBit, from Promega), was added to the HiBit tag to form an active Nanoluciferase enzyme whose activity can be precisely measured. In this way, the levels of a fusion protein with the HiBit tag can be quantified in cell lysates.

Lentiviral vectors, based on the Invitrogen™ pLenti6.2/V5 DEST backbone were constructed that places the HiBit tag upstream of WIZ and expressed the fusion protein from an HSVTK promotor.

To ensure moderate and consistent expression of the HiBit-WIZ fusion protein across all cells in the population, stable cell lines were constructed from cells harboring a single copy of the construct. Lentivirus packaged with the constructs were made using the ViraPower™kit from Invitrogen™. 293T cells from ATCC (Catalog number: CRL-3216), were infected with the virus at low multiplicity of infection and selected by 5 μg/mL blasticidin in culture media for 2 weeks.

The levels of HiBit-WIZ tagged fusion proteins in compound-treated cell lines were measured as follows:

On day 1, cells were diluted to 1.0×10⁶ cells/ml in normal growth medium. 20 μL of cell suspension were plated in each well of a solid white 384-well plate. Plates were incubated overnight in a 37° C. and 5% CO₂ humidified tissue culture incubator.

On day 2, serial dilutions of compounds were made in 384-well plates. Compound plates were set up with DMSO in columns 1, 2, 23, 24, and 10-point compound dilution series in column 3-12 and column 13-22. 10 mM stock solution of compound were placed into column 3 or 13 and a 1:5 serial dilution was carried out until there was a 10-point dilution series per compound. 50 nL of diluted compounds were transferred into the plated cells by Echo® (Labcyte) acoustic transfer. The highest concentration of compound was 25 μM. Plates were incubated overnight (about 18 hours) in a 37° C. and 5% CO₂ humidified tissue culture incubator.

On day 3, plates were removed from the incubator and allowed to equilibrate at room temperature for 60 minutes. HiBit substrate (Nano-Glo® HiBit Lytic Detection System, Promega Catalogue number: N3050) was added as described by the manufacturers protocols. Plates were incubated at room temperature for 30 minutes and luminescence was read using an EnVision® reader (PerkinElmer®). Data was analyzed and visualized using the Spotfire® software package.

### Wiz Degradation Activity of Compounds (Table 1)

Table 1 shows WIZ degradation activity of compounds of the disclosure in the WIZ HiBit assay in 293T cells. WIZ Amax reflects the DMSO-normalized, curve-fitted percentage of WIZ-HiBit remaining at 25 uM. It was calculated by normalizing DMSO controls to 100%, parametric curve fitting of the dose response data (10-point, 5-fold), followed by calculation of response at 25 uM using the fitted equation (nd=not determined).

TABLE 1

| Cmpd | WIZ AC₅₀ | WIZ | % degradation of |
|---|---|---|---|
| No. | (μM) | Amax | WIZ (100-Amax) |
| 1a | 0.16 | 15 | 85 |
| 1b | 0.21 | 12 | 88 |
| 1 | 0.0072 | 9 | 91 |
| 2 | 0.0012 | 8 | 92 |
| 3 | 0.010 | 8 | 92 |
| 4 | 0.033 | 8 | 92 |
| 5 | 0.014 | 9 | 91 |
| 6 | 0.030 | 9 | 91 |
| 7 | 0.018 | 9 | 91 |
| 8 | 0.016 | 9 | 91 |
| 9 | 0.056 | 9 | 91 |
| 10 | 0.045 | 10 | 90 |
| 11 | 0.011 | 10 | 90 |
| 12 | 0.044 | 10 | 90 |
| 13 | 0.039 | 11 | 89 |
| 14 | 0.014 | 10 | 90 |
| 21 | 0.019 | 10 | 90 |
| 22 | 0.010 | 9 | 91 |
| 23 | 0.004 | 9 | 91 |
| 24 | 0.037 | 11 | 89 |
| 25 | 0.13 | 13 | 87 |
| 26 | 0.19 | 14 | 86 |
| 27 | 0.21 | 14 | 86 |
| 28 | 0.0054 | 10 | 90 |
| 29 | 0.0064 | 11 | 89 |
| 30 | 0.0045 | 9 | 91 |
| 31 | 0.0069 | 9 | 91 |
| 32 | 0.099 | 9 | 91 |
| 33 | 0.032 | 12 | 88 |
| 34 | 0.040 | 11 | 89 |
| 37 | 0.26 | 20 | 80 |
| 38 | 0.14 | 24 | 76 |
| 42 | 1.009 | 18 | 82 |
| 43 | 0.940 | 22 | 78 |
| 44 | 0.475 | 23 | 77 |
| 45 | 0.240 | 23 | 77 |
| 46 | 0.197 | 23 | 77 |
| 47 | 0.397 | 15 | 85 |
| 48 | 1.157 | 23 | 77 |
| 49 | 0.279 | 13 | 87 |
| 50 | 0.448 | 22 | 78 |
| 51 | 0.392 | 13 | 87 |
| 52 | 0.057 | 14 | 86 |
| 53 | 0.004 | 13 | 87 |
| 54 | 0.196 | 8 | 92 |
| 55 | 3.199 | 30 | 70 |
| 56 | 0.251 | 55 | 45 |
| 57 | 0.649 | 20 | 80 |
| 58 | 0.020 | 11 | 89 |
| 59 | 0.025 | 16 | 84 |
| 60 | 0.009 | 10 | 90 |
| 61 | 0.026 | 15 | 85 |
| 62 | 0.024 | 11 | 89 |
| 63 | 0.028 | 16 | 84 |
| 64 | 0.229 | 25 | 75 |
| 65 | 0.089 | 16 | 84 |
| 66 | 0.045 | 14 | 86 |
| 67 | 0.009 | 11 | 89 |
| 68 | 0.040 | 17 | 83 |
| 69 | 0.045 | 17 | 83 |
| 70 | 0.300 | 22 | 78 |
| 71 | 0.034 | 18 | 82 |
| 72 | 0.098 | 19 | 81 |
| 73 | 0.110 | 17 | 83 |
| 74 | 0.437 | 24 | 76 |
| 75 | 0.132 | 22 | 78 |
| 76 | 0.115 | 22 | 78 |
| 77 | 0.022 | 18 | 82 |
| 78 | 0.037 | 20 | 80 |
| 79 | 0.016 | 8 | 92 |
| 80 | 0.014 | 7 | 93 |
| 81 | 0.026 | 10 | 90 |
| 82 | 0.077 | 5 | 95 |
| 83 | 0.093 | 17 | 83 |
| 84 | 0.145 | 18 | 82 |
| 85 | 0.218 | 18 | 82 |
| 86 | 0.011 | 14 | 86 |
| 87 | 0.006 | 17 | 83 |
| 88 | 0.008 | 15 | 85 |
| 89 | 0.028 | 17 | 83 |
| 90 | 0.007 | 15 | 85 |
| 91 | 0.298 | 19 | 81 |
| 92 | 0.004 | 18 | 82 |
| 93 | 2.178 | 39 | 61 |
| 94 | 0.364 | 24 | 76 |
| 95 | 0.188 | 18 | 82 |
| 96 | 1.090 | 34 | 66 |
| 97 | 0.192 | 16 | 84 |
| 98 | 0.050 | 19 | 81 |
| 99 | 0.450 | 26 | 74 |
| 100 | 0.034 | 24 | 76 |
| 101 | 0.013 | 22 | 78 |
| 102 | 0.462 | 20 | 80 |
| 103 | 0.068 | 14 | 86 |
| 104 | 0.181 | 15 | 85 |
| 105 | 0.373 | 25 | 75 |
| 106 | 0.109 | 14 | 86 |
| 107 | 0.056 | 14 | 86 |
| 108 | 0.577 | 31 | 69 |
| 109 | 0.355 | 27 | 73 |
| 110 | 0.065 | 15 | 85 |
| 111 | 0.061 | 15 | 85 |
| 112 | 0.045 | 13 | 87 |
| 113 | 0.256 | 34 | 66 |
| 114 | 0.115 | 15 | 85 |
| 115 | 0.053 | 22 | 78 |
| 116 | 0.193 | 22 | 78 |
| 117 | 0.578 | 13 | 87 |
| 118 | 0.208 | 20 | 80 |
| 119 | 0.189 | 14 | 86 |
| 120 | 0.538 | 17 | 83 |
| 121 | 0.317 | 14 | 86 |
| 122 | 0.044 | 9 | 91 |
| 123 | 0.059 | 11 | 89 |
| 124 | 0.520 | 15 | 85 |
| 125 | 0.120 | 15 | 85 |
| 126 | 0.134 | 17 | 83 |
| 127 | 0.149 | 9 | 91 |
| 128 | 0.007 | 12 | 88 |
| 129 | 0.009 | 14 | 86 |
| 130 | 0.006 | 14 | 86 |

### Example 132: Small Molecule HbF Induction Assay

Cryopreserved primary human CD34⁺ hematopoietic stem and progenitor cells were obtained from AllCells, LLC. The CD34⁺ cells were isolated from the peripheral blood of healthy donors after mobilization by administration of granulocyte colony-stimulating factor. Cells were differentiated ex vivo toward the erythroid lineage using a 2-phase culture method. In the first phase, cells were cultured in StemSpan™ Serum-Free Expansion Media (SFEM) (STEMCELL Technologies Inc.) supplemented with rhSCF (50 ng/mL, Peprotech®, Inc.), rhIL-6 (50 ng/mL, Peprotech®, Inc.), rhIL-3 (50 ng/mL, Peprotech®, Inc.), and rhFlt3L (50 ng/mL, Peprotech®, Inc.), and 1× antibiotic-antimycotic (Life Technologies, Thermo Fisher Scientific) for 6 days at 37° C. with 5% CO₂. During the second phase, cells were cultured in erythroid differentiation media at 5,000 cells/mL in the presence of compound for 7 days at 37° C. with 5% CO₂. Erythroid Differentiation Media is comprised of IMDM (Life Technologies) supplemented with insulin (10 μg/mL, Sigma Aldrich), heparin (2 U/mL Sigma Aldrich), holo-transferrin (330 μg/mL, Sigma Aldrich), human serum AB (5%, Sigma Aldrich), hydrocortisone (1 μM, STEMCELL Technologies), rhSCF (100 ng/mL, Peprotech®, Inc.), rhIL-3 (5 ng/mL, Peprotech®, Inc.), rhEPO (3 U/mL, Peprotech®, Inc.), and 1× antibiotic-antimycotic. All compounds were dissolved and diluted into dimethylsulfoxide (DMSO) and were added to culture media for a final concentration of 0.3% DMSO for testing in a 7-point, 1:3 dilution series starting at 30 uM.

### Staining and Flow Cytometry

For viability analysis, samples were washed and resuspended in phosphate-buffered saline (PBS) and stained with LIVE/DEAD™ Fixable Violet Dead Cell Stain Kit (Life Technologies, L34963) for 20 minutes. Cells were then washed again with PBS and resuspended in PBS supplemented with 2% fetal bovine serum (FBS), and 2 mM EDTA to prepare for cell surface marker analysis. Cells were labeled with allophycocyanin-conjugated CD235a (1:100, BD Biosciences, 551336) and Brilliant Violet-conjugated CD71 (1:100, BD Biosciences, 563767) antibodies for 20 minutes. For analysis of cytoplasmic Fetal Hemoglobin (HbF), cells were fixed and permeabilized using the Fixation (BioLegend®, 420801) and Permeabilization Wash (BioLegend®, 421002) Buffers according to the manufacturer's protocol. During the permeabilization step, cells were stained with phycoerythrin-conjugated or FITC-conjugated HbF-specific antibody (1:10-1:25, Invitrogen™, MHFH04-4) for 30 minutes. Stained cells were washed with phosphate-buffered saline before analysis on the FACSCanto™ I1 flow cytometer or LSRFortessa™ (BD Biosciences). Data analysis was performed with FlowJo™ Software (BD Biosciences).

### Hbf Induction Activity of Compounds (Table 2)

mPB CD34+ cells were expanded for 6 days, then erythroid differentiated in the presence of compound for 7 days. Cells were fixed, stained and analyzed by flow cytometry. Table 2 shows HbF induction activity of the compounds. HbF Amax=the highest percentage of cells staining positive for HbF (% HbF+ cells) in the fitted dose-response curve. The baseline % HbF+ cells for DMSO-treated cells is approximately 30-40%.

TABLE 2

| Cmpd no. | HbF EC₅₀ (μM) | HbF Amax |
|---|---|---|
| 29 | 0.045 | 80 |
| 65 | 0.172 | 76 |

### Example 133: Cell Culture for shRNA and CRISPR Assays

HEK293T cells were maintained in DMEM high glucose complete media with sodium pyruvate, non-essential amino acids, 10% FBS, 2 mM L-glutamine, 100 U/mL pen/strep, 25 mM HEPES. Unless stated otherwise, all reagents for culturing HEK293T cells were obtained from Invitrogen™.

Mobilized peripheral blood (mPB) CD34+ cells (AIICells, LLC) were maintained in StemSpan™ serum-free expansion media (SFEM) (STEMCELL Technologies Inc.) supplemented with 50 ng/mL each of rhTPO, rhIL-6, rhFLT3L, rhSCF for 2-3 days prior to shRNA transduction or targeted ribonucleoprotein (RNP) electroporation targeting WIZ. All cytokines were obtained from Peprotech®, Inc. Cell cultures were maintained at 37° C. and 5% CO₂ in a humidified tissue culture incubator.

### Generation of shRNA Lentiviral Clones Targeting WIZ

5′-phosphorylated sense and anti-sense complementary single-stranded DNA oligos of the respective shRNA against WIZ were synthesized by Integrated DNA Technologies, Inc. (IDT). Each DNA oligonucleotide was designed with Pmel/Ascl restriction overhangs on 5′- and 3′-ends, respectively, for subsequent compatible ligation into the lentiviral vector backbone. Equimolar of each of the complementary oligonucleotides were annealed in NEB Buffer 2 (New England Biolabs® Inc.) by heating on a heating block at 98° C. for 5 minutes followed by cooling to room temperature on the bench top. Annealed double-stranded DNA oligonucleotides were ligated into pHAGE lentiviral backbone digested with Pmel/Ascl using T4 DNA ligase kit (New England Biolabs). Ligation reactions were transformed into chemically competent Stbl3 cells (Invitrogen™) according to the manufacturer's protocol. Positive clones were verified using the sequencing primer (5′-ctacattttacatgatagg-3′; SEQ ID NO: 2) and plasmids were purified by Alta Biotech LLC.

Lentivirus particles for the respective shRNA constructs were generated by co-transfection of HEK293T cells with pCMV-dR8.91 and pCMV-VSV-G expressing envelope plasmid using Lipofectamine 3000 reagent in 150 mm tissue culture dish format as per manufacturer's instructions (Invitrogen™). Lentivirus supernatant was harvested 48 hours after co-transfection, filtered through a 0.45 μm filter (Millipore) and concentrated using Amicon Ultra 15 with Ultracel-100 membrane (Millipore). Infectious units of each of the lentivirus particle was determined by flow cytometry using eGFP expression as marker of transduction after serial dilution and infection of HEK293T cells.

The shRNA sequences are as follows:

shWIZ_#1
(SEQ ID NO: 3)
5′-AGCCCACAATGCCACGGAAAT-3′;
shWIZ_#2
(SEQ ID NO: 4)
5′-GCAACATCTACACCCTCAAAT-3′;
shWIZ_#4
(SEQ ID NO: 5)
5′-TGACCGAGTGGTACGTCAATG-3′;
shWIZ_#5
(SEQ ID NO: 6)
5′-AGCGGCAGAACATCAACAAAT-3′.

Lentiviral shRNA Transduction and FACS of mPB CD34+ Cells

mPB CD34+ transduction was performed on retronectin coated non-tissue culture treated 96 well-flat bottom plates (Corning, Inc.). Briefly, plates were coated with 100 μL of RetroNectin® (1 μg/mL) (TAKARABIO, Inc.), sealed and incubated at 4° C. overnight. RetroNectin®was then removed and plates were incubated with BSA (bovine serum albumin) (1%) in PBS for 30 minutes at room temperature. Subsequently, BSA (bovine serum albumin) was aspirated and replaced with 100 μL of lentiviral concentrate and centrifuged at 2000×g for 2 hours at room temperature. Next, residual supernatant was gently pipetted out and ready for transductions of mPB CD34+ cells. Ten thousand cells were plated in 150 μL of StemSpan™ Serum-free Expansion Medium (SFEM) supplemented with 50 ng/mL each of rhTPO, rhIL-6, rhFLT3L, rhSCF to initiate transduction. Cells were cultured for 72 hours prior to assessing transduction efficiencies using eGFP expression as a marker.

eGFP-positive cells were sorted on an FACSAria™ III (BD Biosciences). Briefly, the transduced mPB CD34+ cell population was washed and re-suspended with FACS buffer containing 1× Hank's buffered saline solution, EDTA (1 mM) and FBS (2%). Sorted eGFP-positive cells were used for the erythroid differentiation assay.

### Targeting CRISPR Knockout of WIZ

Alt-R CRISPR-Cas9 crRNA and tracrRNA (5′-AGCAUAGCAAGUUAAAAUAAGGCUAGUCCGUUAUCAACUUGAAAAAGUGGCACCGAGUC GGUGCUUU-3′; SEQ ID NO: 7) were purchased from Integrated DNA Technologies, Inc.. Equimolar tracrRNA was annealed with WIZ targeting crRNA (Table 3) in Tris buffer (10 mM, pH 7.5) by heating at 95° C. for 5 minutes using a polymerase chain reaction (PCR) machine (Bio-Rad) followed by cooling to room temperature on the benchtop. Subsequently, a ribonucleoprotein (RNP) complex was generated by mixing annealed tracrRNA:crRNA with 6 μg of Cas9 at 37° C. for 5 minutes in 1× buffer containing HEPES (100 mM), KCl (50 mM), MgCl₂ (2.5 mM), glycerol (0.03%), DTT (1 mM) and Tris pH 7.5 (2 mM).

Electroporation of the RNP complex was performed on a 4D-Nucleofector™ (Lonza) as per manufacturer's recommendation. Briefly, 50,000 mPB CD34+ cells resuspended in Primary Cell P3 Buffer with supplement (Lonza) were pre-mixed with 5 μL of RNP complex per well in nucleocuvettes and incubated for 5 minutes at room temperature. Subsequently, the mixture was electroporated using the CM-137 program. Cells were cultured for 72 hours post-RNP electroporation before initiating erythroid differentiation. The crRNA sequences are shown in Table 3 below.

TABLE 3

| | | | | SEQ |
|---|---|---|---|---|
| Name | Sequence (5′ to 3′) | Target genomic region | Strand | ID NO |
| rg_0111 | ACGGAGGCTAAGCGTCGCAA | random guide, non- | | 8 |
| | | targeting | | |
| WIZ_6 | AACATCTTTCGGGCCGTAGG | chr19: 15427143- | (+) | 9 |
| | | 15427163 | | |
| WIZ_9 | GACATCCGCTGCGAGTTCTG | chr19: 15427488- | (−) | 10 |
| | | 15427510 | | |
| WIZ_12 | TGCAGCGTCCCGGGCAGAGC | chr19: 15425751- | (−) | 11 |
| | | 15425773 | | |
| WIZ_14 | CAAGCCGTGCCTCATCAAGA | chr19: 15425571- | (−) | 12 |
| | | 15425593 | | |
| WIZ_15 | CGGGCACACCTGCGGCAGTT | chr19: 15424942- | (−) | 13 |
| | | 15424964 | | |
| WIZ_18 | AGTGGGTGCGGCACTTACAG | chr19 :15423169- | (−) | 14 |
| | | 15423191 | | |

Erythroid Differentiation of shRNA Transduced or RNP Electroporated mPB CD34+ Cells

Erythroid differentiation was initiated by plating 8,000 RNP-electroporated or FACS sorted eGFP+ mPB CD34+ cells per well in 96-well tissue culture plate. Base differentiation media consists of IMDM (Iscove's Modified Dulbecco's Medium), human AB serum (5%), transferrin (330 μg/mL), Insulin (10 μg/mL) and Heparin (2 IU/mL). Differentiation media was supplemented with rhSCF (100 ng/mL), rhIL-3 (10 ng/mL), rhEPO (2.5 U/mL) and hydrocortisone (1 μM). After 4 days of differentiation, the cells were split (1:4) in fresh media to maintain optimal growth density. Cells were cultured for additional 3 days and utilized for assessment of fetal hemoglobin (HbF) expression.

### Analysis of HbF Gene Expression by RNA-Seq

Two independent, targeted CRISPR/Cas9 knockout (KO) of WIZ was done using WIZ_6 and WIZ_18 gRNAs or a non-targeting scrambled gRNA negative control in mPB CD34+ HSCs. Cells from KO and negative control were then cultured for 7 days for erythroid differentiation and used for total RNA isolation (Zymo Research, catalogue #R1053). The quality of isolated RNA was determined before sequencing using Agilent RNA 6000 Pico Kit (Agilent, catalogue #5067-1513). RNA sequencing libraries were prepared using the Illumina TruSeq Stranded mRNA Sample Prep protocol and sequenced using the Illumina NovaSeq6000 platform (Illumina). Samples were sequenced to a length of 2×76 base-pairs. For each sample, salmon version 0.8.2 (Patro et al. 2017; doi: 10.1038/nmeth.4197) was used to map sequenced fragments to annotated transcripts in the human reference genome hg38 provided by the ENSEMBL database. Per-gene expression levels were obtained by summing the counts of transcript-level counts using tximport (Soneson et al. 2015; doi: 10.12688/fl000research.7563.1). DESeq2 was used to normalize for library size and transcript length differences, and to test for differential expression between samples treated with the gRNAs targeting WIZ and the samples treated with the scrambled gRNA controls (Love et al. 2014; doi: 10.1186/s13059-014-0550-8). Data were visualized using ggplot2 (Wickham H (2016). ggplot2: Elegant Graphics for Data Analysis. Springer-Verlag New York. ISBN 978-3-319-24277-4; https://ggplot2.tidyverse.org).

### Hbf Intracellular Staining

One hundred thousand cells were aliquoted into U-bottom 96-well plate and stained for 20 min in the dark with diluted LIVE/DEAD fixable violet viability dye as per manufacturer's recommendation (Invitrogen). Cells were washed with FACS staining buffer and subsequently stained with anti-CD71-BV711 (BD Biosciences) and anti-CD235a-APC (BD Biosciences) for 20 mins in the dark. After two rounds of washes with three volumes of 1×PBS, cells were fixed and permeabilized with 1×BD Cytofix/Cytoperm (BD Biosciences) for 30 minutes at room temperature in the dark. Subsequently, cells were washed twice with three volumes of 1× Perm/wash buffer (BD Biosciences). Anti-HbF-FITC (ThermoScientific) was diluted (1:25) in 1× perm/wash buffer, added to permeabalized cells and incubated for 30 minutes at room temperature in the dark. Next, cells were washed twice with three volumes of 1× perm/wash buffer and analyzed by flow cytometry using LSR Fortessa (BD Biosciences). Data was analyzed with FlowJo software.

### Results

### WIZ KO Upregulates HBG1/2 Expression Upon Erythroid Differentiation

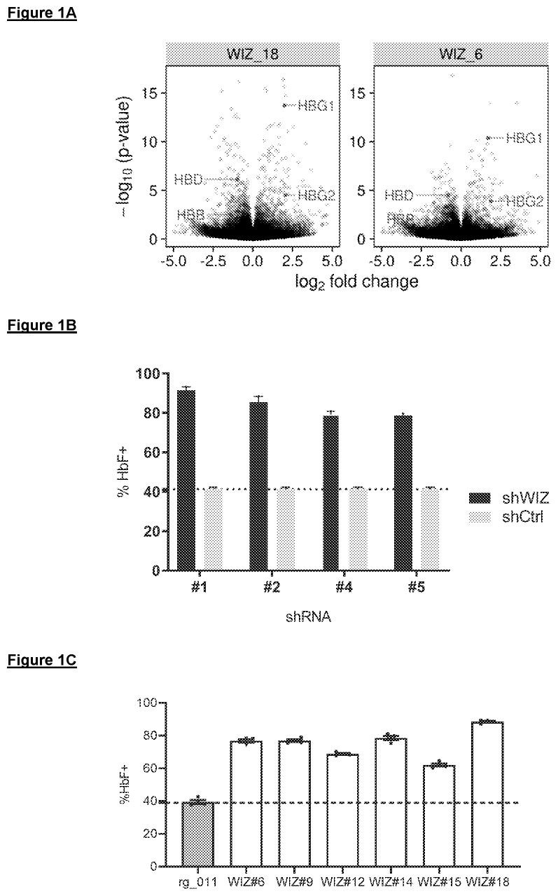

Targeted KO of WIZ using two independent gRNAs (WIZ_6 and WIZ_18) demonstrated upregulation of fetal hemoglobin genes (HBG1/2), as presented in FIG. 1A.

### Loss of WIZ Induces Fetal Hemoglobin Expression in mPB CD34⁺ Derived Erythroid Cells

In order to validate whether WIZ is a negative regulator of HbF expression, shRNA and CRISPR-Cas9-mediated knockdown and knockout functional genetics approaches were employed. mPB CD34^{′} cells were treated with shRNA or CRISPR-Cas9 reagents and erythroid differentiated for 7 days prior to flow cytometry analysis. Targeted knockdown of WIZ transcript results in 78-91% HbF+ cells compared to 40% for the negative control scrambled shRNA. Error bars represent standard error of two biological replicates with three technical replicates each (FIG. 1B). CRISPR/Cas9-mediated targeted loss of WIZ results in 62-88% HbF+ cells compared to 39% for random guide crRNA. Error bars represent standard error of one biological sample with four technical replicates (FIG. 1C). To summarize, the results indicate that loss of WIZ induces HbF in human primary erythroid cells. As such, the zinc finger transcription factor Widely Interspaced Zinc Finger Motifs (WIZ) was identified as a novel target for HbF induction. These data provide genetic evidence that WIZ is a regulator of fetal hemoglobin expression and represents a novel target for the treatment of sickle cell disease and beta-thalassemia.

Having thus described several aspects of several embodiments, it is to be appreciated various alterations, modifications, and improvements will readily occur to those skilled in the art. Such alterations, modifications, and improvements are intended to be part of this disclosure, and are intended to be within the spirit and scope of the disclosure. Accordingly, the foregoing description and drawings are by way of example only.

Those skilled in the art will recognize, or be able to ascertain, using no more than routine experimentation, numerous equivalents to the specific embodiments described specifically herein. Such equivalents are intended to be encompassed in the scope of the following claims.

1. A compound of formula (I″) or a pharmaceutically acceptable salt thereof, wherein: 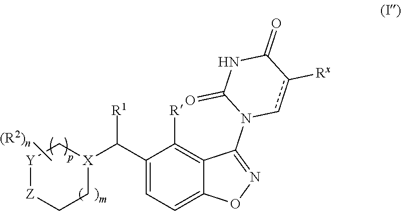 [symbol]
is a single bond or a double bond;
X is selected from CH, CF, and N;
Y is selected from CH₂, CR^{Y}R² and N—R³;
Z is selected from CH₂, CR^{Y}R² and N—R³,
wherein at least one of Y and Z is N—R³ such that when Y is N—R³, Z is selected from CH₂, and CR^{Y}R², and when Z is N—R³, Y is selected from CH₂, and CR^{Y}R²,
and wherein when R² of CR^{Y}R² of Y or Z is oxo, R^{Y} is absent;
R^{x} is selected from hydrogen, C₁-C₆alkyl, halo, C₁-C₆alkoxyl, and C₃-C₈cycloalkyl;
R^{Y} is selected from hydrogen and C₁-C₆alkyl,
R′ is selected from hydrogen and C₁-C₆alkyl;
R¹ is selected from hydrogen and C₁-C₆alkyl;
each R² is independently selected from C₁-C₆alkyl, C₁-C₆haloalkyl, halo, and oxo, wherein the C₁-C₆alkyl is substituted with 0-1 occurrence of R^{2a}; or 2 R² on non-adjacent carbon atoms together with the non-adjacent carbon atoms to which they are attached form a bridging ring;
R^{2a} is selected from C₁-C₆alkoxyl and hydroxyl;
R³ is selected from hydrogen, C₁-C₈alkyl, C₂-C₆alkenyl, —SO₂R⁴, C₁-C₆haloalkyl, a 4- to 6-membered heterocyclyl comprising 1-2 heteroatoms independently selected from N, O and S, C₃-C₁₀cycloalkyl, —C(═O)—O—(R⁵) and —C(═O)—(R⁶), wherein the C₁-C₈alkyl and C₁-C₆haloalkyl are independently substituted with 0-3 occurrences of R^{3a};
or
R³ together with the nitrogen atom to which it is attached and R² together with the carbon atom to which it is attached form a 5- or 6-membered heterocyclyl comprising 0-1 additional heteroatom selected from N, O and S;
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S, C₆-C₁₀aryl, C₁-C₆alkoxyl, hydroxyl, and —C(═O)—NR⁷R⁸, wherein the C₃-C₁₀cycloalkyl, 4- to 6-membered heterocyclyl, 5- to 10-membered heteroaryl and C₆-C₁₀aryl are substituted with 0-4 occurrences of R^{3b};
each R^{3b} is independently selected from C₁-C₆alkoxyl, halo, C₁-C₆haloalkyl, C₁-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁵ is selected from C₁-C₆alkyl, C₃-C₈cycloalkyl, and C₆-C₁₀aryl;
R⁶ is selected from C₁-C₆alkyl, C₃-C₈cycloalkyl, NR⁷R⁸, and C₆-C₁₀aryl, wherein the C₁-C₆alkyl is substituted with 0-1 occurrence of R^{6a} and the C₃-C₈cycloalkyl is substituted with 0-1 occurrence of R^{6b};
R^{6a} is selected from C₆-C₁₀aryl and C₃-C₈cycloalkyl;
R^{6b} is selected from halo, C₁-C₆haloalkyl, C₁-C₆haloalkoxyl, and C₁-C₆alkyl;
R⁷ is selected from hydrogen and C₁-C₆alkyl;
R⁸ is selected from hydrogen, C₃-C₈cycloalkyl and C₁-C₆alkyl;
or
R⁷ and R⁸ together with the nitrogen atom to which they are attached form a 5- or 6-membered heterocyclyl comprising 0-1 additional heteroatom selected from N, O, and S;
n is 0, 1, 2, 3 or 4;
m is 0, 1 or 2; and
p is 0 or 1.

2-4. (canceled)

5. The compound of claim 1, or a pharmaceutically acceptable salt thereof, having a Formula (I′), wherein: 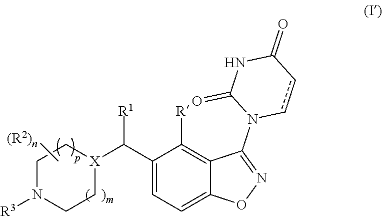 [symbol]
is a single bond or a double bond;
X is selected from CH, CF, and N;
R′ is selected from hydrogen and C₁-C₆alkyl;
R¹ is selected from hydrogen and C₁-C₆alkyl;
each R² is independently selected from C₁-C₆alkyl, C₁-C₆haloalkyl, halo, and oxo, wherein the C₁-C₆alkyl is substituted with 0-1 occurrence of R^{2a}; or 2 R² on non-adjacent carbon atoms together with the non-adjacent carbon atoms to which they are attached form a bridging ring;
R^{2a} is selected from C₁-C₆alkoxyl and hydroxyl;
R³ is selected from hydrogen, C₁-C₈alkyl, C₂-C₆alkenyl, —SO₂R⁴, C₁-C₆haloalkyl, —C(═O)—O—(R⁵) and —C(═O)—(R⁶), wherein the C₁-C₈alkyl and C₁-C₆haloalkyl are independently substituted with 0-3 occurrences of R^{3a};
or
R³ together with the nitrogen atom to which it is attached and R² together with the carbon atom to which it is attached form a 5- or 6-membered heterocyclyl comprising 0-1 additional heteroatom selected from N, O and S;
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S, C₆-C₁₀aryl, C₁-C₆alkoxyl, hydroxyl, and —C(═O)—NR⁷R⁸, wherein the C₃-C₁₀cycloalkyl, 4- to 6-membered heterocyclyl, 5- to 10-membered heteroaryl and C₆-C₁₀aryl are substituted with 0-4 occurrences of R^{3b};
each R^{3b} is independently selected from C₁-C₆alkoxyl, halo, C₁-C₆haloalkyl, C₁-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁵ is selected from C₁-C₆alkyl, C₃-C₈cycloalkyl, and C₆-C₁₀aryl;
R⁶ is selected from C₁-C₆alkyl, C₃-C₈cycloalkyl, and C₆-C₁₀aryl, wherein the C₁-C₆alkyl is substituted with 0-1 occurrence of R^{6a} and the C₃-C₈cycloalkyl is substituted with 0-1 occurrence of R^{6b};
R^{6a} is selected from C₆-C₁₀aryl and C₃-C₈cycloalkyl;
R^{6b} is selected from halo, C₁-C₆haloalkyl, C₁-C₆haloalkoxyl, and C₁-C₆alkyl;
R⁷ is selected from hydrogen and C₁-C₆alkyl;
R⁸ is selected from hydrogen and C₁-C₆alkyl;
or
R⁷ and R⁸ together with the nitrogen atom to which they are attached form a 5- or 6-membered heterocyclyl comprising 0-1 additional heteroatom selected from N, O, and S;
n is 0, 1, 2, 3 or 4;
m is 0, 1 or 2; and
p is 0 or 1.

6. The compound of claim 1, or a pharmaceutically acceptable salt thereof, having a Formula (I), wherein: 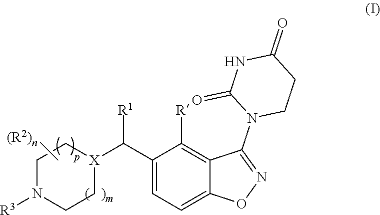
X is selected from CH, CF, and N;
R′ is selected from hydrogen and C₁-C₆alkyl;
R¹ is selected from hydrogen and C₁-C₆alkyl;
each R² is independently selected from C₁-C₆alkyl, C₁-C₆haloalkyl, halo, and oxo, wherein the C₁-C₆alkyl is substituted with 0-1 occurrence of R^{2a}; or 2 R² on non-adjacent carbon atoms together with the non-adjacent carbon atoms to which they are attached form a bridging ring;
R^{2a} is selected from C₁-C₆alkoxyl and hydroxyl;
R³ is selected from hydrogen, C₁-C₈alkyl, C₂-C₆alkenyl, —SO₂R⁴, C₁-C₆haloalkyl, —C(═O)—O—(R⁵) and —C(═O)—(R⁶), wherein the C₁-C₈alkyl and C₁-C₆haloalkyl are independently substituted with 0-3 occurrences of R^{3a};
or
R³ together with the nitrogen atom to which it is attached and R² together with the carbon atom to which it is attached form a 5- or 6-membered heterocyclyl comprising 0-1 additional heteroatom selected from N, O and S;
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S, C₆-C₁₀aryl, C₁-C₆alkoxyl, hydroxyl, and —C(═O)—NR⁷R⁸, wherein the C₃-C₁₀cycloalkyl, 4- to 6-membered heterocyclyl, 5- to 10-membered heteroaryl and C₆-C₁₀aryl are substituted with 0-4 occurrences of R^{3b};
each R^{3b} is independently selected from C₁-C₆alkoxyl, halo, C₁-C₆haloalkyl, C₁-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁵ is selected from C₁-C₆alkyl, C₃-C₈cycloalkyl, and C₆-C₁₀aryl;
R⁶ is selected from C₁-C₆alkyl, C₃-C₈cycloalkyl, and C₆-C₁₀aryl, wherein the C₁-C₆alkyl is substituted with 0-1 occurrence of R^{6a} and the C₃-C₈cycloalkyl is substituted with 0-1 occurrence of R^{6b};
R^{6a} is selected from C₆-C₁₀aryl and C₃-C₈cycloalkyl;
R^{6b} is selected from halo, C₁-C₆haloalkyl, C₁-C₆haloalkoxyl, and C₁-C₆alkyl;
R⁷ is selected from hydrogen and C₁-C₆alkyl;
R⁸ is selected from hydrogen and C₁-C₆alkyl;
or
R⁷ and R⁸ together with the nitrogen atom to which they are attached form a 5- or 6-membered heterocyclyl comprising 0-1 additional heteroatom selected from N, O, and S;
n is 0, 1, 2, 3 or 4;
m is 0, 1 or 2; and
p is 0 or 1.

7. The compound of claim 1, or a pharmaceutically acceptable salt thereof, wherein
X is selected from CH and N;
R′ is hydrogen;
R¹ is selected from hydrogen and C₁-C₃alkyl;
each R² is independently selected from unsubstituted C₁-C₆alkyl, C₁-C₆haloalkyl and halo; or 2 R² on non-adjacent carbon atoms together with the non-adjacent carbon atoms to which they are attached form a bridging ring;
R³ is selected from hydrogen, C₁-C₈alkyl, C₂-C₆alkenyl, —SO₂R⁴, C₁-C₆haloalkyl and —C(═O)—(R⁶), wherein the C₁-C₈alkyl and C₁-C₆haloalkyl are independently substituted with 0-3 occurrences of R^{3a};
or
R³ together with the nitrogen atom to which it is attached and R² together with the carbon atom to which it is attached form a 5- or 6-membered heterocyclyl comprising 0-1 additional heteroatom selected from N and 0;
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S, C₆-C₁₀aryl, C₁-C₆alkoxyl, hydroxyl, and —C(═O)—NR⁷R⁸, wherein the C₃-C₁₀cycloalkyl, 4- to 6-membered heterocyclyl, 5- to 10-membered heteroaryl and C₆-C₁₀aryl are substituted with 0-4 occurrences of R^{3b};
each R^{3b} is independently selected from C₁-C₆alkoxyl, halo, C₁-C₆haloalkyl, C₁-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁶ is selected from C₁-C₆alkyl, C₃-C₈cycloalkyl, and C₆-C₁₀aryl, wherein the C₁-C₆alkyl is substituted with 0-1 occurrence of R^{6a} and the C₃-C₈cycloalkyl is substituted with 0-1 occurrence of R^{6b};
R^{6a} is selected from C₆-C₁₀aryl and C₃-C₈cycloalkyl;
R^{6b} is selected from chloro, fluoro, C₁-C₆haloalkyl, C₁-C₆haloalkoxyl, and C₁-C₆alkyl;
R⁷ is selected from hydrogen and C₁-C₆alkyl;
R⁸ is selected from hydrogen and C₁-C₆alkyl;
or
R⁷ and R⁸ together with the nitrogen atom to which they are attached form a 5- or 6-membered heterocyclyl comprising 0-1 additional heteroatom selected from N, O, and S;
n is 0, 1, 2 or 3;
m is 0, 1 or 2; and
p is 0 or 1.

8. The compound of claim 1, or a pharmaceutically acceptable salt thereof, wherein
X is selected from CH and N;
R′ is hydrogen;
R¹ is hydrogen;
each R² is independently selected from unsubstituted C₁-C₆alkyl and halo; or 2 R² on non-adjacent carbon atoms together with the non-adjacent carbon atoms to which they are attached form a C₁-C₂alkylene bridging ring;
R³ is selected from C₁-C₈alkyl, —SO₂R⁴ C₁-C₆haloalkyl and —C(═O)—(R⁶), wherein the C₁-C₈alkyl and C₁-C₆haloalkyl are independently substituted with 0-3 occurrences of R^{3a};
or
R³ together with the nitrogen atom to which it is attached and R² together with the carbon atom to which it is attached form a 5- or 6-membered heterocyclyl comprising 0-1 additional O heteroatom;
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S and phenyl, wherein the C₃-C₁₀cycloalkyl, 4- to 6-membered heterocyclyl, 5- to 10-membered heteroaryl and phenyl are substituted with 0-4 occurrences of R^{3b};
each R^{3b} is independently selected from C₁-C₆alkoxyl, halo, C₁-C₆haloalkyl, C₁-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁶ is selected from C₁-C₆alkyl, C₃-C₈cycloalkyl, and C₆-C₁₀aryl;
R⁷ is selected from hydrogen and C₁-C₆alkyl;
R⁸ is selected from hydrogen and C₁-C₆alkyl;
or
R⁷ and R⁸ together with the nitrogen atom to which they are attached form a 5- or 6-membered heterocyclyl comprising 0-1 additional heteroatom selected from N, O, and S;
n is 0, 1, 2 or 3;
m is 0, 1 or 2; and
p is 0 or 1.

9. The compound of claim 1, or a pharmaceutically acceptable salt thereof, wherein
X is selected from CH and N;
R′ is hydrogen;
R¹ is hydrogen;
each R² is independently selected from unsubstituted C₁-C₆alkyl and fluoro;
R³ is selected from C₁-C₈alkyl, —SO₂R⁴ and C₁-C₆haloalkyl, wherein the C₁-C₈alkyl is substituted with 0-2 occurrences of R^{3a} and the C₁-C₆haloalkyl is substituted with 0-1 occurrence of R^{3a};
each R^{3a} is independently selected from C₃-C₈cycloalkyl, a 4- to 6-membered heterocyclyl comprising 1-2 heteroatoms independently selected from N and O, a 5- to 6-membered heteroaryl comprising 1-3 heteroatoms independently selected from N, O, and S, and phenyl, wherein the C₃-C₈cycloalkyl, 4- to 6-membered heterocyclyl, 5- to 6-membered heteroaryl and phenyl are substituted with 0-3 occurrences of R^{3b};
each R^{3b} is independently selected from halo, C₁-C₆haloalkyl, C₁-C₆haloalkoxyl, and C₁-C₆alkyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
n is 0, 1 or 2;
m is 1 or 2; and
p is 0 or 1.

10. The compound of claim 1, or a pharmaceutically acceptable salt thereof, wherein
X is selected from CH and N;
R′ is hydrogen;
R¹ is hydrogen;
each R² is independently C₁-C₆alkyl;
R³ is selected from C₁-C₈alkyl, —SO₂R⁴ and unsubstituted C₁-C₆haloalkyl, wherein the C₁-C₈alkyl is substituted with 0-2 occurrences of R^{3a};
each R^{3a} is independently selected from C₃-C₆cycloalkyl, a 4- to 6-membered heterocyclyl comprising 1 O heteroatom, a 5- to 6-membered heteroaryl comprising 1-3 heteroatoms independently selected from N, O, and S, and phenyl, wherein the C₃-C₆cycloalkyl, 4- to 6-membered heterocyclyl, 5- to 6-membered heteroaryl and phenyl are substituted with 0-2 occurrences of R^{3b};
each R^{3b} is independently selected from halo, C₁-C₆haloalkyl, C₁-C₆haloalkoxyl, and C₁-C₆alkyl;
R⁴ is selected from C₃-C₈cycloalkyl, C₁-C₆alkyl, and phenyl;
n is 0, 1 or 2;
m is 1 or 2; and
p is 1.

11. The compound of claim 1, or a pharmaceutically acceptable salt thereof, wherein
X is selected from CH and N;
R′ is hydrogen;
R¹ is hydrogen;
each R² is independently selected from unsubstituted C₁-C₃alkyl;
R³ is selected from C₁-C₆alkyl, —SO₂R⁴ and unsubstituted C₁-C₆haloalkyl, wherein the C₁-C₆alkyl is substituted with 0-2 occurrences of R^{3a};
each R^{3a} is independently selected from C₃-C₆cycloalkyl, a 4- to 6-membered heterocyclyl comprising 1 O heteroatom, a 6-membered heteroaryl comprising 1-2 N heteroatoms, and phenyl, wherein the C₃-C₆cycloalkyl, 4- to 6-membered heterocyclyl, 6-membered heteroaryl and phenyl are substituted with 0-2 occurrences of R^{3b};
each R^{3b} is independently selected from chloro, fluoro, C₁-C₆haloalkyl, and C₁-C₆alkyl;
R⁴ is C₁-C₆alkyl;
n is 0, 1 or 2;
m is 1; and
p is 1.

12. The compound of claim 1, or a pharmaceutically acceptable salt thereof, having a Formula (Ia):

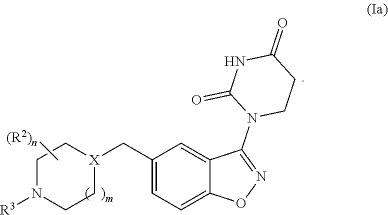

13. The compound of claim 1, or a pharmaceutically acceptable salt thereof, having a Formula (Ib):

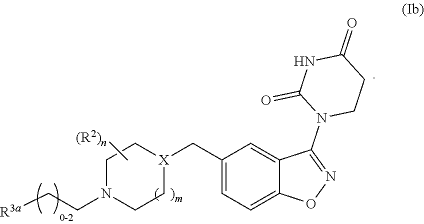

14. The compound of claim 1, or a pharmaceutically acceptable salt thereof, having a Formula (Ic), wherein: 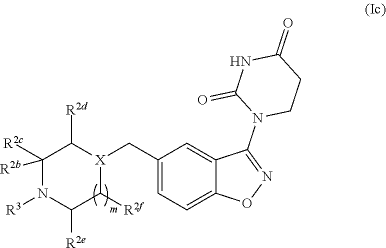
X is selected from CH, C—F and N;
R^{2b} is selected from hydrogen, C₁-C₆alkyl, C₁-C₆haloalkyl, and halo, wherein the C₁-C₆alkyl is substituted with 0-1 occurrence of R^{2a};
R^{2c} is selected from hydrogen and C₁-C₆alkyl, wherein the C₁-C₆alkyl is substituted with 0-1 occurrence of R^{2a};
or R^{2b} and R^{2c} together with the carbon atoms to which they are attached form an oxo group;
each of R^{2d} and R^{2e} is independently selected from hydrogen, C₁-C₆alkyl, C₁-C₆haloalkyl, halo, and oxo, wherein the C₁-C₆alkyl is substituted with 0-1 occurrence of R^{2a};
R^{2f} is hydrogen;
or R^{2b} and R^{2e} or R^{2b} and R^{2f} together with the carbon atoms to which they are attached form a bridging ring;
R^{2a} is selected from C₁-C₆alkoxyl and hydroxyl; and
R³ is defined according to any of the preceding claims.

15. The compound of claim 14 or a pharmaceutically acceptable salt thereof, wherein
X is selected from CH and N;
R^{2b} is selected from hydrogen, C₁-C₃alkyl, C₁-C₃haloalkyl, and halo;
R^{2c} is selected from hydrogen and C₁-C₃alkyl;
each of R^{2d} and R^{2e} is independently selected from hydrogen, C₁-C₃alkyl, C₁-C₃haloalkyl, and halo;
R^{2f} is hydrogen;
or R^{2b} and R^{2e} or R^{2b} and R^{f} together with the carbon atoms to which they are attached form a C₁-C₂alkylene bridging ring;
R³ is selected from C₁-C₈alkyl, C₂-C₆alkenyl, —SO₂R⁴, C₁-C₆haloalkyl, and —C(═O)—(R⁶), wherein the C₁-C₈alkyl and C₁-C₆haloalkyl are independently substituted with 0-3 occurrences of R^{3a};
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S, C₆-C₁₀aryl, C₁-C₆alkoxyl, hydroxyl, and —C(═O)—NR⁷R⁸, wherein the C₃-C₁₀cycloalkyl, 4- to 6-membered heterocyclyl, 5- to 10-membered heteroaryl and C₆-C₁₀aryl are substituted with 0-4 occurrences of R^{3b};
each R^{3b} is independently selected from C₁-C₆alkoxyl, halo, C₁-C₆haloalkyl, C₁-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁶ is selected from C₁-C₆alkyl, C₃-C₈cycloalkyl, and C₆-C₁₀aryl, wherein the C₁-C₆alkyl is substituted with 0-1 occurrence of R^{6a} and the C₃-C₈cycloalkyl is substituted with 0-1 occurrence of R^{6b};
R^{6a} is selected from C₆-C₁₀aryl and C₃-C₈cycloalkyl;
R^{6b} is selected from halo, C₁-C₆haloalkyl, C₁-C₆haloalkoxyl, and C₁-C₆alkyl;
R⁷ is selected from hydrogen and C₁-C₆alkyl;
R⁸ is selected from hydrogen and C₁-C₆alkyl;
or
R⁷ and R⁸ together with the nitrogen atom to which they are attached form a 5- or 6-membered heterocyclyl comprising 0-1 additional heteroatom selected from N, O, and S; and
m is 1 or 2.

16. The compound of claim 14, or a pharmaceutically acceptable salt thereof, wherein
X is selected from CH and N;
each of R^{2b}, R^{2c}, R^{2d} and R^{2e} is independently selected from hydrogen and unsubstituted C₁-C₃alkyl;
R^{2f} is hydrogen;
R³ is selected from C₁-C₈alkyl, —SO₂R⁴, and C₁-C₆haloalkyl, wherein the C₁-C₈alkyl and C₁-C₆haloalkyl are independently substituted with 0-3 occurrences of R^{3a};
each R^{3a} is independently selected from C₃-C₁₀cycloalkyl, a 4- to 6-membered heterocyclyl comprising 1-2 heteroatoms independently selected from N, O and S, a 5- to 10-membered heteroaryl comprising 1-4 heteroatoms independently selected from N, O, and S, and phenyl, wherein the C₃-C₁₀cycloalkyl, 4- to 6-membered heterocyclyl, 5- to 10-membered heteroaryl and phenyl are substituted with 0-4 occurrences of R^{3b};
each R^{3b} is independently selected from C₁-C₆alkoxyl, halo, C₁-C₆haloalkyl, C₁-C₆haloalkoxyl, C₁-C₆alkyl, —CN, —SO₂NR⁷R⁸, —SO₂R⁴, and hydroxyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl, wherein the C₁-C₆alkyl is substituted with 0-1 occurrence of R^{4a};
R^{4a} is selected from C₃-C₈cycloalkyl, C₆-C₁₀aryl, and C₁-C₆alkoxyl;
R⁷ is selected from hydrogen and C₁-C₆alkyl;
R⁸ is selected from hydrogen and C₁-C₆alkyl;
or
R⁷ and R⁸ together with the nitrogen atom to which they are attached form a 5- or 6-membered heterocyclyl comprising 0-1 additional heteroatom selected from N, O, and S; and
m is 1 or 2.

17. The compound of claim 14, or a pharmaceutically acceptable salt thereof, wherein
X is selected from CH and N;
each of R^{2b}, R^{2d} and R^{2e} is independently selected from hydrogen and unsubstituted C₁-C₃alkyl;
R^{2c} is hydrogen;
R^{2f} is hydrogen;
R³ is selected from C₁-C₈alkyl, —SO₂R⁴, and C₁-C₆haloalkyl, wherein the C₁-C₈alkyl is substituted with 0-2 occurrences of R^{3a} and the C₁-C₆haloalkyl is substituted with 0-1 occurrence of R^{3a};
each R^{3a} is independently selected from C₃-C₈cycloalkyl, a 4- to 6-membered heterocyclyl comprising 1-2 heteroatoms independently selected from N, O and S, a 5- to 10-membered heteroaryl comprising 1-3 heteroatoms independently selected from N, O, and S and phenyl, wherein the C₃-C₈cycloalkyl, 4- to 6-membered heterocyclyl, 5- to 10-membered heteroaryl and phenyl are substituted with 0-3 occurrences of R^{3b};
each R^{3b} is independently selected from halo, C₁-C₆haloalkyl, and C₁-C₆alkyl;
R⁴ is selected from C₃-C₈cycloalkyl, C₁-C₆alkyl, a 4- to 6-membered heterocyclyl comprising 1-2 heteroatoms independently selected from N, O and S, and C₆-C₁₀aryl,
wherein the C₁-C₆alkyl is substituted with 0-1 occurrence of R^{4a};
R^{4a} is selected from C₃-C₈cycloalkyl, C₆-C₁₀aryl, and C₁-C₆alkoxyl; and
m is 1.

18. The compound of claim 1, or a pharmaceutically acceptable salt thereof, having a Formula (Id), 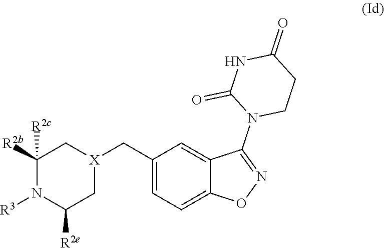
wherein, R^{2b}, R^{2c} and R^{2e} are defined according to any one of claims 14 to 17.

19. The compound of claim 1, or a pharmaceutically acceptable salt thereof, having a Formula (Ie), 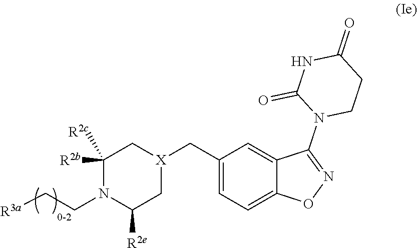
wherein, R^{2b}, R^{2c} and R^{2e} are defined according to any one of claims 14 to 17.

20-34. (canceled)

35. The compound of claim 1 or a pharmaceutically acceptable salt thereof, selected from the group consisting of: 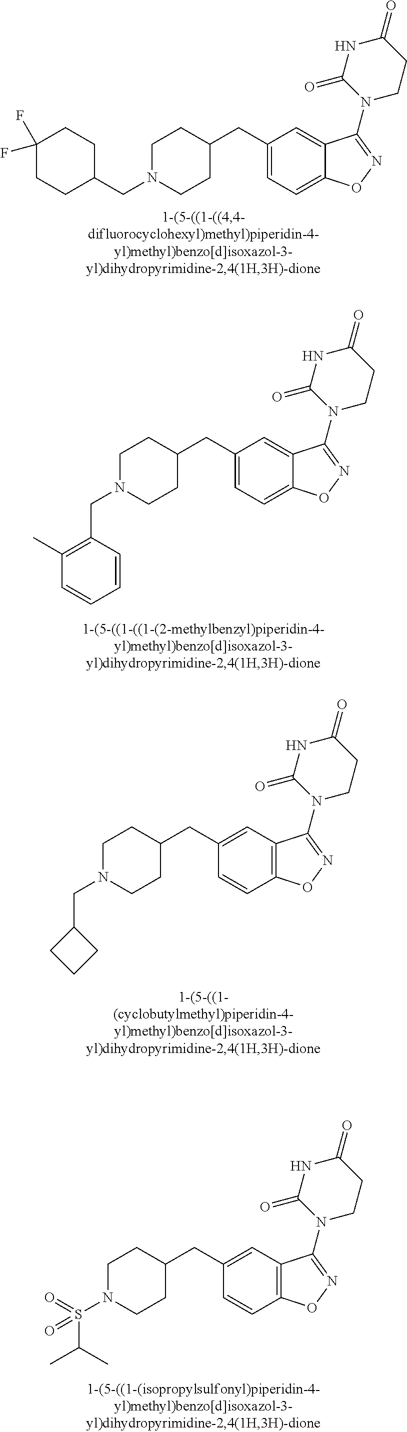 [SMILES: O=C1CCN(c2noc3ccc(CC4CCN(CC5CCC(F)(F)CC5)CC4)cc23)C(=O)N1 ; Cc1ccccc1CN1CCC(Cc2ccc3onc(N4CCC(=O)NC4=O)c3c2)CC1 ; O=C1CCN(c2noc3ccc(CC4CCN(CC5CCC5)CC4)cc23)C(=O)N1 ; CC(C)S(=O)(=O)N1CCC(Cc2ccc3onc(N4CCC(=O)NC4=O)c3c2)CC1] 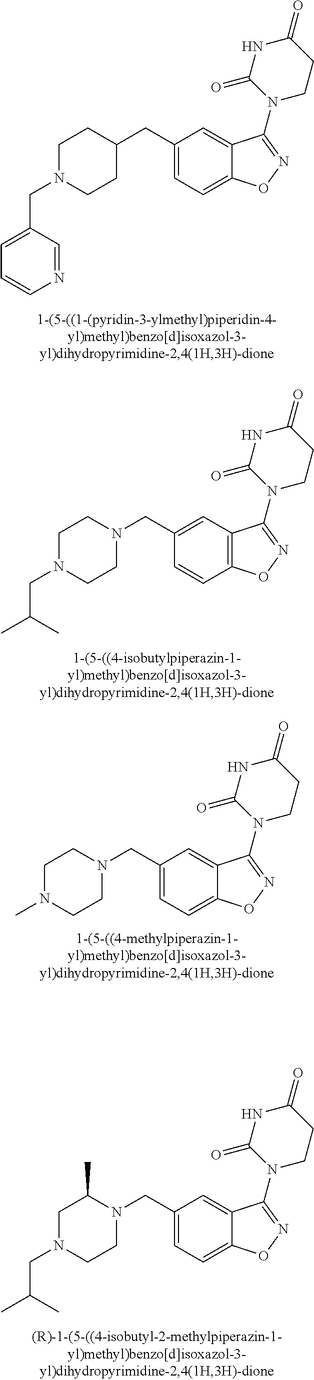 [SMILES: O=C1CCN(c2noc3ccc(CC4CCN(Cc5cccnc5)CC4)cc23)C(=O)N1 ; CC(C)CN1CCN(Cc2ccc3onc(N4CCC(=O)NC4=O)c3c2)CC1 ; CN1CCN(Cc2ccc3onc(N4CCC(=O)NC4=O)c3c2)CC1 ; CC(C)CN1CCN(Cc2ccc3onc(N4CCC(=O)NC4=O)c3c2)[C@H](C)C1] 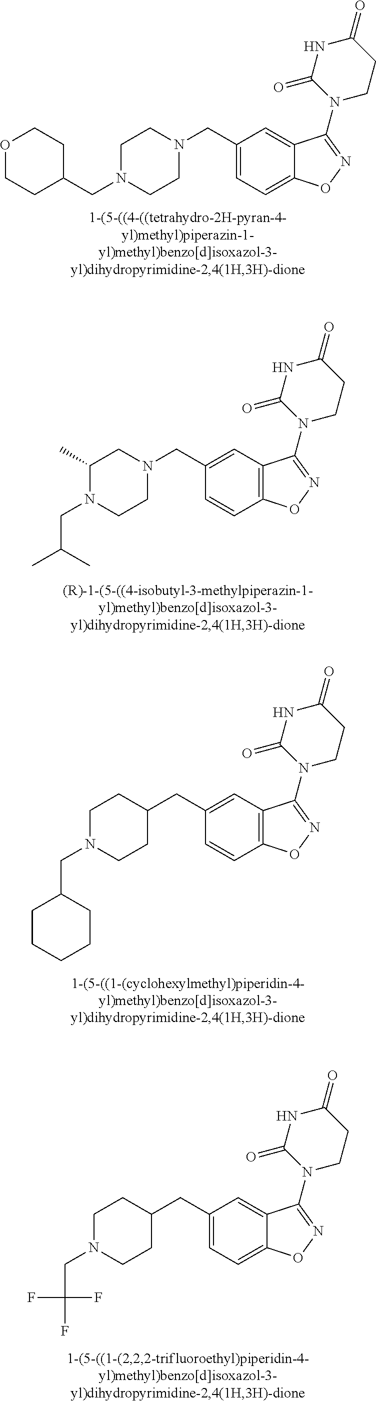 [SMILES: O=C1CCN(c2noc3ccc(CN4CCN(CC5CCOCC5)CC4)cc23)C(=O)N1 ; CC(C)CN1CCN(Cc2ccc3onc(N4CCC(=O)NC4=O)c3c2)C[C@H]1C ; O=C1CCN(c2noc3ccc(CC4CCN(CC5CCCCC5)CC4)cc23)C(=O)N1 ; O=C1CCN(c2noc3ccc(CC4CCN(CC(F)(F)F)CC4)cc23)C(=O)N1] 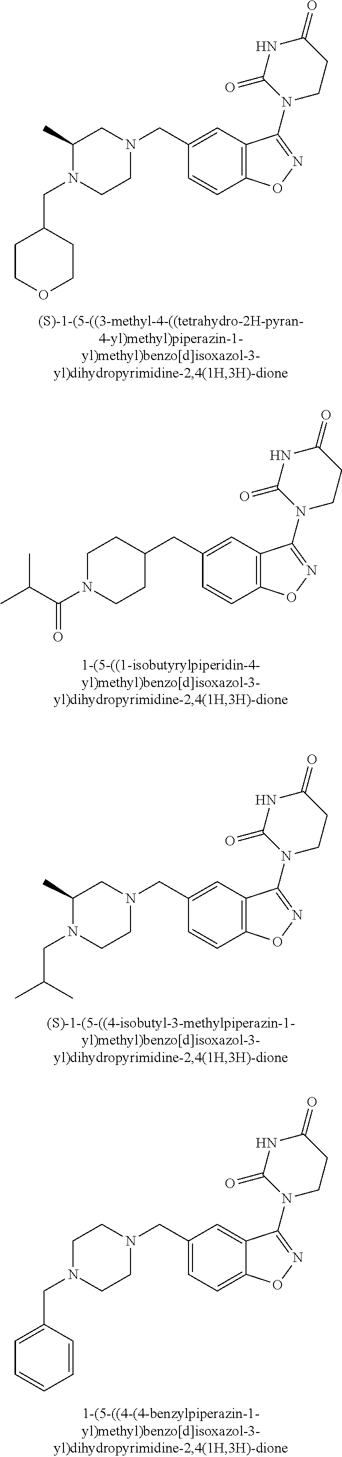 [SMILES: C[C@H]1CN(Cc2ccc3onc(N4CCC(=O)NC4=O)c3c2)CCN1CC1CCOCC1 ; CC(C)C(=O)N1CCC(Cc2ccc3onc(N4CCC(=O)NC4=O)c3c2)CC1 ; CC(C)CN1CCN(Cc2ccc3onc(N4CCC(=O)NC4=O)c3c2)C[C@@H]1C ; O=C1CCN(c2noc3ccc(CN4CCN(Cc5ccccc5)CC4)cc23)C(=O)N1] 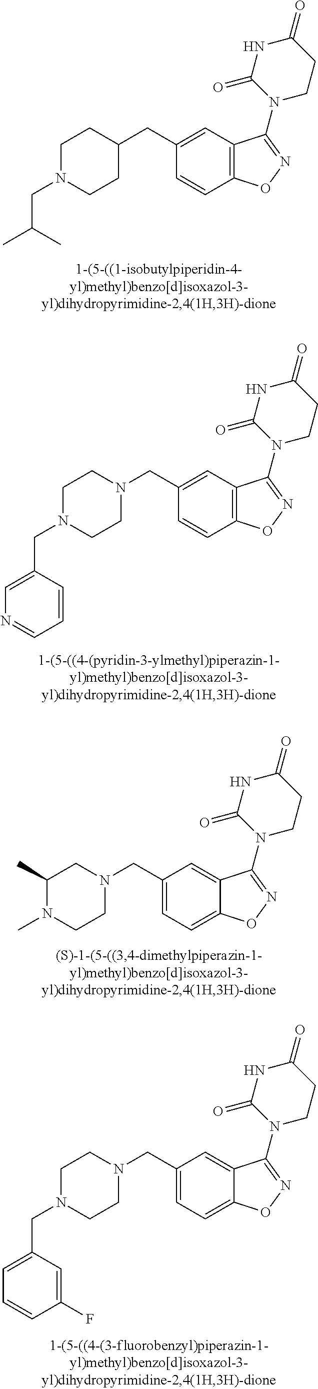 [SMILES: CC(C)CN1CCC(Cc2ccc3onc(N4CCC(=O)NC4=O)c3c2)CC1 ; O=C1CCN(c2noc3ccc(CN4CCN(Cc5cccnc5)CC4)cc23)C(=O)N1 ; C[C@H]1CN(Cc2ccc3onc(N4CCC(=O)NC4=O)c3c2)CCN1C ; O=C1CCN(c2noc3ccc(CN4CCN(Cc5cccc(F)c5)CC4)cc23)C(=O)N1] 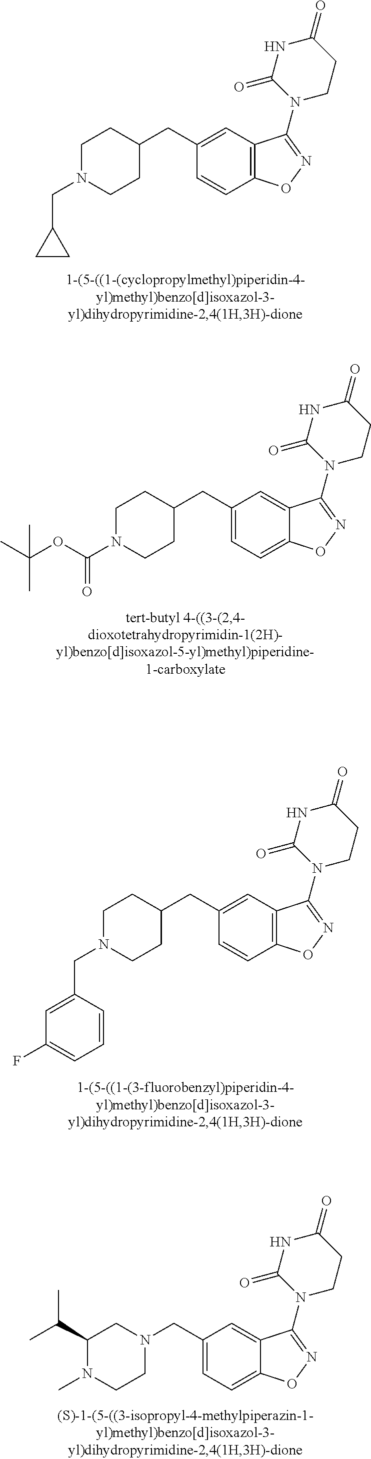 [SMILES: O=C1CCN(c2noc3ccc(CC4CCN(CC5CC5)CC4)cc23)C(=O)N1 ; CC(C)(C)OC(=O)N1CCC(Cc2ccc3onc(N4CCC(=O)NC4=O)c3c2)CC1 ; O=C1CCN(c2noc3ccc(CC4CCN(Cc5cccc(F)c5)CC4)cc23)C(=O)N1 ; CC(C)[C@H]1CN(Cc2ccc3onc(N4CCC(=O)NC4=O)c3c2)CCN1C] 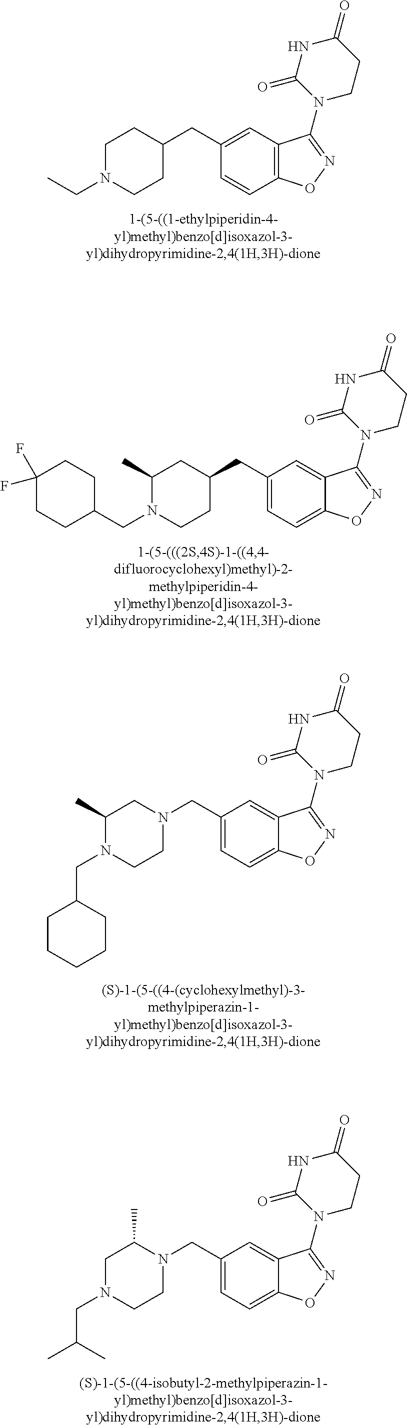 [SMILES: CCN1CCC(Cc2ccc3onc(N4CCC(=O)NC4=O)c3c2)CC1 ; C[C@H]1C[C@@H](Cc2ccc3onc(N4CCC(=O)NC4=O)c3c2)CCN1CC1CCC(F)(F)CC1 ; C[C@H]1CN(Cc2ccc3onc(N4CCC(=O)NC4=O)c3c2)CCN1CC1CCCCC1 ; CC(C)CN1CCN(Cc2ccc3onc(N4CCC(=O)NC4=O)c3c2)[C@@H](C)C1] 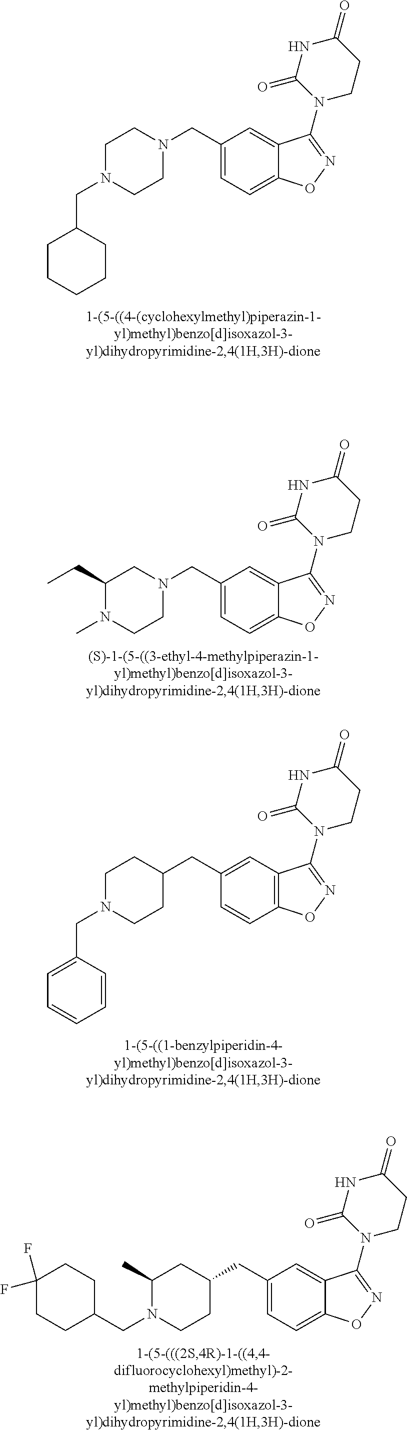 [SMILES: O=C1CCN(c2noc3ccc(CN4CCN(CC5CCCCC5)CC4)cc23)C(=O)N1 ; CC[C@H]1CN(Cc2ccc3onc(N4CCC(=O)NC4=O)c3c2)CCN1C ; O=C1CCN(c2noc3ccc(CC4CCN(Cc5ccccc5)CC4)cc23)C(=O)N1 ; C[C@H]1C[C@H](Cc2ccc3onc(N4CCC(=O)NC4=O)c3c2)CCN1CC1CCC(F)(F)CC1] 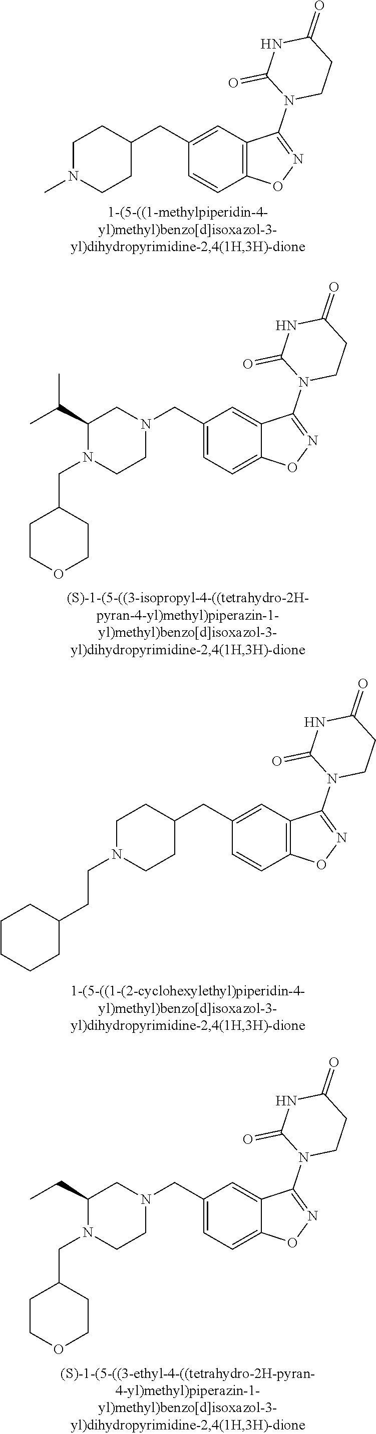 [SMILES: CN1CCC(Cc2ccc3onc(N4CCC(=O)NC4=O)c3c2)CC1 ; CC(C)[C@H]1CN(Cc2ccc3onc(N4CCC(=O)NC4=O)c3c2)CCN1CC1CCOCC1 ; O=C1CCN(c2noc3ccc(CC4CCN(CCC5CCCCC5)CC4)cc23)C(=O)N1 ; CC[C@H]1CN(Cc2ccc3onc(N4CCC(=O)NC4=O)c3c2)CCN1CC1CCOCC1] 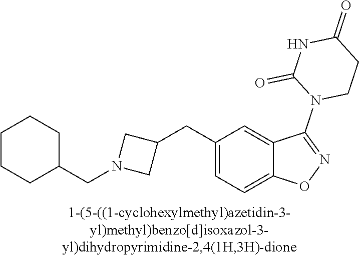 [SMILES: O=C1CCN(c2noc3ccc(CC4CN(CC5CCCCC5)C4)cc23)C(=O)N1] 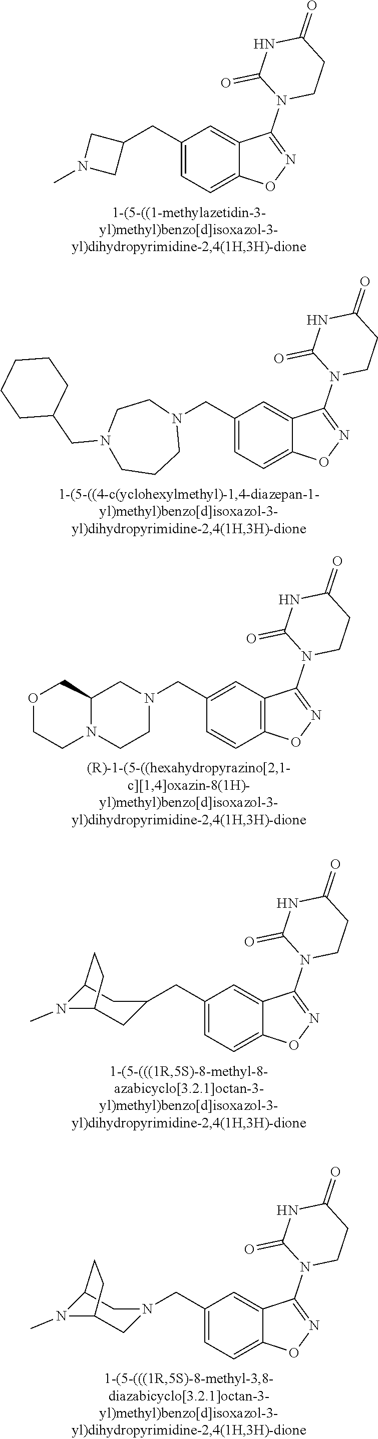 [SMILES: CN1CC(Cc2ccc3onc(N4CCC(=O)NC4=O)c3c2)C1 ; O=C1CCN(c2noc3ccc(CN4CCCN(CC5CCCCC5)CC4)cc23)C(=O)N1 ; O=C1CCN(c2noc3ccc(CN4CCN5CCOC[C@H]5C4)cc23)C(=O)N1 ; CN1C2CCC1CC(Cc1ccc3onc(N4CCC(=O)NC4=O)c3c1)C2 ; CN1C2CCC1CN(Cc1ccc3onc(N4CCC(=O)NC4=O)c3c1)C2] 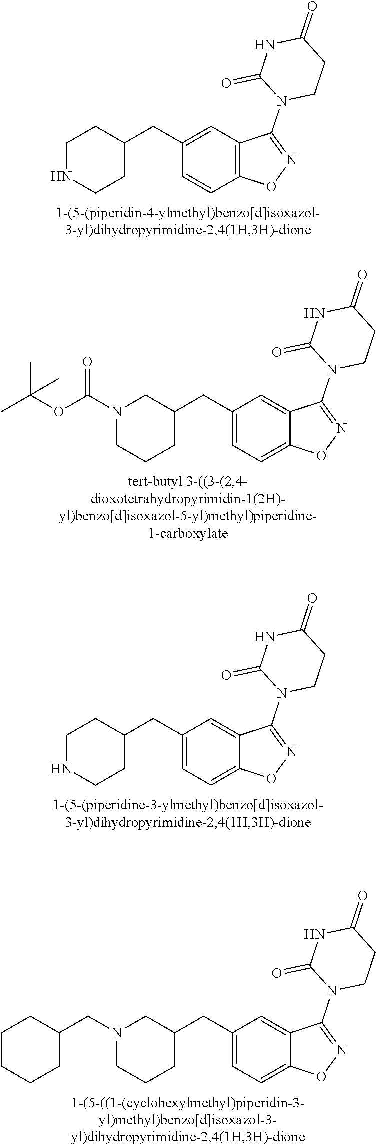 [SMILES: O=C1CCN(c2noc3ccc(CC4CCNCC4)cc23)C(=O)N1 ; CC(C)(C)OC(=O)N1CCCC(Cc2ccc3onc(N4CCC(=O)NC4=O)c3c2)C1 ; O=C1CCN(c2noc3ccc(CC4CCNCC4)cc23)C(=O)N1 ; O=C1CCN(c2noc3ccc(CC4CCCN(CC5CCCCC5)C4)cc23)C(=O)N1] 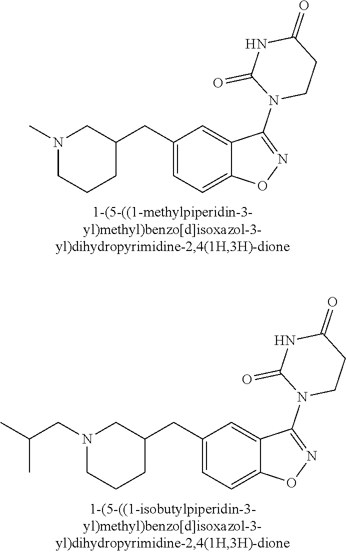 [SMILES: CN1CCCC(Cc2ccc3onc(N4CCC(=O)NC4=O)c3c2)C1 ; CC(C)CN1CCCC(Cc2ccc3onc(N4CCC(=O)NC4=O)c3c2)C1] 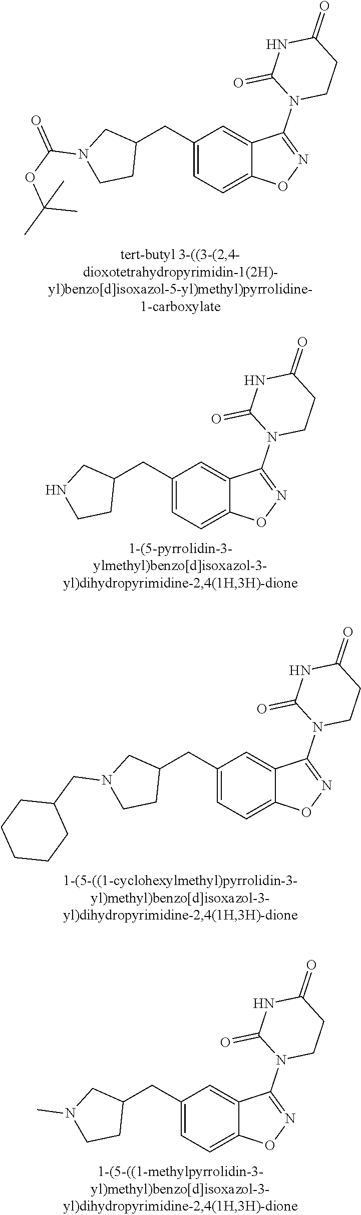 [SMILES: CC(C)(C)OC(=O)N1CCC(Cc2ccc3onc(N4CCC(=O)NC4=O)c3c2)C1 ; O=C1CCN(c2noc3ccc(CC4CCNC4)cc23)C(=O)N1 ; O=C1CCN(c2noc3ccc(CC4CCN(CC5CCCCC5)C4)cc23)C(=O)N1 ; CN1CCC(Cc2ccc3onc(N4CCC(=O)NC4=O)c3c2)C1] 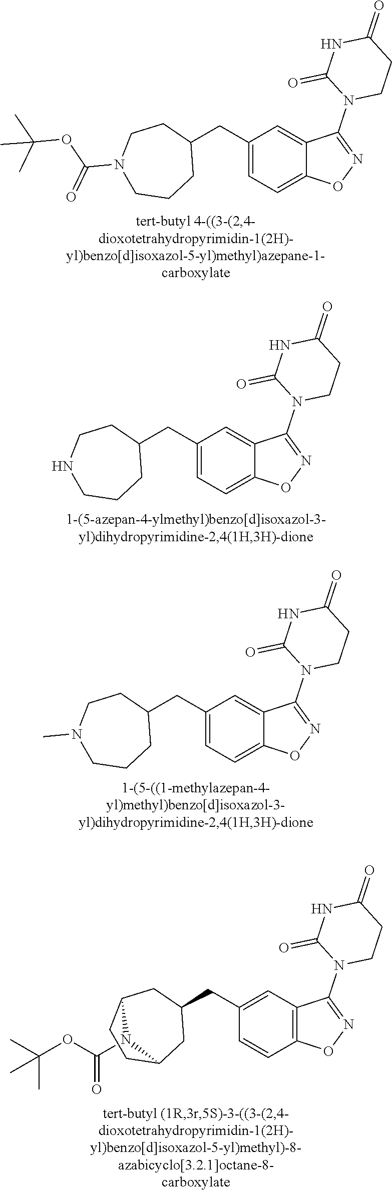 [SMILES: CC(C)(C)OC(=O)N1CCCC(Cc2ccc3onc(N4CCC(=O)NC4=O)c3c2)CC1 ; O=C1CCN(c2noc3ccc(CC4CCCNCC4)cc23)C(=O)N1 ; CN1CCCC(Cc2ccc3onc(N4CCC(=O)NC4=O)c3c2)CC1 ; CC(C)(C)OC(=O)N1[C@@H]2CC[C@H]1C[C@@H](Cc1ccc3onc(N4CCC(=O)NC4=O)c3c1)C2] 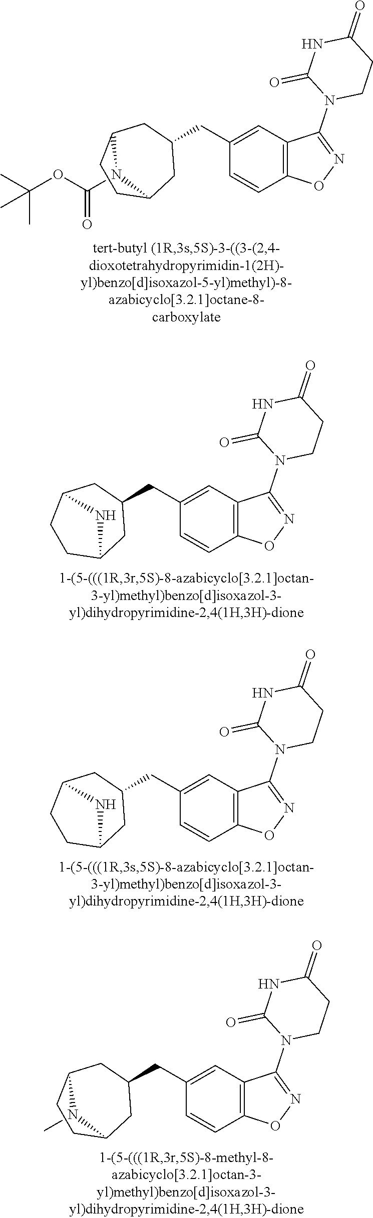 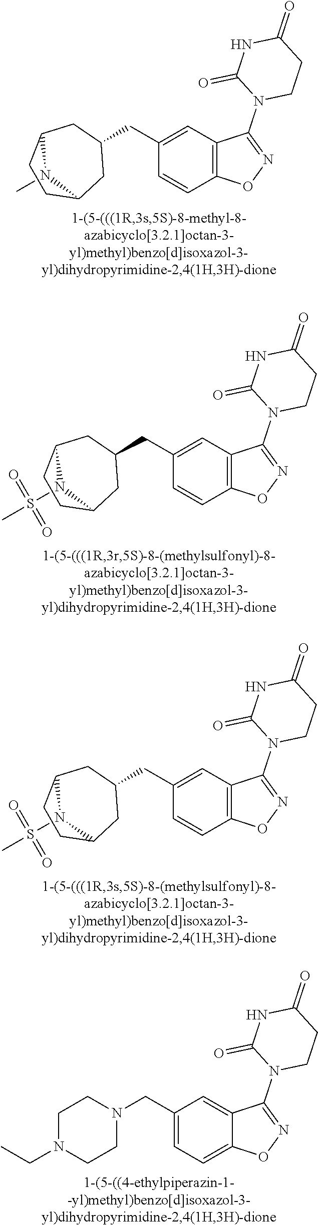 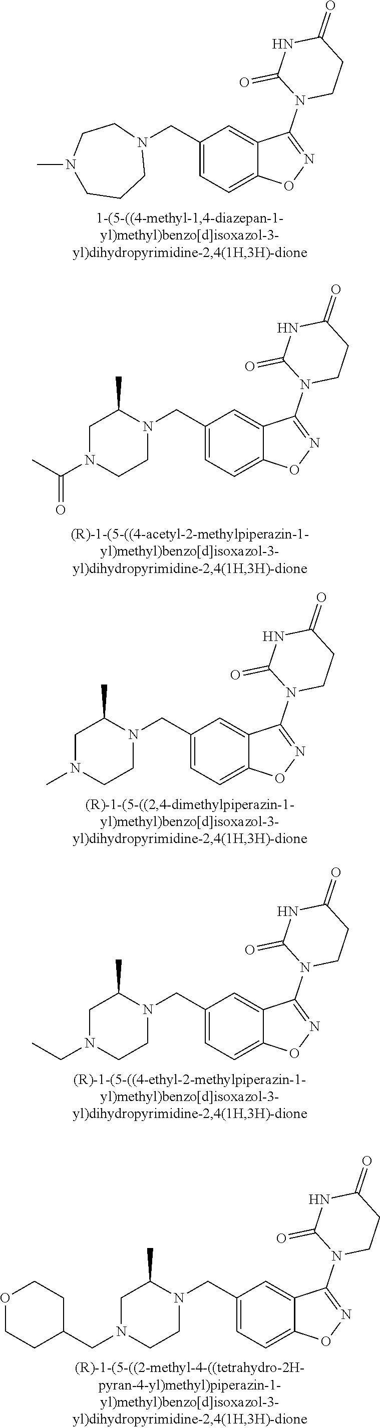 [SMILES: CN1CCCN(Cc2ccc3onc(N4CCC(=O)NC4=O)c3c2)CC1 ; CC(=O)N1CCN(Cc2ccc3onc(N4CCC(=O)NC4=O)c3c2)[C@H](C)C1 ; C[C@@H]1CN(C)CCN1Cc1ccc2onc(N3CCC(=O)NC3=O)c2c1 ; CCN1CCN(Cc2ccc3onc(N4CCC(=O)NC4=O)c3c2)[C@H](C)C1 ; C[C@@H]1CN(CC2CCOCC2)CCN1Cc1ccc2onc(N3CCC(=O)NC3=O)c2c1] 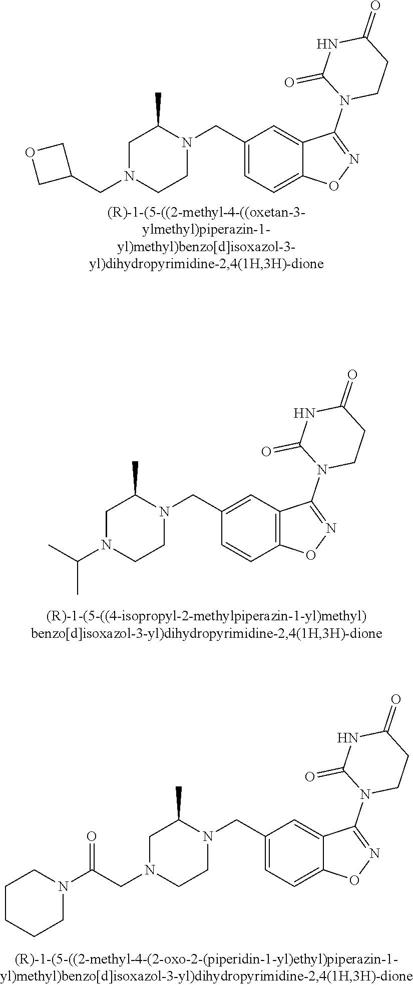 [SMILES: C[C@@H]1CN(CC2COC2)CCN1Cc1ccc2onc(N3CCC(=O)NC3=O)c2c1 ; CC(C)N1CCN(Cc2ccc3onc(N4CCC(=O)NC4=O)c3c2)[C@H](C)C1 ; C[C@@H]1CN(CC(=O)N2CCCCC2)CCN1Cc1ccc2onc(N3CCC(=O)NC3=O)c2c1] 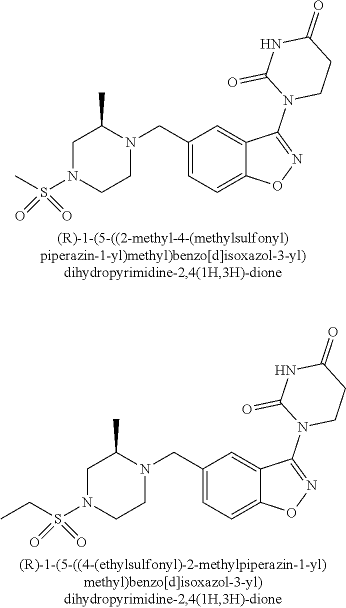 [SMILES: C[C@@H]1CN(S(C)(=O)=O)CCN1Cc1ccc2onc(N3CCC(=O)NC3=O)c2c1 ; CCS(=O)(=O)N1CCN(Cc2ccc3onc(N4CCC(=O)NC4=O)c3c2)[C@H](C)C1] 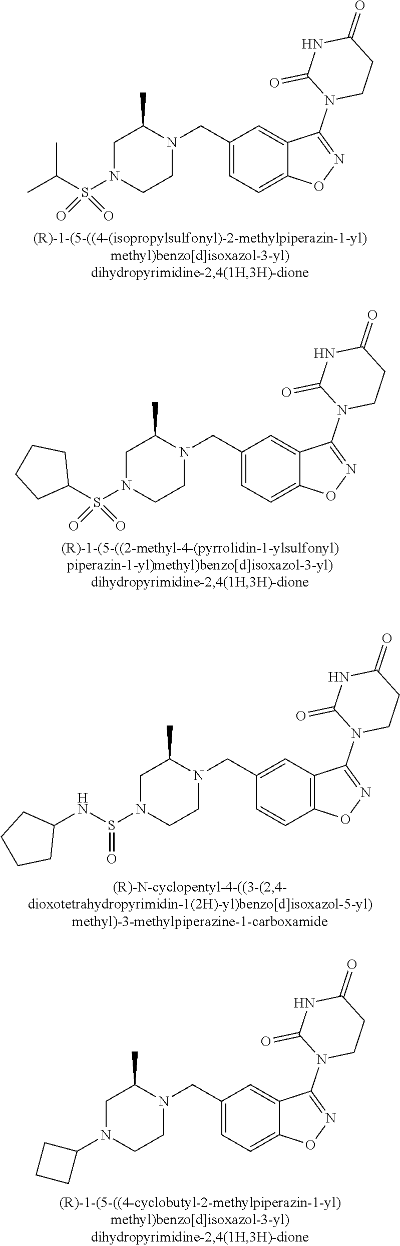 [SMILES: CC(C)S(=O)(=O)N1CCN(Cc2ccc3onc(N4CCC(=O)NC4=O)c3c2)[C@H](C)C1 ; C[C@@H]1CN(S(=O)(=O)C2CCCC2)CCN1Cc1ccc2onc(N3CCC(=O)NC3=O)c2c1 ; C[C@@H]1CN(S(=O)NC2CCCC2)CCN1Cc1ccc2onc(N3CCC(=O)NC3=O)c2c1 ; C[C@@H]1CN(C2CCC2)CCN1Cc1ccc2onc(N3CCC(=O)NC3=O)c2c1] 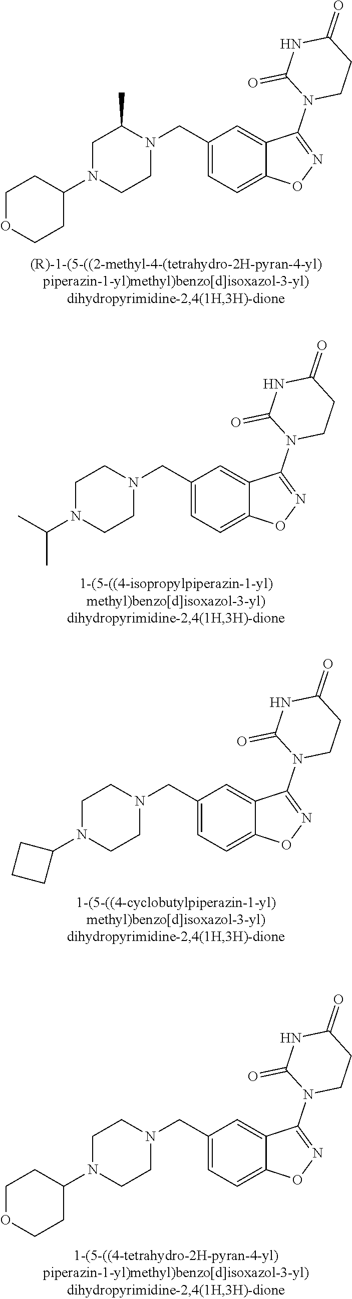 [SMILES: C[C@@H]1CN(C2CCOCC2)CCN1Cc1ccc2onc(N3CCC(=O)NC3=O)c2c1 ; CC(C)N1CCN(Cc2ccc3onc(N4CCC(=O)NC4=O)c3c2)CC1 ; O=C1CCN(c2noc3ccc(CN4CCN(C5CCC5)CC4)cc23)C(=O)N1 ; O=C1CCN(c2noc3ccc(CN4CCN(C5CCOCC5)CC4)cc23)C(=O)N1] 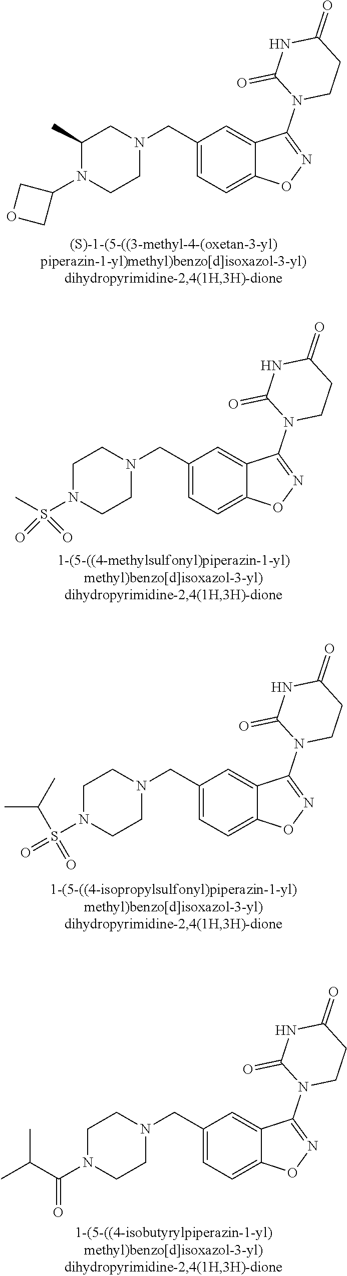 [SMILES: C[C@H]1CN(Cc2ccc3onc(N4CCC(=O)NC4=O)c3c2)CCN1C1COC1 ; CS(=O)(=O)N1CCN(Cc2ccc3onc(N4CCC(=O)NC4=O)c3c2)CC1 ; CC(C)S(=O)(=O)N1CCN(Cc2ccc3onc(N4CCC(=O)NC4=O)c3c2)CC1 ; CC(C)C(=O)N1CCN(Cc2ccc3onc(N4CCC(=O)NC4=O)c3c2)CC1] 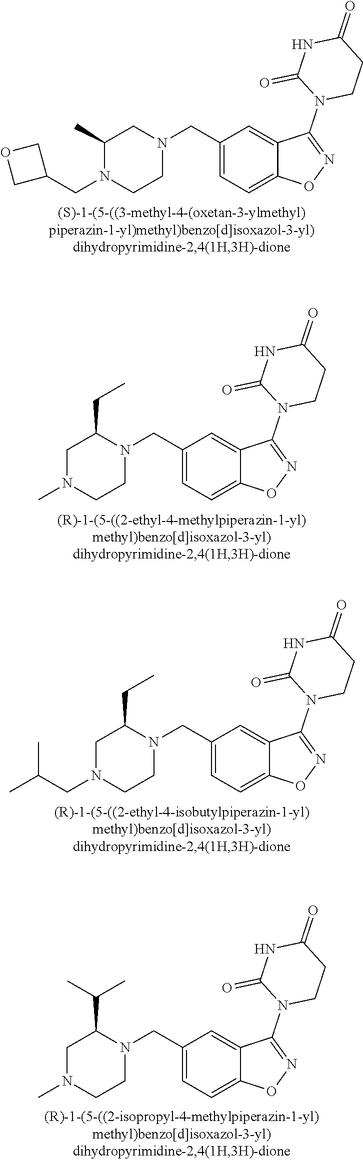 [SMILES: C[C@H]1CN(Cc2ccc3onc(N4CCC(=O)NC4=O)c3c2)CCN1CC1COC1 ; CC[C@@H]1CN(C)CCN1Cc1ccc2onc(N3CCC(=O)NC3=O)c2c1 ; CC[C@@H]1CN(CC(C)C)CCN1Cc1ccc2onc(N3CCC(=O)NC3=O)c2c1 ; CC(C)[C@@H]1CN(C)CCN1Cc1ccc2onc(N3CCC(=O)NC3=O)c2c1] 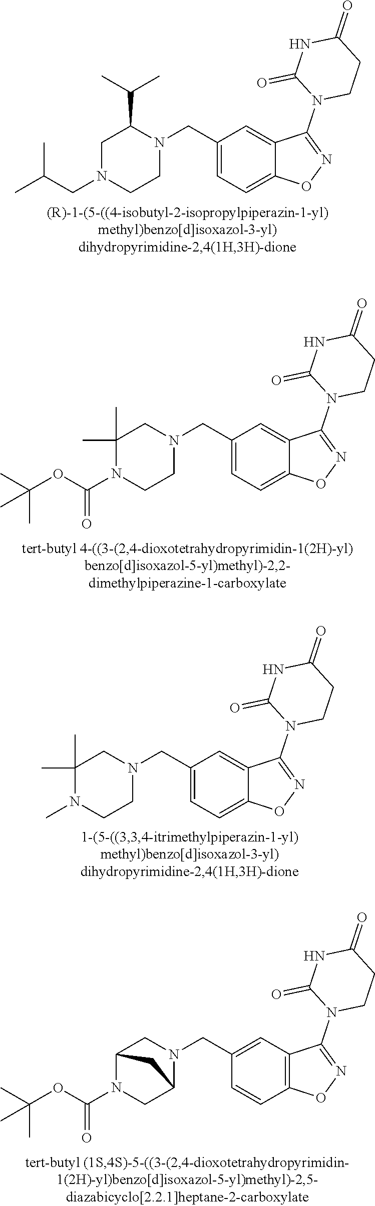 [SMILES: CC(C)CN1CCN(Cc2ccc3onc(N4CCC(=O)NC4=O)c3c2)[C@H](C(C)C)C1 ; CC(C)(C)OC(=O)N1CCN(Cc2ccc3onc(N4CCC(=O)NC4=O)c3c2)CC1(C)C ; CN1CCN(Cc2ccc3onc(N4CCC(=O)NC4=O)c3c2)CC1(C)C ; CC(C)(C)OC(=O)N1C[C@@H]2C[C@H]1CN2Cc1ccc2onc(N3CCC(=O)NC3=O)c2c1] 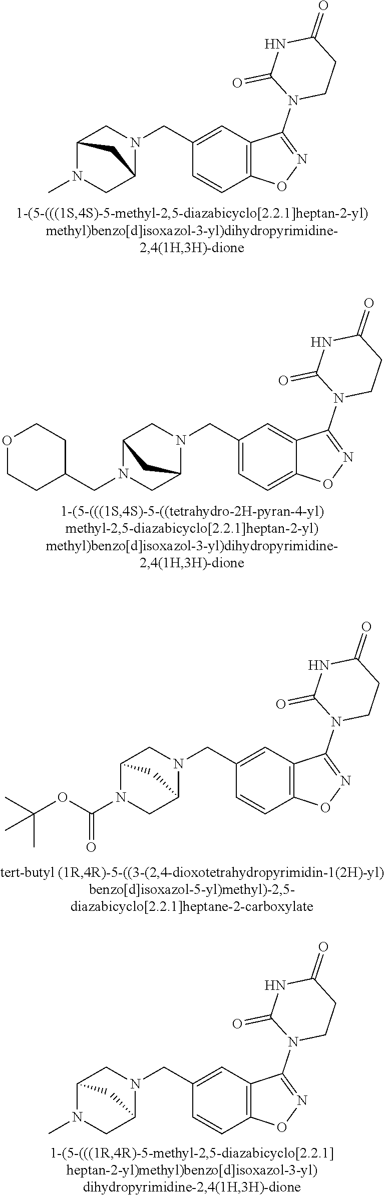 [SMILES: CN1C[C@@H]2C[C@H]1CN2Cc1ccc2onc(N3CCC(=O)NC3=O)c2c1 ; O=C1CCN(c2noc3ccc(CN4C[C@@H]5C[C@H]4CN5CC4CCOCC4)cc23)C(=O)N1 ; CC(C)(C)OC(=O)N1C[C@H]2C[C@@H]1CN2Cc1ccc2onc(N3CCC(=O)NC3=O)c2c1 ; CN1C[C@H]2C[C@@H]1CN2Cc1ccc2onc(N3CCC(=O)NC3=O)c2c1] 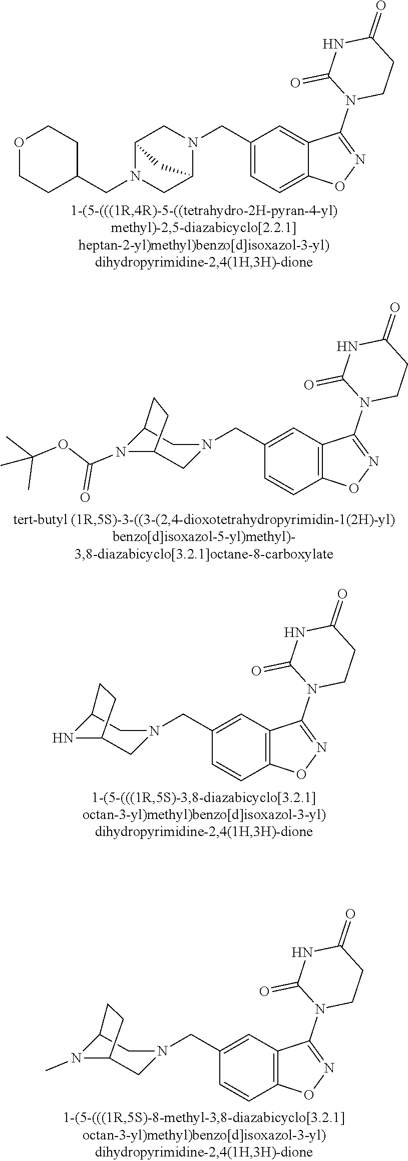 [SMILES: O=C1CCN(c2noc3ccc(CN4C[C@H]5C[C@@H]4CN5CC4CCOCC4)cc23)C(=O)N1 ; CC(C)(C)OC(=O)N1C2CCC1CN(Cc1ccc3onc(N4CCC(=O)NC4=O)c3c1)C2 ; O=C1CCN(c2noc3ccc(CN4CC5CCC(C4)N5)cc23)C(=O)N1 ; CN1C2CCC1CN(Cc1ccc3onc(N4CCC(=O)NC4=O)c3c1)C2] 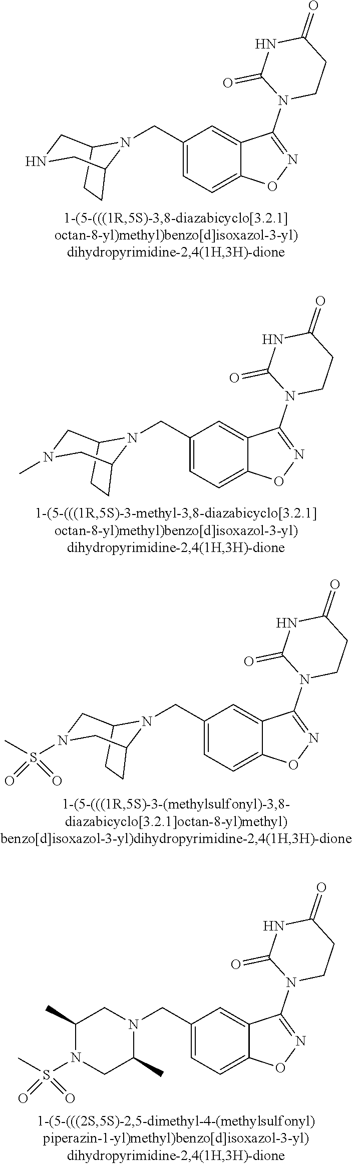 [SMILES: O=C1CCN(c2noc3ccc(CN4C5CCC4CNC5)cc23)C(=O)N1 ; CN1CC2CCC(C1)N2Cc1ccc2onc(N3CCC(=O)NC3=O)c2c1 ; CS(=O)(=O)N1CC2CCC(C1)N2Cc1ccc2onc(N3CCC(=O)NC3=O)c2c1 ; C[C@H]1CN(S(C)(=O)=O)[C@@H](C)CN1Cc1ccc2onc(N3CCC(=O)NC3=O)c2c1] 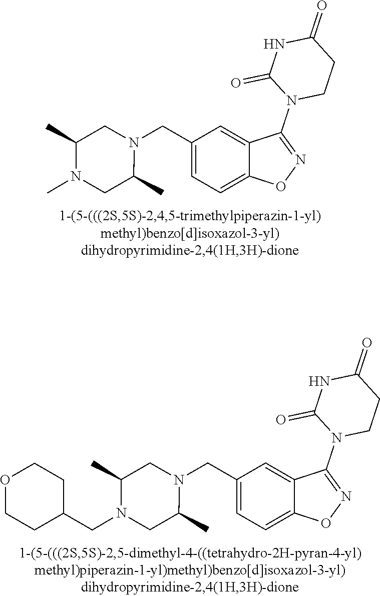 [SMILES: C[C@H]1CN(Cc2ccc3onc(N4CCC(=O)NC4=O)c3c2)[C@@H](C)CN1C ; C[C@H]1CN(CC2CCOCC2)[C@@H](C)CN1Cc1ccc2onc(N3CCC(=O)NC3=O)c2c1] 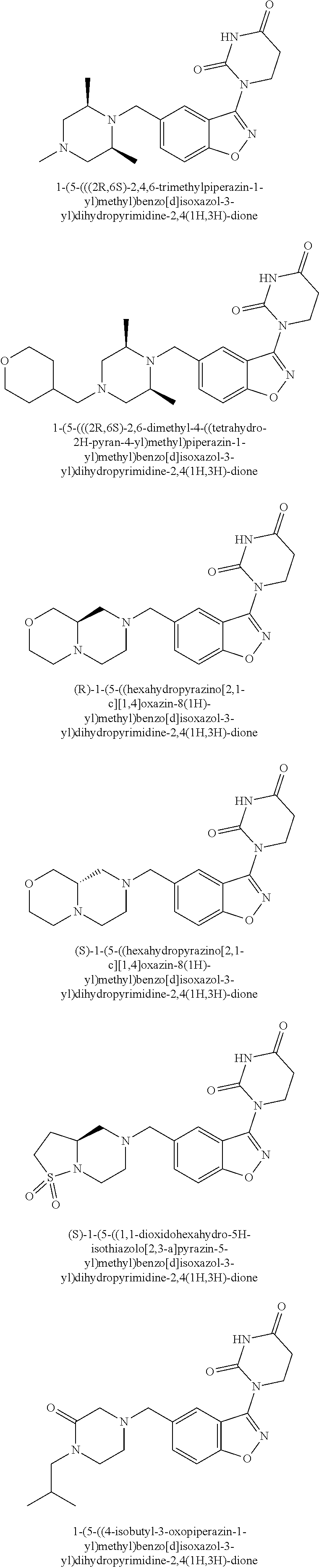 [SMILES: C[C@@H]1CN(C)C[C@H](C)N1Cc1ccc2onc(N3CCC(=O)NC3=O)c2c1 ; C[C@@H]1CN(CC2CCOCC2)C[C@H](C)N1Cc1ccc2onc(N3CCC(=O)NC3=O)c2c1 ; O=C1CCN(c2noc3ccc(CN4CCN5CCOC[C@H]5C4)cc23)C(=O)N1 ; O=C1CCN(c2noc3ccc(CN4CCN5CCOC[C@@H]5C4)cc23)C(=O)N1 ; O=C1CCN(c2noc3ccc(CN4CCN5[C@@H](CCS5(=O)=O)C4)cc23)C(=O)N1 ; CC(C)CN1CCN(Cc2ccc3onc(N4CCC(=O)NC4=O)c3c2)CC1=O] 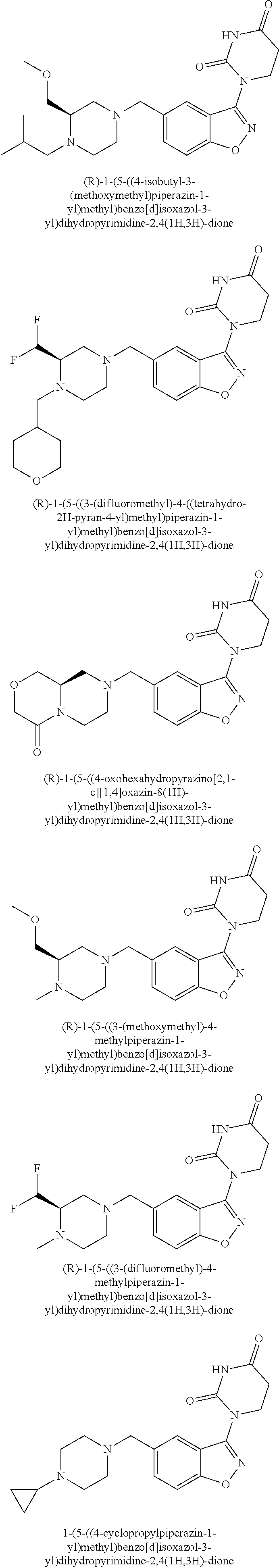 [SMILES: COC[C@H]1CN(Cc2ccc3onc(N4CCC(=O)NC4=O)c3c2)CCN1CC(C)C ; O=C1CCN(c2noc3ccc(CN4CCN(CC5CCOCC5)[C@@H](C(F)F)C4)cc23)C(=O)N1 ; O=C1CCN(c2noc3ccc(CN4CCN5C(=O)COC[C@H]5C4)cc23)C(=O)N1 ; COC[C@H]1CN(Cc2ccc3onc(N4CCC(=O)NC4=O)c3c2)CCN1C ; CN1CCN(Cc2ccc3onc(N4CCC(=O)NC4=O)c3c2)C[C@@H]1C(F)F ; O=C1CCN(c2noc3ccc(CN4CCN(C5CC5)CC4)cc23)C(=O)N1] 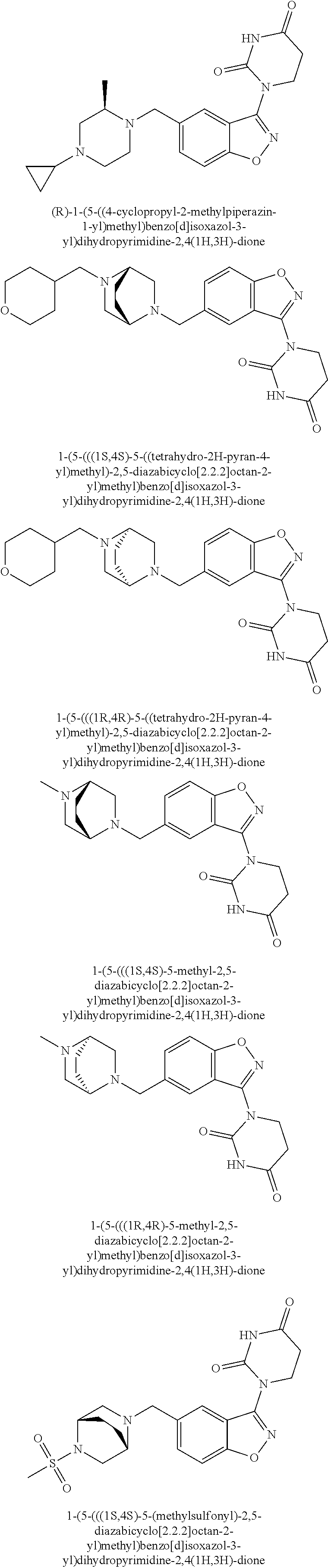 [SMILES: C[C@@H]1CN(C2CC2)CCN1Cc1ccc2onc(N3CCC(=O)NC3=O)c2c1 ; O=C1CCN(c2noc3ccc(CN4C[C@@H]5CC[C@H]4CN5CC4CCOCC4)cc23)C(=O)N1 ; O=C1CCN(c2noc3ccc(CN4C[C@H]5CC[C@@H]4CN5CC4CCOCC4)cc23)C(=O)N1 ; CN1C[C@@H]2CC[C@H]1CN2Cc1ccc2onc(N3CCC(=O)NC3=O)c2c1 ; CN1C[C@H]2CC[C@@H]1CN2Cc1ccc2onc(N3CCC(=O)NC3=O)c2c1 ; CS(=O)(=O)N1C[C@@H]2CC[C@H]1CN2Cc1ccc2onc(N3CCC(=O)NC3=O)c2c1] 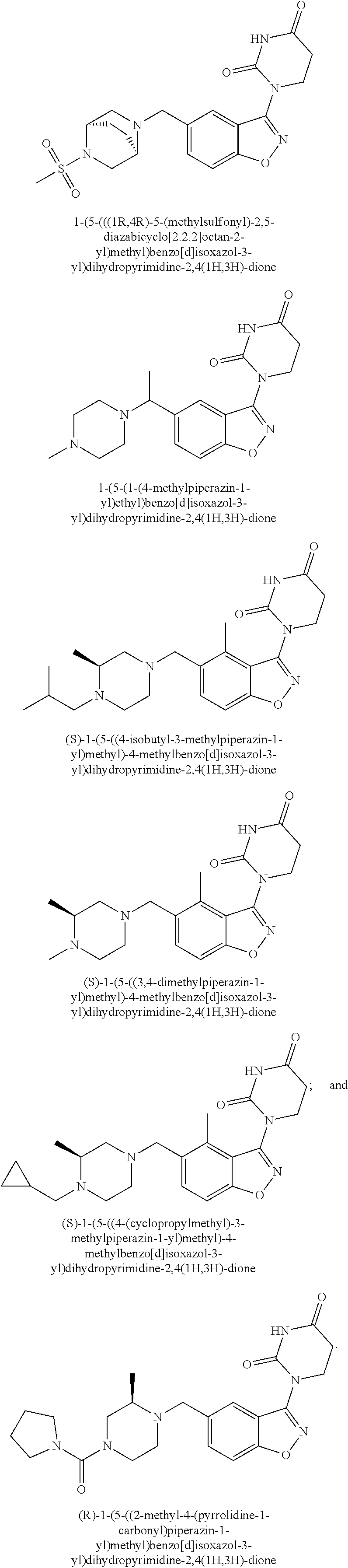 [SMILES: CS(=O)(=O)N1C[C@H]2CC[C@@H]1CN2Cc1ccc2onc(N3CCC(=O)NC3=O)c2c1 ; CC(c1ccc2onc(N3CCC(=O)NC3=O)c2c1)N1CCN(C)CC1 ; Cc1c(CN2CCN(CC(C)C)[C@@H](C)C2)ccc2onc(N3CCC(=O)NC3=O)c12 ; Cc1c(CN2CCN(C)[C@@H](C)C2)ccc2onc(N3CCC(=O)NC3=O)c12 ; Cc1c(CN2CCN(CC3CC3)[C@@H](C)C2)ccc2onc(N3CCC(=O)NC3=O)c12 ; C[C@@H]1CN(C(=O)N2CCCC2)CCN1Cc1ccc2onc(N3CCC(=O)NC3=O)c2c1]
and pharmaceutically acceptable salts thereof.

36. (canceled)

37. A pharmaceutical composition comprising a therapeutically effective amount of a compound of claim 1, or a pharmaceutically acceptable salt thereof, and a pharmaceutically acceptable carrier or excipient.

38. (canceled)

39. A method of treating or preventing a disease or disorder in a subject in need thereof, the method comprising administering to the subject a therapeutically effective amount of a compound of claim 1, or a pharmaceutically acceptable salt, thereof.

40. A method of treating or preventing a disorder that is affected by the reduction or modulation of WIZ protein levels, in a subject in need thereof, the method comprising administering to the subject a therapeutically effective amount of a compound of claim 1, or a pharmaceutically acceptable salt thereof.

41. (canceled)

42. A method of inhibiting WIZ protein expression, degrading WIZ protein, or reducing WIZ protein levels in a subject in need thereof, the method comprising administering to the subject a therapeutically effective amount of a compound of claim 1, or a pharmaceutically acceptable salt thereof.

43. (canceled)

44. A method of inhibiting, reducing, or eliminating the activity of WIZ protein or WIZ protein expression, the method comprising administering to the subject a compound of claim 1, or a pharmaceutically acceptable salt thereof.

45. A method of inducing or promoting fetal hemoglobin, reactivating fetal hemoglobin production or expression, or increasing fetal hemoglobin expression in a subject in need thereof, the method comprising administering to the subject a therapeutically effective amount of a compound of claim 1, or a pharmaceutically acceptable salt thereof.

46-47. (canceled)

48. A method of treating a hemoglobinopathy, in a subject in need thereof, the method comprising administering to the subject a therapeutically effective amount of a compound of claim 1, or a pharmaceutically acceptable salt thereof.

49. A method of treating a sickle cell disease in a subject in need thereof, the method comprising administering to the subject a therapeutically effective amount of a compound of claim 1, or a pharmaceutically acceptable salt thereof.

50. A method of treating beta-thalassemia in a subject in need thereof, the method comprising administering to the subject a therapeutically effective amount of a compound of claim 1, or a pharmaceutically acceptable salt thereof.

51-77. (canceled)

78. A pharmaceutical combination comprising a compound of claim 1, or a pharmaceutically acceptable salt thereof, and one or more additional therapeutic agent(s).

79. A compound of formula (X-1) or a salt thereof, 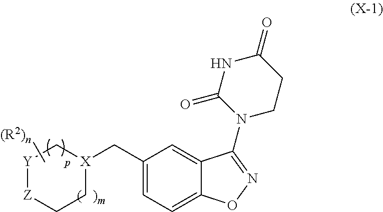
wherein:
X is selected from CH, CF, and N;
Y is selected from CH₂, CR^{Y}R² and N—R^{N};
Z is selected from CH₂, CR^{Y}R² and N—R^{N};
wherein at least one of Y and Z is N—R^{N} such that when Y is N—R^{N}, Z is selected from CH₂, and CR^{Y}R², and when Z is N—R^{N}, Y is selected from CH₂, and CR^{Y}R²,
and wherein when R² of CR^{Y}R² of Y or Z is oxo, R^{Y} is absent;
R^{Y} is selected from hydrogen and C₁-C₆alkyl;
R^{N} is selected from hydrogen and a nitrogen protecting group;
each R² is independently selected from C₁-C₆alkyl, C₁-C₆haloalkyl, halo, and oxo, wherein the C₁-C₆alkyl is substituted with 0-1 occurrence of R^{2a}; or 2 R² on non-adjacent carbon atoms together with the non-adjacent carbon atoms to which they are attached form a bridging ring;
R^{2a} is selected from C₁-C₆alkoxyl and hydroxyl;
n is 0, 1, 2, 3 or 4;
m is 0, 1 or 2; and
p is 0 or 1.
